SEQUENCE LISTING (31 sequences)

SEQ ID NO: 1 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 118 aa, synthetic construct [sequence omitted]

SEQ ID NO: 4 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 5 - protein, 10 aa, synthetic construct
  VARIANT 6: note = MISC_FEATURE - Xaa is Asp or Gly
GFTFSXSWIH

SEQ ID NO: 6 - protein, 18 aa, synthetic construct
  VARIANT 4: note = MISC_FEATURE - Xaa is Ser or Leu
  VARIANT 10: note = MISC_FEATURE - Xaa is Thr or Ser
AWIXPYGGSXYYADSVKG

SEQ ID NO: 7 - protein, 9 aa, synthetic construct
RHWPGGFDY

SEQ ID NO: 8 - protein, 25 aa, synthetic construct [sequence omitted]

SEQ ID NO: 9 - protein, 13 aa, synthetic construct
WVRQAPGKGLEWV

SEQ ID NO: 10 - protein, 32 aa, synthetic construct
RFTISADTSKNTAYLQMNSLRAEDTAVYYCAR

SEQ ID NO: 11 - protein, 11 aa, synthetic construct
WGQGTLVTVSS

SEQ ID NO: 12 - protein, 11 aa, synthetic construct
  VARIANT 9: note = MISC_FEATURE - Xaa is Ala or Phe
  VARIANT 6: note = MISC_FEATURE - Xaa is Val or Ile
  VARIANT 7: note = MISC_FEATURE - Xaa is Ser or Asn
  VARIANT 5: note = MISC_FEATURE - Xaa is Asp or Val
  VARIANT 10: note = MISC_FEATURE - Xaa is Val or Leu
RASQXXXTXXA

SEQ ID NO: 13 - protein, 7 aa, synthetic construct
  VARIANT 4: note = MISC_FEATURE - Xaa is Phe or Thr
  VARIANT 6: note = MISC_FEATURE - Xaa is Tyr or Ala
SASXLXS

SEQ ID NO: 14 - protein, 9 aa, synthetic construct
  VARIANT 8: note = MISC_FEATURE - Xaa is Ala, Trp, Arg, Pro, or Thr
  VARIANT 5: note = MISC_FEATURE - Xaa is Tyr, Asn, Ala, Thr, Gly, Phe, or Ile
  VARIANT 3: note = MISC_FEATURE - Xaa is Tyr, Gly, Phe, or Ser
  VARIANT 6: note = MISC_FEATURE - Xaa is His, Val, Pro, Thr, or Ile
  VARIANT 4: note = MISC_FEATURE - Xaa is Leu, Tyr, Phe, or Trp
QQXXXXPXT

SEQ ID NO: 15 - protein, 23 aa, synthetic construct
DIQMTQSPSSLSASVGDRVTITC

SEQ ID NO: 16 - protein, 15 aa, synthetic construct
WYQQKPGKAPKLLIY

SEQ ID NO: 17 - protein, 32 aa, synthetic construct
GVPSRFSGSGSGTDFTLTISSLQPEDFATYYC

SEQ ID NO: 18 - protein, 11 aa, synthetic construct
FGQGTKVEIKR

SEQ ID NO: 19 - protein, 10 aa, synthetic construct
GFTFSDSWIH

SEQ ID NO: 20 - protein, 18 aa, synthetic construct
AWISPYGGSTYYADSVKG

SEQ ID NO: 21 - protein, 9 aa, synthetic construct
RHWPGGFDY

SEQ ID NO: 22 - protein, 11 aa, synthetic construct
RASQDVSTAVA

SEQ ID NO: 23 - protein, 7 aa, synthetic construct
SASFLYS

SEQ ID NO: 24 - protein, 9 aa, synthetic construct
QQYLYHPAT

SEQ ID NO: 25 - protein, 122 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTK

SEQ ID NO: 26 - protein, 10 aa, synthetic construct
FGQGTKVEIK

SEQ ID NO: 27 - protein, 30 aa, synthetic construct
EVQLVESGGGLVQPGGSLRLSCAASGFTFS

SEQ ID NO: 28 - protein, 14 aa, synthetic construct
WVRQAPGKGLEWVA

SEQ ID NO: 29 - protein, 15 aa, synthetic construct
WGQGTLVTVSSASTK

SEQ ID NO: 30 - protein, 447 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0312723 A1
(43) Pub. Date: Oct. 5, 2023

(54) METHODS AND COMPOSITIONS FOR NON-SMALL CELL LUNG CANCER IMMUNOTHERAPY

(71) Applicant: Genentech, Inc., South San Francisco, CA (US)

(72) Inventors: Mark Lawrence MCCLELAND, Pacifica, CA (US); Simonetta MOCCI, Belmont, CA (US); Wei ZOU, San Carlos, CA (US); Yu DENG, South San Francisco, CA (US); Hiroshi KURIKI, South San Francisco, CA (US)

(21) Appl. No.: 18/050,236

(22) Filed: Oct. 27, 2022

Related U.S. Application Data
(63) Continuation of application No. PCT/US2021/029288, filed on Apr. 27, 2021.
(60) Provisional application No. 63/016,529, filed on Apr. 28, 2020.

(51) Int. Cl. C07K 16/28; C12Q 1/6886; G01N 33/574; A61K 31/519; A61K 33/243; A61K 31/282; A61P 35/00

(52) U.S. Cl. CPC C07K 16/2827; C12Q 1/6886; G01N 33/57492; A61K 31/519; A61K 33/243; A61K 31/282; A61P 35/00; C12Q 2600/106; C12Q 2600/156; G01N 2333/70596; C07K 2317/24

(57) ABSTRACT

The disclosure provides methods and compositions for treating non-small cell lung cancer (NSCLC; e.g., squamous or non-squamous NSCLC, including stage IV NSCLC) in a subject, for example, by administering a treatment regimen that includes a PD-1 axis binding antagonist (e.g., atezolizumab) in combination with a platinum-based chemotherapy (e.g., cisplatin or carboplatin and gemcitabine) to the subject. Exemplary subjects that may be treated using the compositions and methods of the disclosure include those that exhibits a heightened blood tumor mutational burden (bTMB) score relative to a reference bTMB score. Also provided are compositions (e.g., a PD-1 axis binding antagonist (e.g., atezolizumab) and/or a platinum-based chemotherapy (e.g., cisplatin or carboplatin and gemcitabine), pharmaceutical compositions thereof, kits thereof, and articles of manufacture thereof) for use in treating NSCLC (e.g., squamous or non-squamous NSCLC, including stage IV NSCLC) in a subject, such as a subject having an elevated bTMB score prior to the onset of treatment.

### SEQUENCE LISTING

This application contains a Sequence Listing which has been submitted electronically in XML format and is hereby incorporated by reference in its entirety. Said XML copy, created on Oct. 24, 2022, is named 50474-211003 Sequence Listing_10_25_22.XML and is 35,366 bytes in size.

### FIELD OF THE INVENTION

This disclosure relates to methods and compositions for use in treating non-small cell lung cancer (NSCLC; e.g., squamous or non-squamous NSCLC, including stage IV NSCLC) in a subject. The PD-1 axis binding antagonist may be, for example, a PD-L1 binding antagonist, such as an anti-PD-L1 antibody, such as atezolizumab.

### BACKGROUND OF THE INVENTION

Cancer remains one of the deadliest threats to human health. Cancers, or malignant tumors, metastasize and grow rapidly in an uncontrolled manner, making timely detection and treatment extremely difficult. Programmed death-ligand 1 (PD-L1) is a protein that has been implicated in the suppression of immune system responses during cancer, chronic infections, pregnancy, tissue allografts, and autoimmune diseases. PD-L1 regulates the immune response by binding to an inhibitory receptor, known as programmed death 1 (PD-1), which is expressed on the surface of T-cells, B-cells, and monocytes. PD-L1 negatively regulates T-cell function also through interaction with another receptor, B7-1. Formation of the PD-L1/PD-1 and PD-L1/B7-1 complexes negatively regulates T-cell receptor signaling, resulting in the subsequent downregulation of T-cell activation and suppression of anti-tumor immune activity.

Despite the significant advancement in the treatment of cancer (e.g., non-small cell lung cancer (NSCLC; e.g., squamous and non-squamous NSCLC, including stage IV NSCLC), improved therapies are still being sought.

### SUMMARY OF THE INVENTION

The present disclosure relates to, inter alia, methods of treating non-small cell lung cancer (NSCLC; e.g., squamous and non-squamous NSCLC, including stage IV NSCLC) in a subject, as well as compositions (e.g., a PD-1 axis binding antagonist, or a pharmaceutical composition thereof) for use in treating NSCLC (e.g., squamous and non-squamous NSCLC, including stage IV NSCLC) in a subject. Also provided are related kits and articles of manufacture.

In a first aspect, the disclosure features a method of identifying a subject having squamous NSCLC who may benefit from a treatment comprising a PD-1 axis binding antagonist. The method may include, for example, determining a blood tumor mutational burden (bTMB) score from a sample from the subject, wherein a bTMB score from the sample that is at or above a reference bTMB score identifies the subject as one who may benefit from a treatment comprising a PD-1 axis binding antagonist.

In another aspect, the disclosure features a method of selecting a therapy for a subject having squamous NSCLC. The method may include, for example, determining a bTMB score from a sample from the subject, wherein a bTMB score from the sample that is at or above a reference bTMB score identifies the subject as one who may benefit from a treatment comprising a PD-1 axis binding antagonist.

In some embodiments, the bTMB score determined from the sample is at or above the reference bTMB score. In such instances, the method may further include administering to the subject an effective amount of a PD-1 axis binding antagonist. In some embodiments, the bTMB score determined from the sample is below the reference bTMB score. In such instances, the method may include administering to the subject an effective amount of a therapy that does not contain a PD-1 axis binding antagonist.

In another aspect, the disclosure features a method of treating squamous NSCLC in a subject in need thereof. The method may include:
(a) determining a bTMB score from a sample from the subject, wherein the bTMB score determined from the sample is at or above a reference bTMB score; and
(b) administering to the subject an effective amount of a PD-1 axis binding antagonist.

In another aspect, the disclosure features a method of treating squamous NSCLC in a subject in need thereof by administering to the subject an effective amount of a PD-1 axis binding antagonist, wherein, prior to administration of the PD-1 axis binding antagonist to the subject, a bTMB score from a sample from the subject has been determined to be at or above a reference bTMB score.

In some embodiments of any of the foregoing aspects of the disclosure, the reference bTMB score is a bTMB score in a reference population. The reference population may be, for example, a population of subjects having squamous NSCLC. The population of subjects having squamous NSCLC may include a first subset of subjects who have been treated with a PD-1 axis binding antagonist and a second subset of subjects who have been treated with therapy that does not comprise a PD-1 axis binding antagonist. In such instances, the reference bTMB score may significantly separate each of the first and second subsets of subjects based on a significant difference between a subject's responsiveness to treatment with the PD-1 axis binding antagonist and a subject's responsiveness to treatment with the therapy that does not comprise a PD-1 axis binding antagonist at or above the reference bTMB score, wherein the subject's responsiveness to treatment with the PD-1 axis binding antagonist is significantly improved relative to the subject's responsiveness to treatment with the therapy that does not comprise a PD-1 axis binding antagonist. In some embodiments, the reference bTMB score significantly separates each of the first and second subsets of subjects based on a significant difference between a subject's responsiveness to treatment with the PD-1 axis binding antagonist and a subject's responsiveness to treatment with the therapy that does not comprise a PD-1 axis binding antagonist below the bTMB score, wherein the subject's responsiveness to treatment with the therapy that does not comprise a PD-1 axis binding antagonist is significantly improved relative to the subject's responsiveness to treatment with the PD-1 axis binding antagonist. In some embodiments, the therapy that does not comprise a PD-1 axis binding antagonist comprises an anti-neoplastic agent, a chemotherapeutic agent, a growth inhibitory agent, an anti-angiogenic agent, a radiation therapy, a cytotoxic agent, or a combination thereof. For example, in some embodiments, the therapy that does not comprise a PD-1 axis binding antagonist comprises a chemotherapeutic agent. In the context of any of the foregoing embodiments, the responsiveness to treatment may include an increase in progression-free survival (PFS) and/or an increase in overall survival (OS).

In some embodiments, the bTMB score from the sample has a prevalence of greater than, or equal to, about 5% in the reference population. In some embodiments, the bTMB score from the sample has a prevalence of from about 5% to about 75% in the reference population. In some embodiments, the bTMB score from the sample has a prevalence of from about 20% to about 30% in the reference population.

In some embodiments of any of the above aspects or embodiments of the disclosure, the reference bTMB score is a pre-assigned bTMB score. For example, the reference bTMB score may be from 4 to 30 (e.g., a reference bTMB score of 4, 5, 6, 7, 8, 9, 10, 11, 12, 13, 14, 15, 16, 17, 18, 19, 20, 21, 22, 23, 24, 25, 26, 27, 28, 29, or 30). In some embodiments, the reference bTMB score is from 5 to 58 (e.g., a reference bTMB score of 5, 6, 7, 8, 9, 10, 11, 12, 13, 14, 15, 16, 17, 18, 19, 20, 21, 22, 23, 24, 25, 26, 27, or 28). In some embodiments, the reference bTMB score is from 6 to 26 (e.g., a reference bTMB score of 6, 7, 8, 9, 10, 11, 12, 13, 14, 15, 16, 17, 18, 19, 20, 21, 22, 23, 24, 25, or 26). In some embodiments, the reference bTMB score is from 7 to 24 (e.g., a reference bTMB score of 7, 8, 9, 10, 11, 12, 13, 14, 15, 16, 17, 18, 19, 20, 21, 22, 23, or 24). In some embodiments, the reference bTMB score is from 8 to 22 (e.g., a reference bTMB score of 8, 9, 10, 11, 12, 13, 14, 15, 16, 17, 18, 19, 20, 21, or 22).

For example, in some embodiments of any of the above aspects or embodiments of the disclosure, the reference bTMB score is from 7 to 13 (e.g., a reference bTMB score of 7, 8, 9, 10, 11, 12, or 13). In some embodiments, the reference bTMB score is from 8 to 12 (e.g., a reference bTMB score of 8, 9, 10, 11, or 12). In some embodiments, the reference bTMB score is from 9 to 11 (e.g., a reference bTMB score of 9, 10, or 11). In some embodiments, the reference bTMB score is 10.

In some embodiments of any of the above aspects or embodiments of the disclosure, the reference bTMB score is from 13 to 19 (e.g., a reference bTMB score of 13, 14, 15, 16, 17, 18, or 19). In some embodiments, the reference bTMB score is from 14 to 18 (e.g., a reference bTMB score of 14, 15, 16, 17, or 18). In some embodiments, the reference bTMB score is from 5 to 17 (e.g., a reference bTMB score of 15, 16, or 17). In some embodiments, the reference bTMB score is 16.

In some embodiments of any of the above aspects or embodiments of the disclosure, the reference bTMB score is from 16 to 24 (e.g., a reference bTMB score of 16, 17, 18, 19, 20, 21, 22, 23, or 24). In some embodiments, the reference bTMB score is from 17 to 23 (e.g., a reference bTMB score of 17, 18, 19, 20, 21, 22, or 23). In some embodiments, the reference bTMB score is from 18 to 22 (e.g., a reference bTMB score of 18, 19, 20, 21, or 22). In some embodiments, the reference bTMB score is from 19 to 21 (e.g., a reference bTMB score of 19, 20, or 21). In some embodiments, the reference bTMB score is 20.

In some embodiments of any of the above aspects or embodiments of the disclosure, the bTMB score determined from the sample is greater than, or equal to, 4. For example, the reference bTMB score may be 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, or more.

For example, in some embodiments, the reference bTMB score is from 4 to 100 (e.g., a reference bTMB score of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100).

In some embodiments, the reference bTMB score is greater than, or equal to, 6. For example, the reference bTMB score may be 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, or more.

In some embodiments, the reference bTMB score is from 6 to 100 (e.g., a reference bTMB score of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100).

In some embodiments, the reference bTMB score is greater than, or equal to, 8. For example, the reference bTMB score may be 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, or more.

In some embodiments, the reference bTMB score is from 8 to 100 (e.g., a reference bTMB score of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100).

In some embodiments, the reference bTMB score is greater than, or equal to, 10. For example, the reference bTMB score may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, or more.

In some embodiments, the reference bTMB score is from 10 to 100 (e.g., a reference bTMB score of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100).

In some embodiments, the reference bTMB score is greater than, or equal to, 12. For example, the reference bTMB score may be 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, or more.

In some embodiments, the reference bTMB score is from 12 to 100 (e.g., a reference bTMB score of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100).

In some embodiments, the reference bTMB score is greater than, or equal to, 14. For example, the reference bTMB score may be 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, or more.

In some embodiments, the reference bTMB score is from 14 to 100 (e.g., a reference bTMB score of 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100).

In some embodiments, the reference bTMB score is greater than, or equal to, 16. For example, the reference bTMB score may be 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, or more.

In some embodiments, the reference bTMB score is from 16 to 100 (e.g., a reference bTMB score of 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100).

In some embodiments, the reference bTMB score is greater than, or equal to, 18. For example, the reference bTMB score may be 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, or more.

In some embodiments, the reference bTMB score is from 18 to 100 (e.g., a reference bTMB score of 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100).

In some embodiments, the reference bTMB score is greater than, or equal to, 20. For example, the reference bTMB score may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, or more.

In some embodiments, the reference bTMB score is from 20 to 100 (e.g., a reference bTMB score of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100). In some embodiments of any of the foregoing aspects or embodiments of the disclosure, the bTMB score determined from the sample is represented as the number of somatic mutations counted over a defined number of sequenced bases, as assessed, for example, using the FOUNDATIONONE CDX™ panel or the FOUNDATIONONE® panel. For example, in some embodiments, the defined number of sequenced bases used in the calculation of the bTMB score determined from the sample is from about 100 kb to about 10 Mb (e.g., the defined number of sequenced bases may be 100 kb, 150 kb, 200 kb, 250 kb, 300 kb, 350 kb, 400 kb, 450 kb, 500 kb, 550 kb, 600 kb, 650 kb, 700 kb, 750 kb, 800 kb, 850 kb, 900 kb, 950 kb, 1.0 Mb, 1.2 Mb, 1.3 Mb, 1.4 Mb, 1.5 Mb, 1.6 Mb, 1.7 Mb, 1.8 Mb, 1.9 Mb, 2.0 Mb, 2.1 Mb, 2.2 Mb, 2.3 Mb, 2.4 Mb, 2.5 Mb, 2.6 Mb, 2.7 Mb, 2.8 Mb, 2.9 Mb, 3.0 Mb, 3.1 Mb, 3.2 Mb, 3.3 Mb, 3.4 Mb, 3.5 Mb, 3.6 Mb, 3.7 Mb, 3.8 Mb, 3.9 Mb, 4.0 Mb, 4.1 Mb, 4.2 Mb, 4.3 Mb, 4.4 Mb, 4.5 Mb, 4.6 Mb, 4.7 Mb, 4.8 Mb, 4.9 Mb, 5.0 Mb, 5.1 Mb, 5.2 Mb, 5.3 Mb, 5.4 Mb, 5.5 Mb, 5.6 Mb, 5.7 Mb, 5.8 Mb, 5.9 Mb, 6.0 Mb, 6.1 Mb, 6.2 Mb, 6.3 Mb, 6.4 Mb, 6.5 Mb, 6.6 Mb, 6.7 Mb, 6.8 Mb, 6.9 Mb, 7.0 Mb, 7.1 Mb, 7.2 Mb, 7.3 Mb, 7.4 Mb, 7.5 Mb, 7.6 Mb, 7.7 Mb, 7.8 Mb, 7.9 Mb, 8.0 Mb, 8.1 Mb, 8.2 Mb, 8.3 Mb, 8.4 Mb, 8.5 Mb, 8.6 Mb, 8.7 Mb, 8.8 Mb, 8.9 Mb, 9.0 Mb, 9.1 Mb, 9.2 Mb, 9.3 Mb, 9.4 Mb, 9.5 Mb, 9.6 Mb, 9.7 Mb, 9.8 Mb, 9.9 Mb, or 10.0 Mb). In some embodiments, the defined number of sequenced bases used in the calculation of the bTMB score determined from the sample is from about 0.5 Mb to about 1.5 Mb (e.g., the defined number of sequenced bases may be 500 kb, 550 kb, 600 kb, 650 kb, 700 kb, 750 kb, 800 kb, 850 kb, 900 kb, 950 kb, 1.0 Mb, 1.2 Mb, 1.3 Mb, 1.4 Mb, or 1.5 Mb). In some embodiments, the defined number of sequenced bases used in the calculation of the bTMB score determined from the sample is from about 0.7 Mb to about 1.3 Mb (e.g., the defined number of sequenced bases may be 700 kb, 750 kb, 800 kb, 850 kb, 900 kb, 950 kb, 1.0 Mb, 1.2 Mb, or 1.3 Mb). In some embodiments, the defined number of sequenced bases used in the calculation of the bTMB score determined from the sample is from about 0.8 Mb to about 1.2 Mb (e.g., the defined number of sequenced bases may be 800 kb, 850 kb, 900 kb, 950 kb, 1.0 Mb, or 1.2 Mb). In some embodiments, the defined number of sequenced bases used in the calculation of the bTMB score determined from the sample is about 1.1 Mb. The number of somatic mutations used in the calculation of the bTMB score determined from the sample may be, for example, (i) the number of single nucleotide variants (SNVs) counted or (ii) a sum of the number of SNVs counted and the number of indel mutations counted. In some embodiments, the number of somatic mutations used in the calculation of the bTMB score determined from the sample is the number of SNVs counted. In some embodiments, the number of somatic mutations used in the calculation of the bTMB score determined from the sample is the number of synonymous and non-synonymous SNVs and/or indels. In some embodiments, the bTMB score determined from the sample is an equivalent bTMB value, for example, as determined by whole-exome sequencing. In some embodiments, the somatic mutations used in the calculation of the bTMB score determined from the sample are counted in one or more genes set forth in Table 1.

In some embodiments of any of the foregoing aspects or embodiments of the disclosure, the reference bTMB score is represented as the number of somatic mutations counted over a defined number of sequenced bases, as assessed, for example, using the FOUNDATIONONE CDX™ panel or the FOUNDATIONONE® panel. In some embodiments, the defined number of sequenced bases used in the calculation of the reference bTMB score is from about 100 kb to about 10 Mb (e.g., the defined number of sequenced bases may be 100 kb, 150 kb, 200 kb, 250 kb, 300 kb, 350 kb, 400 kb, 450 kb, 500 kb, 550 kb, 600 kb, 650 kb, 700 kb, 750 kb, 800 kb, 850 kb, 900 kb, 950 kb, 1.0 Mb, 1.2 Mb, 1.3 Mb, 1.4 Mb, 1.5 Mb, 1.6 Mb, 1.7 Mb, 1.8 Mb, 1.9 Mb, 2.0 Mb, 2.1 Mb, 2.2 Mb, 2.3 Mb, 2.4 Mb, 2.5 Mb, 2.6 Mb, 2.7 Mb, 2.8 Mb, 2.9 Mb, 3.0 Mb, 3.1 Mb, 3.2 Mb, 3.3 Mb, 3.4 Mb, 3.5 Mb, 3.6 Mb, 3.7 Mb, 3.8 Mb, 3.9 Mb, 4.0 Mb, 4.1 Mb, 4.2 Mb, 4.3 Mb, 4.4 Mb, 4.5 Mb, 4.6 Mb, 4.7 Mb, 4.8 Mb, 4.9 Mb, 5.0 Mb, 5.1 Mb, 5.2 Mb, 5.3 Mb, 5.4 Mb, 5.5 Mb, 5.6 Mb, 5.7 Mb, 5.8 Mb, 5.9 Mb, 6.0 Mb, 6.1 Mb, 6.2 Mb, 6.3 Mb, 6.4 Mb, 6.5 Mb, 6.6 Mb, 6.7 Mb, 6.8 Mb, 6.9 Mb, 7.0 Mb, 7.1 Mb, 7.2 Mb, 7.3 Mb, 7.4 Mb, 7.5 Mb, 7.6 Mb, 7.7 Mb, 7.8 Mb, 7.9 Mb, 8.0 Mb, 8.1 Mb, 8.2 Mb, 8.3 Mb, 8.4 Mb, 8.5 Mb, 8.6 Mb, 8.7 Mb, 8.8 Mb, 8.9 Mb, 9.0 Mb, 9.1 Mb, 9.2 Mb, 9.3 Mb, 9.4 Mb, 9.5 Mb, 9.6 Mb, 9.7 Mb, 9.8 Mb, 9.9 Mb, or 10.0 Mb). In some embodiments, the defined number of sequenced bases used in the calculation of the reference bTMB score is from about 0.5 Mb to about 1.5 Mb (e.g., the defined number of sequenced bases may be 500 kb, 550 kb, 600 kb, 650 kb, 700 kb, 750 kb, 800 kb, 850 kb, 900 kb, 950 kb, 1.0 Mb, 1.2 Mb, 1.3 Mb, 1.4 Mb, or 1.5 Mb). In some embodiments, the defined number of sequenced bases used in the calculation of the reference bTMB score is from about 0.7 Mb to about 1.3 Mb (e.g., the defined number of sequenced bases may be 700 kb, 750 kb, 800 kb, 850 kb, 900 kb, 950 kb, 1.0 Mb, 1.2 Mb, or 1.3 Mb). In some embodiments, the defined number of sequenced bases used in the calculation of the reference bTMB score is from about 0.8 Mb to about 1.2 Mb (e.g., the defined number of sequenced bases may be 800 kb, 850 kb, 900 kb, 950 kb, 1.0 Mb, or 1.2 Mb). In some embodiments, the defined number of sequenced bases used in the calculation of the reference bTMB score is about 1.1 Mb. The number of somatic mutations used in the calculation of the reference bTMB score may be, for example, (i) the number of single nucleotide variants (SNVs) counted or (ii) a sum of the number of SNVs counted and the number of indel mutations counted. In some embodiments, the number of somatic mutations used in the calculation of the reference bTMB score is the number of SNVs counted.

In some embodiments, the number of somatic mutations used in the calculation of the reference bTMB score is the number of synonymous and non-synonymous SNVs and/or indels. In some embodiments, the reference bTMB score is an equivalent bTMB value, for example, as determined by whole-exome sequencing. In some embodiments, the somatic mutations used in the calculation of the reference bTMB score are counted in one or more genes set forth in Table 1.

In some embodiments of any of the above aspects or embodiments of the disclosure, the method further comprises determining a maximum somatic allele frequency (MSAF) from a sample obtained from the subject. The MSAF may be, for example, greater than, or equal to, 1%. In some embodiments of any of the above aspects or embodiments of the disclosure, prior to being administered a PD-1 axis binding antagonist, a sample obtained from the subject has been determined to have an MSAF of greater than, or equal to, 1%.

In some embodiments, the method further comprises determining an MSAF from a sample obtained from the subject, wherein the MSAF from the sample is less than 1%. In some embodiments, prior to being administered a PD-1 axis binding antagonist, a sample obtained from the subject has been determined to have an MSAF of less than 1%.

In some embodiments, the method further comprises determining an MSAF from a sample obtained from the subject, wherein the MSAF from the sample has been determined to be greater than, or equal to, 1%, and the method further comprises administering to the individual an effective amount of an anti-cancer therapy other than, or in addition to, a PD-1 axis binding antagonist.

In some embodiments, the method further comprises determining an MSAF from a sample obtained from the subject, wherein the MSAF from the sample has been determined to be less than 1%, and the method further comprises administering an effective amount of a PD-1 axis binding antagonist to the individual.

In some embodiments, benefit from treatment containing a PD-1 axis binding antagonist includes an increase in OS. In some embodiments, benefit from treatment containing a PD-1 axis binding antagonist includes an increase in PFS.

In some embodiments, administration of the PD-1 axis binding antagonist to the subject extends the subject's OS as compared to administration of a platinum-based chemotherapy without the PD-1 axis binding antagonist. For example, administration of the PD-1 axis binding antagonist to the subject may extend the subject's OS by from about 4 months to about 10 months, by from about 5 months to about 9 months, by from about 6 months to about 8 months, by from about 6.5 months to about 7.5 months, or by from about 6.8 months to about 7.4 months as compared to administration of the platinum-based chemotherapy without the PD-1 axis binding antagonist (e.g., by about 4 months, 4.1 months, 4.2 months, 4.3 months, 4.4 months, 4.5 months, 4.6 months, 4.7 months, 4.8 months, 4.9 months, 5 months, 5.1 months, 5.2 months, 5.3 months, 5.4 months, 5.5 months, 5.6 months, 5.7 months, 5.8 months, 5.9 months, 6 months, 6.1 months, 6.2 months, 6.3 months, 6.4 months, 6.5 months, 6.6 months, 6.7 months, 6.8 months, 6.9 months, 7 months, 7.1 months, 7.2 months, 7.3 months, 7.4 months, 7.5 months, 7.6 months, 7.7 months, 7.8 months, 7.9 months, 8 months, 8.1 months, 8.2 months, 8.3 months, 8.4 months, 8.5 months, 8.6 months, 8.7 months, 8.8 months, 8.9 months, 9 months, 9.1 months, 9.2 months, 9.3 months, 9.4 months, 9.5 months, 9.6 months, 9.7 months, 9.8 months, 9.9 months, or 10 months). In some embodiments, administration of the PD-1 axis binding antagonist to the subject may extend the subject's OS by about 7.1 months as compared to administration of the platinum-based chemotherapy without the PD-1 axis binding antagonist.

In some embodiments of the disclosure, administration of the PD-1 axis binding antagonist to the subject extends the subject's PFS as compared to administration of the platinum-based chemotherapy without the PD-1 axis binding antagonist. For example, administration of the PD-1 axis binding antagonist to the subject may extend the subject's PFS by from about 1 month to about 5 months, by from about 2 months to about 4 months, by from about 2.1 months to about 3.9 months, by from about 2.5 months to about 3.5 months, or by from about 2.8 months to about 3.4 months as compared to administration of the platinum-based chemotherapy without the PD-1 axis binding antagonist (e.g., by about 1 month, 1.1 months, 1.2 months, 1.3 months, 1.4 months, 1.5 months, 1.6 months, 1.7 months, 1.8 months, 1.9 months, 2 months, 2.1 months, 2.2 months, 2.3 months, 2.4 months, 2.5 months, 2.6 months, 2.7 months, 2.8 months, 2.9 months, 3 months, 3.1 months, 3.2 months, 3.3 months, 3.4 months, 3.5 months, 3.6 months, 3.7 months, 3.8 months, 3.9 months, 4 months, 4.1 months, 4.2 months, 4.3 months, 4.4 months, 4.5 months, 4.6 months, 4.7 months, 4.8 months, 4.9 months, or 5 months). In some embodiments, administration of the PD-1 axis binding antagonist to the subject may extend the subject's OS by about 3.1 months as compared to administration of the platinum-based chemotherapy without the PD-1 axis binding antagonist.

In some embodiments, administration of the PD-1 axis binding antagonist to the subject increases the subject's likelihood of having an objective response and/or extends the subject's duration of response (DOR) as compared to administration of the platinum-based chemotherapy without the PD-1 axis binding antagonist.

In some embodiments, administration of the PD-1 axis binding antagonist to the subject increases the subject's likelihood of having an objective response and/or extends the subject's objective response rate (ORR) as compared to administration of the platinum-based chemotherapy without the PD-1 axis binding antagonist.

In some embodiments, administration of the PD-1 axis binding antagonist to the subject increases the subject's likelihood of having a complete response (CR) as compared to administration of the platinum-based chemotherapy without the PD-1 axis binding antagonist.

In some embodiments, the platinum-based chemotherapy comprises a platinum-based chemotherapeutic agent and a nucleoside analog.

In some embodiments, the platinum-based chemotherapeutic agent is cisplatin, carboplatin, or oxaliplatin.

In some embodiments, the platinum-based chemotherapeutic agent is cisplatin.

In other embodiments, the platinum-based chemotherapeutic agent is carboplatin.

In some embodiments, the nucleoside analog is gemcitabine.

In some embodiments, the platinum-based chemotherapy comprises cisplatin and gemcitabine or carboplatin and gemcitabine.

In some embodiments, the platinum-based chemotherapy comprises cisplatin and gemcitabine.

In some embodiments, the platinum-based chemotherapy comprises carboplatin and gemcitabine.

In some embodiments, the platinum-based chemotherapy comprises cisplatin and premetrexed or carboplatin and premetrexed.

In some embodiments, the platinum-based chemotherapy comprises cisplatin and premetrexed.

In some embodiments, the platinum-based chemotherapy comprises carboplatin and premetrexed.

In some embodiments, the PD-1 axis binding antagonist is administered to the subject during one or more dosing cycles, such as from 1 to 10 dosing cycles (e.g., from 2 to 8 dosing cycles, from 3 to 7 dosing cycles, or from 4 to 6 dosing cycles). In some embodiments, the PD-1 axis binding antagonist is administered to the subject during from 4 to 6 dosing cycles. The PD-1 axis binding antagonist may be administered to the subject one or more times during each dosing cycle, such as once per dosing cycle.

In some embodiments, the dosing cycles continue for up to 58 months. For example, the dosing cycles may continue for from 1 month to 100 months, such as from 2 months to 99 months, from 3 months to 98 months, from 4 months to 97 months, from 5 months to 96 months, from 6 months to 95 months, from 7 months to 94 months, from 8 months to 93 months, from 9 months to 92 months, from 10 months to 91 months, from 11 months to 90 months, from 12 months to 89 months, from 13 months to 88 months, from 14 months to 87 months, from 15 months to 86 months, from 16 months to 85 months, from 17 months to 84 months, from 18 months to 83 months, from 19 months to 82 months, from 20 months to 81 months, from 21 months to 80 months, from 22 months to 79 months, from 23 months to 78 months, from 24 months to 77 months, from 25 months to 76 months, from 26 months to 75 months, from 27 months to 74 months, from 28 months to 73 months, from 29 months to 72 months, from 30 months to 71 months, from 31 months to 70 months, from 32 months to 69 months, from 33 months to 68 months, from 34 months to 67 months, from 35 months to 66 months, from 36 months to 65 months, from 37 months to 64 months, from 36 months to 63 months, from 37 months to 62 months, from 38 months to 61 months, from 39 months to 60 months, from 50 months to 70 months, from 51 months to 69 months, from 52 months to 68 months, from 53 months to 67 months, from 54 months to 66 months, from 55 months to 61 months, from 56 months to 60 months, or from 57 months to 59 months.

In some embodiments, each dosing cycle is about 21 days.

In some embodiments, the PD-1 axis binding antagonist is administered to the subject as a monotherapy. In some embodiments, the PD-1 axis binding antagonist is administered to the subject in combination with a platinum-based chemotherapy, such as a platinum-based chemotherapy that includes a platinum-based chemotherapeutic agent and a nucleoside analog. In some embodiments, the platinum-based chemotherapeutic agent administered to the subject is cisplatin, carboplatin, or oxaliplatin. In some embodiments, the platinum-based chemotherapeutic agent administered to the subject is cisplatin. In other embodiments, the platinum-based chemotherapeutic agent administered to the subject is carboplatin. In some embodiments, the nucleoside analog administered to the subject is gemcitabine. In some embodiments, the platinum-based chemotherapy administered to the subject comprises cisplatin and gemcitabine or carboplatin and gemcitabine. In some embodiments, the platinum-based chemotherapy administered to the subject comprises cisplatin and gemcitabine. In some embodiments, the platinum-based chemotherapy administered to the subject comprises carboplatin and gemcitabine. In some embodiments, the platinum-based chemotherapy administered to the subject comprises cisplatin and premetrexed or carboplatin and premetrexed. In some embodiments, the platinum-based chemotherapy administered to the subject comprises cisplatin and premetrexed. In some embodiments, the platinum-based chemotherapy administered to the subject comprises carboplatin and premetrexed.

In some embodiments, cisplatin is administered to the subject intravenously at a dose of about 75 mg/m² on Day −2 to Day 4 of a 21-day dosing cycle (e.g., on Day −2, Day −1, Day 0, Day 1, Day 2, Day 3, or Day 4 of a 21-day dosing cycle). In some embodiments, cisplatin is administered to the subject intravenously at a dose of about 75 mg/m² on Day 1 of a 21-day dosing cycle.

In some embodiments, carboplatin is administered to the subject intravenously at an area under the curve (AUC) of about 5 on Day −2 to Day 4 of a 21-day dosing cycle (e.g., on Day −2, Day −1, Day 0, Day 1, Day 2, Day 3, or Day 4 of a 21-day dosing cycle). In some embodiments, carboplatin is administered to the subject intravenously at an AUC of about 5 on Day 1 of a 21-day dosing cycle.

In some embodiments, carboplatin is administered to the subject intravenously at an AUC of about 6 on Day −2 to Day 4 of a 21-day dosing cycle (e.g., on Day −2, Day −1, Day 0, Day 1, Day 2, Day 3, or Day 4 of a 21-day dosing cycle). In some embodiments, carboplatin is administered to the subject intravenously at an AUC of about 6 on Day 1 of a 21-day dosing cycle.

In some embodiments, gemcitabine is administered to the subject intravenously at a dose of about 1000 mg/m² on Day −2 to Day 4 (e.g., on Day −2, Day −1, Day 0, Day 1, Day 2, Day 3, or Day 4) and on Day 7 to Day 11 (e.g., on Day 7, Day 8, Day 9, Day 10, or Day 11) of a 21-day dosing cycle. In some embodiments, gemcitabine is administered to the subject intravenously at a dose of about 1000 mg/m² on Day 1 and Day 8 of a 21-day dosing cycle.

In some embodiments, gemcitabine is administered to the subject intravenously at a dose of about 1250 mg/m² on Day −2 to Day 4 (e.g., on Day −2, Day −1, Day 0, Day 1, Day 2, Day 3, or Day 4) and on Day 7 to Day 11 (e.g., on Day 7, Day 8, Day 9, Day 10, or Day 11) of a 21-day dosing cycle. In some embodiments, gemcitabine is administered to the subject intravenously at a dose of about 1250 mg/m² on Day 1 and Day 8 of a 21-day dosing cycle.

In some embodiments, premetrexed is administered to the subject intravenously at a dose of about 500 mg/m² on Day −2 to Day 4 of a 21-day dosing cycle (e.g., on Day −2, Day −1, Day 0, Day 1, Day 2, Day 3, or Day 4 of a 21-day dosing cycle). In some embodiments, premetrexed is administered to the subject intravenously at a dose of about 500 mg/m² on Day 1 of a 21-day dosing cycle.

In some embodiments, the PD-1 axis binding antagonist is selected from the group consisting of a PD-L1 binding antagonist, a PD-1 binding antagonist, and a PD-L2 binding antagonist.

In some embodiments, the PD-1 axis binding antagonist is a PD-L1 binding antagonist.

In some embodiments, the PD-L1 binding antagonist inhibits the binding of PD-L1 to one or more of its ligand binding partners.

In some embodiments, the PD-L1 binding antagonist inhibits the binding of PD-L1 to PD-1, B7-1, or both PD-1 and B7-1.

In some embodiments, the PD-L1 binding antagonist is an anti-PD-L1 antibody.

In some embodiments, the anti-PD-L1 antibody is atezolizumab (TECENTRIQ®), MDX-1105, MED14736 (durvalumab), or MSB0010718C (avelumab).

In some embodiments, the anti-PD-L1 antibody comprises the following hypervariable regions (HVRs): (a) an HVR-H1 sequence of GFTFSDSWIH (SEQ ID NO: 19); (b) an HVR-H2 sequence of AWISPYGGSTYYADSVKG (SEQ ID NO: 20); (c) an HVR-H3 sequence of RHWPGGFDY (SEQ ID NO: 21); (d) an HVR-L1 sequence of RASQDVSTAVA (SEQ ID NO: 22); (e) an HVR-L2 sequence of SASFLYS (SEQ ID NO: 23); and (f) an HVR-L3 sequence of QQYLYHPAT (SEQ ID NO: 24).

In some embodiments, the anti-PD-L1 antibody comprises: (a) a heavy chain variable (VH) domain comprising an amino acid sequence having at least 90% sequence identity to the amino acid sequence of SEQ ID NO: 3; (b) a light chain variable (VL) domain comprising an amino acid sequence having at least 90% sequence identity to the amino acid sequence of SEQ ID NO: 4; or (c) a VH domain as in (a) and a VL domain as in (b). In some embodiments, the anti-PD-L1 antibody comprises: (a) a VH domain comprising an amino acid sequence having at least 95% sequence identity to the amino acid sequence of SEQ ID NO: 3; (b) a VL domain comprising an amino acid sequence having at least 95% sequence identity to the amino acid sequence of SEQ ID NO: 4; or (c) a VH domain as in (a) and a VL domain as in (b). In some embodiments, the anti-PD-L1 antibody comprises: (a) a VH domain comprising an amino acid sequence having at least 96% sequence identity to the amino acid sequence of SEQ ID NO: 3; (b) a VL domain comprising an amino acid sequence having at least 96% sequence identity to the amino acid sequence of SEQ ID NO: 4; or (c) a VH domain as in (a) and a VL domain as in (b). In some embodiments, the anti-PD-L1 antibody comprises: (a) a VH domain comprising an amino acid sequence having at least 97% sequence identity to the amino acid sequence of SEQ ID NO: 3; (b) a VL domain comprising an amino acid sequence having at least 97% sequence identity to the amino acid sequence of SEQ ID NO: 4; or (c) a VH domain as in (a) and a VL domain as in (b). In some embodiments, the anti-PD-L1 antibody comprises: (a) a VH domain comprising an amino acid sequence having at least 98% sequence identity to the amino acid sequence of SEQ ID NO: 3; (b) a VL domain comprising an amino acid sequence having at least 98% sequence identity to the amino acid sequence of SEQ ID NO: 4; or (c) a VH domain as in (a) and a VL domain as in (b). In some embodiments, the anti-PD-L1 antibody comprises: (a) a VH domain comprising an amino acid sequence having at least 99% sequence identity to the amino acid sequence of SEQ ID NO: 3; (b) a VL domain comprising an amino acid sequence having at least 99% sequence identity to the amino acid sequence of SEQ ID NO: 4; or (c) a VH domain as in (a) and a VL domain as in (b). In some embodiments, the anti-PD-L1 antibody comprises: (a) a VH domain comprising the amino acid sequence of SEQ ID NO: 3; (b) a VL domain comprising the amino acid sequence of SEQ ID NO: 4; or (c) a VH domain as in (a) and a VL domain as in (b). In some embodiments, the anti-PD-L1 antibody comprises: (a) a VH domain comprising the amino acid sequence of SEQ ID NO: 3; and (b) a VL domain comprising the amino acid sequence of SEQ ID NO: 4.

In some embodiments, the anti-PD-L1 antibody is atezolizumab.

In some embodiments, atezolizumab is administered to the subject intravenously at a dose of about 840 mg every 2 weeks, about 1200 mg every 3 weeks, or about 1680 mg every 4 weeks.

In some embodiments, atezolizumab is administered to the subject intravenously at a dose of about 1200 mg every 3 weeks.

In some embodiments, atezolizumab is administered to the subject intravenously at a dose of about 1200 mg on Day −2 to Day 4 of a 21-day dosing cycle.

In some embodiments, atezolizumab is administered to the subject intravenously at a dose of about 1200 mg on Day 1 of a 21-day dosing cycle.

In some embodiments, the PD-1 axis binding antagonist is a PD-1 binding antagonist.

In some embodiments, the PD-1 binding antagonist inhibits the binding of PD-1 to one or more of its ligand binding partners.

In some embodiments, the PD-1 binding antagonist inhibits the binding of PD-1 to PD-L1, PD-L2, or both PD-L1 and PD-L2.

In some embodiments, the PD-1 binding antagonist is an anti-PD-1 antibody.

In some embodiments, the anti-PD-1 antibody is MDX-1106 (nivolumab), MK-3475 (pembrolizumab), MEDI-0680 (AMP-514), PDR001, REGN2810, or BGB-108.

In some embodiments, the PD-1 binding antagonist is an Fc fusion protein.

In some embodiments, the Fc fusion protein is AMP-224.

In some embodiments, the subject is chemotherapy naïve. For example, the subject may be one that has not previously been administered chemotherapy for treatment of the NSCLC. In some embodiments, the subject has not previously been administered systemic therapy for treatment of the NSCLC. In some embodiments, the subject has not previously been administered any therapy for treatment of the NSCLC.

In some embodiments, the NSCLC is stage IV NSCLC.

In some embodiments, the NSCLC is metastatic NSCLC.

In some embodiments, a tumor sample obtained from the subject has been determined to have a detectable expression level of PD-L1 in 1% or more of the tumor cells in the tumor sample. For example, in some embodiments, the tumor sample obtained from the subject has been determined to have a detectable expression level of PD-L1 in from about 1% to about 100% of the tumor cells in the tumor sample, from about 2% to about 100% of the tumor cells in the tumor sample, from about 3% to about 100% of the tumor cells in the tumor sample, from about 4% to about 100% of the tumor cells in the tumor sample, from about 5% to about 100% of the tumor cells in the tumor sample, from about 6% to about 100% of the tumor cells in the tumor sample, from about 7% to about 100% of the tumor cells in the tumor sample, from about 8% to about 100% of the tumor cells in the tumor sample, from about 9% to about 100% of the tumor cells in the tumor sample, from about 10% to about 100% of the tumor cells in the tumor sample, from about 11% to about 100% of the tumor cells in the tumor sample, from about 12% to about 100% of the tumor cells in the tumor sample, from about 13% to about 100% of the tumor cells in the tumor sample, from about 14% to about 100% of the tumor cells in the tumor sample, from about 15% to about 100% of the tumor cells in the tumor sample, from about 16% to about 100% of the tumor cells in the tumor sample, from about 17% to about 100% of the tumor cells in the tumor sample, from about 18% to about 100% of the tumor cells in the tumor sample, from about 19% to about 100% of the tumor cells in the tumor sample, from about 20% to about 100% of the tumor cells in the tumor sample, from about 21% to about 100% of the tumor cells in the tumor sample, from about 22% to about 100% of the tumor cells in the tumor sample, from about 23% to about 100% of the tumor cells in the tumor sample, from about 24% to about 100% of the tumor cells in the tumor sample, from about 25% to about 100% of the tumor cells in the tumor sample, from about 26% to about 100% of the tumor cells in the tumor sample, from about 27% to about 100% of the tumor cells in the tumor sample, from about 28% to about 100% of the tumor cells in the tumor sample, from about 29% to about 100% of the tumor cells in the tumor sample, from about 30% to about 100% of the tumor cells in the tumor sample, from about 31% to about 100% of the tumor cells in the tumor sample, from about 32% to about 100% of the tumor cells in the tumor sample, from about 33% to about 100% of the tumor cells in the tumor sample, from about 34% to about 100% of the tumor cells in the tumor sample, from about 35% to about 100% of the tumor cells in the tumor sample, from about 36% to about 100% of the tumor cells in the tumor sample, from about 37% to about 100% of the tumor cells in the tumor sample, from about 38% to about 100% of the tumor cells in the tumor sample, from about 39% to about 100% of the tumor cells in the tumor sample, from about 40% to about 100% of the tumor cells in the tumor sample, from about 41% to about 100% of the tumor cells in the tumor sample, from about 42% to about 100% of the tumor cells in the tumor sample, from about 43% to about 100% of the tumor cells in the tumor sample, from about 44% to about 100% of the tumor cells in the tumor sample, from about 45% to about 100% of the tumor cells in the tumor sample, from about 46% to about 100% of the tumor cells in the tumor sample, from about 47% to about 100% of the tumor cells in the tumor sample, from about 48% to about 100% of the tumor cells in the tumor sample, from about 49% to about 100% of the tumor cells in the tumor sample, from about 50% to about 100% of the tumor cells in the tumor sample, from about 51% to about 100% of the tumor cells in the tumor sample, from about 52% to about 100% of the tumor cells in the tumor sample, from about 53% to about 100% of the tumor cells in the tumor sample, from about 54% to about 100% of the tumor cells in the tumor sample, from about 55% to about 100% of the tumor cells in the tumor sample, from about 56% to about 100% of the tumor cells in the tumor sample, from about 57% to about 100% of the tumor cells in the tumor sample, from about 58% to about 100% of the tumor cells in the tumor sample, from about 59% to about 100% of the tumor cells in the tumor sample, from about 60% to about 100% of the tumor cells in the tumor sample, from about 61% to about 100% of the tumor cells in the tumor sample, from about 62% to about 100% of the tumor cells in the tumor sample, from about 63% to about 100% of the tumor cells in the tumor sample, from about 64% to about 100% of the tumor cells in the tumor sample, from about 65% to about 100% of the tumor cells in the tumor sample, from about 66% to about 100% of the tumor cells in the tumor sample, from about 67% to about 100% of the tumor cells in the tumor sample, from about 68% to about 100% of the tumor cells in the tumor sample, from about 69% to about 100% of the tumor cells in the tumor sample, from about 70% to about 100% of the tumor cells in the tumor sample, from about 71% to about 100% of the tumor cells in the tumor sample, from about 72% to about 100% of the tumor cells in the tumor sample, from about 73% to about 100% of the tumor cells in the tumor sample, from about 74% to about 100% of the tumor cells in the tumor sample, from about 75% to about 100% of the tumor cells in the tumor sample, from about 76% to about 100% of the tumor cells in the tumor sample, from about 77% to about 100% of the tumor cells in the tumor sample, from about 78% to about 100% of the tumor cells in the tumor sample, from about 79% to about 100% of the tumor cells in the tumor sample, from about 80% to about 100% of the tumor cells in the tumor sample, from about 81% to about 100% of the tumor cells in the tumor sample, from about 82% to about 100% of the tumor cells in the tumor sample, from about 83% to about 100% of the tumor cells in the tumor sample, from about 84% to about 100% of the tumor cells in the tumor sample, from about 85% to about 100% of the tumor cells in the tumor sample, from about 86% to about 100% of the tumor cells in the tumor sample, from about 87% to about 100% of the tumor cells in the tumor sample, from about 88% to about 100% of the tumor cells in the tumor sample, from about 89% to about 100% of the tumor cells in the tumor sample, from about 90% to about 100% of the tumor cells in the tumor sample, from about 91% to about 100% of the tumor cells in the tumor sample, from about 92% to about 100% of the tumor cells in the tumor sample, from about 93% to about 100% of the tumor cells in the tumor sample, from about 94% to about 100% of the tumor cells in the tumor sample, from about 95% to about 100% of the tumor cells in the tumor sample, from about 96% to about 100% of the tumor cells in the tumor sample, from about 97% to about 100% of the tumor cells in the tumor sample, from about 98% to about 100% of the tumor cells in the tumor sample, or from about 99% to about 100% of the tumor cells in the tumor sample (e.g., about 1% of the tumor cells in the tumor sample, about 2% of the tumor cells in the tumor sample, about 3% of the tumor cells in the tumor sample, about 4% of the tumor cells in the tumor sample, about 5% of the tumor cells in the tumor sample, about 6% of the tumor cells in the tumor sample, about 7% of the tumor cells in the tumor sample, about 8% of the tumor cells in the tumor sample, about 9% of the tumor cells in the tumor sample, about 10% of the tumor cells in the tumor sample, about 11% of the tumor cells in the tumor sample, about 12% of the tumor cells in the tumor sample, about 13% of the tumor cells in the tumor sample, about 14% of the tumor cells in the tumor sample, about 15% of the tumor cells in the tumor sample, about 16% of the tumor cells in the tumor sample, about 17% of the tumor cells in the tumor sample, about 18% of the tumor cells in the tumor sample, about 19% of the tumor cells in the tumor sample, about 20% of the tumor cells in the tumor sample, about 21% of the tumor cells in the tumor sample, about 22% of the tumor cells in the tumor sample, about 23% of the tumor cells in the tumor sample, about 24% of the tumor cells in the tumor sample, about 25% of the tumor cells in the tumor sample, about 26% of the tumor cells in the tumor sample, about 27% of the tumor cells in the tumor sample, about 28% of the tumor cells in the tumor sample, about 29% of the tumor cells in the tumor sample, about 30% of the tumor cells in the tumor sample, about 31% of the tumor cells in the tumor sample, about 32% of the tumor cells in the tumor sample, about 33% of the tumor cells in the tumor sample, about 34% of the tumor cells in the tumor sample, about 35% of the tumor cells in the tumor sample, about 36% of the tumor cells in the tumor sample, about 37% of the tumor cells in the tumor sample, about 38% of the tumor cells in the tumor sample, about 39% of the tumor cells in the tumor sample, about 40% of the tumor cells in the tumor sample, about 41% of the tumor cells in the tumor sample, about 42% of the tumor cells in the tumor sample, about 43% of the tumor cells in the tumor sample, about 44% of the tumor cells in the tumor sample, about 45% of the tumor cells in the tumor sample, about 46% of the tumor cells in the tumor sample, about 47% of the tumor cells in the tumor sample, about 48% of the tumor cells in the tumor sample, about 49% of the tumor cells in the tumor sample, about 50% of the tumor cells in the tumor sample, about 51% of the tumor cells in the tumor sample, about 52% of the tumor cells in the tumor sample, about 53% of the tumor cells in the tumor sample, about 54% of the tumor cells in the tumor sample, about 55% of the tumor cells in the tumor sample, about 56% of the tumor cells in the tumor sample, about 57% of the tumor cells in the tumor sample, about 58% of the tumor cells in the tumor sample, about 59% of the tumor cells in the tumor sample, about 60% of the tumor cells in the tumor sample, about 61% of the tumor cells in the tumor sample, about 62% of the tumor cells in the tumor sample, about 63% of the tumor cells in the tumor sample, about 64% of the tumor cells in the tumor sample, about 65% of the tumor cells in the tumor sample, about 66% of the tumor cells in the tumor sample, about 67% of the tumor cells in the tumor sample, about 68% of the tumor cells in the tumor sample, about 69% of the tumor cells in the tumor sample, about 70% of the tumor cells in the tumor sample, about 71% of the tumor cells in the tumor sample, about 72% of the tumor cells in the tumor sample, about 73% of the tumor cells in the tumor sample, about 74% of the tumor cells in the tumor sample, about 75% of the tumor cells in the tumor sample, about 76% of the tumor cells in the tumor sample, about 77% of the tumor cells in the tumor sample, about 78% of the tumor cells in the tumor sample, about 79% of the tumor cells in the tumor sample, about 80% of the tumor cells in the tumor sample, about 81% of the tumor cells in the tumor sample, about 82% of the tumor cells in the tumor sample, about 83% of the tumor cells in the tumor sample, about 84% of the tumor cells in the tumor sample, about 85% of the tumor cells in the tumor sample, about 86% of the tumor cells in the tumor sample, about 87% of the tumor cells in the tumor sample, about 88% of the tumor cells in the tumor sample, about 89% of the tumor cells in the tumor sample, about 90% of the tumor cells in the tumor sample, about 91% of the tumor cells in the tumor sample, about 92% of the tumor cells in the tumor sample, about 93% of the tumor cells in the tumor sample, about 94% of the tumor cells in the tumor sample, about 95% of the tumor cells in the tumor sample, about 96% of the tumor cells in the tumor sample, about 97% of the tumor cells in the tumor sample, about 98% of the tumor cells in the tumor sample, about 99% of the tumor cells in the tumor sample, or about 100% of the tumor cells in the tumor sample).

In some embodiments, a tumor sample obtained from the subject has been determined to have a detectable expression level of PD-L1 in tumor-infiltrating immune cells that comprise 1% or more of the tumor sample. For example, in some embodiments the tumor sample obtained from the subject has been determined to have a detectable expression level of PD-L1 in tumor-infiltrating immune cells that comprise from about 1% to about 100% of the tumor sample, from about 5% to about 100% of the tumor sample, from about 10% to about 100% of the tumor sample, from about 15% to about 100% of the tumor sample, from about 20% to about 100% of the tumor sample, from about 30% to about 100% of the tumor sample, from about 40% to about 100% of the tumor sample, from about 50% to about 100% of the tumor sample, from about 60% to about 100% of the tumor sample, from about 70% to about 100% of the tumor sample, from about 80% to about 100% of the tumor sample, or from about 90% to about 100% of the tumor sample (e.g., about 1% of the tumor sample, about 2% of the tumor sample, about 3% of the tumor sample, about 4% of the tumor sample, about 5% of the tumor sample, about 6% of the tumor sample, about 7% of the tumor sample, about 8% of the tumor sample, about 9% of the tumor sample, about 10% of the tumor sample, about 11% of the tumor sample, about 12% of the tumor sample, about 13% of the tumor sample, about 14% of the tumor sample, about 15% of the tumor sample, about 16% of the tumor sample, about 17% of the tumor sample, about 18% of the tumor sample, about 19% of the tumor sample, about 20% of the tumor sample, about 21% of the tumor sample, about 22% of the tumor sample, about 23% of the tumor sample, about 24% of the tumor sample, about 25% of the tumor sample, about 26% of the tumor sample, about 27% of the tumor sample, about 28% of the tumor sample, about 29% of the tumor sample, about 30% of the tumor sample, about 31% of the tumor sample, about 32% of the tumor sample, about 33% of the tumor sample, about 34% of the tumor sample, about 35% of the tumor sample, about 36% of the tumor sample, about 37% of the tumor sample, about 38% of the tumor sample, about 39% of the tumor sample, about 40% of the tumor sample, about 41% of the tumor sample, about 42% of the tumor sample, about 43% of the tumor sample, about 44% of the tumor sample, about 45% of the tumor sample, about 46% of the tumor sample, about 47% of the tumor sample, about 48% of the tumor sample, about 49% of the tumor sample, about 50% of the tumor sample, about 51% of the tumor sample, about 52% of the tumor sample, about 53% of the tumor sample, about 54% of the tumor sample, about 55% of the tumor sample, about 56% of the tumor sample, about 57% of the tumor sample, about 58% of the tumor sample, about 59% of the tumor sample, about 60% of the tumor sample, about 61% of the tumor sample, about 62% of the tumor sample, about 63% of the tumor sample, about 64% of the tumor sample, about 65% of the tumor sample, about 66% of the tumor sample, about 67% of the tumor sample, about 68% of the tumor sample, about 69% of the tumor sample, about 70% of the tumor sample, about 71% of the tumor sample, about 72% of the tumor sample, about 73% of the tumor sample, about 74% of the tumor sample, about 75% of the tumor sample, about 76% of the tumor sample, about 77% of the tumor sample, about 78% of the tumor sample, about 79% of the tumor sample, about 80% of the tumor sample, about 81% of the tumor sample, about 82% of the tumor sample, about 83% of the tumor sample, about 84% of the tumor sample, about 85% of the tumor sample, about 86% of the tumor sample, about 87% of the tumor sample, about 88% of the tumor sample, about 89% of the tumor sample, about 90% of the tumor sample, about 91% of the tumor sample, about 92% of the tumor sample, about 93% of the tumor sample, about 94% of the tumor sample, about 95% of the tumor sample, about 96% of the tumor sample, about 97% of the tumor sample, about 98% of the tumor sample, about 99% of the tumor sample, or about 100% of the tumor sample).

In some embodiments, a tumor sample obtained from the subject has been determined to have a detectable expression level of PD-L1 in from about 1% to less than 5% of the tumor cells in the tumor sample. For example, in some embodiments, the tumor sample obtained from the subject has been determined to have a detectable expression level of PD-L1 in about 1% of the tumor cells in the tumor sample, about 2% of the tumor cells in the tumor sample, about 3% of the tumor cells in the tumor sample, or about 4% of the tumor cells in the tumor sample.

In some embodiments, a tumor sample obtained from the subject has been determined to have a detectable expression level of PD-L1 in tumor-infiltrating immune cells that comprise from about 1% to less than 5% of the tumor cells in the tumor sample. For example, in some embodiments, the tumor sample obtained from the subject has been determined to have a detectable expression level of PD-L1 in tumor-infiltrating immune cells that comprise about 1% of the tumor cells in the tumor sample, about 2% of the tumor cells in the tumor sample, about 3% of the tumor cells in the tumor sample, or about 4% of the tumor cells in the tumor sample.

In some embodiments, a tumor sample obtained from the subject has been determined to have a detectable expression level of PD-L1 in 5% or more of the tumor cells in the tumor sample. For example, in some embodiments, the tumor sample obtained from the subject has been determined to have a detectable expression level of PD-L1 in from about 5% to about 100% of the tumor cells in the tumor sample, from about 6% to about 100% of the tumor cells in the tumor sample, from about 7% to about 100% of the tumor cells in the tumor sample, from about 8% to about 100% of the tumor cells in the tumor sample, from about 9% to about 100% of the tumor cells in the tumor sample, from about 10% to about 100% of the tumor cells in the tumor sample, from about 11% to about 100% of the tumor cells in the tumor sample, from about 12% to about 100% of the tumor cells in the tumor sample, from about 13% to about 100% of the tumor cells in the tumor sample, from about 14% to about 100% of the tumor cells in the tumor sample, from about 15% to about 100% of the tumor cells in the tumor sample, from about 16% to about 100% of the tumor cells in the tumor sample, from about 17% to about 100% of the tumor cells in the tumor sample, from about 18% to about 100% of the tumor cells in the tumor sample, from about 19% to about 100% of the tumor cells in the tumor sample, from about 20% to about 100% of the tumor cells in the tumor sample, from about 21% to about 100% of the tumor cells in the tumor sample, from about 22% to about 100% of the tumor cells in the tumor sample, from about 23% to about 100% of the tumor cells in the tumor sample, from about 24% to about 100% of the tumor cells in the tumor sample, from about 25% to about 100% of the tumor cells in the tumor sample, from about 26% to about 100% of the tumor cells in the tumor sample, from about 27% to about 100% of the tumor cells in the tumor sample, from about 28% to about 100% of the tumor cells in the tumor sample, from about 29% to about 100% of the tumor cells in the tumor sample, from about 30% to about 100% of the tumor cells in the tumor sample, from about 31% to about 100% of the tumor cells in the tumor sample, from about 32% to about 100% of the tumor cells in the tumor sample, from about 33% to about 100% of the tumor cells in the tumor sample, from about 34% to about 100% of the tumor cells in the tumor sample, from about 35% to about 100% of the tumor cells in the tumor sample, from about 36% to about 100% of the tumor cells in the tumor sample, from about 37% to about 100% of the tumor cells in the tumor sample, from about 38% to about 100% of the tumor cells in the tumor sample, from about 39% to about 100% of the tumor cells in the tumor sample, from about 40% to about 100% of the tumor cells in the tumor sample, from about 41% to about 100% of the tumor cells in the tumor sample, from about 42% to about 100% of the tumor cells in the tumor sample, from about 43% to about 100% of the tumor cells in the tumor sample, from about 44% to about 100% of the tumor cells in the tumor sample, from about 45% to about 100% of the tumor cells in the tumor sample, from about 46% to about 100% of the tumor cells in the tumor sample, from about 47% to about 100% of the tumor cells in the tumor sample, from about 48% to about 100% of the tumor cells in the tumor sample, from about 49% to about 100% of the tumor cells in the tumor sample, from about 50% to about 100% of the tumor cells in the tumor sample, from about 51% to about 100% of the tumor cells in the tumor sample, from about 52% to about 100% of the tumor cells in the tumor sample, from about 53% to about 100% of the tumor cells in the tumor sample, from about 54% to about 100% of the tumor cells in the tumor sample, from about 55% to about 100% of the tumor cells in the tumor sample, from about 56% to about 100% of the tumor cells in the tumor sample, from about 57% to about 100% of the tumor cells in the tumor sample, from about 58% to about 100% of the tumor cells in the tumor sample, from about 59% to about 100% of the tumor cells in the tumor sample, from about 60% to about 100% of the tumor cells in the tumor sample, from about 61% to about 100% of the tumor cells in the tumor sample, from about 62% to about 100% of the tumor cells in the tumor sample, from about 63% to about 100% of the tumor cells in the tumor sample, from about 64% to about 100% of the tumor cells in the tumor sample, from about 65% to about 100% of the tumor cells in the tumor sample, from about 66% to about 100% of the tumor cells in the tumor sample, from about 67% to about 100% of the tumor cells in the tumor sample, from about 68% to about 100% of the tumor cells in the tumor sample, from about 69% to about 100% of the tumor cells in the tumor sample, from about 70% to about 100% of the tumor cells in the tumor sample, from about 71% to about 100% of the tumor cells in the tumor sample, from about 72% to about 100% of the tumor cells in the tumor sample, from about 73% to about 100% of the tumor cells in the tumor sample, from about 74% to about 100% of the tumor cells in the tumor sample, from about 75% to about 100% of the tumor cells in the tumor sample, from about 76% to about 100% of the tumor cells in the tumor sample, from about 77% to about 100% of the tumor cells in the tumor sample, from about 78% to about 100% of the tumor cells in the tumor sample, from about 79% to about 100% of the tumor cells in the tumor sample, from about 80% to about 100% of the tumor cells in the tumor sample, from about 81% to about 100% of the tumor cells in the tumor sample, from about 82% to about 100% of the tumor cells in the tumor sample, from about 83% to about 100% of the tumor cells in the tumor sample, from about 84% to about 100% of the tumor cells in the tumor sample, from about 85% to about 100% of the tumor cells in the tumor sample, from about 86% to about 100% of the tumor cells in the tumor sample, from about 87% to about 100% of the tumor cells in the tumor sample, from about 88% to about 100% of the tumor cells in the tumor sample, from about 89% to about 100% of the tumor cells in the tumor sample, from about 90% to about 100% of the tumor cells in the tumor sample, from about 91% to about 100% of the tumor cells in the tumor sample, from about 92% to about 100% of the tumor cells in the tumor sample, from about 93% to about 100% of the tumor cells in the tumor sample, from about 94% to about 100% of the tumor cells in the tumor sample, from about 95% to about 100% of the tumor cells in the tumor sample, from about 96% to about 100% of the tumor cells in the tumor sample, from about 97% to about 100% of the tumor cells in the tumor sample, from about 98% to about 100% of the tumor cells in the tumor sample, or from about 99% to about 100% of the tumor cells in the tumor sample (e.g., about 5% of the tumor cells in the tumor sample, about 6% of the tumor cells in the tumor sample, about 7% of the tumor cells in the tumor sample, about 8% of the tumor cells in the tumor sample, about 9% of the tumor cells in the tumor sample, about 10% of the tumor cells in the tumor sample, about 11% of the tumor cells in the tumor sample, about 12% of the tumor cells in the tumor sample, about 13% of the tumor cells in the tumor sample, about 14% of the tumor cells in the tumor sample, about 15% of the tumor cells in the tumor sample, about 16% of the tumor cells in the tumor sample, about 17% of the tumor cells in the tumor sample, about 18% of the tumor cells in the tumor sample, about 19% of the tumor cells in the tumor sample, about 20% of the tumor cells in the tumor sample, about 21% of the tumor cells in the tumor sample, about 22% of the tumor cells in the tumor sample, about 23% of the tumor cells in the tumor sample, about 24% of the tumor cells in the tumor sample, about 25% of the tumor cells in the tumor sample, about 26% of the tumor cells in the tumor sample, about 27% of the tumor cells in the tumor sample, about 28% of the tumor cells in the tumor sample, about 29% of the tumor cells in the tumor sample, about 30% of the tumor cells in the tumor sample, about 31% of the tumor cells in the tumor sample, about 32% of the tumor cells in the tumor sample, about 33% of the tumor cells in the tumor sample, about 34% of the tumor cells in the tumor sample, about 35% of the tumor cells in the tumor sample, about 36% of the tumor cells in the tumor sample, about 37% of the tumor cells in the tumor sample, about 38% of the tumor cells in the tumor sample, about 39% of the tumor cells in the tumor sample, about 40% of the tumor cells in the tumor sample, about 41% of the tumor cells in the tumor sample, about 42% of the tumor cells in the tumor sample, about 43% of the tumor cells in the tumor sample, about 44% of the tumor cells in the tumor sample, about 45% of the tumor cells in the tumor sample, about 46% of the tumor cells in the tumor sample, about 47% of the tumor cells in the tumor sample, about 48% of the tumor cells in the tumor sample, about 49% of the tumor cells in the tumor sample, about 50% of the tumor cells in the tumor sample, about 51% of the tumor cells in the tumor sample, about 52% of the tumor cells in the tumor sample, about 53% of the tumor cells in the tumor sample, about 54% of the tumor cells in the tumor sample, about 55% of the tumor cells in the tumor sample, about 56% of the tumor cells in the tumor sample, about 57% of the tumor cells in the tumor sample, about 58% of the tumor cells in the tumor sample, about 59% of the tumor cells in the tumor sample, about 60% of the tumor cells in the tumor sample, about 61% of the tumor cells in the tumor sample, about 62% of the tumor cells in the tumor sample, about 63% of the tumor cells in the tumor sample, about 64% of the tumor cells in the tumor sample, about 65% of the tumor cells in the tumor sample, about 66% of the tumor cells in the tumor sample, about 67% of the tumor cells in the tumor sample, about 68% of the tumor cells in the tumor sample, about 69% of the tumor cells in the tumor sample, about 70% of the tumor cells in the tumor sample, about 71% of the tumor cells in the tumor sample, about 72% of the tumor cells in the tumor sample, about 73% of the tumor cells in the tumor sample, about 74% of the tumor cells in the tumor sample, about 75% of the tumor cells in the tumor sample, about 76% of the tumor cells in the tumor sample, about 77% of the tumor cells in the tumor sample, about 78% of the tumor cells in the tumor sample, about 79% of the tumor cells in the tumor sample, about 80% of the tumor cells in the tumor sample, about 81% of the tumor cells in the tumor sample, about 82% of the tumor cells in the tumor sample, about 83% of the tumor cells in the tumor sample, about 84% of the tumor cells in the tumor sample, about 85% of the tumor cells in the tumor sample, about 86% of the tumor cells in the tumor sample, about 87% of the tumor cells in the tumor sample, about 88% of the tumor cells in the tumor sample, about 89% of the tumor cells in the tumor sample, about 90% of the tumor cells in the tumor sample, about 91% of the tumor cells in the tumor sample, about 92% of the tumor cells in the tumor sample, about 93% of the tumor cells in the tumor sample, about 94% of the tumor cells in the tumor sample, about 95% of the tumor cells in the tumor sample, about 96% of the tumor cells in the tumor sample, about 97% of the tumor cells in the tumor sample, about 98% of the tumor cells in the tumor sample, about 99% of the tumor cells in the tumor sample, or about 100% of the tumor cells in the tumor sample).

In some embodiments, the tumor sample obtained from the subject has been determined to have a detectable expression level of PD-L1 in tumor-infiltrating immune cells that comprise 5% or more of the tumor sample. For example, in some embodiments the tumor sample obtained from the subject has been determined to have a detectable expression level of PD-L1 in tumor-infiltrating immune cells that comprise from about 5% to about 100% of the tumor sample, from about 6% to about 100% of the tumor sample, from about 7% to about 100% of the tumor sample, from about 8% to about 100% of the tumor sample, from about 9% to about 100% of the tumor sample, from about 10% to about 100% of the tumor sample, from about 20% to about 100% of the tumor sample, from about 30% to about 100% of the tumor sample, from about 40% to about 100% of the tumor sample, from about 50% to about 100% of the tumor sample, from about 60% to about 100% of the tumor sample, from about 70% to about 100% of the tumor sample, from about 80% to about 100% of the tumor sample, or from about 90% to about 100% of the tumor sample (e.g., about 10% of the tumor sample, about 11% of the tumor sample, about 12% of the tumor sample, about 13% of the tumor sample, about 14% of the tumor sample, about 15% of the tumor sample, about 16% of the tumor sample, about 17% of the tumor sample, about 18% of the tumor sample, about 19% of the tumor sample, about 20% of the tumor sample, about 21% of the tumor sample, about 22% of the tumor sample, about 23% of the tumor sample, about 24% of the tumor sample, about 25% of the tumor sample, about 26% of the tumor sample, about 27% of the tumor sample, about 28% of the tumor sample, about 29% of the tumor sample, about 30% of the tumor sample, about 31% of the tumor sample, about 32% of the tumor sample, about 33% of the tumor sample, about 34% of the tumor sample, about 35% of the tumor sample, about 36% of the tumor sample, about 37% of the tumor sample, about 38% of the tumor sample, about 39% of the tumor sample, about 40% of the tumor sample, about 41% of the tumor sample, about 42% of the tumor sample, about 43% of the tumor sample, about 44% of the tumor sample, about 45% of the tumor sample, about 46% of the tumor sample, about 47% of the tumor sample, about 48% of the tumor sample, about 49% of the tumor sample, about 50% of the tumor sample, about 51% of the tumor sample, about 52% of the tumor sample, about 53% of the tumor sample, about 54% of the tumor sample, about 55% of the tumor sample, about 56% of the tumor sample, about 57% of the tumor sample, about 58% of the tumor sample, about 59% of the tumor sample, about 60% of the tumor sample, about 61% of the tumor sample, about 62% of the tumor sample, about 63% of the tumor sample, about 64% of the tumor sample, about 65% of the tumor sample, about 66% of the tumor sample, about 67% of the tumor sample, about 68% of the tumor sample, about 69% of the tumor sample, about 70% of the tumor sample, about 71% of the tumor sample, about 72% of the tumor sample, about 73% of the tumor sample, about 74% of the tumor sample, about 75% of the tumor sample, about 76% of the tumor sample, about 77% of the tumor sample, about 78% of the tumor sample, about 79% of the tumor sample, about 80% of the tumor sample, about 81% of the tumor sample, about 82% of the tumor sample, about 83% of the tumor sample, about 84% of the tumor sample, about 85% of the tumor sample, about 86% of the tumor sample, about 87% of the tumor sample, about 88% of the tumor sample, about 89% of the tumor sample, about 90% of the tumor sample, about 91% of the tumor sample, about 92% of the tumor sample, about 93% of the tumor sample, about 94% of the tumor sample, about 95% of the tumor sample, about 96% of the tumor sample, about 97% of the tumor sample, about 98% of the tumor sample, about 99% of the tumor sample, or about 100% of the tumor sample).

In some embodiments, a tumor sample obtained from the subject has been determined to have a detectable expression level of PD-L1 in from about 5% to less than 50% of the tumor cells in the tumor sample. For example, in some embodiments, the tumor sample obtained from the subject has been determined to have a detectable expression level of PD-L1 in about 5% of the tumor cells in the tumor sample, about 6% of the tumor cells in the tumor sample, about 7% of the tumor cells in the tumor sample, about 8% of the tumor cells in the tumor sample, about 9% of the tumor cells in the tumor sample, about 10% of the tumor cells in the tumor sample, about 11% of the tumor cells in the tumor sample, about 12% of the tumor cells in the tumor sample, about 13% of the tumor cells in the tumor sample, about 14% of the tumor cells in the tumor sample, about 15% of the tumor cells in the tumor sample, about 16% of the tumor cells in the tumor sample, about 17% of the tumor cells in the tumor sample, about 18% of the tumor cells in the tumor sample, about 19% of the tumor cells in the tumor sample, about 20% of the tumor cells in the tumor sample, about 21% of the tumor cells in the tumor sample, about 22% of the tumor cells in the tumor sample, about 23% of the tumor cells in the tumor sample, about 24% of the tumor cells in the tumor sample, about 25% of the tumor cells in the tumor sample, about 26% of the tumor cells in the tumor sample, about 27% of the tumor cells in the tumor sample, about 28% of the tumor cells in the tumor sample, about 29% of the tumor cells in the tumor sample, about 30% of the tumor cells in the tumor sample, about 31% of the tumor cells in the tumor sample, about 32% of the tumor cells in the tumor sample, about 33% of the tumor cells in the tumor sample, about 34% of the tumor cells in the tumor sample, about 35% of the tumor cells in the tumor sample, about 36% of the tumor cells in the tumor sample, about 37% of the tumor cells in the tumor sample, about 38% of the tumor cells in the tumor sample, about 39% of the tumor cells in the tumor sample, about 40% of the tumor cells in the tumor sample, about 41% of the tumor cells in the tumor sample, about 42% of the tumor cells in the tumor sample, about 43% of the tumor cells in the tumor sample, about 44% of the tumor cells in the tumor sample, about 45% of the tumor cells in the tumor sample, about 46% of the tumor cells in the tumor sample, about 47% of the tumor cells in the tumor sample, about 48% of the tumor cells in the tumor sample, or about 49% of the tumor cells in the tumor sample.

In some embodiments, a tumor sample obtained from the subject has been determined to have a detectable expression level of PD-L1 in tumor-infiltrating immune cells that comprise from about 5% to less than 10% of the tumor cells in the tumor sample. For example, in some embodiments, the tumor sample obtained from the subject has been determined to have a detectable expression level of PD-L1 in tumor-infiltrating immune cells that comprise about 5% of the tumor cells in the tumor sample, about 6% of the tumor cells in the tumor sample, about 7% of the tumor cells in the tumor sample, about 8% of the tumor cells in the tumor sample, or about 9% of the tumor cells in the tumor sample.

In some embodiments, a tumor sample obtained from the subject has been determined to have a detectable expression level of PD-L1 in 50% or more of the tumor cells in the tumor sample. For example, in some embodiments, the tumor sample obtained from the subject has been determined to have a detectable expression level of PD-L1 in from about 50% to about 100% of the tumor cells in the tumor sample, from about 60% to about 100% of the tumor cells in the tumor sample, from about 70% to about 100% of the tumor cells in the tumor sample, from about 80% to about 100% of the tumor cells in the tumor sample, or from about 90% to about 100% of the tumor cells in the tumor sample (e.g., about 50% of the tumor cells in the tumor sample, about 51% of the tumor cells in the tumor sample, about 52% of the tumor cells in the tumor sample, about 53% of the tumor cells in the tumor sample, about 54% of the tumor cells in the tumor sample, about 55% of the tumor cells in the tumor sample, about 56% of the tumor cells in the tumor sample, about 57% of the tumor cells in the tumor sample, about 58% of the tumor cells in the tumor sample, about 59% of the tumor cells in the tumor sample, about 60% of the tumor cells in the tumor sample, about 61% of the tumor cells in the tumor sample, about 62% of the tumor cells in the tumor sample, about 63% of the tumor cells in the tumor sample, about 64% of the tumor cells in the tumor sample, about 65% of the tumor cells in the tumor sample, about 66% of the tumor cells in the tumor sample, about 67% of the tumor cells in the tumor sample, about 68% of the tumor cells in the tumor sample, about 69% of the tumor cells in the tumor sample, about 70% of the tumor cells in the tumor sample, about 71% of the tumor cells in the tumor sample, about 72% of the tumor cells in the tumor sample, about 73% of the tumor cells in the tumor sample, about 74% of the tumor cells in the tumor sample, about 75% of the tumor cells in the tumor sample, about 76% of the tumor cells in the tumor sample, about 77% of the tumor cells in the tumor sample, about 78% of the tumor cells in the tumor sample, about 79% of the tumor cells in the tumor sample, about 80% of the tumor cells in the tumor sample, about 81% of the tumor cells in the tumor sample, about 82% of the tumor cells in the tumor sample, about 83% of the tumor cells in the tumor sample, about 84% of the tumor cells in the tumor sample, about 85% of the tumor cells in the tumor sample, about 86% of the tumor cells in the tumor sample, about 87% of the tumor cells in the tumor sample, about 88% of the tumor cells in the tumor sample, about 89% of the tumor cells in the tumor sample, about 90% of the tumor cells in the tumor sample, about 91% of the tumor cells in the tumor sample, about 92% of the tumor cells in the tumor sample, about 93% of the tumor cells in the tumor sample, about 94% of the tumor cells in the tumor sample, about 95% of the tumor cells in the tumor sample, about 96% of the tumor cells in the tumor sample, about 97% of the tumor cells in the tumor sample, about 98% of the tumor cells in the tumor sample, about 99% of the tumor cells in the tumor sample, or about 100% of the tumor cells in the tumor sample).

In some embodiments, the tumor sample obtained from the subject has been determined to have a detectable expression level of PD-L1 in tumor-infiltrating immune cells that comprise 10% or more of the tumor sample. For example, in some embodiments the tumor sample obtained from the subject has been determined to have a detectable expression level of PD-L1 in tumor-infiltrating immune cells that comprise from about 10% to about 100% of the tumor sample, from about 20% to about 100% of the tumor sample, from about 30% to about 100% of the tumor sample, from about 40% to about 100% of the tumor sample, from about 50% to about 100% of the tumor sample, from about 60% to about 100% of the tumor sample, from about 70% to about 100% of the tumor sample, from about 80% to about 100% of the tumor sample, or from about 90% to about 100% of the tumor sample (e.g., about 10% of the tumor sample, about 11% of the tumor sample, about 12% of the tumor sample, about 13% of the tumor sample, about 14% of the tumor sample, about 15% of the tumor sample, about 16% of the tumor sample, about 17% of the tumor sample, about 18% of the tumor sample, about 19% of the tumor sample, about 20% of the tumor sample, about 21% of the tumor sample, about 22% of the tumor sample, about 23% of the tumor sample, about 24% of the tumor sample, about 25% of the tumor sample, about 26% of the tumor sample, about 27% of the tumor sample, about 28% of the tumor sample, about 29% of the tumor sample, about 30% of the tumor sample, about 31% of the tumor sample, about 32% of the tumor sample, about 33% of the tumor sample, about 34% of the tumor sample, about 35% of the tumor sample, about 36% of the tumor sample, about 37% of the tumor sample, about 38% of the tumor sample, about 39% of the tumor sample, about 40% of the tumor sample, about 41% of the tumor sample, about 42% of the tumor sample, about 43% of the tumor sample, about 44% of the tumor sample, about 45% of the tumor sample, about 46% of the tumor sample, about 47% of the tumor sample, about 48% of the tumor sample, about 49% of the tumor sample, about 50% of the tumor sample, about 51% of the tumor sample, about 52% of the tumor sample, about 53% of the tumor sample, about 54% of the tumor sample, about 55% of the tumor sample, about 56% of the tumor sample, about 57% of the tumor sample, about 58% of the tumor sample, about 59% of the tumor sample, about 60% of the tumor sample, about 61% of the tumor sample, about 62% of the tumor sample, about 63% of the tumor sample, about 64% of the tumor sample, about 65% of the tumor sample, about 66% of the tumor sample, about 67% of the tumor sample, about 68% of the tumor sample, about 69% of the tumor sample, about 70% of the tumor sample, about 71% of the tumor sample, about 72% of the tumor sample, about 73% of the tumor sample, about 74% of the tumor sample, about 75% of the tumor sample, about 76% of the tumor sample, about 77% of the tumor sample, about 78% of the tumor sample, about 79% of the tumor sample, about 80% of the tumor sample, about 81% of the tumor sample, about 82% of the tumor sample, about 83% of the tumor sample, about 84% of the tumor sample, about 85% of the tumor sample, about 86% of the tumor sample, about 87% of the tumor sample, about 88% of the tumor sample, about 89% of the tumor sample, about 90% of the tumor sample, about 91% of the tumor sample, about 92% of the tumor sample, about 93% of the tumor sample, about 94% of the tumor sample, about 95% of the tumor sample, about 96% of the tumor sample, about 97% of the tumor sample, about 98% of the tumor sample, about 99% of the tumor sample, or about 100% of the tumor sample).

PD-L1 expression may be determined, for example, using immunohistochemistry (IHC), among other techniques described herein. In some embodiments, PD-L1 expression is detected using an anti-PD-L1 antibody. Any suitable anti-PD-L1 antibody may be used, including, e.g., SP142, SP263, 22C3, 28-8, El L3N, 4059, h5H1, and 9A11. In some embodiments, the anti-PD-L1 antibody is SP142. In some embodiments, the anti-PD-L1 antibody is SP263. In some embodiments, the anti-PD-L1 antibody is 22C3.

In some embodiments, the subject is a human, such as a human that (i) does not have a sensitizing mutation in a gene encoding epidermal growth factor receptor (EGFR) and/or (ii) does not have an anaplastic lymphoma receptor tyrosine kinase (ALK) fusion oncogene. For example, the subject may be a human that has no EGFR or ALK genomic tumor aberrations.

In a further aspect, the disclosure features a method of treating squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, and wherein a tumor sample obtained from the subject has been determined to have:
(i) a detectable expression level of PD-L1 in 1% or more of the tumor cells in the tumor sample; and/or
(ii) a detectable expression level of PD-L1 in tumor-infiltrating immune cells that comprise 1% or more of the tumor sample.

In a further aspect, the disclosure features a method of treating squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 840 mg every 2 weeks, about 1200 mg every 3 weeks, or about 1680 mg every 4 weeks, and wherein a tumor sample obtained from the subject has been determined to have:
(i) a detectable expression level of PD-L1 in 1% or more of the tumor cells in the tumor sample; and/or
(ii) a detectable expression level of PD-L1 in tumor-infiltrating immune cells that comprise 1% or more of the tumor sample.

In a further aspect, the disclosure features a method of treating squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in 1% or more of the tumor cells in the tumor sample; and/or
(ii) a detectable expression level of PD-L1 in tumor-infiltrating immune cells that comprise 1% or more of the tumor sample.

In a further aspect, the disclosure features a method of treating stage IV squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in 1% or more of the tumor cells in the tumor sample; and/or
(ii) a detectable expression level of PD-L1 in tumor-infiltrating immune cells that comprise 1% or more of the tumor sample.

In a further aspect, the disclosure features a method of treating stage IV squamous NSCLC in a human subject in need thereof, the method comprising administering to the subject an effective amount of atezolizumab during one or more dosing cycles, wherein the subject is chemotherapy-naïve, wherein the subject does not have a sensitizing mutation in a gene encoding EGFR and does not have an ALK fusion oncogene, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in 1% or more of the tumor cells in the tumor sample; and/or
(ii) a detectable expression level of PD-L1 in tumor-infiltrating immune cells that comprise 1% or more of the tumor sample.

In a further aspect, the disclosure features a method of treating squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, and wherein a tumor sample obtained from the subject has been determined to have:
(i) a detectable expression level of PD-L1 in from 1% to less than 5% of the tumor cells in the tumor sample; and/or
(ii) a detectable expression level of PD-L1 in tumor-infiltrating immune cells that comprise from 1% to less than 5% of the tumor sample.

In a further aspect, the disclosure features a method of treating squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 840 mg every 2 weeks, about 1200 mg every 3 weeks, or about 1680 mg every 4 weeks, and wherein a tumor sample obtained from the subject has been determined to have:
(i) a detectable expression level of PD-L1 in from 1% to less than 5% of the tumor cells in the tumor sample; and/or
(ii) a detectable expression level of PD-L1 in tumor-infiltrating immune cells that comprise from 1% to less than 5% of the tumor sample.

In a further aspect, the disclosure features a method of treating squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in from 1% to less than 5% of the tumor cells in the tumor sample; and/or
(ii) a detectable expression level of PD-L1 in tumor-infiltrating immune cells that comprise from 1% to less than 5% of the tumor sample.

In a further aspect, the disclosure features a method of treating stage IV squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in from 1% to less than 5% of the tumor cells in the tumor sample; and/or
(ii) a detectable expression level of PD-L1 in tumor-infiltrating immune cells that comprise from 1% to less than 5% of the tumor sample.

In a further aspect, the disclosure features a method of treating stage IV squamous NSCLC in a human subject in need thereof, the method comprising administering to the subject an effective amount of atezolizumab during one or more dosing cycles, wherein the subject is chemotherapy-naïve, wherein the subject does not have a sensitizing mutation in a gene encoding EGFR and does not have an ALK fusion oncogene, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in from 1% to less than 5% of the tumor cells in the tumor sample; and/or
(ii) a detectable expression level of PD-L1 in tumor-infiltrating immune cells that comprise from 1% to less than 5% of the tumor sample.

In a further aspect, the disclosure features a method of treating squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, and wherein a tumor sample obtained from the subject has been determined to have:
(i) a detectable expression level of PD-L1 in 5% or more of the tumor cells in the tumor sample; and/or
(ii) a detectable expression level of PD-L1 in tumor-infiltrating immune cells that comprise 5% or more of the tumor sample.

In a further aspect, the disclosure features a method of treating squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 840 mg every 2 weeks, about 1200 mg every 3 weeks, or about 1680 mg every 4 weeks, and wherein a tumor sample obtained from the subject has been determined to have:
(i) a detectable expression level of PD-L1 in 5% or more of the tumor cells in the tumor sample; and/or
(ii) a detectable expression level of PD-L1 in tumor-infiltrating immune cells that comprise 5% or more of the tumor sample.

In a further aspect, the disclosure features a method of treating squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in 5% or more of the tumor cells in the tumor sample; and/or
(ii) a detectable expression level of PD-L1 in tumor-infiltrating immune cells that comprise 5% or more of the tumor sample.

In a further aspect, the disclosure features a method of treating stage IV squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in 5% or more of the tumor cells in the tumor sample; and/or
(ii) a detectable expression level of PD-L1 in tumor-infiltrating immune cells that comprise 5% or more of the tumor sample.

In a further aspect, the disclosure features a method of treating stage IV squamous NSCLC in a human subject in need thereof, the method comprising administering to the subject an effective amount of atezolizumab during one or more dosing cycles, wherein the subject is chemotherapy-naïve, wherein the subject does not have a sensitizing mutation in a gene encoding EGFR and does not have an ALK fusion oncogene, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in 5% or more of the tumor cells in the tumor sample; and/or
(ii) a detectable expression level of PD-L1 in tumor-infiltrating immune cells that comprise 5% or more of the tumor sample.

In a further aspect, the disclosure features a method of treating squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, and wherein a tumor sample obtained from the subject has been determined to have:
(i) a detectable expression level of PD-L1 in from 5% to less than 50% of the tumor cells in the tumor sample; and/or
(ii) a detectable expression level of PD-L1 in tumor-infiltrating immune cells that comprise from 5% to less than 10% of the tumor sample.

In a further aspect, the disclosure features a method of treating squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 840 mg every 2 weeks, about 1200 mg every 3 weeks, or about 1680 mg every 4 weeks, and wherein a tumor sample obtained from the subject has been determined to have:
(i) a detectable expression level of PD-L1 in from 5% to less than 50% of the tumor cells in the tumor sample; and/or
(ii) a detectable expression level of PD-L1 in tumor-infiltrating immune cells that comprise from 5% to less than 10% of the tumor sample.

In a further aspect, the disclosure features a method of treating squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in from 5% to less than 50% of the tumor cells in the tumor sample; and/or
(ii) a detectable expression level of PD-L1 in tumor-infiltrating immune cells that comprise from 5% to less than 10% of the tumor sample.

In a further aspect, the disclosure features a method of treating stage IV squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in from 5% to less than 50% of the tumor cells in the tumor sample; and/or
(ii) a detectable expression level of PD-L1 in tumor-infiltrating immune cells that comprise from 5% to less than 10% of the tumor sample.

In a further aspect, the disclosure features a method of treating stage IV squamous NSCLC in a human subject in need thereof, the method comprising administering to the subject an effective amount of atezolizumab during one or more dosing cycles, wherein the subject is chemotherapy-naïve, wherein the subject does not have a sensitizing mutation in a gene encoding EGFR and does not have an ALK fusion oncogene, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in from 5% to less than 50% of the tumor cells in the tumor sample; and/or
(ii) a detectable expression level of PD-L1 in tumor-infiltrating immune cells that comprise from 5% to less than 10% of the tumor sample.

In a further aspect, the disclosure features a method of treating squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, and wherein a tumor sample obtained from the subject has been determined to have:
(i) a detectable expression level of PD-L1 in 50% or more of the tumor cells in the tumor sample; and/or
(ii) a detectable expression level of PD-L1 in tumor-infiltrating immune cells that comprise 10% or more of the tumor sample.

In a further aspect, the disclosure features a method of treating squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 840 mg every 2 weeks, about 1200 mg every 3 weeks, or about 1680 mg every 4 weeks, and wherein a tumor sample obtained from the subject has been determined to have:
(i) a detectable expression level of PD-L1 in 50% or more of the tumor cells in the tumor sample; and/or
(ii) a detectable expression level of PD-L1 in tumor-infiltrating immune cells that comprise 10% or more of the tumor sample.

In a further aspect, the disclosure features a method of treating squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in 50% or more of the tumor cells in the tumor sample; and/or
(ii) a detectable expression level of PD-L1 in tumor-infiltrating immune cells that comprise 10% or more of the tumor sample.

In a further aspect, the disclosure features a method of treating stage IV squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in 50% or more of the tumor cells in the tumor sample; and/or
(ii) a detectable expression level of PD-L1 in tumor-infiltrating immune cells that comprise 10% or more of the tumor sample.

In a further aspect, the disclosure features a method of treating stage IV squamous NSCLC in a human subject in need thereof, the method comprising administering to the subject an effective amount of atezolizumab during one or more dosing cycles, wherein the subject is chemotherapy-naïve, wherein the subject does not have a sensitizing mutation in a gene encoding EGFR and does not have an ALK fusion oncogene, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in 50% or more of the tumor cells in the tumor sample; and/or
(ii) a detectable expression level of PD-L1 in tumor-infiltrating immune cells that comprise 10% or more of the tumor sample.

In a further aspect, the disclosure features a PD-1 axis binding antagonist for use in accordance with the method of any of the above aspects or embodiments of the disclosure.

In another aspect, the disclosure features a use of a PD-1 axis binding antagonist in the manufacture of a medicament for treating squamous NSCLC in accordance with the method of any of the above aspects or embodiments of the disclosure.

In another aspect, the disclosure features a kit containing a PD-1 axis binding antagonist and a package insert instructing a user of the kit to administer the PD-1 axis binding antagonist to a subject in accordance with the method of any of the above aspects or embodiments of the disclosure.

In a further aspect, the disclosure features a method of identifying a subject having non-squamous NSCLC who may benefit from a treatment comprising a PD-1 axis binding antagonist. The method may include, for example, determining a blood tumor mutational burden (bTMB) score from a sample from the subject, wherein a bTMB score from the sample that is at or above a reference bTMB score identifies the subject as one who may benefit from a treatment comprising a PD-1 axis binding antagonist.

In another aspect, the disclosure features a method of selecting a therapy for a subject having non-squamous NSCLC. The method may include, for example, determining a bTMB score from a sample from the subject, wherein a bTMB score from the sample that is at or above a reference bTMB score identifies the subject as one who may benefit from a treatment comprising a PD-1 axis binding antagonist.

In some embodiments, the bTMB score determined from the sample is at or above the reference bTMB score. In such instances, the method may further include administering to the subject an effective amount of a PD-1 axis binding antagonist. In some embodiments, the bTMB score determined from the sample is below the reference bTMB score. In such instances, the method may include administering to the subject an effective amount of a therapy that does not contain a PD-1 axis binding antagonist.

In another aspect, the disclosure features a method of treating non-squamous NSCLC in a subject in need thereof. The method may include:
(a) determining a bTMB score from a sample from the subject, wherein the bTMB score determined from the sample is at or above a reference bTMB score; and
(b) administering to the subject an effective amount of a PD-1 axis binding antagonist.

In another aspect, the disclosure features a method of treating non-squamous NSCLC in a subject in need thereof by administering to the subject an effective amount of a PD-1 axis binding antagonist, wherein, prior to administration of the PD-1 axis binding antagonist to the subject, a bTMB score from a sample from the subject has been determined to be at or above a reference bTMB score.

In some embodiments of any of the foregoing aspects of the disclosure, the reference bTMB score is a bTMB score in a reference population. The reference population may be, for example, a population of subjects having non-squamous NSCLC. The population of subjects having non-squamous NSCLC may include a first subset of subjects who have been treated with a PD-1 axis binding antagonist and a second subset of subjects who have been treated with therapy that does not comprise a PD-1 axis binding antagonist. In such instances, the reference bTMB score may significantly separate each of the first and second subsets of subjects based on a significant difference between a subject's responsiveness to treatment with the PD-1 axis binding antagonist and a subject's responsiveness to treatment with the therapy that does not comprise a PD-1 axis binding antagonist at or above the reference bTMB score, wherein the subject's responsiveness to treatment with the PD-1 axis binding antagonist is significantly improved relative to the subject's responsiveness to treatment with the therapy that does not comprise a PD-1 axis binding antagonist. In some embodiments, the reference bTMB score significantly separates each of the first and second subsets of subjects based on a significant difference between a subject's responsiveness to treatment with the PD-1 axis binding antagonist and a subject's responsiveness to treatment with the therapy that does not comprise a PD-1 axis binding antagonist below the bTMB score, wherein the subject's responsiveness to treatment with the therapy that does not comprise a PD-1 axis binding antagonist is significantly improved relative to the subject's responsiveness to treatment with the PD-1 axis binding antagonist. In some embodiments, the therapy that does not comprise a PD-1 axis binding antagonist comprises an anti-neoplastic agent, a chemotherapeutic agent, a growth inhibitory agent, an anti-angiogenic agent, a radiation therapy, a cytotoxic agent, or a combination thereof. For example, in some embodiments, the therapy that does not comprise a PD-1 axis binding antagonist comprises a chemotherapeutic agent. In the context of any of the foregoing embodiments, the responsiveness to treatment may include an increase in progression-free survival (PFS) and/or an increase in overall survival (OS).

In some embodiments, the bTMB score from the sample has a prevalence of greater than, or equal to, about 5% in the reference population. In some embodiments, the bTMB score from the sample has a prevalence of from about 5% to about 75% in the reference population. In some embodiments, the bTMB score from the sample has a prevalence of from about 20% to about 30% in the reference population.

In some embodiments of any of the above aspects or embodiments of the disclosure, the reference bTMB score is a pre-assigned bTMB score. For example, the reference bTMB score may be from 4 to 30 (e.g., a reference bTMB score of 4, 5, 6, 7, 8, 9, 10, 11, 12, 13, 14, 15, 16, 17, 18, 19, 20, 21, 22, 23, 24, 25, 26, 27, 28, 29, or 30). In some embodiments, the reference bTMB score is from 5 to 58 (e.g., a reference bTMB score of 5, 6, 7, 8, 9, 10, 11, 12, 13, 14, 15, 16, 17, 18, 19, 20, 21, 22, 23, 24, 25, 26, 27, or 28). In some embodiments, the reference bTMB score is from 6 to 26 (e.g., a reference bTMB score of 6, 7, 8, 9, 10, 11, 12, 13, 14, 15, 16, 17, 18, 19, 20, 21, 22, 23, 24, 25, or 26). In some embodiments, the reference bTMB score is from 7 to 24 (e.g., a reference bTMB score of 7, 8, 9, 10, 11, 12, 13, 14, 15, 16, 17, 18, 19, 20, 21, 22, 23, or 24). In some embodiments, the reference bTMB score is from 8 to 22 (e.g., a reference bTMB score of 8, 9, 10, 11, 12, 13, 14, 15, 16, 17, 18, 19, 20, 21, or 22).

For example, in some embodiments of any of the above aspects or embodiments of the disclosure, the reference bTMB score is from 7 to 13 (e.g., a reference bTMB score of 7, 8, 9, 10, 11, 12, or 13). In some embodiments, the reference bTMB score is from 8 to 12 (e.g., a reference bTMB score of 8, 9, 10, 11, or 12). In some embodiments, the reference bTMB score is from 9 to 11 (e.g., a reference bTMB score of 9, 10, or 11). In some embodiments, the reference bTMB score is 10.

In some embodiments of any of the above aspects or embodiments of the disclosure, the reference bTMB score is from 13 to 19 (e.g., a reference bTMB score of 13, 14, 15, 16, 17, 18, or 19). In some embodiments, the reference bTMB score is from 14 to 18 (e.g., a reference bTMB score of 14, 15, 16, 17, or 18). In some embodiments, the reference bTMB score is from 5 to 17 (e.g., a reference bTMB score of 15, 16, or 17). In some embodiments, the reference bTMB score is 16.

In some embodiments of any of the above aspects or embodiments of the disclosure, the reference bTMB score is from 16 to 24 (e.g., a reference bTMB score of 16, 17, 18, 19, 20, 21, 22, 23, or 24). In some embodiments, the reference bTMB score is from 17 to 23 (e.g., a reference bTMB score of 17, 18, 19, 20, 21, 22, or 23). In some embodiments, the reference bTMB score is from 18 to 22 (e.g., a reference bTMB score of 18, 19, 20, 21, or 22). In some embodiments, the reference bTMB score is from 19 to 21 (e.g., a reference bTMB score of 19, 20, or 21). In some embodiments, the reference bTMB score is 20.

In some embodiments of any of the above aspects or embodiments of the disclosure, the bTMB score determined from the sample is greater than, or equal to, 4. For example, the reference bTMB score may be 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, or more.

For example, in some embodiments, the reference bTMB score is from 4 to 100 (e.g., a reference bTMB score of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100).

In some embodiments, the reference bTMB score is greater than, or equal to, 6. For example, the reference bTMB score may be 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, or more.

In some embodiments, the reference bTMB score is from 6 to 100 (e.g., a reference bTMB score of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100).

In some embodiments, the reference bTMB score is greater than, or equal to, 8. For example, the reference bTMB score may be 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, or more.

In some embodiments, the reference bTMB score is from 8 to 100 (e.g., a reference bTMB score of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100).

In some embodiments, the reference bTMB score is greater than, or equal to, 10. For example, the reference bTMB score may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, or more.

In some embodiments, the reference bTMB score is from 10 to 100 (e.g., a reference bTMB score of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100).

In some embodiments, the reference bTMB score is greater than, or equal to, 12. For example, the reference bTMB score may be 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, or more.

In some embodiments, the reference bTMB score is from 12 to 100 (e.g., a reference bTMB score of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100).

In some embodiments, the reference bTMB score is greater than, or equal to, 14. For example, the reference bTMB score may be 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, or more.

In some embodiments, the reference bTMB score is from 14 to 100 (e.g., a reference bTMB score of 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100).

In some embodiments, the reference bTMB score is greater than, or equal to, 16. For example, the reference bTMB score may be 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, or more.

In some embodiments, the reference bTMB score is from 16 to 100 (e.g., a reference bTMB score of 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100).

In some embodiments, the reference bTMB score is greater than, or equal to, 18. For example, the reference bTMB score may be 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, or more.

In some embodiments, the reference bTMB score is from 18 to 100 (e.g., a reference bTMB score of 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100).

In some embodiments, the reference bTMB score is greater than, or equal to, 20. For example, the reference bTMB score may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, or more.

In some embodiments, the reference bTMB score is from 20 to 100 (e.g., a reference bTMB score of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100).

In some embodiments of any of the foregoing aspects or embodiments of the disclosure, the bTMB score determined from the sample is represented as the number of somatic mutations counted over a defined number of sequenced bases, as assessed, for example, using the FOUNDATIONONE CDX™ panel or the FOUNDATIONONE® panel. For example, in some embodiments, the defined number of sequenced bases used in the calculation of the bTMB score determined from the sample is from about 100 kb to about 10 Mb (e.g., the defined number of sequenced bases may be 100 kb, 150 kb, 200 kb, 250 kb, 300 kb, 350 kb, 400 kb, 450 kb, 500 kb, 550 kb, 600 kb, 650 kb, 700 kb, 750 kb, 800 kb, 850 kb, 900 kb, 950 kb, 1.0 Mb, 1.2 Mb, 1.3 Mb, 1.4 Mb, 1.5 Mb, 1.6 Mb, 1.7 Mb, 1.8 Mb, 1.9 Mb, 2.0 Mb, 2.1 Mb, 2.2 Mb, 2.3 Mb, 2.4 Mb, 2.5 Mb, 2.6 Mb, 2.7 Mb, 2.8 Mb, 2.9 Mb, 3.0 Mb, 3.1 Mb, 3.2 Mb, 3.3 Mb, 3.4 Mb, 3.5 Mb, 3.6 Mb, 3.7 Mb, 3.8 Mb, 3.9 Mb, 4.0 Mb, 4.1 Mb, 4.2 Mb, 4.3 Mb, 4.4 Mb, 4.5 Mb, 4.6 Mb, 4.7 Mb, 4.8 Mb, 4.9 Mb, 5.0 Mb, 5.1 Mb, 5.2 Mb, 5.3 Mb, 5.4 Mb, 5.5 Mb, 5.6 Mb, 5.7 Mb, 5.8 Mb, 5.9 Mb, 6.0 Mb, 6.1 Mb, 6.2 Mb, 6.3 Mb, 6.4 Mb, 6.5 Mb, 6.6 Mb, 6.7 Mb, 6.8 Mb, 6.9 Mb, 7.0 Mb, 7.1 Mb, 7.2 Mb, 7.3 Mb, 7.4 Mb, 7.5 Mb, 7.6 Mb, 7.7 Mb, 7.8 Mb, 7.9 Mb, 8.0 Mb, 8.1 Mb, 8.2 Mb, 8.3 Mb, 8.4 Mb, 8.5 Mb, 8.6 Mb, 8.7 Mb, 8.8 Mb, 8.9 Mb, 9.0 Mb, 9.1 Mb, 9.2 Mb, 9.3 Mb, 9.4 Mb, 9.5 Mb, 9.6 Mb, 9.7 Mb, 9.8 Mb, 9.9 Mb, or 10.0

Mb). In some embodiments, the defined number of sequenced bases used in the calculation of the bTMB score determined from the sample is from about 0.5 Mb to about 1.5 Mb (e.g., the defined number of sequenced bases may be 500 kb, 550 kb, 600 kb, 650 kb, 700 kb, 750 kb, 800 kb, 850 kb, 900 kb, 950 kb, 1.0 Mb, 1.2 Mb, 1.3 Mb, 1.4 Mb, or 1.5 Mb). In some embodiments, the defined number of sequenced bases used in the calculation of the bTMB score determined from the sample is from about 0.7 Mb to about 1.3 Mb (e.g., the defined number of sequenced bases may be 700 kb, 750 kb, 800 kb, 850 kb, 900 kb, 950 kb, 1.0 Mb, 1.2 Mb, or 1.3 Mb). In some embodiments, the defined number of sequenced bases used in the calculation of the bTMB score determined from the sample is from about 0.8 Mb to about 1.2 Mb (e.g., the defined number of sequenced bases may be 800 kb, 850 kb, 900 kb, 950 kb, 1.0 Mb, or 1.2 Mb). In some embodiments, the defined number of sequenced bases used in the calculation of the bTMB score determined from the sample is about 1.1 Mb. The number of somatic mutations used in the calculation of the bTMB score determined from the sample may be, for example, (i) the number of single nucleotide variants (SNVs) counted or (ii) a sum of the number of SNVs counted and the number of indel mutations counted. In some embodiments, the number of somatic mutations used in the calculation of the bTMB score determined from the sample is the number of SNVs counted. In some embodiments, the number of somatic mutations used in the calculation of the bTMB score determined from the sample is the number of synonymous and non-synonymous SNVs and/or indels. In some embodiments, the bTMB score determined from the sample is an equivalent bTMB value, for example, as determined by whole-exome sequencing. In some embodiments, the somatic mutations used in the calculation of the bTMB score determined from the sample are counted in one or more genes set forth in Table 1.

In some embodiments of any of the foregoing aspects or embodiments of the disclosure, the reference bTMB score is represented as the number of somatic mutations counted over a defined number of sequenced bases, as assessed, for example, using the FOUNDATIONONE CDX™ panel or the FOUNDATIONONE® panel. In some embodiments, the defined number of sequenced bases used in the calculation of the reference bTMB score is from about 100 kb to about 10 Mb (e.g., the defined number of sequenced bases may be 100 kb, 150 kb, 200 kb, 250 kb, 300 kb, 350 kb, 400 kb, 450 kb, 500 kb, 550 kb, 600 kb, 650 kb, 700 kb, 750 kb, 800 kb, 850 kb, 900 kb, 950 kb, 1.0 Mb, 1.2 Mb, 1.3 Mb, 1.4 Mb, 1.5 Mb, 1.6 Mb, 1.7 Mb, 1.8 Mb, 1.9 Mb, 2.0 Mb, 2.1 Mb, 2.2 Mb, 2.3 Mb, 2.4 Mb, 2.5 Mb, 2.6 Mb, 2.7 Mb, 2.8 Mb, 2.9 Mb, 3.0 Mb, 3.1 Mb, 3.2 Mb, 3.3 Mb, 3.4 Mb, 3.5 Mb, 3.6 Mb, 3.7 Mb, 3.8 Mb, 3.9 Mb, 4.0 Mb, 4.1 Mb, 4.2 Mb, 4.3 Mb, 4.4 Mb, 4.5 Mb, 4.6 Mb, 4.7 Mb, 4.8 Mb, 4.9 Mb, 5.0 Mb, 5.1 Mb, 5.2 Mb, 5.3 Mb, 5.4 Mb, 5.5 Mb, 5.6 Mb, 5.7 Mb, 5.8 Mb, 5.9 Mb, 6.0 Mb, 6.1 Mb, 6.2 Mb, 6.3 Mb, 6.4 Mb, 6.5 Mb, 6.6 Mb, 6.7 Mb, 6.8 Mb, 6.9 Mb, 7.0 Mb, 7.1 Mb, 7.2 Mb, 7.3 Mb, 7.4 Mb, 7.5 Mb, 7.6 Mb, 7.7 Mb, 7.8 Mb, 7.9 Mb, 8.0 Mb, 8.1 Mb, 8.2 Mb, 8.3 Mb, 8.4 Mb, 8.5 Mb, 8.6 Mb, 8.7 Mb, 8.8 Mb, 8.9 Mb, 9.0 Mb, 9.1 Mb, 9.2 Mb, 9.3 Mb, 9.4 Mb, 9.5 Mb, 9.6 Mb, 9.7 Mb, 9.8 Mb, 9.9 Mb, or 10.0 Mb). In some embodiments, the defined number of sequenced bases used in the calculation of the reference bTMB score is from about 0.5 Mb to about 1.5 Mb (e.g., the defined number of sequenced bases may be 500 kb, 550 kb, 600 kb, 650 kb, 700 kb, 750 kb, 800 kb, 850 kb, 900 kb, 950 kb, 1.0 Mb, 1.2 Mb, 1.3 Mb, 1.4 Mb, or 1.5 Mb). In some embodiments, the defined number of sequenced bases used in the calculation of the reference bTMB score is from about 0.7 Mb to about 1.3 Mb (e.g., the defined number of sequenced bases may be 700 kb, 750 kb, 800 kb, 850 kb, 900 kb, 950 kb, 1.0 Mb, 1.2 Mb, or 1.3 Mb). In some embodiments, the defined number of sequenced bases used in the calculation of the reference bTMB score is from about 0.8 Mb to about 1.2 Mb (e.g., the defined number of sequenced bases may be 800 kb, 850 kb, 900 kb, 950 kb, 1.0 Mb, or 1.2 Mb). In some embodiments, the defined number of sequenced bases used in the calculation of the reference bTMB score is about 1.1 Mb. The number of somatic mutations used in the calculation of the reference bTMB score may be, for example, (i) the number of single nucleotide variants (SNVs) counted or (ii) a sum of the number of SNVs counted and the number of indel mutations counted. In some embodiments, the number of somatic mutations used in the calculation of the reference bTMB score is the number of SNVs counted. In some embodiments, the number of somatic mutations used in the calculation of the reference bTMB score is the number of synonymous and non-synonymous SNVs and/or indels. In some embodiments, the reference bTMB score is an equivalent bTMB value, for example, as determined by whole-exome sequencing. In some embodiments, the somatic mutations used in the calculation of the reference bTMB score are counted in one or more genes set forth in Table 1.

In some embodiments of any of the above aspects or embodiments of the disclosure, the method further comprises determining a maximum somatic allele frequency (MSAF) from a sample obtained from the subject. The MSAF may be, for example, greater than, or equal to, 1%. In some embodiments of any of the above aspects or embodiments of the disclosure, prior to being administered a PD-1 axis binding antagonist, a sample obtained from the subject has been determined to have an MSAF of greater than, or equal to, 1%.

In some embodiments, the method further comprises determining an MSAF from a sample obtained from the subject, wherein the MSAF from the sample is less than 1%. In some embodiments, prior to being administered a PD-1 axis binding antagonist, a sample obtained from the subject has been determined to have an MSAF of less than 1%.

In some embodiments, the method further comprises determining an MSAF from a sample obtained from the subject, wherein the MSAF from the sample has been determined to be greater than, or equal to, 1%, and the method further comprises administering to the individual an effective amount of an anti-cancer therapy other than, or in addition to, a PD-1 axis binding antagonist.

In some embodiments, the method further comprises determining an MSAF from a sample obtained from the subject, wherein the MSAF from the sample has been determined to be less than 1%, and the method further comprises administering an effective amount of a PD-1 axis binding antagonist to the individual.

In some embodiments, benefit from treatment containing a PD-1 axis binding antagonist includes an increase in OS. In some embodiments, benefit from treatment containing a PD-1 axis binding antagonist includes an increase in PFS.

In some embodiments, administration of the PD-1 axis binding antagonist to the subject extends the subject's OS as compared to administration of a platinum-based chemotherapy without the PD-1 axis binding antagonist. For example, administration of the PD-1 axis binding antagonist to the subject may extend the subject's OS by from about 4 months to about 10 months, by from about 5 months to about 9 months, by from about 6 months to about 8 months, by from about 6.5 months to about 7.5 months, or by from about 6.8 months to about 7.4 months as compared to administration of the platinum-based chemotherapy without the PD-1 axis binding antagonist (e.g., by about 4 months, 4.1 months, 4.2 months, 4.3 months, 4.4 months, 4.5 months, 4.6 months, 4.7 months, 4.8 months, 4.9 months, 5 months, 5.1 months, 5.2 months, 5.3 months, 5.4 months, 5.5 months, 5.6 months, 5.7 months, 5.8 months, 5.9 months, 6 months, 6.1 months, 6.2 months, 6.3 months, 6.4 months, 6.5 months, 6.6 months, 6.7 months, 6.8 months, 6.9 months, 7 months, 7.1 months, 7.2 months, 7.3 months, 7.4 months, 7.5 months, 7.6 months, 7.7 months, 7.8 months, 7.9 months, 8 months, 8.1 months, 8.2 months, 8.3 months, 8.4 months, 8.5 months, 8.6 months, 8.7 months, 8.8 months, 8.9 months, 9 months, 9.1 months, 9.2 months, 9.3 months, 9.4 months, 9.5 months, 9.6 months, 9.7 months, 9.8 months, 9.9 months, or 10 months). In some embodiments, administration of the PD-1 axis binding antagonist to the subject may extend the subject's OS by about 7.1 months as compared to administration of the platinum-based chemotherapy without the PD-1 axis binding antagonist.

In some embodiments of the disclosure, administration of the PD-1 axis binding antagonist to the subject extends the subject's PFS as compared to administration of the platinum-based chemotherapy without the PD-1 axis binding antagonist. For example, administration of the PD-1 axis binding antagonist to the subject may extend the subject's PFS by from about 1 month to about 5 months, by from about 2 months to about 4 months, by from about 2.1 months to about 3.9 months, by from about 2.5 months to about 3.5 months, or by from about 2.8 months to about 3.4 months as compared to administration of the platinum-based chemotherapy without the PD-1 axis binding antagonist (e.g., by about 1 month, 1.1 months, 1.2 months, 1.3 months, 1.4 months, 1.5 months, 1.6 months, 1.7 months, 1.8 months, 1.9 months, 2 months, 2.1 months, 2.2 months, 2.3 months, 2.4 months, 2.5 months, 2.6 months, 2.7 months, 2.8 months, 2.9 months, 3 months, 3.1 months, 3.2 months, 3.3 months, 3.4 months, 3.5 months, 3.6 months, 3.7 months, 3.8 months, 3.9 months, 4 months, 4.1 months, 4.2 months, 4.3 months, 4.4 months, 4.5 months, 4.6 months, 4.7 months, 4.8 months, 4.9 months, or 5 months). In some embodiments, administration of the PD-1 axis binding antagonist to the subject may extend the subject's OS by about 3.1 months as compared to administration of the platinum-based chemotherapy without the PD-1 axis binding antagonist.

In some embodiments, administration of the PD-1 axis binding antagonist to the subject increases the subject's likelihood of having an objective response and/or extends the subject's duration of response (DOR) as compared to administration of the platinum-based chemotherapy without the PD-1 axis binding antagonist.

In some embodiments, administration of the PD-1 axis binding antagonist to the subject increases the subject's likelihood of having an objective response and/or extends the subject's objective response rate (ORR) as compared to administration of the platinum-based chemotherapy without the PD-1 axis binding antagonist.

In some embodiments, administration of the PD-1 axis binding antagonist to the subject increases the subject's likelihood of having a complete response (CR) as compared to administration of the platinum-based chemotherapy without the PD-1 axis binding antagonist.

In some embodiments, the platinum-based chemotherapy comprises a platinum-based chemotherapeutic agent and a nucleoside analog.

In some embodiments, the platinum-based chemotherapeutic agent is cisplatin, carboplatin, or oxaliplatin.

In some embodiments, the platinum-based chemotherapeutic agent is cisplatin.

In other embodiments, the platinum-based chemotherapeutic agent is carboplatin.

In some embodiments, the nucleoside analog is gemcitabine.

In some embodiments, the platinum-based chemotherapy comprises cisplatin and gemcitabine or carboplatin and gemcitabine.

In some embodiments, the platinum-based chemotherapy comprises cisplatin and gemcitabine.

In some embodiments, the platinum-based chemotherapy comprises carboplatin and gemcitabine.

In some embodiments, the platinum-based chemotherapy comprises cisplatin and premetrexed or carboplatin and premetrexed.

In some embodiments, the platinum-based chemotherapy comprises cisplatin and premetrexed.

In some embodiments, the platinum-based chemotherapy comprises carboplatin and premetrexed.

In some embodiments, the PD-1 axis binding antagonist is administered to the subject during one or more dosing cycles, such as from 1 to 10 dosing cycles (e.g., from 2 to 8 dosing cycles, from 3 to 7 dosing cycles, or from 4 to 6 dosing cycles). In some embodiments, the PD-1 axis binding antagonist is administered to the subject during from 4 to 6 dosing cycles. The PD-1 axis binding antagonist may be administered to the subject one or more times during each dosing cycle, such as once per dosing cycle.

In some embodiments, the dosing cycles continue for up to 58 months. For example, the dosing cycles may continue for from 1 month to 100 months, such as from 2 months to 99 months, from 3 months to 98 months, from 4 months to 97 months, from 5 months to 96 months, from 6 months to 95 months, from 7 months to 94 months, from 8 months to 93 months, from 9 months to 92 months, from 10 months to 91 months, from 11 months to 90 months, from 12 months to 89 months, from 13 months to 88 months, from 14 months to 87 months, from 15 months to 86 months, from 16 months to 85 months, from 17 months to 84 months, from 18 months to 83 months, from 19 months to 82 months, from 20 months to 81 months, from 21 months to 80 months, from 22 months to 79 months, from 23 months to 78 months, from 24 months to 77 months, from 25 months to 76 months, from 26 months to 75 months, from 27 months to 74 months, from 28 months to 73 months, from 29 months to 72 months, from 30 months to 71 months, from 31 months to 70 months, from 32 months to 69 months, from 33 months to 68 months, from 34 months to 67 months, from 35 months to 66 months, from 36 months to 65 months, from 37 months to 64 months, from 36 months to 63 months, from 37 months to 62 months, from 38 months to 61 months, from 39 months to 60 months, from 50 months to 70 months, from 51 months to 69 months, from 52 months to 68 months, from 53 months to 67 months, from 54 months to 66 months, from 55 months to 61 months, from 56 months to 60 months, or from 57 months to 59 months.

In some embodiments, each dosing cycle is about 21 days.

In some embodiments, the PD-1 axis binding antagonist is administered to the subject as a monotherapy. In some embodiments, the PD-1 axis binding antagonist is administered to the subject in combination with a platinum-based chemotherapy, such as a platinum-based chemotherapy that includes a platinum-based chemotherapeutic agent and a nucleoside analog. In some embodiments, the platinum-based chemotherapeutic agent administered to the subject is cisplatin, carboplatin, or oxaliplatin. In some embodiments, the platinum-based chemotherapeutic agent administered to the subject is cisplatin. In other embodiments, the platinum-based chemotherapeutic agent administered to the subject is carboplatin. In some embodiments, the nucleoside analog administered to the subject is gemcitabine. In some embodiments, the platinum-based chemotherapy administered to the subject comprises cisplatin and gemcitabine or carboplatin and gemcitabine. In some embodiments, the platinum-based chemotherapy administered to the subject comprises cisplatin and gemcitabine. In some embodiments, the platinum-based chemotherapy administered to the subject comprises carboplatin and gemcitabine. In some embodiments, the platinum-based chemotherapy administered to the subject comprises cisplatin and premetrexed or carboplatin and premetrexed. In some embodiments, the platinum-based chemotherapy administered to the subject comprises cisplatin and premetrexed. In some embodiments, the platinum-based chemotherapy administered to the subject comprises carboplatin and premetrexed.

In some embodiments, cisplatin is administered to the subject intravenously at a dose of about 75 mg/m² on Day −2 to Day 4 of a 21-day dosing cycle (e.g., on Day −2, Day −1, Day 0, Day 1, Day 2, Day 3, or Day 4 of a 21-day dosing cycle). In some embodiments, cisplatin is administered to the subject intravenously at a dose of about 75 mg/m² on Day 1 of a 21-day dosing cycle.

In some embodiments, carboplatin is administered to the subject intravenously at an area under the curve (AUC) of about 5 on Day −2 to Day 4 of a 21-day dosing cycle (e.g., on Day −2, Day −1, Day 0, Day 1, Day 2, Day 3, or Day 4 of a 21-day dosing cycle). In some embodiments, carboplatin is administered to the subject intravenously at an AUC of about 5 on Day 1 of a 21-day dosing cycle.

In some embodiments, carboplatin is administered to the subject intravenously at an AUC of about 6 on Day −2 to Day 4 of a 21-day dosing cycle (e.g., on Day −2, Day −1, Day 0, Day 1, Day 2, Day 3, or Day 4 of a 21-day dosing cycle). In some embodiments, carboplatin is administered to the subject intravenously at an AUC of about 6 on Day 1 of a 21-day dosing cycle.

In some embodiments, gemcitabine is administered to the subject intravenously at a dose of about 1000 mg/m² on Day −2 to Day 4 (e.g., on Day −2, Day −1, Day 0, Day 1, Day 2, Day 3, or Day 4) and on Day 7 to Day 11 (e.g., on Day 7, Day 8, Day 9, Day 10, or Day 11) of a 21-day dosing cycle. In some embodiments, gemcitabine is administered to the subject intravenously at a dose of about 1000 mg/m² on Day 1 and Day 8 of a 21-day dosing cycle.

In some embodiments, gemcitabine is administered to the subject intravenously at a dose of about 1250 mg/m² on Day −2 to Day 4 (e.g., on Day −2, Day −1, Day 0, Day 1, Day 2, Day 3, or Day 4) and on Day 7 to Day 11 (e.g., on Day 7, Day 8, Day 9, Day 10, or Day 11) of a 21-day dosing cycle. In some embodiments, gemcitabine is administered to the subject intravenously at a dose of about 1250 mg/m² on Day 1 and Day 8 of a 21-day dosing cycle.

In some embodiments, premetrexed is administered to the subject intravenously at a dose of about 500 mg/m² on Day −2 to Day 4 of a 21-day dosing cycle (e.g., on Day −2, Day −1, Day 0, Day 1, Day 2, Day 3, or Day 4 of a 21-day dosing cycle). In some embodiments, premetrexed is administered to the subject intravenously at a dose of about 500 mg/m² on Day 1 of a 21-day dosing cycle.

In some embodiments, the PD-1 axis binding antagonist is selected from the group consisting of a PD-L1 binding antagonist, a PD-1 binding antagonist, and a PD-L2 binding antagonist.

In some embodiments, the PD-1 axis binding antagonist is a PD-L1 binding antagonist.

In some embodiments, the PD-L1 binding antagonist inhibits the binding of PD-L1 to one or more of its ligand binding partners.

In some embodiments, the PD-L1 binding antagonist inhibits the binding of PD-L1 to PD-1, B7-1, or both PD-1 and B7-1.

In some embodiments, the PD-L1 binding antagonist is an anti-PD-L1 antibody.

In some embodiments, the anti-PD-L1 antibody is atezolizumab (TECENTRIQ®), MDX-1105, MED14736 (durvalumab), or MSB0010718C (avelumab).

In some embodiments, the anti-PD-L1 antibody comprises the following hypervariable regions (HVRs): (a) an HVR-H1 sequence of GFTFSDSWIH (SEQ ID NO: 19); (b) an HVR-H2 sequence of AWISPYGGSTYYADSVKG (SEQ ID NO: 20); (c) an HVR-H3 sequence of RHWPGGFDY (SEQ ID NO: 21); (d) an HVR-L1 sequence of RASQDVSTAVA (SEQ ID NO: 22); (e) an HVR-L2 sequence of SASFLYS (SEQ ID NO: 23); and (f) an HVR-L3 sequence of QQYLYHPAT (SEQ ID NO: 24).

In some embodiments, the anti-PD-L1 antibody comprises: (a) a heavy chain variable (VH) domain comprising an amino acid sequence having at least 90% sequence identity to the amino acid sequence of SEQ ID NO: 3; (b) a light chain variable (VL) domain comprising an amino acid sequence having at least 90% sequence identity to the amino acid sequence of SEQ ID NO: 4; or (c) a VH domain as in (a) and a VL domain as in (b). In some embodiments, the anti-PD-L1 antibody comprises: (a) a VH domain comprising an amino acid sequence having at least 95% sequence identity to the amino acid sequence of SEQ ID NO: 3; (b) a VL domain comprising an amino acid sequence having at least 95% sequence identity to the amino acid sequence of SEQ ID NO: 4; or (c) a VH domain as in (a) and a VL domain as in (b). In some embodiments, the anti-PD-L1 antibody comprises: (a) a VH domain comprising an amino acid sequence having at least 96% sequence identity to the amino acid sequence of SEQ ID NO: 3; (b) a VL domain comprising an amino acid sequence having at least 96% sequence identity to the amino acid sequence of SEQ ID NO: 4; or (c) a VH domain as in (a) and a VL domain as in (b). In some embodiments, the anti-PD-L1 antibody comprises: (a) a VH domain comprising an amino acid sequence having at least 97% sequence identity to the amino acid sequence of SEQ ID NO: 3; (b) a VL domain comprising an amino acid sequence having at least 97% sequence identity to the amino acid sequence of SEQ ID NO: 4; or (c) a VH domain as in (a) and a VL domain as in (b). In some embodiments, the anti-PD-L1 antibody comprises: (a) a VH domain comprising an amino acid sequence having at least 98% sequence identity to the amino acid sequence of SEQ ID NO: 3; (b) a VL domain comprising an amino acid sequence having at least 98% sequence identity to the amino acid sequence of SEQ ID NO: 4; or (c) a VH domain as in (a) and a VL domain as in (b). In some embodiments, the anti-PD-L1 antibody comprises: (a) a VH domain comprising an amino acid sequence having at least 99% sequence identity to the amino acid sequence of SEQ ID NO: 3; (b) a VL domain comprising an amino acid sequence having at least 99% sequence identity to the amino acid sequence of SEQ ID NO: 4; or (c) a VH domain as in (a) and a VL domain as in (b). In some embodiments, the anti-PD-L1 antibody comprises: (a) a VH domain comprising the amino acid sequence of SEQ ID NO: 3; (b) a VL domain comprising the amino acid sequence of SEQ ID NO: 4; or (c) a VH domain as in (a) and a VL domain as in (b). In some embodiments, the anti-PD-L1 antibody comprises: (a) a VH domain comprising the amino acid sequence of SEQ ID NO: 3; and (b) a VL domain comprising the amino acid sequence of SEQ ID NO: 4.

In some embodiments, the anti-PD-L1 antibody is atezolizumab.

In some embodiments, atezolizumab is administered to the subject intravenously at a dose of about 840 mg every 2 weeks, about 1200 mg every 3 weeks, or about 1680 mg every 4 weeks.

In some embodiments, atezolizumab is administered to the subject intravenously at a dose of about 1200 mg every 3 weeks.

In some embodiments, atezolizumab is administered to the subject intravenously at a dose of about 1200 mg on Day −2 to Day 4 of a 21-day dosing cycle.

In some embodiments, atezolizumab is administered to the subject intravenously at a dose of about 1200 mg on Day 1 of a 21-day dosing cycle.

In some embodiments, the PD-1 axis binding antagonist is a PD-1 binding antagonist.

In some embodiments, the PD-1 binding antagonist inhibits the binding of PD-1 to one or more of its ligand binding partners.

In some embodiments, the PD-1 binding antagonist inhibits the binding of PD-1 to PD-L1, PD-L2, or both PD-L1 and PD-L2.

In some embodiments, the PD-1 binding antagonist is an anti-PD-1 antibody.

In some embodiments, the anti-PD-1 antibody is MDX-1106 (nivolumab), MK-3475 (pembrolizumab), MEDI-0680 (AMP-514), PDR001, REGN2810, or BGB-108.

In some embodiments, the PD-1 binding antagonist is an Fc fusion protein.

In some embodiments, the Fc fusion protein is AMP-224.

In some embodiments, the subject is chemotherapy naïve. For example, the subject may be one that has not previously been administered chemotherapy for treatment of the NSCLC. In some embodiments, the subject has not previously been administered systemic therapy for treatment of the NSCLC. In some embodiments, the subject has not previously been administered any therapy for treatment of the NSCLC.

In some embodiments, the NSCLC is stage IV NSCLC.

In some embodiments, the NSCLC is metastatic NSCLC.

In some embodiments, a tumor sample obtained from the subject has been determined to have a detectable expression level of PD-L1 in 1% or more of the tumor cells in the tumor sample. For example, in some embodiments, the tumor sample obtained from the subject has been determined to have a detectable expression level of PD-L1 in from about 1% to about 100% of the tumor cells in the tumor sample, from about 2% to about 100% of the tumor cells in the tumor sample, from about 3% to about 100% of the tumor cells in the tumor sample, from about 4% to about 100% of the tumor cells in the tumor sample, from about 5% to about 100% of the tumor cells in the tumor sample, from about 6% to about 100% of the tumor cells in the tumor sample, from about 7% to about 100% of the tumor cells in the tumor sample, from about 8% to about 100% of the tumor cells in the tumor sample, from about 9% to about 100% of the tumor cells in the tumor sample, from about 10% to about 100% of the tumor cells in the tumor sample, from about 11% to about 100% of the tumor cells in the tumor sample, from about 12% to about 100% of the tumor cells in the tumor sample, from about 13% to about 100% of the tumor cells in the tumor sample, from about 14% to about 100% of the tumor cells in the tumor sample, from about 15% to about 100% of the tumor cells in the tumor sample, from about 16% to about 100% of the tumor cells in the tumor sample, from about 17% to about 100% of the tumor cells in the tumor sample, from about 18% to about 100% of the tumor cells in the tumor sample, from about 19% to about 100% of the tumor cells in the tumor sample, from about 20% to about 100% of the tumor cells in the tumor sample, from about 21% to about 100% of the tumor cells in the tumor sample, from about 22% to about 100% of the tumor cells in the tumor sample, from about 23% to about 100% of the tumor cells in the tumor sample, from about 24% to about 100% of the tumor cells in the tumor sample, from about 25% to about 100% of the tumor cells in the tumor sample, from about 26% to about 100% of the tumor cells in the tumor sample, from about 27% to about 100% of the tumor cells in the tumor sample, from about 28% to about 100% of the tumor cells in the tumor sample, from about 29% to about 100% of the tumor cells in the tumor sample, from about 30% to about 100% of the tumor cells in the tumor sample, from about 31% to about 100% of the tumor cells in the tumor sample, from about 32% to about 100% of the tumor cells in the tumor sample, from about 33% to about 100% of the tumor cells in the tumor sample, from about 34% to about 100% of the tumor cells in the tumor sample, from about 35% to about 100% of the tumor cells in the tumor sample, from about 36% to about 100% of the tumor cells in the tumor sample, from about 37% to about 100% of the tumor cells in the tumor sample, from about 38% to about 100% of the tumor cells in the tumor sample, from about 39% to about 100% of the tumor cells in the tumor sample, from about 40% to about 100% of the tumor cells in the tumor sample, from about 41% to about 100% of the tumor cells in the tumor sample, from about 42% to about 100% of the tumor cells in the tumor sample, from about 43% to about 100% of the tumor cells in the tumor sample, from about 44% to about 100% of the tumor cells in the tumor sample, from about 45% to about 100% of the tumor cells in the tumor sample, from about 46% to about 100% of the tumor cells in the tumor sample, from about 47% to about 100% of the tumor cells in the tumor sample, from about 48% to about 100% of the tumor cells in the tumor sample, from about 49% to about 100% of the tumor cells in the tumor sample, from about 50% to about 100% of the tumor cells in the tumor sample, from about 51% to about 100% of the tumor cells in the tumor sample, from about 52% to about 100% of the tumor cells in the tumor sample, from about 53% to about 100% of the tumor cells in the tumor sample, from about 54% to about 100% of the tumor cells in the tumor sample, from about 55% to about 100% of the tumor cells in the tumor sample, from about 56% to about 100% of the tumor cells in the tumor sample, from about 57% to about 100% of the tumor cells in the tumor sample, from about 58% to about 100% of the tumor cells in the tumor sample, from about 59% to about 100% of the tumor cells in the tumor sample, from about 60% to about 100% of the tumor cells in the tumor sample, from about 61% to about 100% of the tumor cells in the tumor sample, from about 62% to about 100% of the tumor cells in the tumor sample, from about 63% to about 100% of the tumor cells in the tumor sample, from about 64% to about 100% of the tumor cells in the tumor sample, from about 65% to about 100% of the tumor cells in the tumor sample, from about 66% to about 100% of the tumor cells in the tumor sample, from about 67% to about 100% of the tumor cells in the tumor sample, from about 68% to about 100% of the tumor cells in the tumor sample, from about 69% to about 100% of the tumor cells in the tumor sample, from about 70% to about 100% of the tumor cells in the tumor sample, from about 71% to about 100% of the tumor cells in the tumor sample, from about 72% to about 100% of the tumor cells in the tumor sample, from about 73% to about 100% of the tumor cells in the tumor sample, from about 74% to about 100% of the tumor cells in the tumor sample, from about 75% to about 100% of the tumor cells in the tumor sample, from about 76% to about 100% of the tumor cells in the tumor sample, from about 77% to about 100% of the tumor cells in the tumor sample, from about 78% to about 100% of the tumor cells in the tumor sample, from about 79% to about 100% of the tumor cells in the tumor sample, from about 80% to about 100% of the tumor cells in the tumor sample, from about 81% to about 100% of the tumor cells in the tumor sample, from about 82% to about 100% of the tumor cells in the tumor sample, from about 83% to about 100% of the tumor cells in the tumor sample, from about 84% to about 100% of the tumor cells in the tumor sample, from about 85% to about 100% of the tumor cells in the tumor sample, from about 86% to about 100% of the tumor cells in the tumor sample, from about 87% to about 100% of the tumor cells in the tumor sample, from about 88% to about 100% of the tumor cells in the tumor sample, from about 89% to about 100% of the tumor cells in the tumor sample, from about 90% to about 100% of the tumor cells in the tumor sample, from about 91% to about 100% of the tumor cells in the tumor sample, from about 92% to about 100% of the tumor cells in the tumor sample, from about 93% to about 100% of the tumor cells in the tumor sample, from about 94% to about 100% of the tumor cells in the tumor sample, from about 95% to about 100% of the tumor cells in the tumor sample, from about 96% to about 100% of the tumor cells in the tumor sample, from about 97% to about 100% of the tumor cells in the tumor sample, from about 98% to about 100% of the tumor cells in the tumor sample, or from about 99% to about 100% of the tumor cells in the tumor sample (e.g., about 1% of the tumor cells in the tumor sample, about 2% of the tumor cells in the tumor sample, about 3% of the tumor cells in the tumor sample, about 4% of the tumor cells in the tumor sample, about 5% of the tumor cells in the tumor sample, about 6% of the tumor cells in the tumor sample, about 7% of the tumor cells in the tumor sample, about 8% of the tumor cells in the tumor sample, about 9% of the tumor cells in the tumor sample, about 10% of the tumor cells in the tumor sample, about 11% of the tumor cells in the tumor sample, about 12% of the tumor cells in the tumor sample, about 13% of the tumor cells in the tumor sample, about 14% of the tumor cells in the tumor sample, about 15% of the tumor cells in the tumor sample, about 16% of the tumor cells in the tumor sample, about 17% of the tumor cells in the tumor sample, about 18% of the tumor cells in the tumor sample, about 19% of the tumor cells in the tumor sample, about 20% of the tumor cells in the tumor sample, about 21% of the tumor cells in the tumor sample, about 22% of the tumor cells in the tumor sample, about 23% of the tumor cells in the tumor sample, about 24% of the tumor cells in the tumor sample, about 25% of the tumor cells in the tumor sample, about 26% of the tumor cells in the tumor sample, about 27% of the tumor cells in the tumor sample, about 28% of the tumor cells in the tumor sample, about 29% of the tumor cells in the tumor sample, about 30% of the tumor cells in the tumor sample, about 31% of the tumor cells in the tumor sample, about 32% of the tumor cells in the tumor sample, about 33% of the tumor cells in the tumor sample, about 34% of the tumor cells in the tumor sample, about 35% of the tumor cells in the tumor sample, about 36% of the tumor cells in the tumor sample, about 37% of the tumor cells in the tumor sample, about 38% of the tumor cells in the tumor sample, about 39% of the tumor cells in the tumor sample, about 40% of the tumor cells in the tumor sample, about 41% of the tumor cells in the tumor sample, about 42% of the tumor cells in the tumor sample, about 43% of the tumor cells in the tumor sample, about 44% of the tumor cells in the tumor sample, about 45% of the tumor cells in the tumor sample, about 46% of the tumor cells in the tumor sample, about 47% of the tumor cells in the tumor sample, about 48% of the tumor cells in the tumor sample, about 49% of the tumor cells in the tumor sample, about 50% of the tumor cells in the tumor sample, about 51% of the tumor cells in the tumor sample, about 52% of the tumor cells in the tumor sample, about 53% of the tumor cells in the tumor sample, about 54% of the tumor cells in the tumor sample, about 55% of the tumor cells in the tumor sample, about 56% of the tumor cells in the tumor sample, about 57% of the tumor cells in the tumor sample, about 58% of the tumor cells in the tumor sample, about 59% of the tumor cells in the tumor sample, about 60% of the tumor cells in the tumor sample, about 61% of the tumor cells in the tumor sample, about 62% of the tumor cells in the tumor sample, about 63% of the tumor cells in the tumor sample, about 64% of the tumor cells in the tumor sample, about 65% of the tumor cells in the tumor sample, about 66% of the tumor cells in the tumor sample, about 67% of the tumor cells in the tumor sample, about 68% of the tumor cells in the tumor sample, about 69% of the tumor cells in the tumor sample, about 70% of the tumor cells in the tumor sample, about 71% of the tumor cells in the tumor sample, about 72% of the tumor cells in the tumor sample, about 73% of the tumor cells in the tumor sample, about 74% of the tumor cells in the tumor sample, about 75% of the tumor cells in the tumor sample, about 76% of the tumor cells in the tumor sample, about 77% of the tumor cells in the tumor sample, about 78% of the tumor cells in the tumor sample, about 79% of the tumor cells in the tumor sample, about 80% of the tumor cells in the tumor sample, about 81% of the tumor cells in the tumor sample, about 82% of the tumor cells in the tumor sample, about 83% of the tumor cells in the tumor sample, about 84% of the tumor cells in the tumor sample, about 85% of the tumor cells in the tumor sample, about 86% of the tumor cells in the tumor sample, about 87% of the tumor cells in the tumor sample, about 88% of the tumor cells in the tumor sample, about 89% of the tumor cells in the tumor sample, about 90% of the tumor cells in the tumor sample, about 91% of the tumor cells in the tumor sample, about 92% of the tumor cells in the tumor sample, about 93% of the tumor cells in the tumor sample, about 94% of the tumor cells in the tumor sample, about 95% of the tumor cells in the tumor sample, about 96% of the tumor cells in the tumor sample, about 97% of the tumor cells in the tumor sample, about 98% of the tumor cells in the tumor sample, about 99% of the tumor cells in the tumor sample, or about 100% of the tumor cells in the tumor sample).

In some embodiments, a tumor sample obtained from the subject has been determined to have a detectable expression level of PD-L1 in tumor-infiltrating immune cells that comprise 1% or more of the tumor sample. For example, in some embodiments the tumor sample obtained from the subject has been determined to have a detectable expression level of PD-L1 in tumor-infiltrating immune cells that comprise from about 1% to about 100% of the tumor sample, from about 5% to about 100% of the tumor sample, from about 10% to about 100% of the tumor sample, from about 15% to about 100% of the tumor sample, from about 20% to about 100% of the tumor sample, from about 30% to about 100% of the tumor sample, from about 40% to about 100% of the tumor sample, from about 50% to about 100% of the tumor sample, from about 60% to about 100% of the tumor sample, from about 70% to about 100% of the tumor sample, from about 80% to about 100% of the tumor sample, or from about 90% to about 100% of the tumor sample (e.g., about 1% of the tumor sample, about 2% of the tumor sample, about 3% of the tumor sample, about 4% of the tumor sample, about 5% of the tumor sample, about 6% of the tumor sample, about 7% of the tumor sample, about 8% of the tumor sample, about 9% of the tumor sample, about 10% of the tumor sample, about 11% of the tumor sample, about 12% of the tumor sample, about 13% of the tumor sample, about 14% of the tumor sample, about 15% of the tumor sample, about 16% of the tumor sample, about 17% of the tumor sample, about 18% of the tumor sample, about 19% of the tumor sample, about 20% of the tumor sample, about 21% of the tumor sample, about 22% of the tumor sample, about 23% of the tumor sample, about 24% of the tumor sample, about 25% of the tumor sample, about 26% of the tumor sample, about 27% of the tumor sample, about 28% of the tumor sample, about 29% of the tumor sample, about 30% of the tumor sample, about 31% of the tumor sample, about 32% of the tumor sample, about 33% of the tumor sample, about 34% of the tumor sample, about 35% of the tumor sample, about 36% of the tumor sample, about 37% of the tumor sample, about 38% of the tumor sample, about 39% of the tumor sample, about 40% of the tumor sample, about 41% of the tumor sample, about 42% of the tumor sample, about 43% of the tumor sample, about 44% of the tumor sample, about 45% of the tumor sample, about 46% of the tumor sample, about 47% of the tumor sample, about 48% of the tumor sample, about 49% of the tumor sample, about 50% of the tumor sample, about 51% of the tumor sample, about 52% of the tumor sample, about 53% of the tumor sample, about 54% of the tumor sample, about 55% of the tumor sample, about 56% of the tumor sample, about 57% of the tumor sample, about 58% of the tumor sample, about 59% of the tumor sample, about 60% of the tumor sample, about 61% of the tumor sample, about 62% of the tumor sample, about 63% of the tumor sample, about 64% of the tumor sample, about 65% of the tumor sample, about 66% of the tumor sample, about 67% of the tumor sample, about 68% of the tumor sample, about 69% of the tumor sample, about 70% of the tumor sample, about 71% of the tumor sample, about 72% of the tumor sample, about 73% of the tumor sample, about 74% of the tumor sample, about 75% of the tumor sample, about 76% of the tumor sample, about 77% of the tumor sample, about 78% of the tumor sample, about 79% of the tumor sample, about 80% of the tumor sample, about 81% of the tumor sample, about 82% of the tumor sample, about 83% of the tumor sample, about 84% of the tumor sample, about 85% of the tumor sample, about 86% of the tumor sample, about 87% of the tumor sample, about 88% of the tumor sample, about 89% of the tumor sample, about 90% of the tumor sample, about 91% of the tumor sample, about 92% of the tumor sample, about 93% of the tumor sample, about 94% of the tumor sample, about 95% of the tumor sample, about 96% of the tumor sample, about 97% of the tumor sample, about 98% of the tumor sample, about 99% of the tumor sample, or about 100% of the tumor sample).

In some embodiments, a tumor sample obtained from the subject has been determined to have a detectable expression level of PD-L1 in from about 1% to less than 5% of the tumor cells in the tumor sample. For example, in some embodiments, the tumor sample obtained from the subject has been determined to have a detectable expression level of PD-L1 in about 1% of the tumor cells in the tumor sample, about 2% of the tumor cells in the tumor sample, about 3% of the tumor cells in the tumor sample, or about 4% of the tumor cells in the tumor sample.

In some embodiments, a tumor sample obtained from the subject has been determined to have a detectable expression level of PD-L1 in tumor-infiltrating immune cells that comprise from about 1% to less than 5% of the tumor cells in the tumor sample. For example, in some embodiments, the tumor sample obtained from the subject has been determined to have a detectable expression level of PD-L1 in tumor-infiltrating immune cells that comprise about 1% of the tumor cells in the tumor sample, about 2% of the tumor cells in the tumor sample, about 3% of the tumor cells in the tumor sample, or about 4% of the tumor cells in the tumor sample.

In some embodiments, a tumor sample obtained from the subject has been determined to have a detectable expression level of PD-L1 in 5% or more of the tumor cells in the tumor sample. For example, in some embodiments, the tumor sample obtained from the subject has been determined to have a detectable expression level of PD-L1 in from about 5% to about 100% of the tumor cells in the tumor sample, from about 6% to about 100% of the tumor cells in the tumor sample, from about 7% to about 100% of the tumor cells in the tumor sample, from about 8% to about 100% of the tumor cells in the tumor sample, from about 9% to about 100% of the tumor cells in the tumor sample, from about 10% to about 100% of the tumor cells in the tumor sample, from about 11% to about 100% of the tumor cells in the tumor sample, from about 12% to about 100% of the tumor cells in the tumor sample, from about 13% to about 100% of the tumor cells in the tumor sample, from about 14% to about 100% of the tumor cells in the tumor sample, from about 15% to about 100% of the tumor cells in the tumor sample, from about 16% to about 100% of the tumor cells in the tumor sample, from about 17% to about 100% of the tumor cells in the tumor sample, from about 18% to about 100% of the tumor cells in the tumor sample, from about 19% to about 100% of the tumor cells in the tumor sample, from about 20% to about 100% of the tumor cells in the tumor sample, from about 21% to about 100% of the tumor cells in the tumor sample, from about 22% to about 100% of the tumor cells in the tumor sample, from about 23% to about 100% of the tumor cells in the tumor sample, from about 24% to about 100% of the tumor cells in the tumor sample, from about 25% to about 100% of the tumor cells in the tumor sample, from about 26% to about 100% of the tumor cells in the tumor sample, from about 27% to about 100% of the tumor cells in the tumor sample, from about 28% to about 100% of the tumor cells in the tumor sample, from about 29% to about 100% of the tumor cells in the tumor sample, from about 30% to about 100% of the tumor cells in the tumor sample, from about 31% to about 100% of the tumor cells in the tumor sample, from about 32% to about 100% of the tumor cells in the tumor sample, from about 33% to about 100% of the tumor cells in the tumor sample, from about 34% to about 100% of the tumor cells in the tumor sample, from about 35% to about 100% of the tumor cells in the tumor sample, from about 36% to about 100% of the tumor cells in the tumor sample, from about 37% to about 100% of the tumor cells in the tumor sample, from about 38% to about 100% of the tumor cells in the tumor sample, from about 39% to about 100% of the tumor cells in the tumor sample, from about 40% to about 100% of the tumor cells in the tumor sample, from about 41% to about 100% of the tumor cells in the tumor sample, from about 42% to about 100% of the tumor cells in the tumor sample, from about 43% to about 100% of the tumor cells in the tumor sample, from about 44% to about 100% of the tumor cells in the tumor sample, from about 45% to about 100% of the tumor cells in the tumor sample, from about 46% to about 100% of the tumor cells in the tumor sample, from about 47% to about 100% of the tumor cells in the tumor sample, from about 48% to about 100% of the tumor cells in the tumor sample, from about 49% to about 100% of the tumor cells in the tumor sample, from about 50% to about 100% of the tumor cells in the tumor sample, from about 51% to about 100% of the tumor cells in the tumor sample, from about 52% to about 100% of the tumor cells in the tumor sample, from about 53% to about 100% of the tumor cells in the tumor sample, from about 54% to about 100% of the tumor cells in the tumor sample, from about 55% to about 100% of the tumor cells in the tumor sample, from about 56% to about 100% of the tumor cells in the tumor sample, from about 57% to about 100% of the tumor cells in the tumor sample, from about 58% to about 100% of the tumor cells in the tumor sample, from about 59% to about 100% of the tumor cells in the tumor sample, from about 60% to about 100% of the tumor cells in the tumor sample, from about 61% to about 100% of the tumor cells in the tumor sample, from about 62% to about 100% of the tumor cells in the tumor sample, from about 63% to about 100% of the tumor cells in the tumor sample, from about 64% to about 100% of the tumor cells in the tumor sample, from about 65% to about 100% of the tumor cells in the tumor sample, from about 66% to about 100% of the tumor cells in the tumor sample, from about 67% to about 100% of the tumor cells in the tumor sample, from about 68% to about 100% of the tumor cells in the tumor sample, from about 69% to about 100% of the tumor cells in the tumor sample, from about 70% to about 100% of the tumor cells in the tumor sample, from about 71% to about 100% of the tumor cells in the tumor sample, from about 72% to about 100% of the tumor cells in the tumor sample, from about 73% to about 100% of the tumor cells in the tumor sample, from about 74% to about 100% of the tumor cells in the tumor sample, from about 75% to about 100% of the tumor cells in the tumor sample, from about 76% to about 100% of the tumor cells in the tumor sample, from about 77% to about 100% of the tumor cells in the tumor sample, from about 78% to about 100% of the tumor cells in the tumor sample, from about 79% to about 100% of the tumor cells in the tumor sample, from about 80% to about 100% of the tumor cells in the tumor sample, from about 81% to about 100% of the tumor cells in the tumor sample, from about 82% to about 100% of the tumor cells in the tumor sample, from about 83% to about 100% of the tumor cells in the tumor sample, from about 84% to about 100% of the tumor cells in the tumor sample, from about 85% to about 100% of the tumor cells in the tumor sample, from about 86% to about 100% of the tumor cells in the tumor sample, from about 87% to about 100% of the tumor cells in the tumor sample, from about 88% to about 100% of the tumor cells in the tumor sample, from about 89% to about 100% of the tumor cells in the tumor sample, from about 90% to about 100% of the tumor cells in the tumor sample, from about 91% to about 100% of the tumor cells in the tumor sample, from about 92% to about 100% of the tumor cells in the tumor sample, from about 93% to about 100% of the tumor cells in the tumor sample, from about 94% to about 100% of the tumor cells in the tumor sample, from about 95% to about 100% of the tumor cells in the tumor sample, from about 96% to about 100% of the tumor cells in the tumor sample, from about 97% to about 100% of the tumor cells in the tumor sample, from about 98% to about 100% of the tumor cells in the tumor sample, or from about 99% to about 100% of the tumor cells in the tumor sample (e.g., about 5% of the tumor cells in the tumor sample, about 6% of the tumor cells in the tumor sample, about 7% of the tumor cells in the tumor sample, about 8% of the tumor cells in the tumor sample, about 9% of the tumor cells in the tumor sample, about 10% of the tumor cells in the tumor sample, about 11% of the tumor cells in the tumor sample, about 12% of the tumor cells in the tumor sample, about 13% of the tumor cells in the tumor sample, about 14% of the tumor cells in the tumor sample, about 15% of the tumor cells in the tumor sample, about 16% of the tumor cells in the tumor sample, about 17% of the tumor cells in the tumor sample, about 18% of the tumor cells in the tumor sample, about 19% of the tumor cells in the tumor sample, about 20% of the tumor cells in the tumor sample, about 21% of the tumor cells in the tumor sample, about 22% of the tumor cells in the tumor sample, about 23% of the tumor cells in the tumor sample, about 24% of the tumor cells in the tumor sample, about 25% of the tumor cells in the tumor sample, about 26% of the tumor cells in the tumor sample, about 27% of the tumor cells in the tumor sample, about 28% of the tumor cells in the tumor sample, about 29% of the tumor cells in the tumor sample, about 30% of the tumor cells in the tumor sample, about 31% of the tumor cells in the tumor sample, about 32% of the tumor cells in the tumor sample, about 33% of the tumor cells in the tumor sample, about 34% of the tumor cells in the tumor sample, about 35% of the tumor cells in the tumor sample, about 36% of the tumor cells in the tumor sample, about 37% of the tumor cells in the tumor sample, about 38% of the tumor cells in the tumor sample, about 39% of the tumor cells in the tumor sample, about 40% of the tumor cells in the tumor sample, about 41% of the tumor cells in the tumor sample, about 42% of the tumor cells in the tumor sample, about 43% of the tumor cells in the tumor sample, about 44% of the tumor cells in the tumor sample, about 45% of the tumor cells in the tumor sample, about 46% of the tumor cells in the tumor sample, about 47% of the tumor cells in the tumor sample, about 48% of the tumor cells in the tumor sample, about 49% of the tumor cells in the tumor sample, about 50% of the tumor cells in the tumor sample, about 51% of the tumor cells in the tumor sample, about 52% of the tumor cells in the tumor sample, about 53% of the tumor cells in the tumor sample, about 54% of the tumor cells in the tumor sample, about 55% of the tumor cells in the tumor sample, about 56% of the tumor cells in the tumor sample, about 57% of the tumor cells in the tumor sample, about 58% of the tumor cells in the tumor sample, about 59% of the tumor cells in the tumor sample, about 60% of the tumor cells in the tumor sample, about 61% of the tumor cells in the tumor sample, about 62% of the tumor cells in the tumor sample, about 63% of the tumor cells in the tumor sample, about 64% of the tumor cells in the tumor sample, about 65% of the tumor cells in the tumor sample, about 66% of the tumor cells in the tumor sample, about 67% of the tumor cells in the tumor sample, about 68% of the tumor cells in the tumor sample, about 69% of the tumor cells in the tumor sample, about 70% of the tumor cells in the tumor sample, about 71% of the tumor cells in the tumor sample, about 72% of the tumor cells in the tumor sample, about 73% of the tumor cells in the tumor sample, about 74% of the tumor cells in the tumor sample, about 75% of the tumor cells in the tumor sample, about 76% of the tumor cells in the tumor sample, about 77% of the tumor cells in the tumor sample, about 78% of the tumor cells in the tumor sample, about 79% of the tumor cells in the tumor sample, about 80% of the tumor cells in the tumor sample, about 81% of the tumor cells in the tumor sample, about 82% of the tumor cells in the tumor sample, about 83% of the tumor cells in the tumor sample, about 84% of the tumor cells in the tumor sample, about 85% of the tumor cells in the tumor sample, about 86% of the tumor cells in the tumor sample, about 87% of the tumor cells in the tumor sample, about 88% of the tumor cells in the tumor sample, about 89% of the tumor cells in the tumor sample, about 90% of the tumor cells in the tumor sample, about 91% of the tumor cells in the tumor sample, about 92% of the tumor cells in the tumor sample, about 93% of the tumor cells in the tumor sample, about 94% of the tumor cells in the tumor sample, about 95% of the tumor cells in the tumor sample, about 96% of the tumor cells in the tumor sample, about 97% of the tumor cells in the tumor sample, about 98% of the tumor cells in the tumor sample, about 99% of the tumor cells in the tumor sample, or about 100% of the tumor cells in the tumor sample).

In some embodiments, the tumor sample obtained from the subject has been determined to have a detectable expression level of PD-L1 in tumor-infiltrating immune cells that comprise 5% or more of the tumor sample. For example, in some embodiments the tumor sample obtained from the subject has been determined to have a detectable expression level of PD-L1 in tumor-infiltrating immune cells that comprise from about 5% to about 100% of the tumor sample, from about 6% to about 100% of the tumor sample, from about 7% to about 100% of the tumor sample, from about 8% to about 100% of the tumor sample, from about 9% to about 100% of the tumor sample, from about 10% to about 100% of the tumor sample, from about 20% to about 100% of the tumor sample, from about 30% to about 100% of the tumor sample, from about 40% to about 100% of the tumor sample, from about 50% to about 100% of the tumor sample, from about 60% to about 100% of the tumor sample, from about 70% to about 100% of the tumor sample, from about 80% to about 100% of the tumor sample, or from about 90% to about 100% of the tumor sample (e.g., about 10% of the tumor sample, about 11% of the tumor sample, about 12% of the tumor sample, about 13% of the tumor sample, about 14% of the tumor sample, about 15% of the tumor sample, about 16% of the tumor sample, about 17% of the tumor sample, about 18% of the tumor sample, about 19% of the tumor sample, about 20% of the tumor sample, about 21% of the tumor sample, about 22% of the tumor sample, about 23% of the tumor sample, about 24% of the tumor sample, about 25% of the tumor sample, about 26% of the tumor sample, about 27% of the tumor sample, about 28% of the tumor sample, about 29% of the tumor sample, about 30% of the tumor sample, about 31% of the tumor sample, about 32% of the tumor sample, about 33% of the tumor sample, about 34% of the tumor sample, about 35% of the tumor sample, about 36% of the tumor sample, about 37% of the tumor sample, about 38% of the tumor sample, about 39% of the tumor sample, about 40% of the tumor sample, about 41% of the tumor sample, about 42% of the tumor sample, about 43% of the tumor sample, about 44% of the tumor sample, about 45% of the tumor sample, about 46% of the tumor sample, about 47% of the tumor sample, about 48% of the tumor sample, about 49% of the tumor sample, about 50% of the tumor sample, about 51% of the tumor sample, about 52% of the tumor sample, about 53% of the tumor sample, about 54% of the tumor sample, about 55% of the tumor sample, about 56% of the tumor sample, about 57% of the tumor sample, about 58% of the tumor sample, about 59% of the tumor sample, about 60% of the tumor sample, about 61% of the tumor sample, about 62% of the tumor sample, about 63% of the tumor sample, about 64% of the tumor sample, about 65% of the tumor sample, about 66% of the tumor sample, about 67% of the tumor sample, about 68% of the tumor sample, about 69% of the tumor sample, about 70% of the tumor sample, about 71% of the tumor sample, about 72% of the tumor sample, about 73% of the tumor sample, about 74% of the tumor sample, about 75% of the tumor sample, about 76% of the tumor sample, about 77% of the tumor sample, about 78% of the tumor sample, about 79% of the tumor sample, about 80% of the tumor sample, about 81% of the tumor sample, about 82% of the tumor sample, about 83% of the tumor sample, about 84% of the tumor sample, about 85% of the tumor sample, about 86% of the tumor sample, about 87% of the tumor sample, about 88% of the tumor sample, about 89% of the tumor sample, about 90% of the tumor sample, about 91% of the tumor sample, about 92% of the tumor sample, about 93% of the tumor sample, about 94% of the tumor sample, about 95% of the tumor sample, about 96% of the tumor sample, about 97% of the tumor sample, about 98% of the tumor sample, about 99% of the tumor sample, or about 100% of the tumor sample).

In some embodiments, a tumor sample obtained from the subject has been determined to have a detectable expression level of PD-L1 in from about 5% to less than 50% of the tumor cells in the tumor sample. For example, in some embodiments, the tumor sample obtained from the subject has been determined to have a detectable expression level of PD-L1 in about 5% of the tumor cells in the tumor sample, about 6% of the tumor cells in the tumor sample, about 7% of the tumor cells in the tumor sample, about 8% of the tumor cells in the tumor sample, about 9% of the tumor cells in the tumor sample, about 10% of the tumor cells in the tumor sample, about 11% of the tumor cells in the tumor sample, about 12% of the tumor cells in the tumor sample, about 13% of the tumor cells in the tumor sample, about 14% of the tumor cells in the tumor sample, about 15% of the tumor cells in the tumor sample, about 16% of the tumor cells in the tumor sample, about 17% of the tumor cells in the tumor sample, about 18% of the tumor cells in the tumor sample, about 19% of the tumor cells in the tumor sample, about 20% of the tumor cells in the tumor sample, about 21% of the tumor cells in the tumor sample, about 22% of the tumor cells in the tumor sample, about 23% of the tumor cells in the tumor sample, about 24% of the tumor cells in the tumor sample, about 25% of the tumor cells in the tumor sample, about 26% of the tumor cells in the tumor sample, about 27% of the tumor cells in the tumor sample, about 28% of the tumor cells in the tumor sample, about 29% of the tumor cells in the tumor sample, about 30% of the tumor cells in the tumor sample, about 31% of the tumor cells in the tumor sample, about 32% of the tumor cells in the tumor sample, about 33% of the tumor cells in the tumor sample, about 34% of the tumor cells in the tumor sample, about 35% of the tumor cells in the tumor sample, about 36% of the tumor cells in the tumor sample, about 37% of the tumor cells in the tumor sample, about 38% of the tumor cells in the tumor sample, about 39% of the tumor cells in the tumor sample, about 40% of the tumor cells in the tumor sample, about 41% of the tumor cells in the tumor sample, about 42% of the tumor cells in the tumor sample, about 43% of the tumor cells in the tumor sample, about 44% of the tumor cells in the tumor sample, about 45% of the tumor cells in the tumor sample, about 46% of the tumor cells in the tumor sample, about 47% of the tumor cells in the tumor sample, about 48% of the tumor cells in the tumor sample, or about 49% of the tumor cells in the tumor sample.

In some embodiments, a tumor sample obtained from the subject has been determined to have a detectable expression level of PD-L1 in tumor-infiltrating immune cells that comprise from about 5% to less than 10% of the tumor cells in the tumor sample. For example, in some embodiments, the tumor sample obtained from the subject has been determined to have a detectable expression level of PD-L1 in tumor-infiltrating immune cells that comprise about 5% of the tumor cells in the tumor sample, about 6% of the tumor cells in the tumor sample, about 7% of the tumor cells in the tumor sample, about 8% of the tumor cells in the tumor sample, or about 9% of the tumor cells in the tumor sample.

In some embodiments, a tumor sample obtained from the subject has been determined to have a detectable expression level of PD-L1 in 50% or more of the tumor cells in the tumor sample. For example, in some embodiments, the tumor sample obtained from the subject has been determined to have a detectable expression level of PD-L1 in from about 50% to about 100% of the tumor cells in the tumor sample, from about 60% to about 100% of the tumor cells in the tumor sample, from about 70% to about 100% of the tumor cells in the tumor sample, from about 80% to about 100% of the tumor cells in the tumor sample, or from about 90% to about 100% of the tumor cells in the tumor sample (e.g., about 50% of the tumor cells in the tumor sample, about 51% of the tumor cells in the tumor sample, about 52% of the tumor cells in the tumor sample, about 53% of the tumor cells in the tumor sample, about 54% of the tumor cells in the tumor sample, about 55% of the tumor cells in the tumor sample, about 56% of the tumor cells in the tumor sample, about 57% of the tumor cells in the tumor sample, about 58% of the tumor cells in the tumor sample, about 59% of the tumor cells in the tumor sample, about 60% of the tumor cells in the tumor sample, about 61% of the tumor cells in the tumor sample, about 62% of the tumor cells in the tumor sample, about 63% of the tumor cells in the tumor sample, about 64% of the tumor cells in the tumor sample, about 65% of the tumor cells in the tumor sample, about 66% of the tumor cells in the tumor sample, about 67% of the tumor cells in the tumor sample, about 68% of the tumor cells in the tumor sample, about 69% of the tumor cells in the tumor sample, about 70% of the tumor cells in the tumor sample, about 71% of the tumor cells in the tumor sample, about 72% of the tumor cells in the tumor sample, about 73% of the tumor cells in the tumor sample, about 74% of the tumor cells in the tumor sample, about 75% of the tumor cells in the tumor sample, about 76% of the tumor cells in the tumor sample, about 77% of the tumor cells in the tumor sample, about 78% of the tumor cells in the tumor sample, about 79% of the tumor cells in the tumor sample, about 80% of the tumor cells in the tumor sample, about 81% of the tumor cells in the tumor sample, about 82% of the tumor cells in the tumor sample, about 83% of the tumor cells in the tumor sample, about 84% of the tumor cells in the tumor sample, about 85% of the tumor cells in the tumor sample, about 86% of the tumor cells in the tumor sample, about 87% of the tumor cells in the tumor sample, about 88% of the tumor cells in the tumor sample, about 89% of the tumor cells in the tumor sample, about 90% of the tumor cells in the tumor sample, about 91% of the tumor cells in the tumor sample, about 92% of the tumor cells in the tumor sample, about 93% of the tumor cells in the tumor sample, about 94% of the tumor cells in the tumor sample, about 95% of the tumor cells in the tumor sample, about 96% of the tumor cells in the tumor sample, about 97% of the tumor cells in the tumor sample, about 98% of the tumor cells in the tumor sample, about 99% of the tumor cells in the tumor sample, or about 100% of the tumor cells in the tumor sample).

In some embodiments, the tumor sample obtained from the subject has been determined to have a detectable expression level of PD-L1 in tumor-infiltrating immune cells that comprise 10% or more of the tumor sample. For example, in some embodiments the tumor sample obtained from the subject has been determined to have a detectable expression level of PD-L1 in tumor-infiltrating immune cells that comprise from about 10% to about 100% of the tumor sample, from about 20% to about 100% of the tumor sample, from about 30% to about 100% of the tumor sample, from about 40% to about 100% of the tumor sample, from about 50% to about 100% of the tumor sample, from about 60% to about 100% of the tumor sample, from about 70% to about 100% of the tumor sample, from about 80% to about 100% of the tumor sample, or from about 90% to about 100% of the tumor sample (e.g., about 10% of the tumor sample, about 11% of the tumor sample, about 12% of the tumor sample, about 13% of the tumor sample, about 14% of the tumor sample, about 15% of the tumor sample, about 16% of the tumor sample, about 17% of the tumor sample, about 18% of the tumor sample, about 19% of the tumor sample, about 20% of the tumor sample, about 21% of the tumor sample, about 22% of the tumor sample, about 23% of the tumor sample, about 24% of the tumor sample, about 25% of the tumor sample, about 26% of the tumor sample, about 27% of the tumor sample, about 28% of the tumor sample, about 29% of the tumor sample, about 30% of the tumor sample, about 31% of the tumor sample, about 32% of the tumor sample, about 33% of the tumor sample, about 34% of the tumor sample, about 35% of the tumor sample, about 36% of the tumor sample, about 37% of the tumor sample, about 38% of the tumor sample, about 39% of the tumor sample, about 40% of the tumor sample, about 41% of the tumor sample, about 42% of the tumor sample, about 43% of the tumor sample, about 44% of the tumor sample, about 45% of the tumor sample, about 46% of the tumor sample, about 47% of the tumor sample, about 48% of the tumor sample, about 49% of the tumor sample, about 50% of the tumor sample, about 51% of the tumor sample, about 52% of the tumor sample, about 53% of the tumor sample, about 54% of the tumor sample, about 55% of the tumor sample, about 56% of the tumor sample, about 57% of the tumor sample, about 58% of the tumor sample, about 59% of the tumor sample, about 60% of the tumor sample, about 61% of the tumor sample, about 62% of the tumor sample, about 63% of the tumor sample, about 64% of the tumor sample, about 65% of the tumor sample, about 66% of the tumor sample, about 67% of the tumor sample, about 68% of the tumor sample, about 69% of the tumor sample, about 70% of the tumor sample, about 71% of the tumor sample, about 72% of the tumor sample, about 73% of the tumor sample, about 74% of the tumor sample, about 75% of the tumor sample, about 76% of the tumor sample, about 77% of the tumor sample, about 78% of the tumor sample, about 79% of the tumor sample, about 80% of the tumor sample, about 81% of the tumor sample, about 82% of the tumor sample, about 83% of the tumor sample, about 84% of the tumor sample, about 85% of the tumor sample, about 86% of the tumor sample, about 87% of the tumor sample, about 88% of the tumor sample, about 89% of the tumor sample, about 90% of the tumor sample, about 91% of the tumor sample, about 92% of the tumor sample, about 93% of the tumor sample, about 94% of the tumor sample, about 95% of the tumor sample, about 96% of the tumor sample, about 97% of the tumor sample, about 98% of the tumor sample, about 99% of the tumor sample, or about 100% of the tumor sample).

PD-L1 expression may be determined, for example, using immunohistochemistry (IHC), among other techniques described herein. In some embodiments, PD-L1 expression is detected using an anti-PD-L1 antibody. Any suitable anti-PD-L1 antibody may be used, including, e.g., SP142, SP263, 22C3, 28-8, El L3N, 4059, h5H1, and 9A11. In some embodiments, the anti-PD-L1 antibody is SP142. In some embodiments, the anti-PD-L1 antibody is SP263. In some embodiments, the anti-PD-L1 antibody is 22C3.

In some embodiments, the subject is a human, such as a human that (i) does not have a sensitizing mutation in a gene encoding EGFR and/or (ii) does not have an ALK fusion oncogene. For example, the subject may be a human that has no EGFR or ALK genomic tumor aberrations.

In a further aspect, the disclosure features a method of treating non-squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, and wherein a tumor sample obtained from the subject has been determined to have:
(i) a detectable expression level of PD-L1 in 1% or more of the tumor cells in the tumor sample; and/or
(ii) a detectable expression level of PD-L1 in tumor-infiltrating immune cells that comprise 1% or more of the tumor sample.

In a further aspect, the disclosure features a method of treating non-squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 840 mg every 2 weeks, about 1200 mg every 3 weeks, or about 1680 mg every 4 weeks, and wherein a tumor sample obtained from the subject has been determined to have:
(i) a detectable expression level of PD-L1 in 1% or more of the tumor cells in the tumor sample; and/or
(ii) a detectable expression level of PD-L1 in tumor-infiltrating immune cells that comprise 1% or more of the tumor sample.

In a further aspect, the disclosure features a method of treating non-squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in 1% or more of the tumor cells in the tumor sample; and/or
(ii) a detectable expression level of PD-L1 in tumor-infiltrating immune cells that comprise 1% or more of the tumor sample.

In a further aspect, the disclosure features a method of treating stage IV non-squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in 1% or more of the tumor cells in the tumor sample; and/or
(ii) a detectable expression level of PD-L1 in tumor-infiltrating immune cells that comprise 1% or more of the tumor sample.

In a further aspect, the disclosure features a method of treating stage IV non-squamous NSCLC in a human subject in need thereof, the method comprising administering to the subject an effective amount of atezolizumab during one or more dosing cycles, wherein the subject is chemotherapy-naïve, wherein the subject does not have a sensitizing mutation in a gene encoding EGFR and does not have an ALK fusion oncogene, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in 1% or more of the tumor cells in the tumor sample; and/or
(ii) a detectable expression level of PD-L1 in tumor-infiltrating immune cells that comprise 1% or more of the tumor sample.

In a further aspect, the disclosure features a method of treating non-squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, and wherein a tumor sample obtained from the subject has been determined to have:
(i) a detectable expression level of PD-L1 in from 1% to less than 5% of the tumor cells in the tumor sample; and/or
(ii) a detectable expression level of PD-L1 in tumor-infiltrating immune cells that comprise from 1% to less than 5% of the tumor sample.

In a further aspect, the disclosure features a method of treating non-squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 840 mg every 2 weeks, about 1200 mg every 3 weeks, or about 1680 mg every 4 weeks, and wherein a tumor sample obtained from the subject has been determined to have:
(i) a detectable expression level of PD-L1 in from 1% to less than 5% of the tumor cells in the tumor sample; and/or
(ii) a detectable expression level of PD-L1 in tumor-infiltrating immune cells that comprise from 1% to less than 5% of the tumor sample.

In a further aspect, the disclosure features a method of treating non-squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in from 1% to less than 5% of the tumor cells in the tumor sample; and/or
(ii) a detectable expression level of PD-L1 in tumor-infiltrating immune cells that comprise from 1% to less than 5% of the tumor sample.

In a further aspect, the disclosure features a method of treating stage IV non-squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in from 1% to less than 5% of the tumor cells in the tumor sample; and/or
(ii) a detectable expression level of PD-L1 in tumor-infiltrating immune cells that comprise from 1% to less than 5% of the tumor sample.

In a further aspect, the disclosure features a method of treating stage IV non-squamous NSCLC in a human subject in need thereof, the method comprising administering to the subject an effective amount of atezolizumab during one or more dosing cycles, wherein the subject is chemotherapy-naïve, wherein the subject does not have a sensitizing mutation in a gene encoding EGFR and does not have an ALK fusion oncogene, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in from 1% to less than 5% of the tumor cells in the tumor sample; and/or
(ii) a detectable expression level of PD-L1 in tumor-infiltrating immune cells that comprise from 1% to less than 5% of the tumor sample.

In a further aspect, the disclosure features a method of treating non-squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, and wherein a tumor sample obtained from the subject has been determined to have:
(i) a detectable expression level of PD-L1 in 5% or more of the tumor cells in the tumor sample; and/or
(ii) a detectable expression level of PD-L1 in tumor-infiltrating immune cells that comprise 5% or more of the tumor sample.

In a further aspect, the disclosure features a method of treating non-squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 840 mg every 2 weeks, about 1200 mg every 3 weeks, or about 1680 mg every 4 weeks, and wherein a tumor sample obtained from the subject has been determined to have:
(i) a detectable expression level of PD-L1 in 5% or more of the tumor cells in the tumor sample; and/or
(ii) a detectable expression level of PD-L1 in tumor-infiltrating immune cells that comprise 5% or more of the tumor sample.

In a further aspect, the disclosure features a method of treating non-squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in 5% or more of the tumor cells in the tumor sample; and/or
(ii) a detectable expression level of PD-L1 in tumor-infiltrating immune cells that comprise 5% or more of the tumor sample.

In a further aspect, the disclosure features a method of treating stage IV non-squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in 5% or more of the tumor cells in the tumor sample; and/or
(ii) a detectable expression level of PD-L1 in tumor-infiltrating immune cells that comprise 5% or more of the tumor sample.

In a further aspect, the disclosure features a method of treating stage IV non-squamous NSCLC in a human subject in need thereof, the method comprising administering to the subject an effective amount of atezolizumab during one or more dosing cycles, wherein the subject is chemotherapy-naïve, wherein the subject does not have a sensitizing mutation in a gene encoding EGFR and does not have an ALK fusion oncogene, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in 5% or more of the tumor cells in the tumor sample; and/or
(ii) a detectable expression level of PD-L1 in tumor-infiltrating immune cells that comprise 5% or more of the tumor sample.

In a further aspect, the disclosure features a method of treating non-squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, and wherein a tumor sample obtained from the subject has been determined to have:
(i) a detectable expression level of PD-L1 in from 5% to less than 50% of the tumor cells in the tumor sample; and/or
(ii) a detectable expression level of PD-L1 in tumor-infiltrating immune cells that comprise from 5% to less than 10% of the tumor sample.

In a further aspect, the disclosure features a method of treating non-squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 840 mg every 2 weeks, about 1200 mg every 3 weeks, or about 1680 mg every 4 weeks, and wherein a tumor sample obtained from the subject has been determined to have:
(i) a detectable expression level of PD-L1 in from 5% to less than 50% of the tumor cells in the tumor sample; and/or
(ii) a detectable expression level of PD-L1 in tumor-infiltrating immune cells that comprise from 5% to less than 10% of the tumor sample.

In a further aspect, the disclosure features a method of treating non-squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in from 5% to less than 50% of the tumor cells in the tumor sample; and/or
(ii) a detectable expression level of PD-L1 in tumor-infiltrating immune cells that comprise from 5% to less than 10% of the tumor sample.

In a further aspect, the disclosure features a method of treating stage IV non-squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in from 5% to less than 50% of the tumor cells in the tumor sample; and/or
(ii) a detectable expression level of PD-L1 in tumor-infiltrating immune cells that comprise from 5% to less than 10% of the tumor sample.

In a further aspect, the disclosure features a method of treating stage IV non-squamous NSCLC in a human subject in need thereof, the method comprising administering to the subject an effective amount of atezolizumab during one or more dosing cycles, wherein the subject is chemotherapy-naïve, wherein the subject does not have a sensitizing mutation in a gene encoding EGFR and does not have an ALK fusion oncogene, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in from 5% to less than 50% of the tumor cells in the tumor sample; and/or
(ii) a detectable expression level of PD-L1 in tumor-infiltrating immune cells that comprise from 5% to less than 10% of the tumor sample.

In a further aspect, the disclosure features a method of treating non-squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, and wherein a tumor sample obtained from the subject has been determined to have:
(i) a detectable expression level of PD-L1 in 50% or more of the tumor cells in the tumor sample; and/or
(ii) a detectable expression level of PD-L1 in tumor-infiltrating immune cells that comprise 10% or more of the tumor sample.

In a further aspect, the disclosure features a method of treating non-squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 840 mg every 2 weeks, about 1200 mg every 3 weeks, or about 1680 mg every 4 weeks, and wherein a tumor sample obtained from the subject has been determined to have:
(i) a detectable expression level of PD-L1 in 50% or more of the tumor cells in the tumor sample; and/or
(ii) a detectable expression level of PD-L1 in tumor-infiltrating immune cells that comprise 10% or more of the tumor sample.

In a further aspect, the disclosure features a method of treating non-squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in 50% or more of the tumor cells in the tumor sample; and/or
(ii) a detectable expression level of PD-L1 in tumor-infiltrating immune cells that comprise 10% or more of the tumor sample.

In a further aspect, the disclosure features a method of treating stage IV non-squamous NSCLC in a human subject in need thereof, the method comprising administering to the subject an effective amount of atezolizumab during one or more dosing cycles, wherein the subject is chemotherapy-naïve, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in 50% or more of the tumor cells in the tumor sample; and/or
(ii) a detectable expression level of PD-L1 in tumor-infiltrating immune cells that comprise 10% or more of the tumor sample.

In a further aspect, the disclosure features a method of treating stage IV non-squamous NSCLC in a human subject in need thereof, the method comprising administering to the subject an effective amount of atezolizumab during one or more dosing cycles, wherein the subject is chemotherapy-naïve, wherein the subject does not have a sensitizing mutation in a gene encoding EGFR and does not have an ALK fusion oncogene, wherein a bTMB score from a sample from the subject has been determined to be at or above a reference bTMB score, optionally wherein the reference bTMB score is 16, wherein atezolizumab is administered to the subject intravenously at a dose of about 1200 mg every 3 weeks, and wherein a tumor sample obtained from the subject has been determined to have:
(i) a detectable expression level of PD-L1 in 50% or more of the tumor cells in the tumor sample; and/or
(ii) a detectable expression level of PD-L1 in tumor-infiltrating immune cells that comprise 10% or more of the tumor sample.

In a further aspect, the disclosure features a PD-1 axis binding antagonist for use in accordance with the method of any of the above aspects or embodiments of the disclosure.

In another aspect, the disclosure features a use of a PD-1 axis binding antagonist in the manufacture of a medicament for treating non-squamous NSCLC in accordance with the method of any of the above aspects or embodiments of the disclosure.

In another aspect, the disclosure features a kit containing a PD-1 axis binding antagonist and a package insert instructing a user of the kit to administer the PD-1 axis binding antagonist to a subject in accordance with the method of any of the above aspects or embodiments of the disclosure.

It is to be understood that one, some, or all of the properties of the various embodiments described herein may be combined to form other embodiments of the present disclosure. These and other aspects of the disclosure will become apparent to one of skill in the art. These and other embodiments of the disclosure are further described by the detailed description that follows.

### BRIEF DESCRIPTION OF THE DRAWINGS

FIG. 1 is a schematic diagram summarizing the design of the clinical trial described in Example 1, below. In brief, patients having non-small cell lung cancer (NSCLC) were randomly assigned to one of two cohorts: those that receive atezolizumab monotherapy, and those that receive a platinum-based chemotherapy. Patients were monitored for overall survival (OS), progression-free survival (PFS), and other parameters, as described in Example 1.

FIG. 2 is a Kaplan-Meier curve showing that, among patients in the TC3 or IC3-WT population treated as described in Example 1, below, those patients that were administered atezolizumab exhibited a statistically significant and clinically meaningful improvement in OS relative to those patients that were administered a platinum-based chemotherapy regimen. Details regarding the experimental design are provided in Example 1.

FIG. 3 is a Kaplan-Meier curve showing that, among patients in the TC3 or IC3-WT population treated as described in Example 1, below, those patients that were administered atezolizumab exhibited a clinically meaningful improvement in PFS relative to those patients that were administered a platinum-based chemotherapy regimen. Details regarding the experimental design are provided in Example 1.

FIG. 4 is a graph showing the distribution of PD-L1 expression among patients that enrolled in the clinical study of atezolizumab monotherapy safety and efficacy that is descried in Example 1.

FIG. 5 is a Kaplan-Meier curve showing the effect of atezolizumab monotherapy on overall survival among patients in the TC3 or IC3-WT population treated as described in Example 1, below, as compared to patients that were administered a platinum-based chemotherapy regimen.

FIGS. 6A and 6B provides a series of forest plots showing the effect of atezolizumab monotherapy on overall survival among various subgroups of patients in the TC3 or IC3-WT population treated as described in Example 1, below.

FIG. 7 is a Kaplan-Meier curve showing the effect of atezolizumab monotherapy on overall survival among patients in the TC2/3 or IC2/3-WT population treated as described in Example 1, below, as compared to patients that were administered a platinum-based chemotherapy regimen.

FIG. 8 is a Kaplan-Meier curve showing the effect of atezolizumab monotherapy on overall survival among patients in the TC1/2/3 or IC1/2/3-WT population treated as described in Example 1, below, as compared to patients that were administered a platinum-based chemotherapy regimen.

FIG. 9 is a Kaplan-Meier curve showing the effect of atezolizumab monotherapy on progression-free survival among patients in the TC3 or IC3-WT population treated as described in Example 1, below, as compared to patients that were administered a platinum-based chemotherapy regimen.

FIG. 10 shows a set of Kaplan-Meier curves demonstrating the effect of atezolizumab monotherapy on progression-free survival among patients in the TC2/3 or 102/3 population (left) or TC1/2/3 or 101/2/3 WT population (right) treated as described in Example 1, below, as compared to patients that were administered a platinum-based chemotherapy regimen.

FIG. 11 provides a graph showing the effect of atezolizumab monotherapy on overall response rate (ORR) and duration of response (DOR) among patients in the TC3 or IC3-WT population treated as described in Example 1, below. FIG. 11 also provides a table showing the effect of atezolizumab monotherapy on ORR and DOR among patients in the TC2/3, 102/3, TC1/2/3, or 101/2/3 WT populations treated as described in Example 1, below.

FIG. 12 is a graph summarizing adverse events (AEs) experienced by the various patient groups treated with atezolizumab monotherapy or chemotherapy as described in Example 1, below.

FIGS. 13A-13D are graphs showing the stratification of overall survival by PD-L1 expression among biomarker evaluable patients (BEP) treated with atezolizumab as described in Example 1, below.

FIG. 31A shows the overlap between the 22C3≥50% TPS, SP263≥50% TC and SP142 TC3 or 103 populations. FIG. 13B shows Kaplan-Meier estimates of OS in the 50% TPS WT population per the 22C3 PD-L1 IHC assay. FIG. 13C shows Kaplan-Meier of OS in the 50% TC WT population per the SP263 PD-L1 IHC assay. FIG. 13D shows OS in PD-L1 subgroups defined by SP142 IHC, SP263 and 22C3 assays. Medians were estimated using Kaplan-Meier methodology. Unstratified HRs are shown for SP263 and 22C3 subgroup analyses. The BEPs within the ITT-WT population comprised 534 patients for the 22C3 and SP142 overlap; 546 for the SP263 and SP142 overlap; and 530 for the 22C3 and SP263 overlap. ITT−WT=TC1/2/3 or 101/2/3.

FIGS. 14A-14C are graphs showing the stratification of overall survival and progression-free survival by blood tumor mutational burden (bTMB) score among patients treated with atezolizumab as described in Example 1, below. FIG. 14A shows the overlap between PD-L1—high (defined by 22C3 or SP142 IHC) and bTMB-high populations. FIG. 14B shows OS in bTMB subgroups. bTMB subgroups shown as a percentage of the bTMB-BEP. FIG. 14C shows PFS in bTMB subgroups. bTMB subgroups shown as a percentage of the bTMB-BEP. TC1/2/3 or IC1/2/3=PD-L1 expression <1% TC or IC. The non-BEP group comprised patients who had a bTMB result with an MSAF <1% (n=88), who had a sample that failed quality control at the testing vendor or had median exon coverage <800 (n=39) or who had not submitted a baseline plasma sample (n=38). TC3 or IC3=PD-L1 expression 50% TC or 10% IC.

FIG. 15 is a graph showing the prevalence of PD-L1 expression among patients treated with atezolizumab as described in Example 1, below, as assessed by way of immunohistochemistry. “ITT-WT” denotes TC1/2/3 or IC1/2/3-WT patients. BEP denotes biomarker-evaluable population; IC, tumor-infiltrating immune cell; IHC, immunohistochemistry; TC, tumor cell; TPS, tumor proportion score.

FIGS. 16A-16F are graphs showing the stratification of overall survival by PD-L1 expression as assessed by the SP142, SP263, and 22C3 immunohistochemistry assays among patients treated with atezolizumab as described in Example 1, below. FIG. 16A shows overlap between the 22C3 1% TPS and SP263≥1% TC immunohistochemistry populations in the TC1/2/3 or IC1/2/3-WT population. FIGS. 16B and 16C show OS in PD-L1—positive subgroups by PD-L1 IHC assay. FIGS. 16D-16F show OS in PD-L1—low subgroups by PD-L1 IHC assay. For the 22C3 and SP263 overlap, the BEP within the ITT-WT population was 530 patients. Atezo denotes atezolizumab; chemo, chemotherapy; OS, overall survival; PFS, progression-free survival; TC, tumor cell; TPS, tumor proportion score; WT, wild type.

FIG. 17 is a graph showing progression-free survival among PD-L1 subgroups defined by SP142 IHC, SP263 and 22C3 assay. SP142 BEP-WT=TC1/2/3 or IC1/2/3-WT. Stratified HRs for SP142 ITT-WT and TC3 or IC3-WT; unstratified HRs for all other subgroups. Atezo denotes atezolizumab; BEP, biomarker-evaluable population; chemo, chemotherapy; PFS, progression-free survival; TC, tumor cell; WT, wild type.

### DETAILED DESCRIPTION OF EMBODIMENTS OF THE INVENTION

### I. Introduction

The present disclosure provides therapeutic, diagnostic, and prognostic methods and compositions for the treatment and evaluation of subjects having cancer, for example, non-small cell lung cancer (NSCLC, e.g., squamous and non-squamous NSCLC, including stage IV squamous and non-squamous NSCLC). The compositions and methods of the disclosure may be used to identify subjects having cancer (e.g., squamous and non-squamous NSCLC, including stage IV squamous and non-squamous NSCLC) that are particularly likely to benefit from treatment with a PD-1 axis binding antagonist, such as an anti-PD-L1 antibody (e.g., atezolizumab). For example, using the compositions and methods described herein, a subject having cancer (e.g., squamous and non-squamous NSCLC, including stage IV squamous and non-squamous NSCLC) may be identified as likely to benefit from treatment with a PD-1 axis binding antagonist if the subject exhibits a blood tumor mutational burden (bTMB) score that is greater than, or equal to, a reference bTMB score. The present disclosure is based, in part, on the discovery that subjects having cancer (e.g., squamous and non-squamous NSCLC, including stage IV squamous and non-squamous NSCLC) and that exhibit bTMB scores at or above a threshold level have a high likelihood of responding to PD-1 axis binding antagonist treatment. This discovery is described in further detail in the working examples below.

The compositions and methods of the disclosure provide a series of important clinical benefits. Using the compositions and methods described herein, a subject having cancer (e.g., squamous and non-squamous NSCLC, including stage IV squamous and non-squamous NSCLC) can be assessed for their likelihood of benefiting from PD-1 axis binding antagonist therapy before the onset of treatment. The compositions and methods of the disclosure thus enable subjects that are likely to benefit from PD-1 axis binding antagonist therapy to be identified early and to be treated accordingly. Similarly, subjects that are identified as less likely to benefit from PD-1 axis binding antagonist therapy, for example, on the basis of a bTMB score that is beneath a bTMB reference level, may be administered a therapeutic regimen that does not include a PD-1 axis binding antagonist.

PD-1 axis binding antagonists that may be used in conjunction with the compositions and methods of the disclosure include PD-L1 binding antagonists, PD-1 binding antagonists, and PD-L2 binding antagonists. For example, the PD-1 axis binding antagonist may be a PD-L1 binding antagonist, such as an anti-PD-L1 antibody. In some embodiments, the anti-PD-L1 antibody is atezolizumab (TECENTRIQ®), MDX-1105, MED14736 (durvalumab), or MSB0010718C (avelumab). A subject identified as likely to benefit from atezolizumab treatment may be administered atezolizumab by way of any of the routes of administration and dosing schedules described herein, such as intravenously at a dose of about 840 mg every 2 weeks, about 1200 mg every 3 weeks, or about 1680 mg every 4 weeks. In some embodiments, the PD-1 axis binding antagonist is a PD-1 binding antagonist, such as an anti-PD-1 antibody. Exemplary anti-PD-1 antibodies useful in conjunction with the compositions and methods of the disclosure include MDX-1106 (nivolumab), MK-3475 (pembrolizumab), MEDI-0680 (AMP-514), PDR001, REGN2810, and BGB-108. In some embodiments, the PD-1 binding antagonist is an Fc fusion protein, such as AMP-224.

The sections that follow describe in further detail compositions and methods for determining the likelihood of a subject (e.g., a subject having squamous or non-squamous NSCLC, such as stage IV squamous or non-squamous NSCLC) to benefit from PD-1 axis binding antagonist therapy, as well as compositions and methods for treating the subjects accordingly.

### II. Definitions

Before describing the compositions and methods of the present disclosure in detail, it is to be understood that the disclosure is not limited to particular compositions or biological systems, which can, of course, vary. It is also to be understood that the terminology used herein is for the purpose of describing particular embodiments only, and is not intended to be limiting.

As used in this specification and the appended claims, the singular forms “a,” “an,” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the disclosure described herein include “comprising,” “consisting,” and “consisting essentially of” aspects and embodiments.

As used herein, the terms “blood tumor mutational burden score,” “blood tumor mutation burden score,” and “bTMB score,” each of which may be used interchangeably, refer to a numerical value that reflects the number of somatic mutations detected in a blood sample (e.g., a whole blood sample, a plasma sample, a serum sample, or a combination thereof) obtained from an individual (e.g., an individual at risk of or having a cancer). The bTMB score can be measured, for example, on a whole genome or exome basis, or on the basis of a subset of the genome or exome (e.g., a predetermined set of genes). In some embodiments, a bTMB score can be measured based on intergenic sequences. In some embodiments, the bTMB score measured on the basis of a subset of genome or exome can be extrapolated to determine a whole genome or exome bTMB score. In some embodiments, the predetermined set of genes does not comprise the entire genome or the entire exome. In some embodiments, the set of subgenomic intervals does not comprise the entire genome or the entire exome. In some embodiments, the predetermined set of genes comprise a plurality of genes, which, in mutant form, are associated with an effect on cell division, growth or survival, or are associated with cancer. In some embodiments, the predetermined set of genes comprise at least about 50 or more, about 100 or more, about 150 or more, about 200 or more, about 250 or more, about 300 or more, about 350 or more, about 400 or more, about 450 or more, or about 500 or more genes. In some embodiments, the pre-determined set of genes covers about 1 Mb (e.g., about 1.1 Mb, e.g., about 1.125 Mb).

In some embodiments, the bTMB score is determined from measuring the number of somatic mutations in cell-free DNA (cfDNA) in a sample. In some embodiments, the bTMB score is determined from measuring the number of somatic mutations in circulating tumor DNA (ctDNA) in a sample. In some embodiments, the number of somatic mutations is the number of single nucleotide variants (SNVs) counted or a sum of the number of SNVs and the number of indel mutations counted. In some embodiments, the bTMB score refers to the number of accumulated somatic mutations in a tumor. A bTMB score can therefore be used as a surrogate for the number of neoantigens on oncogenic (e.g., tumor) cells. A bTMB score can also be used as a surrogate for the rate of mutation within a tumor, which is a proxy for the number of neoantigens on oncogenic (e.g., tumor) cells. In some embodiments, a bTMB score at or above a reference bTMB score identifies an individual as one who may benefit from a treatment comprising an immune checkpoint inhibitor, for example, a PD-1 axis binding antagonist (e.g., atezolizumab). In some embodiments, a bTMB score below a reference bTMB score identifies an individual as one who may benefit from a treatment comprising an anti-cancer therapy other than, or in addition to, a PD-1 axis binding antagonist. In some embodiments, a bTMB score can be used to monitor response of an individual having a cancer to a treatment comprising an immune checkpoint inhibitor, for example, a PD-1 axis binding antagonist (e.g., atezolizumab).

As used herein, the term “reference bTMB score” refers to a bTMB score against which another bTMB score is compared, e.g., to make a diagnostic, predictive, prognostic, and/or therapeutic determination. For example, the reference bTMB score may be a bTMB score in a reference sample, a reference population, and/or a pre-determined value. In some instances, the reference bTMB score is a cut-off value that significantly separates a first subset of individuals who have been treated with an immune checkpoint inhibitor, for example, a PD-1 axis binding antagonist therapy, in a reference population and a second subset of individuals who have been treated with a non-PD-1 axis binding antagonist therapy that does not comprise an immune checkpoint inhibitor, for example, a PD-1 axis binding antagonist, in the same reference population based on a significant difference between an individual's responsiveness to treatment with the immune checkpoint inhibitor, for example, a PD-1 axis binding antagonist therapy, and an individual's responsiveness to treatment with the non-PD-1 axis binding antagonist therapy at or above the cut-off value and/or below the cut-off value. In some instances, the individual's responsiveness to treatment with the immune checkpoint inhibitor, for example, a PD-1 axis binding antagonist therapy, is significantly improved relative to the individual's responsiveness to treatment with the non-PD-1 axis binding antagonist therapy at or above the cut-off value. In some instances, the individual's responsiveness to treatment with the non-PD-1 axis binding antagonist therapy is significantly improved relative to the individual's responsiveness to treatment with the immune checkpoint inhibitor, for example, a PD-1 axis binding antagonist therapy, below the cut-off value.

It will be appreciated by one skilled in the art that the numerical value for the reference bTMB score may vary depending on the type of cancer (e.g., a lung cancer (e.g., a non-small cell lung cancer (NSCLC)), a kidney cancer (e.g., a kidney urothelial carcinoma), a bladder cancer (e.g., a bladder urothelial (transitional cell) carcinoma), a breast cancer, a colorectal cancer (e.g., a colon adenocarcinoma), an ovarian cancer, a pancreatic cancer, a gastric carcinoma, an esophageal cancer, a mesothelioma, a melanoma (e.g., a skin melanoma), a head and neck cancer (e.g., a head and neck squamous cell carcinoma (HNSCC)), a thyroid cancer, a sarcoma (e.g., a soft-tissue sarcoma, a fibrosarcoma, a myxosarcoma, a liposarcoma, an osteogenic sarcoma, an osteosarcoma, a chondrosarcoma, an angiosarcoma, an endotheliosarcoma, a lymphangiosarcoma, a lymphangioendotheliosarcoma, a leiomyosarcoma, or a rhabdomyosarcoma), a prostate cancer, a glioblastoma, a cervical cancer, a thymic carcinoma, a leukemia (e.g., an acute lymphocytic leukemia (ALL), an acute myelocytic leukemia (AML), a chronic myelocytic leukemia (CML), a chronic eosinophilic leukemia, or a chronic lymphocytic leukemia (CLL)), a lymphoma (e.g., a Hodgkin lymphoma or a non-Hodgkin lymphoma (NHL)), a myeloma (e.g., a multiple myeloma (MM)), a mycoses fungoides, a merkel cell cancer, a hematologic malignancy, a cancer of hematological tissues, a B cell cancer, a bronchus cancer, a stomach cancer, a brain or central nervous system cancer, a peripheral nervous system cancer, a uterine or endometrial cancer, a cancer of the oral cavity or pharynx, a liver cancer, a testicular cancer, a biliary tract cancer, a small bowel or appendix cancer, a salivary gland cancer, an adrenal gland cancer, an adenocarcinoma, an inflammatory myofibroblastic tumor, a gastrointestinal stromal tumor (GIST), a colon cancer, a myelodysplastic syndrome (MDS), a myeloproliferative disorder (MPD), a polycythemia Vera, a chordoma, a synovioma, an Ewing's tumor, a squamous cell carcinoma, a basal cell carcinoma, an adenocarcinoma, a sweat gland carcinoma, a sebaceous gland carcinoma, a papillary carcinoma, a papillary adenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilms' tumor, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a meningioma, a neuroblastoma, a retinoblastoma, a follicular lymphoma, a diffuse large B-cell lymphoma, a mantle cell lymphoma, a hepatocellular carcinoma, a thyroid cancer, a small cell cancer, an essential thrombocythemia, an agnogenic myeloid metaplasia, a hypereosinophilic syndrome, a systemic mastocytosis, a familiar hypereosinophilia, a neuroendocrine cancer, or a carcinoid tumor), the methodology used to measure a bTMB score, and/or the statistical methods used to generate a bTMB score.

The term “equivalent bTMB value” refers to a numerical value that corresponds to a bTMB score that is represented as the number of somatic mutations counted over a defined number of sequenced bases (e.g., about 1.1 Mb (e.g., about 1.125 Mb), e.g., as assessed by the FOUNDATIONONE® panel). It is to be understood that, in general, the bTMB score is linearly related to the size of the genomic region sequenced. Such equivalent bTMB values indicate an equivalent degree of tumor mutational burden as compared to a bTMB score and can be used interchangeably in the methods described herein, for example, to predict response of a cancer patient to an immune checkpoint inhibitor (e.g., an anti-PD-L1 antibody, e.g., atezolizumab). As an example, in some embodiments, an equivalent bTMB value is a normalized bTMB value that can be calculated by dividing the count of somatic variants (e.g., somatic mutations) by the number of bases sequenced. For example, an equivalent bTMB value can be represented, e.g., as the number of mutations per megabase. For example, a bTMB score of about 25 (as determined as the number of somatic mutations counted over about 1.1 Mb) corresponds to an equivalent bTMB value of about 23 mutations/Mb. It is to be understood that bTMB scores as described herein (e.g., bTMB scores represented as the number of somatic mutations counted over a defined number of sequenced bases (e.g., about 1.1 Mb (e.g., about 1.125 Mb), e.g., as assessed by the FOUNDATIONONE® panel)) encompass equivalent bTMB values obtained using different methodologies (e.g., whole-exome sequencing or whole-genome sequencing). As an example, for a whole exome panel, the target region may be approximately 50 Mb, and a sample with about 500 somatic mutations detected has an equivalent bTMB value of about 10 mutations/Mb. In some embodiments, a bTMB score determined as the number of somatic mutations counted over a defined number of sequenced bases (e.g., about 1.1 Mb (e.g., about 1.125 Mb), e.g., as assessed by the FOUNDATIONONE® panel) in a subset of the genome or exome (e.g., a predetermined set of genes) deviates by less than about 30% (e.g., less than about 30%, about 25%, about 20%, about 15%, about 10%, about 5%, about 4%, about 3%, about 2%, about 1%, or less) from a bTMB score determined by whole-exome sequencing. See, e.g., Chalmers et al. Genome Medicine 9:34, 2017.

As used herein, the terms “maximum somatic allele frequency” and “MSAF,” each of which may be used interchangeably, refer to the highest frequency of an allele (i.e., a variant of a gene having a somatic mutation (e.g., a base substitution in a coding region and/or an indel mutation in a coding region)) less than about 40% (e.g., less than 40%, 30%, 20%, 10%, 5%, or 1%), expressed as a fraction or percentage, that is detected from a sample (e.g., a whole blood sample, a plasma sample, a serum sample, or a combination thereof) from an individual. The allele frequency for somatic mutations may be calculated by dividing the number of sequence reads indicating a somatic mutation against the total reads aligned to a particular region of the human genome. In some instances, the MSAF is derived from the largest somatic allele frequency less than about 20% in a sample. In some embodiments, the value is the fraction of all cfDNA in the sample from the subject that carries that allele. In some embodiments, the value is the fraction of ctDNA in the sample from the subject that carries that allele. In some embodiments, the value is used to estimate the total amount of tumor content in the sample. In some embodiments, the method comprises determining an allele frequency for each somatic alteration detected from the sample. For example, a sample with multiple somatic alterations may present those alterations as a distribution of somatic allele frequencies, likely dependent upon their original clonal frequency in a cancer (e.g., a tumor). In some embodiments, the value is expressed as a function of the predetermined set of genes, e.g., the coding regions of the predetermined set of genes. In other embodiments, the value is expressed as a function of the subgenomic intervals sequenced, e.g., the coding subgenomic intervals sequenced. In some embodiments, the MSAF can be used to provide a prognosis for an individual having a cancer.

The term “somatic mutation” or “somatic alteration” refers to a genetic alteration occurring in the somatic tissues (e.g., cells outside the germline). Examples of genetic alterations include, but are not limited to, point mutations (e.g., the exchange of a single nucleotide for another (e.g., silent mutations, missense mutations, and nonsense mutations)), insertions and deletions (e.g., the addition and/or removal of one or more nucleotides (e.g., indels)), amplifications, gene duplications, copy number alterations (CNAs), rearrangements, and splice variants. In some embodiments, an indel may be a frameshift mutation or in-frame mutations of one or more nucleotides (e.g., about 1-40 nucleotides). The presence of particular mutations can be associated with disease states (e.g., cancer, e.g., a lung cancer (e.g., a non-small cell lung cancer (NSCLC)), a kidney cancer (e.g., a kidney urothelial carcinoma), a bladder cancer (e.g., a bladder urothelial (transitional cell) carcinoma), a breast cancer, a colorectal cancer (e.g., a colon adenocarcinoma), an ovarian cancer, a pancreatic cancer, a gastric carcinoma, an esophageal cancer, a mesothelioma, a melanoma (e.g., a skin melanoma), a head and neck cancer (e.g., a head and neck squamous cell carcinoma (HNSCC)), a thyroid cancer, a sarcoma (e.g., a soft-tissue sarcoma, a fibrosarcoma, a myxosarcoma, a liposarcoma, an osteogenic sarcoma, an osteosarcoma, a chondrosarcoma, an angiosarcoma, an endotheliosarcoma, a lymphangiosarcoma, a lymphangioendotheliosarcoma, a leiomyosarcoma, or a rhabdomyosarcoma), a prostate cancer, a glioblastoma, a cervical cancer, a thymic carcinoma, a leukemia (e.g., an acute lymphocytic leukemia (ALL), an acute myelocytic leukemia (AML), a chronic myelocytic leukemia (CML), a chronic eosinophilic leukemia, or a chronic lymphocytic leukemia (CLL)), a lymphoma (e.g., a Hodgkin lymphoma or a non-Hodgkin lymphoma (NHL)), a myeloma (e.g., a multiple myeloma (MM)), a mycoses fungoides, a merkel cell cancer, a hematologic malignancy, a cancer of hematological tissues, a B cell cancer, a bronchus cancer, a stomach cancer, a brain or central nervous system cancer, a peripheral nervous system cancer, a uterine or endometrial cancer, a cancer of the oral cavity or pharynx, a liver cancer, a testicular cancer, a biliary tract cancer, a small bowel or appendix cancer, a salivary gland cancer, an adrenal gland cancer, an adenocarcinoma, an inflammatory myofibroblastic tumor, a gastrointestinal stromal tumor (GIST), a colon cancer, a myelodysplastic syndrome (MDS), a myeloproliferative disorder (MPD), a polycythemia Vera, a chordoma, a synovioma, an Ewing's tumor, a squamous cell carcinoma, a basal cell carcinoma, an adenocarcinoma, a sweat gland carcinoma, a sebaceous gland carcinoma, a papillary carcinoma, a papillary adenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilms' tumor, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a meningioma, a neuroblastoma, a retinoblastoma, a follicular lymphoma, a diffuse large B-cell lymphoma, a mantle cell lymphoma, a hepatocellular carcinoma, a thyroid cancer, a small cell cancer, an essential thrombocythemia, an agnogenic myeloid metaplasia, a hypereosinophilic syndrome, a systemic mastocytosis, a familiar hypereosinophilia, a neuroendocrine cancer, or a carcinoid tumor).

In certain embodiments, the somatic alteration is a silent mutation (e.g., a synonymous alteration). In other embodiments, the somatic alteration is a non-synonymous single nucleotide variant (SNV). In other embodiments, the somatic alteration is a passenger mutation (e.g., an alteration that has no detectable effect on the fitness of a clone). In certain embodiments, the somatic alteration is a variant of unknown significance (VUS), for example, an alteration, the pathogenicity of which can neither be confirmed nor ruled out. In certain embodiments, the somatic alteration has not been identified as being associated with a cancer phenotype.

In certain embodiments, the somatic alteration is not associated with, or is not known to be associated with, an effect on cell division, growth, or survival. In other embodiments, the somatic alteration is associated with an effect on cell division, growth, or survival.

In certain embodiments, the number of somatic alterations excludes one or more functional alterations in a sub-genomic interval.

As used herein, the terms “sub-genomic interval” and “subgenomic interval,” each of which may be used interchangeably, refers to a portion of a genomic sequence. In some embodiments, a subgenomic interval can be a single nucleotide position, e.g., a nucleotide position variant of which is associated (positively or negatively) with a tumor phenotype. In some embodiments, a subgenomic interval comprises more than one nucleotide position. Such embodiments include sequences of at least 2, 5, 10, 50, 100, 150, or 250 nucleotide positions in length. Subgenomic intervals can comprise an entire gene, or a preselected portion thereof, e.g., the coding region (or portions thereof), a preselected intron (or portion thereof) or exon (or portion thereof). A subgenomic interval can comprise all or a part of a fragment of a naturally occurring, e.g., genomic DNA, nucleic acid. For example, a subgenomic interval can correspond to a fragment of genomic DNA, which is subjected to a sequencing reaction. In certain embodiments, a subgenomic interval is continuous sequence from a genomic source. In other embodiments, a subgenomic interval includes sequences that are not contiguous in the genome, e.g., it can include junctions formed at exon-exon junctions in cDNA.

In an embodiment, a subgenomic interval comprises or consists of: a single nucleotide position; an intragenic region or an intergenic region; an exon or an intron, or a fragment thereof, typically an exon sequence or a fragment thereof; a coding region or a non-coding region, e.g., a promoter, an enhancer, a 5′ untranslated region (5′ UTR), or a 3′ untranslated region (3′ UTR), or a fragment thereof; a cDNA or a fragment thereof; an SNV; an SNP; a somatic mutation, a germline mutation or both; an alteration, e.g., a point or a single mutation; a deletion mutation (e.g., an in-frame deletion, an intragenic deletion, a full gene deletion); an insertion mutation (e.g., intragenic insertion); an inversion mutation (e.g., an intra-chromosomal inversion); a linking mutation; a linked insertion mutation; an inverted duplication mutation; a tandem duplication (e.g., an intrachromosomal tandem duplication); a translocation (e.g., a chromosomal translocation, a non-reciprocal translocation); a rearrangement (e.g., a genomic rearrangement (e.g., a rearrangement of one or more introns, or a fragment thereof; a rearranged intron can include a 5′- and/or 3′-UTR)); a change in gene copy number; a change in gene expression; a change in RNA levels; or a combination thereof.

The “amount” or “number” of somatic mutations associated with an increased clinical benefit to an individual is a detectable level in a biological sample. These can be measured by methods known to one skilled in the art and also disclosed herein. The amount of a somatic mutation assessed can be used to determine the response to the treatment.

The “copy number of a gene” refers to the number of DNA sequences in a cell encoding a particular gene product. Generally, for a given gene, a mammal has two copies of each gene. The copy number can be increased, e.g., by gene amplification or duplication, or reduced by deletion.

In some embodiments, the functional alteration is an alteration that, compared with a reference sequence (e.g., a wild-type or unmutated sequence) has an effect on cell division, growth, or survival (e.g., promotes cell division, growth, or survival). In certain embodiments, the functional alteration is identified as such by inclusion in a database of functional alterations, e.g., the COSMIC database (see Forbes et al. Nuci. Acids Res. 43 (D1): D805-D811, 2015, which is herein incorporated by reference in its entirety). In other embodiments, the functional alteration is an alteration with known functional status (e.g., occurring as a known somatic alteration in the COSMIC database). In certain embodiments, the functional alteration is an alteration with a likely functional status (e.g., a truncation in a tumor suppressor gene). In certain embodiments, the functional alteration is a driver mutation (e.g., an alteration that gives a selective advantage to a clone in its microenvironment, e.g., by increasing cell survival or reproduction). In other embodiments, the functional alteration is an alteration capable of causing clonal expansions. In certain embodiments, the functional alteration is an alteration capable of causing one, two, three, four, five, or all six of the following: (a) self-sufficiency in a growth signal; (b) decreased, e.g., insensitivity, to an antigrowth signal; (c) decreased apoptosis; (d) increased replicative potential; (e) sustained angiogenesis; or (f) tissue invasion or metastasis.

In certain embodiments, the functional alteration is not a passenger mutation (e.g., is not an alteration that has no detectable effect on the fitness of a clone of cells). In certain embodiments, the functional alteration is not a variant of unknown significance (VUS) (e.g., is not an alteration, the pathogenicity of which can neither be confirmed nor ruled out).

In certain embodiments, a plurality (e.g., about 10%, 20%, 30%, 40%, 50%, 60%, 70%, 80%, 90%, or more) of functional alterations in a pre-selected tumor gene in the pre-determined set of genes are excluded. In certain embodiments, all functional alterations in a pre-selected gene (e.g., tumor gene) in the pre-determined set of genes are excluded. In certain embodiments, a plurality of functional alterations in a plurality of pre-selected genes (e.g., tumor genes) in the pre-determined set of genes are excluded. In certain embodiments, all functional alterations in all genes (e.g., tumor genes) in the pre-determined set of genes are excluded.

In certain embodiments, the number of somatic alterations excludes a germline mutation in a sub-genomic interval.

In certain embodiments, the germline alteration is an SNP, a base substitution, an insertion, a deletion, an indel, or a silent mutation (e.g., synonymous mutation).

In certain embodiments, the germline alteration is excluded by use of a method that does not use a comparison with a matched normal sequence. In other embodiments, the germline alteration is excluded by a method comprising the use of an algorithm, for example, the somatic-germline-zygosity (SGZ) algorithm (see Sun et al. Cancer Research 2014; 74(195):1893-1893). In certain embodiments, the germline alteration is identified as such by inclusion in a database of germline alterations, for example, the dbSNP database (see Sherry et al. Nucleic Acids Res. 29(1): 308-311, 2001, which is herein incorporated by reference in its entirety). In other embodiments, the germline alteration is identified as such by inclusion in two or more counts of the ExAC database (see Exome Aggregation Consortium et al. bioRxiv preprint, Oct. 30, 2015, which is herein incorporated by reference in its entirety). In some embodiments, the germline alteration is identified as such by inclusion in the 1000 Genome Project database (McVean et al. Nature 491, 56-65, 2012, which is herein incorporated by reference in its entirety). In some embodiments, the germline alteration is identified as such by inclusion in the ESP database (Exome Variant Server, NHLBI GO Exome Sequencing Project (ESP), Seattle, WA).

The terms “programmed death ligand 1” and “PD-L1” refer herein to a native sequence PD-L1 polypeptide, polypeptide variants, and fragments of a native sequence polypeptide and polypeptide variants (which are further defined herein). The PD-L1 polypeptide described herein may be that which is isolated from a variety of sources, such as from human tissue types or from another source, or prepared by recombinant or synthetic methods.

A “native sequence PD-L1 polypeptide” comprises a polypeptide having the same amino acid sequence as the corresponding PD-L1 polypeptide derived from nature.

A “PD-L1 polypeptide variant,” or variations thereof, means a PD-L1 polypeptide, generally an active PD-L1 polypeptide, as defined herein having at least about 80% amino acid sequence identity with any of the native sequence PD-L1 polypeptide sequences as disclosed herein. Such PD-L1 polypeptide variants include, for instance, PD-L1 polypeptides wherein one or more amino acid residues are added, or deleted, at the N- or C-terminus of a native amino acid sequence. Ordinarily, a PD-L1 polypeptide variant will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a native sequence PD-L1 polypeptide sequence as disclosed herein. Ordinarily, PD-L1 variant polypeptides are at least about 10 amino acids in length, alternatively at least about 20, 30, 40, 50, 60, 70, 80, 90, 100, 110, 120, 130, 140, 150, 160, 170, 180, 190, 200, 210, 220, 230, 240, 250, 260, 270, 280, 281, 282, 283, 284, 285, 286, 287, 288, or 289 amino acids in length, or more. Optionally, PD-L1 variant polypeptides will have no more than one conservative amino acid substitution as compared to a native PD-L1 polypeptide sequence, alternatively no more than 2, 3, 4, 5, 6, 7, 8, 9, or 10 conservative amino acid substitutions as compared to a native PD-L1 polypeptide sequence.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs.

“Oligonucleotide,” as used herein, generally refers to short, single stranded, polynucleotides that are, but not necessarily, less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “primer” refers to a single-stranded polynucleotide that is capable of hybridizing to a nucleic acid and allowing polymerization of a complementary nucleic acid, generally by providing a free 3′—OH group.

The term “detection” includes any means of detecting, including direct and indirect detection.

The term “biomarker” as used herein refers to an indicator, e.g., predictive, diagnostic, and/or prognostic, which can be detected in a sample, for example, PD-L1. The biomarker may serve as an indicator of a particular subtype of a disease or disorder (e.g., cancer) characterized by certain, molecular, pathological, histological, and/or clinical features. In some embodiments, a biomarker is a gene. Biomarkers include, but are not limited to, polynucleotides (e.g., DNA and/or RNA), polynucleotide copy number alterations (e.g., DNA copy numbers), polypeptides, polypeptide and polynucleotide modifications (e.g., post-translational modifications), carbohydrates, and/or glycolipid-based molecular markers.

The “amount” or “level” of a biomarker associated with an increased clinical benefit to an individual is a detectable level in a biological sample. These can be measured by methods known to one skilled in the art and also disclosed herein. The expression level or amount of biomarker assessed can be used to determine the response to the treatment.

The terms “level of expression” or “expression level” in general are used interchangeably and generally refer to the amount of a biomarker in a biological sample. “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

“Increased expression,” “increased expression level,” “increased levels,” “elevated expression,” “elevated expression levels,” or “elevated levels” refers to an increased expression or increased levels of a biomarker in an individual relative to a control, such as an individual or individuals who are not suffering from the disease or disorder (e.g., cancer) or an internal control (e.g., a housekeeping biomarker).

“Decreased expression,” “decreased expression level,” “decreased levels,” “reduced expression,” “reduced expression levels,” or “reduced levels” refers to a decrease expression or decreased levels of a biomarker in an individual relative to a control, such as an individual or individuals who are not suffering from the disease or disorder (e.g., cancer) or an internal control (e.g., a housekeeping biomarker). In some embodiments, reduced expression is little or no expression.

The term “housekeeping biomarker” refers to a biomarker or group of biomarkers (e.g., polynucleotides and/or polypeptides) which are typically similarly present in all cell types. In some embodiments, the housekeeping biomarker is a “housekeeping gene.” A “housekeeping gene” refers herein to a gene or group of genes which encode proteins whose activities are essential for the maintenance of cell function and which are typically similarly present in all cell types.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology, (Stockton Press, N Y, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

The term “multiplex PCR” refers to a single PCR reaction carried out on nucleic acid obtained from a single source (e.g., an individual) using more than one primer set for the purpose of amplifying two or more DNA sequences in a single reaction.

“Quantitative real-time polymerase chain reaction” or “qRT-PCR” refers to a form of PCR wherein the amount of PCR product is measured at each step in a PCR reaction. This technique has been described in various publications including, for example, Cronin et al., Am. J. Pathol. 164(1):35-42 (2004) and Ma et al., Cancer Cell 5:607-616 (2004).

The term “microarray” refers to an ordered arrangement of hybridizable array elements, preferably polynucleotide probes, on a substrate.

The term “diagnosis” is used herein to refer to the identification or classification of a molecular or pathological state, disease or condition (e.g., cancer (e.g., non-small cell lung cancer (NSCLC; e.g., squamous or non-squamous NSCLC, including stage IV NSCLC). For example, “diagnosis” may refer to identification of a particular type of cancer. “Diagnosis” may also refer to the classification of a particular subtype of cancer, for instance, by histopathological criteria, or by molecular features (e.g., a subtype characterized by expression of one or a combination of biomarkers (e.g., particular genes or proteins encoded by said genes)).

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. For instance, a “tumor sample” is a tissue sample obtained from a tumor (e.g., a liver tumor) or other cancerous tissue. The tissue sample may contain a mixed population of cell types (e.g., tumor cells and non-tumor cells, cancerous cells and non-cancerous cells). The tissue sample may contain compounds which are not naturally intermixed with the tissue in nature such as preservatives, anticoagulants, buffers, fixatives, nutrients, antibiotics, or the like.

A “tumor-infiltrating immune cell,” as used herein, refers to any immune cell present in a tumor or a sample thereof. Tumor-infiltrating immune cells include, but are not limited to, intratumoral immune cells, peritumoral immune cells, other tumor stroma cells (e.g., fibroblasts), or any combination thereof. Such tumor-infiltrating immune cells can be, for example, T lymphocytes (such as CD8+T lymphocytes and/or CD4+T lymphocytes), B lymphocytes, or other bone marrow-lineage cells, including granulocytes (e.g., neutrophils, eosinophils, and basophils), monocytes, macrophages, dendritic cells (e.g., interdigitating dendritic cells), histiocytes, and natural killer cells.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample,” “reference cell,” “reference tissue,” “control sample,” “control cell,” or “control tissue,” as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, the reference sample, reference cell, reference tissue, control sample, control cell, or control tissue may be healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

For the purposes herein a “section” of a tissue sample is meant a single part or piece of a tissue sample, for example, a thin slice of tissue or cells cut from a tissue sample (e.g., a tumor sample). It is to be understood that multiple sections of tissue samples may be taken and subjected to analysis, provided that it is understood that the same section of tissue sample may be analyzed at both morphological and molecular levels, or analyzed with respect to polypeptides (e.g., by immunohistochemistry) and/or polynucleotides (e.g., by in situ hybridization).

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

The phrase “based on” when used herein means that the information about one or more biomarkers is used to inform a treatment decision, information provided on a package insert, or marketing/promotional guidance, and the like.

The word “label” when used herein refers to a compound or composition that is conjugated or fused directly or indirectly to a reagent such as a polynucleotide probe or an antibody and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which is detectable. The term is intended to encompass direct labeling of a probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin.

The term “dysfunction,” in the context of immune dysfunction, refers to a state of reduced immune responsiveness to antigenic stimulation. The term includes the common elements of both “exhaustion” and/or “anergy” in which antigen recognition may occur, but the ensuing immune response is ineffective to control infection or tumor growth.

The term “dysfunctional,” as used herein, also includes refractory or unresponsive to antigen recognition, specifically, impaired capacity to translate antigen recognition into down-stream T-cell effector functions, such as proliferation, cytokine production (e.g., IL-2) and/or target cell killing.

The term “anergy” refers to the state of unresponsiveness to antigen stimulation resulting from incomplete or insufficient signals delivered through the T-cell receptor (e.g., increase in intracellular Ca²⁺in the absence of ras-activation). T cell anergy can also result upon stimulation with antigen in the absence of co-stimulation, resulting in the cell becoming refractory to subsequent activation by the antigen even in the context of co-stimulation. The unresponsive state can often be overridden by the presence of interleukin-2. Anergic T-cells do not undergo clonal expansion and/or acquire effector functions.

The term “exhaustion” refers to T cell exhaustion as a state of T cell dysfunction that arises from sustained TCR signaling that occurs during many chronic infections and cancer. It is distinguished from anergy in that it arises not through incomplete or deficient signaling, but from sustained signaling. It is defined by poor effector function, sustained expression of inhibitory receptors and a transcriptional state distinct from that of functional effector or memory T cells. Exhaustion prevents optimal control of infection and tumors. Exhaustion can result from both extrinsic negative regulatory pathways (e.g., immunoregulatory cytokines) as well as cell intrinsic negative regulatory (costimulatory) pathways (PD-1, B7-H3, B7-H4, etc.).

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage and tumor clearance.

“Immunogenicity” refers to the ability of a particular substance to provoke an immune response. Tumors are immunogenic and enhancing tumor immunogenicity aids in the clearance of the tumor cells by the immune response. Examples of enhancing tumor immunogenicity include treatment with a PD-1 axis binding antagonist (e.g., an anti-PD-L1 antibody (e.g., atezolizumab)) or treatment with a PD-1 axis binding antagonist (e.g., an anti-PD-L1 antibody (e.g., atezolizumab)) and a platinum-based chemotherapy (e.g., cisplatin or carboplatin and gemcitabine).

The term “respond to” or “responsive to” in the context of the present disclosure indicates that a patient suffering, suspected to suffer, or prone to suffer from cancer (e.g., NSCLC, such as squamous or non-squamous NSCLC, including stage IV NSCLC), shows a response to a therapy, e.g., an anti-cancer therapy that includes a PD-1 axis binding antagonist (e.g., an anti-PD-L1 antibody (e.g., atezolizumab) or an anti-PD-1 antibody) and/or a platinum-based chemotherapy (e.g., cisplatin or carboplatin and gemcitabine). A skilled person will readily be in a position to determine whether a person treated with an anti-cancer therapy that includes a PD-1 axis binding antagonist (e.g., an anti-PD-L1 antibody (e.g., atezolizumab) or an anti-PD-1 antibody) and/or a platinum-based chemotherapy (e.g., cisplatin or carboplatin and gemcitabine) according to the methods of the disclosure shows a response. For example, a response may be reflected by decreased suffering from cancer, such as a diminished and/or halted tumor growth, reduction of the size of a tumor, and/or amelioration of one or more symptoms of cancer. Preferably, the response may be reflected by decreased or diminished indices of the metastatic conversion of the cancer or indices of the cancer, e.g., the prevention of the formation of metastases or a reduction of number or size of metastases. A response may be, e.g., a complete response, a partial response, an improvement in progression-free survival, an improvement in overall survival, a sustained response, and/or an improvement in duration of response (DOR).

In some embodiments, a bTMB score determined using methods disclosed herein to be at or above a reference bTMB score (e.g., a reference bTMB score between about 4 and about 30, e.g., a reference bTMB score of about 4, 5, 6, 7, 8, 9, 10, 11, 12, 13, 14, 15, 16, 17, 18, 19, 20, 21, 22, 23, 24, 25, 26, 27, 28, 29, or 30, such as a reference bTMB score of 16) is used to identify a patient who is predicted to have an increased likelihood of being responsive to treatment with a medicament (e.g., treatment comprising a PD-1 axis binding antagonist, e.g., an anti-PD-L1 antibody). In some embodiments, a bTMB score determined using methods disclosed herein to be less than a reference bTMB score (e.g., a reference bTMB score between about 4 and about 30, e.g., a reference bTMB score of about 4, 5, 6, 7, 8, 9, 10, 11, 12, 13, 14, 15, 16, 17, 18, 19, 20, 21, 22, 23, 24, 25, 26, 27, 28, 29, or 30, such as a reference bTMB score of 16) is used to identify a patient who is predicted to have an increased likelihood of being responsive to treatment with an anti-cancer therapy other than, or in addition to, a PD-1 axis binding antagonist. In some instances, the bTMB score determined from a sample from an individual is between about 8 and about 100 (e.g., 8, 9, 10, 15, 20, 25, 30, 35, 40, 45, 50, 55, 60, 65, 70, 75, 80, 85, 90, 95, or 100).

In general, the bTMB score (e.g., a reference bTMB score) is linearly related to the size of the genomic region sequenced. The example numbers above refer to bTMB scores obtained by sequencing about 1.1 Mb, e.g., using the FOUNDATIONONE® panel. The bTMB score of a sample when sequencing X times more bases is expected to be about X times higher. In some embodiments, a normalized bTMB value can be calculated by dividing the number of somatic variations (e.g., mutations) counted by the number of bases sequenced, e.g., the number of somatic variations (e.g., mutations) counted per megabase. Accordingly, any of the preceding bTMB scores or reference bTMB scores can be an equivalent bTMB value, for example, an equivalent bTMB value determined by whole-exome sequencing. In some instances, a bTMB score (e.g., a reference bTMB score) may be between about 400 and about 1500 (e.g., a bTMB score of about 400, 500, 600, 700, 800, 900, 1000, 1100, 1200, 1300, 1400, or 1500), for example, in a whole-exome-based assay.

In some embodiments, a combination of a bTMB score and MSAF, determined using methods disclosed herein, is used to identify a patient who is predicted to have an increased likelihood of being responsive to treatment with a medicament (e.g., treatment comprising a PD-1 axis binding antagonist, e.g., an anti-PD-L1 antibody). In some embodiments, a combination of a bTMB score and MSAF, determined using methods disclosed herein, is used to identify a patient who is predicted to have an increased likelihood of being responsive to treatment with an anti-cancer therapy other than, or in addition to, a PD-1 axis binding antagonist.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5X, 2.0X, 2.5X, or 3.0X length of the treatment duration.

As used herein, “reducing or inhibiting cancer relapse” means to reduce or inhibit tumor or cancer relapse or tumor or cancer progression. As disclosed herein, cancer relapse and/or cancer progression include, without limitation, cancer metastasis.

As used herein, “complete response” or “CR” refers to disappearance of all target lesions.

As used herein, “partial response” or “PR” refers to at least a 30% decrease in the sum of the longest diameters (SLD) of target lesions, taking as reference the baseline SLD.

As used herein, “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD recorded since the treatment started or the presence of one or more new lesions.

As used herein, “progression-free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease. In some embodiments, PFS may be defined as the time from randomization or the beginning of treatment to the first documented disease progression as assessed by RECIST v1.1, or death from any cause, whichever occurs first.

As used herein, “objective response rate” or “objective response rate” (ORR) refers to the sum of complete response (CR) rate and partial response (PR) rate. For example, in some embodiments, ORR refers to the proportion of patients with a confirmed objective response, either CR or PR, observed on two assessments greater than or equal to 28 days apart per RECIST v1.1, based on investigator assessment.

As used herein, “overall survival” (OS) refers to the percentage of individuals in a group who are likely to be alive after a particular duration of time.

As used herein, the term “duration of response” (DOR) refers to a length of time from documentation of a tumor response until disease progression or death from any cause, whichever occurs first.

As used herein, the terms “inoperable” and “unresectable” are used interchangeably to refer to a cancer (e.g., NSCLC, such as squamous or non-squamous NSCLC, including stage IV NSCLC) for which surgical resection is not possible or cannot be safely performed.

The term “eligible for treatment with a platinum-based chemotherapy” means that the subject is eligible for treatment with a platinum-based chemotherapy, either in the attending clinician's judgment or according to standardized criteria for eligibility for platinum-based chemotherapy that are known in the art. For example, the criteria set forth in Galsky et al. Lancet Oncol. 12(3):211-4, 2011 may be used to determine whether a subject is eligible for cisplatin-based chemotherapy. In one example, a patient is considered unfit for cisplatin-based chemotherapy if they have one or more of the following: impaired renal function (e.g., glomerular filtration rate (GFR)>30 but <60 mL/min); GFR may be assessed by direct measurement (i.e., creatinine clearance or ethyldediaminetetra-acetate) or, if not available, by calculation from serum/plasma creatinine (Cockcroft-Gault formula)); hearing loss (e.g., National Cancer Institute (NCI) Common Terminology Criteria for Adverse Events (CTCAE) v4.0 Grade 2 audiometric hearing loss of 25 decibels at two contiguous frequencies); peripheral neuropathy (e.g., NCI CTCAE v4.0 Grade 2 peripheral neuropathy (i.e., sensory alteration or paresthesia, including tingling)); and/or ECOG performance status assessment (see Oken et al. Am. J. Clin. Oncol. 5:649-655, 1982) (e.g., an ECOG performance status of 2). In some embodiments, a subject having one of the following may be eligible for carboplatin-based chemotherapy: impaired renal function (e.g., glomerular filtration rate (GFR)>30 but <60 mL/min); GFR may be assessed by direct measurement (i.e., creatinine clearance or ethyldediaminetetra-acetate) or, if not available, by calculation from serum/plasma creatinine (Cockcroft-Gault formula)); hearing loss (e.g., CTCAE v4.0 Grade 2 audiometric hearing loss of 25 decibels at two contiguous frequencies); peripheral neuropathy (e.g., NCI CTCAE v4.0 Grade 2 peripheral neuropathy (i.e., sensory alteration or paresthesia, including tingling)); and/or ECOG performance status assessment (e.g., an ECOG performance status of 2).

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with cancer (e.g., NSCLC, such as squamous or non-squamous NSCLC, including stage IV NSCLC) are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of individuals.

As used herein, “delaying progression” of a disease means to defer, hinder, slow, retard, stabilize, and/or postpone development of the disease (such as cancer (e.g., NSCLC, such as squamous or non-squamous NSCLC, including stage IV NSCLC)). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

An “effective amount” or “therapeutically effective amount,” as used interchangeably herein, is at least the minimum amount required to effect a measurable improvement or prevention of a particular disorder. An effective amount herein may vary according to factors such as the disease state, age, sex, and weight of the patient, and the ability of the agent to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, and enhancing effect of another medication such as via targeting, delaying the progression of the disease, and/or prolonging survival. In the case of a cancer or a tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this disclosure, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

The term “PD-1 axis binding antagonist” refers to a molecule that inhibits the interaction of a PD-1 axis binding partner with either one or more of its binding partner, so as to remove T-cell dysfunction resulting from signaling on the PD-1 signaling axis, with a result being to restore or enhance T-cell function (e.g., proliferation, cytokine production, and/or target cell killing). As used herein, a PD-1 axis binding antagonist includes a PD-L1 binding antagonist, a PD-1 binding antagonist, and a PD-L2 binding antagonist.

The term “PD-L1 binding antagonist” refers to a molecule that decreases, blocks, inhibits, abrogates, or interferes with signal transduction resulting from the interaction of PD-L1 with either one or more of its binding partners, such as PD-1 and/or B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen-binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B7-1. In one embodiment, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some embodiments, a PD-L1 binding antagonist is an anti-PD-L1 antibody. In a specific aspect, an anti-PD-L1 antibody is atezolizumab, marketed as TECENTRIQ® with a WHO Drug Information (International Nonproprietary Names for Pharmaceutical Substances), Proposed INN: List 112, Vol. 28, No. 4, published Jan. 16, 2015 (see page 485) described herein. In another specific aspect, an anti-PD-L1 antibody is MDX-1105 described herein. In still another specific aspect, an anti-PD-L1 antibody is YW243.55.570 described herein. In still another specific aspect, an anti-PD-L1 antibody is MED14736 (durvalumab) described herein. In still another specific aspect, an anti-PD-L1 antibody is MSB0010718C (avelumab) described herein.

The term “PD-1 binding antagonist” refers to a molecule that decreases, blocks, inhibits, abrogates or interferes with signal transduction resulting from the interaction of PD-1 with one or more of its binding partners, such as PD-L1 and/or PD-L2. In some embodiments, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen-binding fragments thereof, immunoadhesins, fusion proteins, oligopeptides, and other molecules that decrease, block, inhibit, abrogate or interfere with signal transduction resulting from the interaction of PD-1 with PD-L1 and/or PD-L2. In one embodiment, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some embodiments, the PD-1 binding antagonist is an anti-PD-1 antibody. In a specific aspect, a PD-1 binding antagonist is MDX-1106 (nivolumab) described herein. In another specific aspect, a PD-1 binding antagonist is MK-3475 (pembrolizumab) described herein. In another specific aspect, a PD-1 binding antagonist is MEDI-0680 (AMP-514) described herein. In another specific aspect, a PD-1 binding antagonist is PDR001 described herein. In another specific aspect, a PD-1 binding antagonist is REGN2810 described herein. In another specific aspect, a PD-1 binding antagonist is BGB-108 described herein.

The term “PD-L2 binding antagonist” refers to a molecule that decreases, blocks, inhibits, abrogates or interferes with signal transduction resulting from the interaction of PD-L2 with either one or more of its binding partners, such as PD-1. In some embodiments, a PD-L2 binding antagonist is a molecule that inhibits the binding of PD-L2 to one or more of its binding partners. In a specific aspect, the PD-L2 binding antagonist inhibits binding of PD-L2 to PD-1. In some embodiments, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one embodiment, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some embodiments, a PD-L2 binding antagonist is an immunoadhesin.

A “disorder” is any condition that would benefit from treatment including, but not limited to, chronic and acute disorders or diseases including those pathological conditions which predispose the mammal to the disorder in question. Exemplary disorders include cancer (e.g., NSCLC, such as squamous or non-squamous NSCLC, including stage IV NSCLC).

The terms “cell proliferative disorder” and “proliferative disorder” refer to disorders that are associated with some degree of abnormal cell proliferation. In one embodiment, the cell proliferative disorder is cancer. In one embodiment, the cell proliferative disorder is a tumor.

The term “tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The terms “cancer” and “cancerous” refer to or describe the physiological condition in mammals that is typically characterized by unregulated cell growth. Included in this definition are benign and malignant cancers. The terms “non-small cell lung cancer” and its abbreviation, “NSCLC,” include, but are not limited to, squamous and non-squamous NSCLC. The methods described herein are suitable for treatment of various stages of cancer, including cancers that are locally advanced and/or metastatic. In cancer staging, locally advanced is generally defined as cancer that has spread from a localized area to nearby tissues and/or lymph nodes. In the Roman numeral staging system, locally advanced usually is classified in Stage II or III. Cancer which is metastatic is a stage where the cancer spreads throughout the body to distant tissues and organs (stage IV).

The term “cytotoxic agent” as used herein refers to any agent that is detrimental to cells (e.g., causes cell death, inhibits proliferation, or otherwise hinders a cellular function). Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; growth inhibitory agents; enzymes and fragments thereof such as nucleolytic enzymes; and toxins such as small molecule toxins or enzymatically active toxins of bacterial, fungal, plant or animal origin, including fragments and/or variants thereof. Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signaling inhibitors, HDAC inhibitors, proteasome inhibitors, and inhibitors of cancer metabolism. In one embodiment, the cytotoxic agent is a platinum-based chemotherapeutic agent. In one embodiment, the cytotoxic agent is an antagonist of EGFR. In one embodiment the cytotoxic agent is N-(3-ethynylphenyl)-6,7-bis(2-methoxyethoxy)quinazolin-4-amine (e.g., erlotinib, TARCEVA™). In one embodiment the cytotoxic agent is a RAF inhibitor. In one embodiment, the RAF inhibitor is a BRAF and/or CRAF inhibitor. In one embodiment the RAF inhibitor is vemurafenib. In one embodiment, the cytotoxic agent is a P13K inhibitor.

As used herein, the term “chemotherapeutic agent” includes compounds useful in the treatment of cancer, such as NSCLC (e.g., squamous or non-squamous NSCLC, including stage IV NSCLC). Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, rapamycin (Sirolimus, RAPAMUNE®, Wyeth), lapatinib (TYKERB®, GSK572016, Glaxo Smith Kline), lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 33:183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxanes; chloranmbucil; GEMZAR® (gemcitabine); 6-thioguanine; mercaptopurine; methotrexate; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids, and derivatives of any of the above.

Chemotherapeutic agents also include “platinum-based” chemotherapeutic agents, which comprise an organic compound which contains platinum as an integral part of the molecule. Typically, platinum-based chemotherapeutic agents are coordination complexes of platinum. Platinum-based chemotherapeutic agents are sometimes called “platins” in the art. Examples of platinum-based chemotherapeutic agents include, but are not limited to, cisplatin, carboplatin, oxaliplatin, nedaplatin, triplatin tetranitrate, phenanthriplatin, picoplatin, lipoplatin, and satraplatin.

A “platinum-based chemotherapy,” as used herein, refers to a chemotherapy regimen that includes a platinum-based chemotherapeutic agent. For example, a platinum-based chemotherapy may include a platinum-based chemotherapeutic agent (e.g., cisplatin or carboplatin) in combination with one or more additional chemotherapeutic agents, e.g., a nucleoside analog (e.g., gemcitabine).

A “nucleoside analog,” as used herein, refers to a nucleoside that includes a nucleic acid analog and a sugar. Nucleoside analogs may function as antimetabolites. Exemplary nucleoside analogues include but are not limited to gemcitabine, cytarabine, fludarabine, and cladribine.

Chemotherapeutic agents also include (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids, and derivatives of any of the above.

Chemotherapeutic agents also include antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the disclosure include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories), which is a recombinant exclusively human-sequence, full-length IgGi A antibody genetically modified to recognize interleukin-12 p40 protein.

Chemotherapeutic agents also include “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533) and variants thereof, such as chimerized 225 (C225 or Cetuximab; ERBUTIX®) and reshaped human 225 (H225) (see, e.g., WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al., Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyOmethoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER-targeted tyrosine kinase inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², and radioactive isotopes of Lu); miscellaneous investigational agents such as thioplatin, PS-341, phenylbutyrate, ET-18-OCH₃, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g., celecoxib or etoricoxib), proteosome inhibitor (e.g., PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as metastatic bone pain, headache and migraine, postoperative pain, mild-to-moderate pain due to inflammation and tissue injury, pyrexia, ileus, and renal colic.

As used herein, the term “chemotherapy-naïve” refers to a patient having cancer (e.g., a cancer described herein, such as NSCLC, including squamous NSCLC and non-squamous NSCLC) that has not received chemotherapy for the treatment of the cancer within the past six months relative to the time at which the subject is administered a specified therapeutic agent, such as a PD-1 axis binding antagonist described herein. In some embodiments, a chemotherapy-naïve subject has also not been administered neo-adjuvant therapy, radiotherapy, or chemoradiotherapy within the past six months relative to the time at which the subject is administered a specified therapeutic agent, such as a PD-1 axis binding antagonist described herein.

A “growth inhibitory agent” when used herein refers to a compound or composition which inhibits growth of a cell either in vitro or in vivo. In one embodiment, a growth inhibitory agent is growth inhibitory antibody that prevents or reduces proliferation of a cell expressing an antigen to which the antibody binds. In another embodiment, the growth inhibitory agent may be one which significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13.

The term “prodrug” as used herein refers to a precursor or derivative form of a pharmaceutically active substance that is less cytotoxic to tumor cells compared to the parent drug and is capable of being enzymatically activated or converted into the more active parent form. See, for example, Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this disclosure include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this disclosure include, but are not limited to, those chemotherapeutic agents described above.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

An “anti-angiogenesis agent” or “angiogenesis inhibitor” refers to a small molecular weight substance, a polynucleotide, a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenesis agent includes those agents that bind and block the angiogenic activity of the angiogenic factor or its receptor.

For example, an anti-angiogenesis agent is an antibody or other antagonist to an angiogenic agent as defined above, e.g., antibodies to VEGF-A or the VEGF-A receptor (e.g., KDR receptor or Flt-1 receptor), anti-PDGFR inhibitors such as GLEEVEC™ (imatinib mesylate). Anti-angiogenesis agents also include native angiogenesis inhibitors, e.g., angiostatin, endostatin, etc. See, for example, Klagsbrun and D′Amore, Annu. Rev. Physiol., 53:217-39 (1991); Streit and Detmar, Oncogene, 22:3172-3179 (2003) (e.g., Table 3 listing anti-angiogenic therapy in malignant melanoma); Ferrara & Alitalo, Nature Medicine 5(12):1359-1364 (1999); Tonini et al., Oncogene, 22:6549-6556 (2003) and, Sato Int. J. Clin. Oncol., 8:200-206 (2003).

The terms a “subject,” an “individual,” or a “patient,” as used interchangeably herein, for purposes of treatment refer to any animal classified as a mammal, including humans, domestic and farm animals, and zoo, sports, or pet animals, such as cats, dogs, horses, cows, and the like. Preferably, the mammal is human.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

An “isolated” antibody is one which has been identified and separated and/or recovered from a component of its natural environment. Contaminant components of its natural environment are materials which would interfere with research, diagnostic or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. An isolated antibody includes the antibody in situ within recombinant cells since at least one component of the antibody's natural environment will not be present. Ordinarily, however, an isolated antibody will be prepared by at least one purification step.

“Native antibodies” are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The term “constant domain” refers to the portion of an immunoglobulin molecule having a more conserved amino acid sequence relative to the other portion of the immunoglobulin, the variable domain, which contains the antigen binding site. The constant domain contains the C_{H}1, C_{H}2 and C_{H}3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “V_{L}.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The “light chains” of antibodies (immunoglobulins) from any mammalian species can be assigned to one of two clearly distinct types, called kappa (“K”) and lambda (“A”), based on the amino acid sequences of their constant domains.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full-length antibody,” “intact antibody,” and “whole antibody” are used herein interchangeably to refer to an antibody in its substantially intact form, not antibody fragments as defined below. The terms particularly refer to an antibody with heavy chains that contain an Fc region.

A “naked antibody” for the purposes herein is an antibody that is not conjugated to a cytotoxic moiety or radiolabel.

“Antibody fragments” comprise a portion of an intact antibody, preferably comprising the antigen-binding region thereof. In some embodiments, the antibody fragment described herein is an antigen-binding fragment. Examples of antibody fragments include Fab, Fab′, F(ab′)2, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (C_{H}1) of the heavy chain. Fab′ fragments differ from Fab fragments by the addition of a few residues at the carboxy terminus of the heavy chain C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckthün, in The Pharmacology of Monoclonal Antibodies, vol. 113, Rosenburg and Moore eds., (Springer-Verlag, New York, 1994), pp. 269-315.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this disclosure. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies and is not to be construed as requiring production of the antibody by any particular method. For example, the monoclonal antibodies to be used in accordance with the disclosure may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg et al., Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATIZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR of the recipient are replaced by residues from an HVR of a non-human species (donor antibody) such as mouse, rat, rabbit, or nonhuman primate having the desired specificity, affinity, and/or capacity. In some embodiments, FR residues of the human immunoglobulin are replaced by corresponding non-human residues.

Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. BioL, 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. PharmacoL, 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

A “species-dependent antibody” is one which has a stronger binding affinity for an antigen from a first mammalian species than it has for a homologue of that antigen from a second mammalian species. Normally, the species-dependent antibody “binds specifically” to a human antigen (e.g., has a binding affinity (Kd) value of no more than about 1×10⁻⁷ M, preferably no more than about 1×10⁻⁸ M and preferably no more than about 1×10⁻⁹ M) but has a binding affinity for a homologue of the antigen from a second nonhuman mammalian species which is at least about 50 fold, or at least about 500 fold, or at least about 1000 fold, weaker than its binding affinity for the human antigen. The species-dependent antibody can be any of the various types of antibodies as defined above, but preferably is a humanized or human antibody.

The term “hypervariable region,” “HVR,” or “HV,” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc., according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

The expression “linear antibodies” refers to the antibodies described in Zapata et al. (1995 Protein Eng, 8(10):1057-1062). Briefly, these antibodies comprise a pair of tandem Fd segments (VH-C_{H}1-VH-C_{H}1) which, together with complementary light chain polypeptides, form a pair of antigen binding regions. Linear antibodies can be bispecific or monospecific.

As used herein, the term “binds,” “specifically binds to,” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of 1 μM. 100 nM, 10 nM, 1 nM, or 0.1 nM. In certain embodiments, an antibody specifically binds to an epitope on a protein that is conserved among the protein from different species. In another embodiment, specific binding can include, but does not require exclusive binding.

“Percent (%) amino acid sequence identity” with respect to the polypeptide sequences identified herein is defined as the percentage of amino acid residues in a candidate sequence that are identical with the amino acid residues in the polypeptide being compared, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program ALIGN-2. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, California. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The amino acid sequences described herein are contiguous amino acid sequences unless otherwise specified.

The term “package insert” is used to refer to instructions customarily included in commercial packages of therapeutic products, that contain information about the indications, usage, dosage, administration, combination therapy, contraindications and/or warnings concerning the use of such therapeutic products.

The terms “pharmaceutical formulation” and “pharmaceutical composition” are used interchangeably herein and refer to a preparation which is in such form as to permit the biological activity of an active ingredient contained therein to be effective, and which contains no additional components which are unacceptably toxic to a subject to which the formulation would be administered. Such formulations are sterile. In a preferred embodiment, the pharmaceutical composition or pharmaceutical formulation is administered to a human subject.

A “sterile” pharmaceutical formulation is aseptic or free or essentially free from all living microorganisms and their spores.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “administering” is meant a method of giving a dosage of a compound (e.g., a PD-1 axis binding antagonist (e.g., an anti-PD-L1 antibody (e.g., atezolizumab) or an anti-PD-1 antibody) and/or a platinum-based chemotherapy (e.g., cisplatin or carboplatin and gemcitabine)) or a composition (e.g., a pharmaceutical composition, e.g., a pharmaceutical composition including a PD-1 axis binding antagonist and/or a platinum-based chemotherapy, optionally also including an additional therapeutic agent) to a subject. The compositions utilized in the methods described herein can be administered, for example, intravitreally, intramuscularly, intravenously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subcutaneously, subconjunctivally, intravesicularly, mucosally, intrapericardially, intraumbilically, intraocularly, intraorbitally, orally, topically, transdermally, periocularly, conjunctivally, subtenonly, intracamerally, subretinally, retrobulbarly, intracanalicularly, by inhalation, by injection, by implantation, by infusion, by continuous infusion, by localized perfusion bathing target cells directly, by catheter, by lavage, in cremes, or in lipid compositions. The compositions utilized in the methods described herein can also be administered systemically or locally. The method of administration can vary depending on various factors (e.g., the compound or composition being administered, and the severity of the condition, disease, or disorder being treated).

As used herein, “in combination with” or “in conjunction with” refers to administration of one treatment modality in addition to another treatment modality, for example, a treatment regimen that includes administration of a PD-1 axis binding antagonist (e.g., an anti-PD-L1 antibody such as atezolizumab) and a platinum-based chemotherapy (e.g., cisplatin or carboplatin and gemcitabine). As such, “in conjunction with” refers to administration of one treatment modality before, during, or after administration of the other treatment modality to the individual.

III. Diagnostic Methods and Compositions for Evaluating Subjects with NSCLC

In particular instances, the methods and assays provided herein may be used to identify an individual having a cancer who may benefit from a treatment including a PD-1 axis binding antagonist, such as an anti-PD-L1 antibody (e.g., atezolizumab), among others described herein. In particular instances, the methods and assays provided herein may be used to select a therapy for an individual having a cancer (e.g., squamous or non-squamous NSCLC), the method including determining a bTMB score from a sample from the individual, wherein a bTMB score from the sample that is at or above a reference bTMB score identifies the individual as one who may benefit from a treatment comprising a PD-1 axis binding antagonist (e.g., an anti-PD-L1 antibody, such as atezolizumab), among others described herein.

The methods provided herein may include determining a bTMB score from a sample (e.g., a whole blood sample, a plasma sample, a serum sample, or a combination thereof) from an individual. The sample from the individual may be an archival sample, a fresh sample, or a frozen sample. The determination step may include determining the total number of somatic mutations (e.g., a base substitution in a coding region and/or an indel mutation in a coding region) occurring in a pre-determined set of genes to derive a bTMB score from the sample from the individual. In some embodiments, the number of somatic mutations is the number of single nucleotide variants (SNVs) counted or a sum of the number of SNVs and the number of indel mutations counted.

The number of somatic mutations can be determined qualitatively and/or quantitatively based on any suitable criterion known in the art, including, but not limited to, the measurement of DNA, mRNA, cDNA, proteins, protein fragments, and/or gene copy number levels in an individual. In some instances, a comprehensive genomic profile of an individual is determined. In some instances, a comprehensive genomic profile of a sample (e.g., a whole blood sample, a plasma sample, a serum sample, or a combination thereof) collected from an individual is determined. In some instances, the determination of the genomic profile comprises applying next-generation sequencing methods, known in the art or described herein, to identify genomic alterations (e.g., somatic mutations (e.g., a base substitution in a coding region and/or an indel mutation in a coding region)). In some instances, the test simultaneously sequences the coding region of about 300 genes (e.g., a diverse set of at least about 300 to about 400 genes, e.g., about 300, 310, 320, 330, 340, 350, 360, 370, 380, 390, or 400 genes) covering at least about 0.05 Mb to about 10 Mb (e.g., 0.05, 0.06. 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10 Mb) to a typical median depth of exon coverage of at least about 500×(e.g., 500×, 550×, 600×, 650×, 700×, 750×, 800×, 850×, 900×, 950×, or 1,000×). In other instances, the test simultaneously sequences the coding regions of about 400 genes, about 425 genes, about 450 genes, about 475 genes, about 500 genes, about 525 genes, about 550 genes, about 575 genes, about 600 genes, about 625 genes, about 650 genes, about 675 genes, about 700 genes, about 725 genes, about 750 genes, about 775 genes, about 800 genes, about 825 genes, about 850 genes, about 875 genes, about 900 genes, about 925 genes, about 950 genes, about 975 genes, about 1000 genes, or greater than 1000 genes. In some instances, the set of genes includes one or more genes (e.g., cancer-related genes) set forth in Table 1. In some instances, the set of genes is the set of genes of the FOUNDATIONONE® panel (see, e.g., Frampton et al. Nat. Biotechnol. 31:1023-31, 2013, which is incorporated herein by reference in its entirety). In some instances, the set of genes is the set of genes of the FOUNDATIONONE® CDx panel. In some embodiments, the test sequences greater than about 10 Mb of the genome of the individual, e.g., greater than about 10 Mb, greater than about 15 Mb, greater than about 20 Mb, greater than about 25 Mb, greater than about 30 Mb, greater than about 35 Mb, greater than about 40 Mb, greater than about 45 Mb, greater than about 50 Mb, greater than about 55 Mb, greater than about 60 Mb, greater than about 65 Mb, greater than about 70 Mb, greater than about 75 Mb, greater than about 80 Mb, greater than about 85 Mb, greater than about 90 Mb, greater than about 95 Mb, greater than about 100 Mb, greater than about 200 Mb, greater than about 300 Mb, greater than about 400 Mb, greater than about 500 Mb, greater than about 600 Mb, greater than about 700 Mb, greater than about 800 Mb, greater than about 900 Mb, greater than about 1 Gb, greater than about 2 Gb, greater than about 3 Gb, or about 3.3 Gb. In some instances, the bTMB score is determined by whole-exome sequencing. In some instances, the bTMB score is determined by whole-genome sequencing. It is presently understood that a bTMB score may be calculated independent of gene identity. In some instances, each covered sequencing read represents a unique DNA fragment to enable the highly sensitive and specific detection of genomic alterations that occur at low frequencies due to tumor heterogeneity, low tumor purity, and small sample volumes. The determination step may include determining the number of somatic mutations in cell free DNA (cfDNA) and/or circulating tumor DNA (ctDNA) isolated from the sample (e.g., a whole blood sample, a plasma sample, a serum sample, or a combination thereof) from the individual to derive a bTMB score. In some embodiments, the amount of cfDNA isolated from the sample is at least about 5 ng (e.g., at least about 5 ng, at least about 10 ng, at least about 15 ng, at least about 20 ng, at least about 25 ng, at least about 30 ng, at least about 35 ng, at least about 40 ng, at least about 45 ng, at least about 50 ng, at least about 75 ng, at least about 100 ng, at least about 200 ng, at least about 300 ng, at least about 400 ng, or more). For example, in some embodiments, the amount of cfDNA isolated from the sample is at least about 20 ng of cfDNA. In some embodiments, the amount of cfDNA isolated from the sample is, for example, from about 5 ng to about 100 ng (e.g., from about 5 ng to about 100 ng, from about 5 ng to about 90 ng, from about 5 ng to about 80 ng, from about 5 ng to about 70 ng, from about 5 ng to about 60 ng, from about 5 ng to about 50 ng, from about 5 ng to about 40 ng, from about 5 ng to about 30 ng, from about 5 ng to about 20 ng, from about 5 ng to about 15 ng, from about 5 ng to about 10 ng, from about 10 ng to about 100 ng, from about 10 ng to about 90 ng, from about 10 ng to about 80 ng, from about 10 ng to about 70 ng, from about 10 ng to about 60 ng, from about 10 ng to about 50 ng, from about 10 ng to about 40 ng, from about 10 ng to about 30 ng, from about 10 ng to about 20 ng, from about 15 ng to about 100 ng, from about 15 ng to about 90 ng, from about 15 ng to about 80 ng, from about 15 ng to about 70 ng, from about 15 ng to about 60 ng, from about 15 ng to about 50 ng, from about 20 ng to about 100 ng, from about 20 ng to about 90 ng, from about 20 ng to about 80 ng, from about 20 ng to about 70 ng, from about 20 ng to about 60 ng, from about 20 ng to about 50 ng, from about 20 ng to about 40 ng, from about 20 ng to about 30 ng, from about 25 ng to about 100 ng, from about 25 ng to about 90 ng, from about 25 ng to about 80 ng, from about 25 ng to about 70 ng, from about 25 ng to about 60 ng, from about 25 ng to about 50 ng, from about 25 ng to about 40 ng, from about 25 ng to about 30 ng, from about 30 ng to about 100 ng, from about 30 ng to about 90 ng, from about 30 ng to about 80 ng, from about 30 ng to about 70 ng, from about 30 ng to about 60 ng, from about 30 ng to about 50 ng, from about 30 ng to about 40 ng, from about 30 ng to about 35 ng, from about 35 ng to about 100 ng, from about 35 ng to about 90 ng, from about 35 ng to about 80 ng, from about 35 ng to about 70 ng, from about 35 ng to about 60 ng, from about 35 ng to about 50 ng, from about 35 ng to about 40 ng, from about 40 ng to about 100 ng, from about 40 ng to about 90 ng, from about 40 ng to about 80 ng, from about 40 ng to about 70 ng, from about 40 ng to about 60 ng, from about 40 ng to about 50 ng, from about 40 ng to about 45 ng, from about 50 ng to about 100 ng, from about 50 ng to about 90 ng, from about 50 ng to about 80 ng, from about 50 ng to about 70 ng, from about 50 ng to about 60 ng, from about 60 ng to about 100 ng, from about 60 ng to about 90 ng, from about 60 ng to about 80 ng, from about 60 ng to about 70 ng, from about 70 ng to about 100 ng, from about 70 ng to about 90 ng, from about 70 ng to about 80 ng, from about 80 ng to about 100 ng, from about 80 ng to about 90 ng, or from 90 ng to about 100 ng). In some embodiments, the amount of cfDNA isolated from the sample is about 100 ng or more (e.g., about 100 ng or more, about 200 ng or more, about 300 ng or more, about 400 ng or more, about 500 ng or more, about 600 ng or more, about 700 ng or more, about 800 ng or more, about 900 ng or more, or higher).

Any suitable sample volume may be used in any of the preceding methods. For example, in some instances, the sample (e.g., a whole blood sample, a plasma sample, a serum sample, or a combination thereof) may have a volume of about 1 mL to about 50 mL, e.g., about 1 mL, about 2 mL, about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, about 22 mL, about 24 mL, about 26 mL, about 28 mL, about 30 mL, about 32 mL, about 34 mL, about 36 mL, about 38 mL, about 40 mL, about 42 mL, about 44 mL, about 46 mL, about 48 mL, or about 50 mL. In some instances, the sample (e.g., a whole blood sample, a plasma sample, a serum sample, or a combination thereof) may have a volume of from about 1 mL to about 50 mL, from about 1 mL to about 40 mL, from about 1 mL to about 30 mL, from about 1 mL to about 20 mL, from about 1 mL to about 10 mL, from about 5 mL to about 50 mL, from about 5 mL to about 40 mL, from about 5 mL to about 30 mL, from about 5 mL to about 20 mL, from about 5 mL to about 10 mL, from about 6 mL to about 50 mL, from about 6 mL to about 40 mL, from about 6 mL to about 30 mL, from about 6 mL to about 20 mL, from about 6 mL to about 10 mL, from about 7 mL to about 50 mL, from about 7 mL to about 40 mL, from about 7 mL to about 30 mL, from about 7 mL to about 20 mL, from about 7 mL to about 10 mL, from about 8 mL to about 50 mL, from about 8 mL to about 40 mL, from about 8 mL to about 30 mL, from about 8 mL to about 20 mL, from about 8 mL to about 10 mL, from about 9 mL to about 50 mL, from about 9 mL to about 40 mL, from about 9 mL to about 30 mL, from about 9 mL to about 20 mL, from about 9 mL to about 10 mL, from about 5 mL to about 15 mL, from about 5 mL to about 14 mL, from about 5 mL to about 13 mL, from about 5 mL to about 12 mL, from about 5 mL to about 11 mL, from about 6 mL to about 15 mL, from about 6 mL to about 14 mL, from about 6 mL to about 13 mL, from about 6 mL to about 12 mL, from about 6 mL to about 11 mL, from about 7 mL to about 15 mL, from about 7 mL to about 14 mL, from about 7 mL to about 13 mL, from about 7 mL to about 12 mL, from about 7 mL to about 11 mL, from about 8 mL to about 15 mL, from about 8 mL to about 14 mL, from about 8 mL to about 13 mL, from about 8 mL to about 12 mL, from about 8 mL to about 11 mL, from about 9 mL to about 15 mL, from about 9 mL to about 14 mL, from about 9 mL to about 13 mL, from about 9 mL to about 12 mL, or from about 9 mL to about 11 mL. In some instances, the sample (e.g., a whole blood sample, a plasma sample, a serum sample, or a combination thereof) has a volume of about 10 mL. For example, in some instances, a plasma sample has a volume of 10 mL.

In some embodiments of any of the preceding methods, the somatic mutations evaluated in the assay each have an allele frequency of about 0.1% or more, e.g., about 0.1% or more, about 0.2% or more, about 0.3% or more, about 0.4% or more, about 0.5% or more, about 0.6% or more, about 0.7% or more, about 0.8% or more, about 0.9% or more, about 1.0% or more, about 1.1% or more, about 1.2% or more, about 1.3% or more, about 1.4% or more, about 1.5% or more, about 1.6% or more, about 1.7% or more, about 1.8% or more, about 1.9% or more, about 2.0% or more, about 2.1% or more, about 2.2% or more, about 2.3% or more, about 2.4% or more, about 2.5% or more, about 2.6% or more, about 2.7% or more, about 2.8% or more, about 2.9% or more, about 3.0% or more, about 3.1% or more, about 3.2% or more, about 3.3% or more, about 3.4% or more, about 3.5% or more, about 3.6% or more, about 3.7% or more, about 3.8% or more, about 3.9% or more, about 4.0% or more, about 4.1% or more, about 4.2% or more, about 4.3% or more, about 4.4% or more, about 4.5% or more, about 4.6% or more, about 4.7% or more, about 4.8% or more, about 4.9% or more, about 5.0% or more, about 6.0% or more, about 7.0% or more, about 8.0% or more, about 9.0% or more, about 10.0% or more, about 11.0% or more, about 12.0% or more, about 13.0% or more, about 14.0% or more, about 15.0% or more about 16.0% or more, about 17.0% or more, about 18.0% or more, about 19.0% or more, about 20.0% or more, or higher. For example, in some embodiments, the somatic mutations evaluated in the assay each have an allele frequency of 0.5% or more.

TABLE 1
Exemplary Cancer-related Genes

| | | | | | | | | | |
|---|---|---|---|---|---|---|---|---|---|
| ABL1 | BTK | CTNNB1 | FAS | HIST1H1C | KDR | MYCN | PDK | RPL13 | SUFU |
| | | | (TNFRSF6) | | | | | | |
| ABI1 | BTLA | CUL4A | FAT3 | CALR | KEAP1 | MYD88 | PHF6 | HOXA3 | SUZ12 |
| ABL2 | c11orf30 | CUL4B | FBXO11 | HIST1H1D | KIT | MYO18A | PIK3C2G | NBEAP1 | SYK |
| | (EMSY) | | | | | | | (BCL8) | |
| ACSL6 | CAD | CUX1 | FBXO31 | HIST1H1E | KLHL6 | NBN | PIK3C3 | NFKB2 | TAF1 |
| ACTB | CAMTA1 | CXCR4 | FBXW7 | HIST1H2AC | KMT2A | NCOR1 | PIK3CA | RPL15 | TBL1XR1 |
| | | | | | (MLL) | | | | |
| AFF1 | CARD11 | CYP17A1 | FGF10 | HIST1H2AG | KMT2B | NCOR2 | PIK3CG | RPL35A | TBX3 |
| | | | | | (MLL2) | | | | |
| AFF4 | CARS | DAXX | FGF12 | HIST1H2AL | KMT2C | NCSTN | PIK3R1 | RPS14 | TCF3 |
| | | | | | (MLL3) | | | | |
| AKT1 | CASP8 | DDIT3 | FGF14 | HIST1H2AM | KRAS | NF1 | PIK3R2 | RPS15 | TCL1A |
| AKT2 | CBFA2T3 | DDR1 | FGF19 | HIST1H2BC | LEF1 | NF2 | PIM1 | RPS19 | TET1 |
| AKT3 | CBFB | DDR2 | FGF23 | HIST1H2BJ | LMO1 | NFE2L2 | PLAG1 | RPS26 | TET2 |
| ALK | CBL | DDX10 | FGF3 | HIST1H2BK | LRP1B | NFKBIA | PLCG2 | RPTOR | TFE3 |
| ALOX12B | CCND1 | DDX3X | FGF4 | HIST1H2BO | LRRK2 | NKX2-1 | PML | RUNX1 | TFG |
| AMER1 | CCND2 | DDX6 | FGF6 | HIST1H3B | MAF | NOD1 | PMS2 | RUNX1T1 | TFPT |
| (FAM123B | | | | | | | | | |
| or WTX) | | | | | | | | | |
| APC | CCND3 | DEK | FGF7 | HLA-A | MAFB | NOTCH1 | PNRC1 | RUNX1T1 | TFRC |
| | | | | | | | | (ETO) | |
| APCDD1 | CCNE1 | DIS3 | FGFR1 | HNF1A | MAGED1 | NOTCH2 | POT1 | RUNX2 | TGFBR2 |
| APH1A | CCT6B | DKC1 | FGFR2 | HRAS | MALT1 | NOTCH3 | POU2AF1 | S1PR2 | TIPARP |
| AR | CD22 | DLEU2 | FGFR3 | HSP90AA1 | MAP2K1 | NOTCH4 | PPP1CB | SBDS | TLL2 |
| ARAF | CD247 | DNM2 | FGFR4 | ICK | MAP2K2 | NPM1 | PPP2R1A | SDHA | TLX1 |
| ARFRP1 | CD274 | DNMT3A | FHIT | ID3 | MAP2K4 | NR4A3 | PRDM1 | SDHB | TLX3 |
| | (PDL1) | | | | | | | | |
| ARHGAP26 | CD36 | DOT1L | FLCN | IDH1 | MAP3K1 | NRAS | PRDM16 | SDHC | TMEM30A |
| ARHGAP26 | CD58 | DTX1 | FLI1 | IDH2 | MAP3K13 | NSD1 | PRKAR1A | SDHD | TMPRSS2 |
| (GRAF) | | | | | | | | | |
| ARHGEF12 | CD70 | DUSP2 | FLT1 | IGF1 | MAP3K14 | NT5C2 | PRKDC | SEC31A | TMSB4XP8 |
| | | | | | | | | | (TMSL3) |
| ARID1A | CD79A | DUSP22 | FLT3 | IGF1R | MAP3K6 | NTRK1 | PRRX1 | SERP2 | TNFAIP3 |
| ARID1B | CD79B | DUSP9 | FLT4 | IGF2 | MAP3K7 | NTRK2 | PRSS8 | SET | TNFRSF11A |
| ARID2 | CDC73 | EBF1 | FLYWCH1 | IGH | MAPK1 | NTRK3 | PSIP1 | SETBP1 | TNFRSF14 |
| ARNT | CDH1 | ECT2L | FNBP1 | IGK | MCL1 | NUMA1 | PTCH1 | SETD2 | TNFRSF17 |
| ASMTL | CDK12 | EED | FOXL2 | IGL | MDM2 | NUP214 | PTEN | SF3B1 | TNFSF9 |
| ASXL1 | CDK4 | EGFR | FOXO1 | IKBKE | MDM4 | NUP93 | PTK7 | SGK1 | TOP1 |
| ATF1 | CDK6 | EIF4A2 | FOXO3 | IKZF | MDS2 | NUP98 | PTPN11 | SH2B3 | TP53 |
| ATG5 | CDK8 | ELF4 | FOXO4 | IKZF2 | MECOM | NUTM2A | PTPN2 | SH3GL1 | TP63 |
| ATIC | CDKN1B | ELL | FOXP1 | IKZF3 | MED12 | OLIG2 | PTPN6 | SLC1A2 | TPM3 |
| | | | | | | | (SHP-1) | | |
| ATM | CDKN2A | ELN | FRS2 | IL21R | MEF2B | OMD | PTPRO | SMAD2 | TPM4 |
| ATR | CDKN2B | ELP2 | FSTL3 | IL3 | MEF2C | P2RY8 | RABEP1 | SMAD4 | TRAF2 |
| ATRX | CDKN2C | EML4 | FUS | IL7R | MEN1 | PAFAH1B2 | RAD21 | SMARCA1 | TRAF3 |
| ATXN1 | CDX2 | EP300 | GADD45B | INHBA | MET | PAG1 | RAD50 | SMARCA4 | TRAF5 |
| AURKA | CEBPA | EPHA3 | GAS7 | INPP4B | MIB1 | PAK3 | RAD51 | SMARCB1 | TRG |
| AURKB | CHD2 | EPHA5 | GATA1 | INPP5D | MITF | PAK7 | RAD51B | SMARCD1 | TRIM24 |
| | | | | (SHIP) | | | | | |
| AXIN1 | CHEK1 | EPHA7 | GATA2 | INSR | MKI67 | PALB2 | RAD51C | SMC1A | TRIP11 |
| AXL | CHEK2 | EPHB1 | GATA3 | IRF1 | MKL1 | PARP1 | RAD51D | SMC3 | TRRAP |
| B2M | CHIC2 | EPOR | GID4 | IRF4 | MKL2 | PARP2 | RAD52 | SMO | TSC1 |
| | | | (c17orf39) | | | | | | |
| BAP1 | CHN1 | EPS15 | GLI1 | IRF8 | MLF1 | PARP3 | RAD4L | SNX29 | TSC2 |
| | | | | | | | | (RUNDC2A) | |
| BARD1 | CHTOP | ERBB2 | GLIS2 | IRS2 | MLH1 | PARP4 | RAF1 | SOCS1 | TSHR |
| | (C1orf77) | | | | | | | | |
| BCL10 | CHUK | ERBB3 | GMPS | ITK | MLLT1 | PASK | RALGDS | SOCS2 | TTL |
| | | | | | (ENL) | | | | |
| BCL11A | CIC | ERBB4 | GNA11 | JAK1 | MLLT10 | PAX3 | RANBP17 | SOCS3 | TUSC3 |
| | | | | | (AF10) | | | | |
| BCL11B | CIITA | ERG | GNA12 | JAK2 | MLLT3 | PAX5 | RAP1GDS1 | SOX10 | TYK2 |
| BCL2 | CKS1B | ESR1 | GNA13 | JAK3 | MLLT4 | PAX7 | RARA | SOX2 | U2AF1 |
| | | | | | (AF6) | | | | |
| BCL2L2 | CLP1 | ETS1 | GNAQ | JARID2 | MLLT6 | PBRM1 | RASGEF1A | SPEN | U2AF2 |
| BCL3 | CLTC | ETV1 | GNAS | JAZF1 | MN1 | PBX1 | RB1 | SPOP | USP6 |
| BCL6 | CLTCL1 | ETV4 | GPHN | JUN | MNX1 | PC | RBM15 | SRC | VHL |
| BCL7A | CNTRL | ETV5 | GPR124 | KAT6A | MPL | PCBP1 | RCOR1 | SRSF2 | WDR90 |
| | (CEP110) | | | (MYST3) | | | | | |
| BCL9 | COL1A1 | ETV6 | GRIN2A | KDM2B | MRE11A | PCLO | REL | SRSF3 | WHSC1 |
| BCOR | CPS1 | EWSR1 | GSK3B | KDM4C | MSH2 | PCM1 | RELN | SS18 | WHSC1 |
| | | | | | | | | | (MMSET |
| | | | | | | | | | or NSD2) |
| BCORL1 | CRBN | EXOSC6 | GTSE1 | KDM5A | MSH3 | PCSK7 | RET | SSX1 | WHSC1L1 |
| BCR | CREB3L1 | EZH2 | HDAC1 | KDM5C | MSH6 | PDCD1 | RHOA | SSX2 | WISP3 |
| BIRC3 | CREB3L2 | FAF1 | HDAC4 | KDM6A | MSI2 | PDCD11 | RHOH | SSX4 | WT1 |
| BLM | CREBBP | FAM46C | HDAC7 | KDSR | MSN | PDCD1LG2 | RICTOR | STAG2 | XBP1 |
| | | | | | | (PDL2) | | | |
| BRAF | CRKL | FANCA | HERPUD1 | KIF5B | MTAP | PDCD1LG2 | RMRP | STAT3 | XPO1 |
| | | | | | | (PDL2) | | | |
| BRCA1 | CRLF2 | FANCC | HEY1 | LASP1 | MTCP1 | PDE4DIP | RNF213 | STAT4 | XRCC3 |
| BRCA2 | CSF1 | FANCD2 | HGF | LCK | MTOR | PDGFB | RNF43 | STAT5A | YPEL5 |
| BRD4 | CSF1R | FANCE | HIP1 | LCP1 | MUC1 | PDGFRA | ROS1 | STAT5B | YY1AP1 |
| BRIP1 | CSF3R | FANCF | HIST1H1A | LMO2 | MUTYH | PDGFRB | RPA1 | STAT6 | ZBTB16 |
| (BACH1) | | | | | | | | | |
| BRSK1 | CTCF | FANCG | HIST1H4I | LPP | MYB | PER1 | RPL11 | STK11 | ZMYM2 |
| BTG1 | CTNNA1 | FANCI | HLF | LTK | MYC | PGAM5 | RPL22 | STL | ZMYM3 |
| BTG2 | FANCM | FANCL | HMGA1 | LYL1 | MYCL | PHF1 | RPN1 | TAF15 | ZNF217 |
| | | | | | (MYCL1) | | | | |
| HSP90AB1 | FCGR2B | HOXA9 | HMGA2 | MAGEA5 | MYH11 | PICALM | TCL6 | TAL1 | ZNF24 |
| | | | | | | | | | (ZSCAN3) |
| HOXD13 | FCRL4 | HOXC11 | HOXA11 | MYH9 | NCOA2 | TEC | TAL2 | TCF3 | ZNF384 |
| | | | | | | | | (E2A) | |
| NFKBIE | FEV | HOXC13 | HOXA13 | NACA | NDRG1 | TCL1A | ZRSR2 | ZNF703 | ZNF521 |
| | | | | | | (TCL1) | | | |
| NIN | FGFR1OP | HOXD11 | | | | | | | |

The determination step may include determining the highest relative frequency of an allele (i.e., a variant of a gene having a somatic mutation (e.g., a base substitution in a coding region and/or an indel mutation in a coding region)) from a sample (e.g., a whole blood sample, a plasma sample, a serum sample, or a combination thereof) from an individual to derive an MSAF. A somatic allele frequency for the next most commonly occurring mutation may also be determined from the sample from the individual. In some instances, a somatic allele frequency is determined for each mutation detected from the sample from the individual. In some instances, samples with multiple somatic mutations will present those mutations as a distribution of somatic allele frequencies, likely dependent upon their original clonal frequency in a cancer (e.g., a tumor). In some instances, somatic allele frequencies greater than 40% (e.g., >40%, ≥50%, ≥60%, ≥70%, ≥80%, ≥90%, or 100%) are discarded, and the variant with the next highest somatic allele frequency below 40% (e.g., ≤40%) is determined to be the MSAF for the sample. In some instances, MSAF is calculated from the largest somatic allele frequency less than 20% in the sample. Germline mutations may be found to have a somatic allele frequency distribution between about 50% and about 100%.

The determination of an MSAF may occur prior to, concurrently with, or after the determination of a bTMB score from a sample from the individual.

In any of the preceding methods, the sample (e.g., blood sample) obtained from the patient is selected from the group consisting of a whole blood, plasma, serum, or a combination thereof. In some instances, the sample is an archival blood sample, a fresh blood sample, or a frozen blood sample.

In any of the preceding instances, the reference bTMB score may be a bTMB score in a reference population of individuals having a cancer (e.g., a lung cancer (e.g., squamous or non-squamous NSCLC)). The population of individuals may include a first subset of individuals who have been treated with an immune checkpoint inhibitor, for example, a PD-1 axis binding antagonist therapy, and a second subset of individuals who have been treated with a non-PD-1 axis binding antagonist therapy, wherein the non-PD-1 axis binding antagonist therapy does not comprise an immune checkpoint inhibitor, for example, a PD-1 axis binding antagonist. In some instances, the reference bTMB score significantly separates each of the first and second subsets of individuals based on a significant difference in responsiveness to treatment with the PD-1 axis binding antagonist therapy relative to responsiveness to treatment with the non-PD-1 axis binding antagonist therapy. In some instances, responsiveness to treatment is an increase in progression-free survival (PFS) and/or an increase in overall survival (OS).

In some instances, the reference bTMB score may be a pre-assigned bTMB score. The reference bTMB score may be between 4 and 30 (e.g., 4, 5, 6, 7, 8, 9, 10, 11, 12, 13, 14, 15, 16, 17, 18, 19, 20, 21, 22, 23, 24, 25, 26, 27, 28, 29, and 30, e.g., between 8 and 30, e.g., between 10 and 16, or, e.g., between 10 and 20). In some instances, the reference bTMB score may be between 10 and 20 (e.g., 10, 11, 12, 13, 14, 15, 16, 17, 18, 19, or 20). In other instances, the reference bTMB score may be between 16 and 20 (e.g., 16, 17, 18, 19, or 20). For example, in some instances, the reference population of individuals has a lung cancer (e.g., squamous or non-squamous NSCLC) and a reference bTMB score greater than, or equal to, 9. For example, in some instances, the reference population of individuals has a lung cancer (e.g., squamous or non-squamous NSCLC) and a reference bTMB score greater than, or equal to, 10. For example, in some instances, the reference population of individuals has a lung cancer (e.g., squamous or non-squamous NSCLC) and a reference bTMB score greater than, or equal to, 11. For example, in some instances, the reference population of individuals has a lung cancer (e.g., squamous or non-squamous NSCLC) and a reference bTMB score greater than, or equal to, 12. For example, in some instances, the reference population of individuals has a lung cancer (e.g., squamous or non-squamous NSCLC) and a reference bTMB score greater than, or equal to, 13. For example, in some instances, the reference population of individuals has a lung cancer (e.g., squamous or non-squamous NSCLC) and a reference bTMB score greater than, or equal to, 14. For example, in some instances, the reference population of individuals has a lung cancer (e.g., squamous or non-squamous NSCLC) and a reference bTMB score greater than, or equal to, 15. For example, in some instances, the reference population of individuals has a lung cancer (e.g., squamous or non-squamous NSCLC) and a reference bTMB score greater than, or equal to, 16. For example, in some instances, the reference population of individuals has a lung cancer (e.g., squamous or non-squamous NSCLC) and a reference bTMB score greater than, or equal to, 17. For example, in some instances, the reference population of individuals has a lung cancer (e.g., squamous or non-squamous NSCLC) and a reference bTMB score greater than, or equal to, 18. For example, in some instances, the reference population of individuals has a lung cancer (e.g., squamous or non-squamous NSCLC) and a reference bTMB score greater than, or equal to, 19. For example, in some instances, the reference population of individuals has a lung cancer (e.g., squamous or non-squamous NSCLC) and a reference bTMB score greater than, or equal to, 20. For example, in some instances, the reference population of individuals has a lung cancer (e.g., squamous or non-squamous NSCLC) and a reference bTMB score greater than, or equal to, 21. For example, in some instances, the reference population of individuals has a lung cancer (e.g., squamous or non-squamous NSCLC) and a reference bTMB score greater than, or equal to, 22. For example, in some instances, the reference population of individuals has a lung cancer (e.g., squamous or non-squamous NSCLC) and a reference bTMB score greater than, or equal to, 23. For example, in some instances, the reference population of individuals has a lung cancer (e.g., squamous or non-squamous NSCLC) and a reference bTMB score greater than, or equal to, 24. For example, in some instances, the reference population of individuals has a lung cancer (e.g., squamous or non-squamous NSCLC) and a reference bTMB score greater than, or equal to, 25.

In any of the preceding instances, the bTMB score from the sample may be greater than, or equal to, 4 (e.g., 4, 5, 6, 7, 8, 9, 10, 11, 12, 13, 14, 15, 16, 17, 18, 19, 20, 21, 22, 23, 24, 25, 26, 27, 28, 29, 30, or more). For example, the bTMB score from the sample may be between about 8 and about 100 (e.g., 8, 9, 10, 20, 30, 40, 50, 60, 70, 80, 90, and 100). In some instances, the bTMB score from the sample may be between about 400 and about 1500 (e.g., a bTMB score of about 400, 500, 600, 700, 800, 900, 1000, 1100, 1200, 1300, 1400, or 1500). In some instances, the bTMB score from the sample may be less than 4 (e.g., 0, 1, 2, or 3) or be undetectable.

In some embodiments of any of the preceding instances, the bTMB score (e.g., reference bTMB score) is represented as the number of somatic mutations counted over a defined number of sequenced bases (e.g., about 1.1 Mb (e.g., about 1.125 Mb), e.g., as assessed by the FOUNDATIONONE® panel). In some embodiments, the bTMB score (e.g., reference bTMB score) is an equivalent bTMB value, for example, as determined by whole-exome sequencing.

In some instances, the bTMB score from the sample from the individual may have a prevalence of greater than, or equal to, about 5%, for example, a prevalence of between about 5% and about 75% (e.g., a prevalence between about 5% and about 15%, about 15% and about 30%, about 30% and about 45%, about 45% and about 60%, or about 60% and 75%; e.g., 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, or 75%) in a reference population.

In some instances, the prevalence of a bTMB score that is greater than, or equal to, a reference cut-off bTMB score is about 5%, for example, a prevalence of between about 5% and about 75% (e.g., a prevalence between about 5% and about 15%, about 15% and about 30%, about 30% and about 45%, about 45% and about 60%, or about 60% and 75%; e.g., 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, or 75%) in a reference population.

In some instances, a bTMB score determined as the number of somatic mutations counted over a defined number of sequenced bases (e.g., about 1.1 Mb (e.g., about 1.125 Mb), e.g., as assessed by the FOUNDATIONONE® panel) in a subset of the genome or exome (e.g., a predetermined set of genes) deviates by less than about 30% (e.g., less than about 30%, about 25%, about 20%, about 15%, about 10%, about 5%, about 4%, about 3%, about 2%, about 1%, or less) from a bTMB score determined by whole-exome sequencing. In some embodiments, a bTMB score determined as the number of somatic mutations counted over a defined number of sequenced bases (e.g., about 1.1 Mb (e.g., about 1.125 Mb), e.g., as assessed by the FOUNDATIONONE® panel) in a subset of the genome or exome (e.g., a predetermined set of genes) deviates about 1% to about 30% (e.g., about 1% to about 30%, about 1% to about 25%, about 1% to about 20%, about 1% to about 15%, about 1% to about 10%, about 5% to about 30%, about 5% to about 25%, about 5% to about 20%, about 5% to about 15%, about 5% to about 10%, about 10% to about 30%, about 10% to about 25%, about 10% to about 20%, about 10% to about 15%, about 15% to about 30%, about 15% to about 25%, about 15% to about 20%, about 20% to about 30%, or about 20% to about 25%) from a bTMB score determined by whole-exome sequencing. In some embodiments, a bTMB score determined as the number of somatic mutations counted over a defined number of sequenced bases (e.g., about 1.1 Mb (e.g., about 1.125 Mb), e.g., as assessed by the FOUNDATIONONE® panel) in a subset of the genome or exome (e.g., a predetermined set of genes) deviates about 10% to about 20% (e.g., about 10%, about 11%, about 12%, about 13%, about 14%, about 15%, about 16%, about 17%, about 18%, about 19%, or about 20%) from a bTMB score determined by whole-exome sequencing. In any of the methods provided here, the benefit from the treatment comprising an immune checkpoint inhibitor, for example, a PD-1 axis binding antagonist, may be an increase in OS, an increase in PFS, or an increase in OS and PFS.

In any of the preceding methods, the PD-1 axis binding antagonist may be any PD-1 axis binding antagonist known in the art or described herein.

In some embodiments, the method further comprises generating a report, e.g., an electronic, web-based, or paper report, to the patient or to another person or entity, a caregiver, a physician, an oncologist, a hospital, clinic, third-party payor, insurance company, a pharmaceutical or biotechnology company, or government office. In some embodiments, the report comprises output from the method which comprises evaluation of the bTMB score.

### IV. Therapeutic Methods and Compositions for the Treatment of NSCLC

Provided herein are methods for treating or delaying progression of NSCLC (e.g., squamous or non-squamous NSCLC, including stage IV NSCLC) in a subject comprising administering to the subject an effective amount of a PD-1 axis binding antagonist (e.g., an anti-PD-L1 antibody (e.g., atezolizumab) or an anti-PD-1 antibody). Also provided herein are methods for treating or delaying progression of NSCLC (e.g., squamous or non-squamous NSCLC, including stage IV NSCLC) in a subject comprising administering to the subject an effective amount of a PD-1 axis binding antagonist (e.g., an anti-PD-L1 antibody (e.g., atezolizumab) or an anti-PD-1 antibody) and a platinum-based chemotherapy (e.g., cisplatin or carboplatin and gemcitabine). The subject may be one that is identified as likely to benefit from treatment with a PD-1 axis binding antagonist, for example, on the basis of a finding that the subject exhibits a bTMB score greater than, or equal to, a reference bTMB score. For example, the subject may be one that is identified as having a bTMB score of greater than, or equal to, 8. In some embodiments, the subject is one that is identified as having a bTMB score of greater than, or equal to, 9. In some embodiments, the subject is one that is identified as having a bTMB score of greater than, or equal to, 10. In some embodiments, the subject is one that is identified as having a bTMB score of greater than, or equal to, 11. In some embodiments, the subject is one that is identified as having a bTMB score of greater than, or equal to, 12. In some embodiments, the subject is one that is identified as having a bTMB score of greater than, or equal to, 13. In some embodiments, the subject is one that is identified as having a bTMB score of greater than, or equal to, 14. In some embodiments, the subject is one that is identified as having a bTMB score of greater than, or equal to, 15. In some embodiments, the subject is one that is identified as having a bTMB score of greater than, or equal to, 16. In some embodiments, the subject is one that is identified as having a bTMB score of greater than, or equal to, 17. In some embodiments, the subject is one that is identified as having a bTMB score of greater than, or equal to, 18. In some embodiments, the subject is one that is identified as having a bTMB score of greater than, or equal to, 19. In some embodiments, the subject is one that is identified as having a bTMB score of greater than, or equal to, 20. In some embodiments, the subject is one that is identified as having a bTMB score of greater than, or equal to, 21. In some embodiments, the subject is one that is identified as having a bTMB score of greater than, or equal to, 22. In some embodiments, the subject is one that is identified as having a bTMB score of greater than, or equal to, 23. In some embodiments, the subject is one that is identified as having a bTMB score of greater than, or equal to, 24. In some embodiments, the subject is one that is identified as having a bTMB score of greater than, or equal to, 25. In some embodiments, the subject is one that is identified as having a bTMB score of greater than, or equal to, 26. In some embodiments, the subject is one that is identified as having a bTMB score of greater than, or equal to, 27. In some embodiments, the subject is one that is identified as having a bTMB score of greater than, or equal to, 28. In some embodiments, the subject is one that is identified as having a bTMB score of greater than, or equal to, 29. In some embodiments, the subject is one that is identified as having a bTMB score of greater than, or equal to, 30.

In some embodiments, the PD-1 axis binding antagonist results in a response in the subject after treatment. For example, in some embodiments, the PD-1 axis binding antagonist increases the subject's likelihood of having an objective response (e.g., a complete response (CR)), extends the subject's progression-free survival (PFS), extends the subject's overall survival (OS), and/or extends the subject's duration of response (DOR), for example, as compared to a reference treatment, e.g., treatment without the PD-1 axis binding antagonist or treatment with a platinum-based chemotherapy without the PD-1 axis binding antagonist. Also provided herein are methods of enhancing immune function in a subject having NSCLC (e.g., squamous or non-squamous NSCLC, including stage IV NSCLC) comprising administering to the subject an effective amount of a PD-1 axis binding antagonist (e.g., an anti-PD-L1 antibody (e.g., atezolizumab) or an anti-PD-1 antibody). Further provided herein are methods of enhancing immune function in a subject having NSCLC (e.g., squamous or non-squamous NSCLC, including stage IV NSCLC) comprising administering to the subject an effective amount of a PD-1 axis binding antagonist (e.g., an anti-PD-L1 antibody (e.g., atezolizumab) or an anti-PD-1 antibody) and a platinum-based chemotherapy (e.g., cisplatin or carboplatin and gemcitabine). Any of the PD-1 axis binding antagonists and/or the platinum-based chemotherapies known in the art or described herein may be used in the methods of the present disclosure.

For example, provided herein is a method of treating NSCLC (e.g., squamous or non-squamous NSCLC, including stage IV NSCLC) in a subject in need thereof, the method comprising administering to the subject an effective amount of a PD-1 axis binding antagonist (e.g., an anti-PD-L1 antibody (e.g., atezolizumab) or an anti-PD-1 antibody) in one or more dosing cycles, wherein the subject is previously untreated for the NSCLC and is eligible for treatment with a platinum-based chemotherapy, and wherein the PD-1 axis binding antagonist results in an improved treatment response as compared to treatment without the PD-1 axis binding antagonist.

In another example, provided herein is a pharmaceutical composition comprising a PD-1 axis binding antagonist for use in treatment of NSCLC (e.g., squamous or non-squamous NSCLC, including stage IV NSCLC) in a subject in need thereof, wherein the treatment comprises administration of a PD-1 axis binding antagonist (e.g., an anti-PD-L1 antibody (e.g., atezolizumab) or an anti-PD-1 antibody) in one or more dosing cycles, wherein the subject is previously untreated for the NSCLC and is eligible for treatment with a platinum-based chemotherapy, and wherein the PD-1 axis binding antagonist results in an improved treatment response as compared to treatment without the PD-1 axis binding antagonist

For example, in some embodiments, the PD-1 axis binding antagonist increases the subject's likelihood of having an objective response (e.g., a CR), extends the subject's progression-free survival (PFS), extends the subject's overall survival (OS), and/or extends the subject's duration of response (DOR) as compared to treatment without the PD-1 axis binding antagonist. In some embodiments, the PD-1 axis binding antagonist increases the subject's likelihood of having an objective response as compared to treatment without the PD-1 axis binding antagonist. In some embodiments, the PD-1 axis binding antagonist increases the subject's likelihood of having a CR as compared to treatment without the PD-1 axis binding antagonist. In some embodiments, the PD-1 axis binding antagonist extends the subject's PFS as compared to treatment without the PD-1 axis binding antagonist. In some embodiments, the PD-1 axis binding antagonist extends the subject's OS as compared to treatment without the PD-1 axis binding antagonist. In some embodiments, the PD-1 axis binding antagonist extends the subject's DOR as compared to treatment without the PD-1 axis binding antagonist.

The subject may be eligible for any suitable platinum-based chemotherapy. Eligibility for a platinum-based chemotherapy may be as described herein or according to criteria known in the art. For example, criteria for defining patients who are cisplatin-eligible or cisplatin-ineligible are known in the art, e.g., as described in Galsky et al. Lancet. Oncol. 12:211-4, 2011, which is incorporated herein by reference in its entirety. In some embodiments, the subject is eligible for treatment with a platinum-based chemotherapy comprising cisplatin. In some embodiments, the subject is eligible for treatment with a platinum-based chemotherapy comprising carboplatin.

In some embodiments, the PD-1 axis binding antagonist is administered as a monotherapy.

In other embodiments, the PD-1 axis binding antagonist is administered in combination with an effective amount of one or more additional therapeutic agents. In some embodiments, the one or more additional therapeutic agents are selected from an anti-neoplastic agent, a chemotherapeutic agent, a growth inhibitory agent, an anti-angiogenic agent, a radiation therapy, or a cytotoxic agent. In some embodiments, the one or more additional therapeutic agents are a platinum-based chemotherapy. In some embodiments, the treatment without the PD-1 axis binding antagonist comprises treatment with a platinum-based chemotherapy.

In any of the preceding examples, each dosing cycle may have any suitable length, e.g., about 7 days, about 14 days, about 21 days, about 28 days, or longer. In some embodiments, each dosing cycle is about 21 days.

In another example, provided herein is a method of treating NSCLC (e.g., squamous or non-squamous NSCLC, including stage IV NSCLC) in a subject in need thereof, the method comprising administering to the subject a an effective amount of a PD-1 axis binding antagonist (e.g., an anti-PD-L1 antibody (e.g., atezolizumab) or an anti-PD-1 antibody) in combination with a platinum-based chemotherapy (e.g., cisplatin or carboplatin and gemcitabine) in one or more dosing cycles, wherein the subject is previously untreated for the NSCLC, and wherein the PD-1 axis binding antagonist results in an improved treatment response as compared to treatment with the platinum-based chemotherapy without the PD-1 axis binding antagonist.

In yet another example, provided herein is a pharmaceutical composition comprising a PD-1 axis binding antagonist (e.g., an anti-PD-L1 antibody (e.g., atezolizumab) or an anti-PD-1 antibody for use in treatment of NSCLC (e.g., squamous or non-squamous NSCLC, including stage IV NSCLC) in a subject in need thereof, the treatment comprises administration of a PD-1 axis binding antagonist in combination with a platinum-based chemotherapy (e.g., cisplatin or carboplatin and gemcitabine) in one or more dosing cycles, wherein the subject is previously untreated for the NSCLC, and wherein the PD-1 axis binding antagonist results in an improved treatment response as compared to treatment with the platinum-based chemotherapy without the PD-1 axis binding antagonist.

For example, in some embodiments, the PD-1 axis binding antagonist increases the subject's likelihood of having an objective response (e.g., a CR), extends the subject's PFS, extends the subject's OS, and/or extends the subject's DOR as compared to treatment with the platinum-based chemotherapy without the PD-1 axis binding antagonist. In some embodiments, the PD-1 axis binding antagonist increases the subject's likelihood of having an objective response as compared to treatment with the platinum-based chemotherapy without the PD-1 axis binding antagonist. In some embodiments, the PD-1 axis binding antagonist increases the subject's likelihood of having a CR as compared to treatment with the platinum-based chemotherapy without the PD-1 axis binding antagonist. In some embodiments, the PD-1 axis binding antagonist extends the subject's PFS as compared to treatment with the platinum-based chemotherapy without the PD-1 axis binding antagonist. In some embodiments, the PD-1 axis binding antagonist extends the subject's OS as compared to treatment with the platinum-based chemotherapy without the PD-1 axis binding antagonist. In some embodiments, the PD-1 axis binding antagonist extends the subject's DOR as compared to treatment with the platinum-based chemotherapy without the PD-1 axis binding antagonist.

In another example, provided herein is a method of treating NSCLC (e.g., squamous or non-squamous NSCLC, including stage IV NSCLC) in a subject in need thereof, the method comprising administering to the subject an effective amount of a PD-1 axis binding antagonist (e.g., an anti-PD-L1 antibody (e.g., atezolizumab) or an anti-PD-1 antibody) in combination with a platinum-based chemotherapy (e.g., cisplatin or carboplatin and gemcitabine) in one or more dosing cycles, wherein the subject is previously untreated for the NSCLC, and wherein the PD-1 axis binding antagonist extends the subject's PFS and/or OS as compared to treatment with the platinum-based chemotherapy without the PD-1 axis binding antagonist.

In a still further example, provided herein is a pharmaceutical composition comprising a PD-1 axis binding antagonist for use in treatment of NSCLC (e.g., squamous or non-squamous NSCLC, including stage IV NSCLC) in a subject in need thereof, the treatment comprises administration of a PD-1 axis binding antagonist (e.g., an anti-PD-L1 antibody (e.g., atezolizumab) or an anti-PD-1 antibody) in combination with a platinum-based chemotherapy (e.g., cisplatin or carboplatin and gemcitabine) in one or more dosing cycles, wherein the subject is previously untreated for the NSCLC, and wherein the PD-1 axis binding antagonist extends the subject's PFS and/or OS as compared to treatment with the platinum-based chemotherapy without the PD-1 axis binding antagonist.

In some examples, the PD-1 axis binding antagonist extends the subject's PFS as compared to treatment with the platinum-based chemotherapy without the PD-1 axis binding antagonist. In some examples, the PD-1 axis binding antagonist extends the subject's OS as compared to treatment with the platinum-based chemotherapy without the PD-1 axis binding antagonist. In some embodiments, the PD-1 axis binding antagonist increases the subject's likelihood of having an objective response (e.g., a CR) and/or extends the subject's DOR as compared to treatment with the platinum-based chemotherapy without the PD-1 axis binding antagonist. In some embodiments, the PD-1 axis binding antagonist increases the subject's likelihood of having an objective response as compared to treatment with the platinum-based chemotherapy without the PD-1 axis binding antagonist. In some embodiments, the PD-1 axis binding antagonist increases the subject's likelihood of having a CR as compared to treatment with the platinum-based chemotherapy without the PD-1 axis binding antagonist. In some embodiments, the PD-1 axis binding antagonist extends the subject's DOR as compared to treatment with the platinum-based chemotherapy without the PD-1 axis binding antagonist.

In another example, provided herein is a method of treating NSCLC in a subject in need thereof, the method comprising administering to the subject an effective amount of a PD-1 axis binding antagonist (e.g., an anti-PD-L1 antibody (e.g., atezolizumab) or an anti-PD-1 antibody) in combination with a platinum-based chemotherapy (e.g., cisplatin or carboplatin and gemcitabine) in one or more dosing cycles, wherein the subject is previously untreated for the NSCLC, and wherein the PD-1 axis binding antagonist extends the subject's PFS as compared to treatment with the platinum-based chemotherapy without the PD-1 axis binding antagonist.

In yet another example, provided herein is a pharmaceutical composition comprising a PD-1 axis binding antagonist for use in treatment of NSCLC in a subject in need thereof, the treatment comprises administration of a PD-1 axis binding antagonist (e.g., an anti-PD-L1 antibody (e.g., atezolizumab) or an anti-PD-1 antibody) in combination with a platinum-based chemotherapy (e.g., cisplatin or carboplatin and gemcitabine) in one or more dosing cycles, wherein the subject is previously untreated for the NSCLC, and wherein the PD-1 axis binding antagonist extends the subject's PFS as compared to treatment with the platinum-based chemotherapy without the PD-1 axis binding antagonist.

In another example, provided herein is a method of treating NSCLC in a subject in need thereof, the method comprising administering to the subject an effective amount of a PD-1 axis binding antagonist (e.g., an anti-PD-L1 antibody (e.g., atezolizumab) or an anti-PD-1 antibody) in combination with a platinum-based chemotherapy (e.g., cisplatin or carboplatin and gemcitabine) in one or more dosing cycles, wherein the subject is previously untreated for the NSCLC, and wherein the PD-1 axis binding antagonist extends the subject's OS as compared to treatment with the platinum-based chemotherapy without the PD-1 axis binding antagonist.

In yet another example, provided herein is a pharmaceutical composition comprising a PD-1 axis binding antagonist for use in treatment of NSCLC in a subject in need thereof, the treatment comprises administration of a PD-1 axis binding antagonist (e.g., an anti-PD-L1 antibody (e.g., atezolizumab) or an anti-PD-1 antibody) in combination with a platinum-based chemotherapy (e.g., cisplatin or carboplatin and gemcitabine) in one or more dosing cycles, wherein the subject is previously untreated for the NSCLC, and wherein the PD-1 axis binding antagonist extends the subject's OS as compared to treatment with the platinum-based chemotherapy without the PD-1 axis binding antagonist.

In any of the preceding examples, each dosing cycle may have any suitable length, e.g., about 7 days, about 14 days, about 21 days, about 28 days, or longer. In some embodiments, each dosing cycle is about 21 days.

Any suitable number of dosing cycles may be used, e.g., 1, 2, 3, 4, 5, 6, 7, 8, 9, 10, 11, 12, 13, 14, 15, 20, 25, 30, 35, 40, 45, 50, or more dosing cycles. In some embodiments, 10 or fewer dosing cycles may be used. In some embodiments, 20 or fewer dosing cycles are used. In some embodiments, 25 or fewer dosing cycles are used.

Any suitable platinum-based chemotherapy may be used, including any platinum-based chemotherapy known in the art or described herein. In some embodiments, the platinum-based chemotherapy comprises a platinum-based chemotherapeutic agent and a nucleoside analog. In some embodiments, the platinum-based chemotherapeutic agent is cisplatin, carboplatin, or oxaliplatin.

For example, in some embodiments, the platinum-based chemotherapeutic agent is cisplatin. Any suitable dosing regimen for cisplatin known in the art may be used. In some embodiments, cisplatin is administered to the subject in a 21-day dosing cycle. In some embodiments, cisplatin is administered to the subject intravenously at a dose of about 35 mg/m² to about 140 mg/m². In some embodiments, cisplatin is administered to the subject intravenously at a dose of about 75 mg/m². In some embodiments, cisplatin is administered to the subject intravenously at a dose of about 75 mg/m² on Day −2 to Day 4 of a 21-day dosing cycle. In some embodiments, cisplatin is administered to the subject intravenously at a dose of about 75 mg/m² on Day 1 of a 21-day dosing cycle.

In another example, in other embodiments, the platinum-based chemotherapeutic agent is carboplatin. Any suitable dosing regimen for carboplatin known in the art may be used. In some embodiments, carboplatin is administered to the subject in a 21-day dosing cycle. In some embodiments, carboplatin is administered to the subject intravenously at an area under the curve (AUC) of about 2 to about 9. In some embodiments, carboplatin is administered to the subject intravenously at an area under the curve (AUC) of about 5. In some embodiments, carboplatin is administered to the subject intravenously at an area under the curve (AUC) of about 5 on Day −2 to Day 4 of a 21-day dosing cycle. In some embodiments, carboplatin is administered to the subject intravenously at an AUC of about 5 on Day 1 of a 21-day dosing cycle.

In any of the preceding examples, the platinum-based chemotherapy may include a nucleoside analog. Any suitable nucleoside analog may be used, including any nucleoside analog known in the art or described herein. Any suitable dosing regimen for gemcitabine known in the art may be used. In some embodiments, the nucleoside analog is gemcitabine. In some embodiments, gemcitabine is administered to the subject in a 21-day dosing cycle. In some embodiments, gemcitabine is administered to the subject intravenously at a dose of about 500 mg/m² to about 2000 mg/m². In some embodiments, gemcitabine is administered to the subject intravenously at a dose of about 1000 mg/m². In some embodiments, gemcitabine is administered to the subject intravenously at a dose of about 1000 mg/m² on Day −2 to Day 4 and on Day 7 to Day 11 of a 21-day dosing cycle. In some embodiments, gemcitabine is administered to the subject intravenously at a dose of about 1000 mg/m² on Day 1 and Day 8 of a 21-day dosing cycle.

In any of the preceding examples, the platinum-based chemotherapy may include cisplatin and gemcitabine. In other examples, the platinum-based chemotherapy may include carboplatin and gemcitabine.

In another example, provided herein is a method of treating NSCLC in a subject in need thereof, the method comprising administering to the subject an effective amount of a PD-1 axis binding antagonist (e.g., an anti-PD-L1 antibody (e.g., atezolizumab) or an anti-PD-1 antibody) in combination with a platinum-based chemotherapy comprising cisplatin or carboplatin and gemcitabine in one or more 21-day dosing cycles, wherein the subject is previously untreated for the NSCLC, and wherein the PD-1 axis binding antagonist extends the subject's PFS as compared to treatment with the platinum-based chemotherapy without the PD-1 axis binding antagonist.

In another example, provided herein is a pharmaceutical composition comprising a PD-1 axis binding antagonist for use in treatment of NSCLC in a subject in need thereof, the treatment comprises administration of a PD-1 axis binding antagonist (e.g., an anti-PD-L1 antibody (e.g., atezolizumab) or an anti-PD-1 antibody) in combination with a platinum-based chemotherapy comprising cisplatin or carboplatin and gemcitabine in one or more 21-day dosing cycles, wherein the subject is previously untreated for the NSCLC, and wherein the PD-1 axis binding antagonist extends the subject's PFS as compared to treatment with the platinum-based chemotherapy without the PD-1 axis binding antagonist.

In another example, provided herein is a method of treating NSCLC in a subject in need thereof, the method comprising administering to the subject an effective amount of a PD-1 axis binding antagonist (e.g., an anti-PD-L1 antibody (e.g., atezolizumab) or an anti-PD-1 antibody) in combination with a platinum-based chemotherapy comprising cisplatin or carboplatin and gemcitabine in one or more 21-day dosing cycles, wherein the subject is previously untreated for the NSCLC, and wherein the PD-1 axis binding antagonist extends the subject's OS as compared to treatment with the platinum-based chemotherapy without the PD-1 axis binding antagonist.

In some embodiments of the disclosure, administration of the PD-1 axis binding antagonist to the subject extends the subject's OS as compared to administration of the platinum-based chemotherapy without the PD-1 axis binding antagonist. For example, administration of the PD-1 axis binding antagonist to the subject may extend the subject's OS by from about 4 months to about 10 months, by from about 5 months to about 9 months, by from about 6 months to about 8 months, by from about 6.5 months to about 7.5 months, or by from about 6.8 months to about 7.4 months as compared to administration of the platinum-based chemotherapy without the PD-1 axis binding antagonist (e.g., by about 4 months, 4.1 months, 4.2 months, 4.3 months, 4.4 months, 4.5 months, 4.6 months, 4.7 months, 4.8 months, 4.9 months, 5 months, 5.1 months, 5.2 months, 5.3 months, 5.4 months, 5.5 months, 5.6 months, 5.7 months, 5.8 months, 5.9 months, 6 months, 6.1 months, 6.2 months, 6.3 months, 6.4 months, 6.5 months, 6.6 months, 6.7 months, 6.8 months, 6.9 months, 7 months, 7.1 months, 7.2 months, 7.3 months, 7.4 months, 7.5 months, 7.6 months, 7.7 months, 7.8 months, 7.9 months, 8 months, 8.1 months, 8.2 months, 8.3 months, 8.4 months, 8.5 months, 8.6 months, 8.7 months, 8.8 months, 8.9 months, 9 months, 9.1 months, 9.2 months, 9.3 months, 9.4 months, 9.5 months, 9.6 months, 9.7 months, 9.8 months, 9.9 months, or 10 months). In some embodiments, administration of the PD-1 axis binding antagonist to the subject may extend the subject's OS by about 7.1 months as compared to administration of the platinum-based chemotherapy without the PD-1 axis binding antagonist.

In some embodiments of the disclosure, administration of the PD-1 axis binding antagonist to the subject extends the subject's PFS as compared to administration of the platinum-based chemotherapy without the PD-1 axis binding antagonist. For example, administration of the PD-1 axis binding antagonist to the subject may extend the subject's PFS by from about 1 month to about 5 months, by from about 2 months to about 4 months, by from about 2.1 months to about 3.9 months, by from about 2.5 months to about 3.5 months, or by from about 2.8 months to about 3.4 months as compared to administration of the platinum-based chemotherapy without the PD-1 axis binding antagonist (e.g., by about 1 month, 1.1 months, 1.2 months, 1.3 months, 1.4 months, 1.5 months, 1.6 months, 1.7 months, 1.8 months, 1.9 months, 2 months, 2.1 months, 2.2 months, 2.3 months, 2.4 months, 2.5 months, 2.6 months, 2.7 months, 2.8 months, 2.9 months, 3 months, 3.1 months, 3.2 months, 3.3 months, 3.4 months, 3.5 months, 3.6 months, 3.7 months, 3.8 months, 3.9 months, 4 months, 4.1 months, 4.2 months, 4.3 months, 4.4 months, 4.5 months, 4.6 months, 4.7 months, 4.8 months, 4.9 months, or 5 months). In some embodiments, administration of the PD-1 axis binding antagonist to the subject may extend the subject's OS by about 3.1 months as compared to administration of the platinum-based chemotherapy without the PD-1 axis binding antagonist.

In some embodiments of the disclosure, administration of the PD-1 axis binding antagonist to the subject extends the subject's OS as compared to administration of the platinum-based chemotherapy without the PD-1 axis binding antagonist. For example, administration of the PD-1 axis binding antagonist to the subject may extend the subject's OS by from about 1 month to about 5.5 months, by from about 2 months to about 5 months, by from about 2.1 months to about 4.5 months, by from about 2.5 months to about 4 months, by from about 3 months to about 3.6 months, by from about 3.1 months to about 3.5 months (e.g., by about 3.3 months) as compared to administration of the platinum-based chemotherapy without the PD-1 axis binding antagonist.

In some embodiments of the disclosure, administration of the PD-1 axis binding antagonist to the subject extends the subject's PFS as compared to administration of the platinum-based chemotherapy without the PD-1 axis binding antagonist. For example, administration of the PD-1 axis binding antagonist to the subject may extend the subject's PFS by from about 1 month to about 4 months or by from about 1.5 months to about 2 months (e.g., by about 1.7 months) as compared to administration of the platinum-based chemotherapy without the PD-1 axis binding antagonist.

In another example, provided herein is a pharmaceutical composition comprising a PD-1 axis binding antagonist for use in treatment of NSCLC in a subject in need thereof, the treatment comprises administration of a PD-1 axis binding antagonist (e.g., an anti-PD-L1 antibody (e.g., atezolizumab) or an anti-PD-1 antibody) in combination with a platinum-based chemotherapy comprising cisplatin or carboplatin and gemcitabine in one or more 21-day dosing cycles, wherein the subject is previously untreated for the NSCLC, and wherein the PD-1 axis binding antagonist extends the subject's OS as compared to treatment with the platinum-based chemotherapy without the PD-1 axis binding antagonist.

Any suitable PD-1 axis binding antagonist may be used. Exemplary PD-1 axis binding antagonists are described herein. Other PD-1 axis binding antagonists are known in the art. In some embodiments, the PD-1 axis binding antagonist is selected from the group consisting of a PD-L1 binding antagonist, a PD-1 binding antagonist, and a PD-L2 binding antagonist.

In some embodiments, the PD-1 axis binding antagonist is a PD-L1 binding antagonist. In some embodiments, the PD-L1 binding antagonist inhibits the binding of PD-L1 to one or more of its ligand binding partners. In some embodiments, the PD-L1 binding antagonist inhibits the binding of PD-L1 to PD-1, B7-1, or both PD-1 and B7-1.

In some embodiments, the PD-L1 binding antagonist is an anti-PD-L1 antibody. Any suitable anti-PD-L1 antibody described herein or known in the art may be used. In some embodiments, the anti-PD-L1 antibody is selected from the group consisting of atezolizumab (TECENTRIQ®), MDX-1105, MED14736 (durvalumab), and MSB0010718C (avelumab). In some embodiments, the anti-PD-L1 antibody comprises the following hypervariable regions (HVRs): (a) an HVR-H1 sequence of GFTFSDSWIH (SEQ ID NO: 19);(b) an HVR-H2 sequence of AWISPYGGSTYYADSVKG (SEQ ID NO: 20);(c) an HVR-H3 sequence of RHWPGGFDY (SEQ ID NO: 21); (d) an HVR-L1 sequence of RASQDVSTAVA (SEQ ID NO: 22); (e) an HVR-L2 sequence of SASFLYS (SEQ ID NO: 23); and (f) an HVR-L3 sequence of QQYLYHPAT (SEQ ID NO: 24). In some embodiments, the anti-PD-L1 antibody comprises: (a) a heavy chain variable (VH) domain comprising an amino acid sequence having at least about 85%, 86%, 87%, 88%, 89%, 90%, 91%, 92%, 93%, 94%, 95%, 96%, 97%, 98%, 99%, or greater sequence identity to the amino acid sequence of SEQ ID NO: 3; (b) a light chain variable (VL) domain comprising an amino acid sequence having at least about 85%, 86%, 87%, 88%, 89%, 90%, 91%, 92%, 93%, 94%, 95%, 96%, 97%, 98%, 99%, or greater sequence identity to the amino acid sequence of SEQ ID NO: 4; or (c) a VH domain as in (a) and a VL domain as in (b). In some embodiments, the anti-PD-L1 antibody comprises: (a) a VH domain comprising an amino acid sequence having at least 95% sequence identity to the amino acid sequence of SEQ ID NO: 3; (b) a VL domain comprising an amino acid sequence having at least 95% sequence identity to the amino acid sequence of SEQ ID NO: 4; or (c) a VH domain as in (a) and a VL domain as in (b). In some embodiments, the anti-PD-L1 antibody comprises: (a) a VH domain comprising an amino acid sequence having at least 96% sequence identity to the amino acid sequence of SEQ ID NO: 3; (b) a VL domain comprising an amino acid sequence having at least 96% sequence identity to the amino acid sequence of SEQ ID NO: 4; or (c) a VH domain as in (a) and a VL domain as in (b). In some embodiments, the anti-PD-L1 antibody comprises: (a) a VH domain comprising an amino acid sequence having at least 97% sequence identity to the amino acid sequence of SEQ ID NO: 3; (b) a VL domain comprising an amino acid sequence having at least 97% sequence identity to the amino acid sequence of SEQ ID NO: 4; or (c) a VH domain as in (a) and a VL domain as in (b). In some embodiments, the anti-PD-L1 antibody comprises: (a) a VH domain comprising an amino acid sequence having at least 98% sequence identity to the amino acid sequence of SEQ ID NO: 3; (b) a VL domain comprising an amino acid sequence having at least 98% sequence identity to the amino acid sequence of SEQ ID NO: 4; or (c) a VH domain as in (a) and a VL domain as in (b). In some embodiments, the anti-PD-L1 antibody comprises: (a) a VH domain comprising an amino acid sequence having at least 99% sequence identity to the amino acid sequence of SEQ ID NO: 3; (b) a VL domain comprising an amino acid sequence having at least 99% sequence identity to the amino acid sequence of SEQ ID NO: 4; or (c) a VH domain as in (a) and a VL domain as in (b). In some embodiments, the anti-PD-L1 antibody comprises: (a) a VH domain comprising the amino acid sequence of SEQ ID NO: 3; (b) a VL domain comprising the amino acid sequence of SEQ ID NO: 4; or (c) a VH domain as in (a) and a VL domain as in (b). In some embodiments, the anti-PD-L1 antibody comprises: (a) a VH domain comprising the amino acid sequence of SEQ ID NO: 3; and (b) a VL domain comprising the amino acid sequence of SEQ ID NO: 4. In some embodiments, the anti-PD-L1 antibody is atezolizumab.

Atezolizumab may be administered to the subject at any suitable dosage. In some embodiments, atezolizumab is administered to the subject intravenously at a dose of about 840 mg every 2 weeks, about 1200 mg every 3 weeks, or about 1680 mg every 4 weeks. In some embodiments, atezolizumab is administered to the subject intravenously at a dose of about 1200 mg every 3 weeks. In some embodiments, atezolizumab is administered to the subject in a 21-day dosing cycle. In some embodiments, atezolizumab is administered to the subject intravenously at a dose of about 1200 mg on Day −2 to Day 4 of a 21-day dosing cycle. In some embodiments, atezolizumab is administered to the subject intravenously at a dose of about 1200 mg on Day 1 of a 21-day dosing cycle.

In a further example, provided herein is a method of treating NSCLC in a subject in need thereof, the method comprising administering to the subject an effective amount of atezolizumab in combination with a platinum-based chemotherapy comprising cisplatin and gemcitabine in one or more 21-day dosing cycles, wherein the subject is previously untreated for the NSCLC, wherein cisplatin is administered to the subject intravenously at a dose of about 75 mg/m² on Day 1 of a 21-day dosing cycle, wherein gemcitabine is administered to the subject intravenously at a dose of about 1000 mg/m² on Day 1 and Day 8 of a 21-day dosing cycle, wherein atezolizumab is administered to the subject intravenously at a dose of about 1200 mg on Day 1 of a 21-day dosing cycle, and wherein the PD-1 axis binding antagonist extends the subject's PFS and/or OS as compared to treatment with the platinum-based chemotherapy without atezolizumab.

In a still further example, provided herein is a pharmaceutical composition comprising a PD-1 axis binding antagonist for use in treatment of NSCLC in a subject in need thereof, the treatment comprises administration of atezolizumab in combination with a platinum-based chemotherapy comprising cisplatin and gemcitabine in one or more 21-day dosing cycles, wherein the subject is previously untreated for the NSCLC, wherein cisplatin is administered to the subject intravenously at a dose of about 75 mg/m² on Day 1 of a 21-day dosing cycle, wherein gemcitabine is administered to the subject intravenously at a dose of about 1000 mg/m² on Day 1 and Day 8 of a 21-day dosing cycle, wherein atezolizumab is administered to the subject intravenously at a dose of about 1200 mg on Day 1 of a 21-day dosing cycle, and wherein the PD-1 axis binding antagonist extends the subject's PFS and/or OS as compared to treatment with the platinum-based chemotherapy without atezolizumab.

In another example, provided herein is a method of treating NSCLC in a subject in need thereof, the method comprising administering to the subject an effective amount of atezolizumab in combination with a platinum-based chemotherapy comprising carboplatin and gemcitabine in one or more 21-day dosing cycles, wherein the subject is previously untreated for the NSCLC, wherein carboplatin is administered to the subject intravenously at an AUC of about 5 on Day 1 of a 21-day dosing cycle, wherein gemcitabine is administered to the subject intravenously at a dose of about 1000 mg/m² on Day 1 and Day 8 of a 21-day dosing cycle, wherein atezolizumab is administered to the subject intravenously at a dose of about 1200 mg on Day 1 of a 21-day dosing cycle, and wherein the PD-1 axis binding antagonist extends the subject's PFS and/or OS as compared to treatment with the platinum-based chemotherapy without atezolizumab.

In yet another example, provided herein is a pharmaceutical composition comprising a PD-1 axis binding antagonist for use in treatment of NSCLC in a subject in need thereof, the treatment comprises administration of atezolizumab in combination with a platinum-based chemotherapy comprising carboplatin and gemcitabine in one or more 21-day dosing cycles, wherein the subject is previously untreated for the NSCLC, wherein carboplatin is administered to the subject intravenously at an AUC of about 5 on Day 1 of a 21-day dosing cycle, wherein gemcitabine is administered to the subject intravenously at a dose of about 1000 mg/m² on Day 1 and Day 8 of a 21-day dosing cycle, wherein atezolizumab is administered to the subject intravenously at a dose of about 1200 mg on Day 1 of a 21-day dosing cycle, and wherein the PD-1 axis binding antagonist extends the subject's PFS and/or OS as compared to treatment with the platinum-based chemotherapy without atezolizumab.

In a further example, provided herein is a method of treating NSCLC in a subject in need thereof, the method comprising administering to the subject an effective amount of atezolizumab in combination with a platinum-based chemotherapy comprising cisplatin and gemcitabine in one or more 21-day dosing cycles, wherein the subject is previously untreated for the NSCLC, wherein cisplatin is administered to the subject intravenously at a dose of about 75 mg/m² on Day 1 of a 21-day dosing cycle, wherein gemcitabine is administered to the subject intravenously at a dose of about 1000 mg/m² on Day 1 and Day 8 of a 21-day dosing cycle, wherein atezolizumab is administered to the subject intravenously at a dose of about 1200 mg on Day 1 of a 21-day dosing cycle, and wherein the PD-1 axis binding antagonist extends the subject's PFS as compared to treatment with the platinum-based chemotherapy without atezolizumab.

In a still further example, provided herein is a pharmaceutical composition comprising a PD-1 axis binding antagonist for use in treatment of NSCLC in a subject in need thereof, the treatment comprises administration of atezolizumab in combination with a platinum-based chemotherapy comprising cisplatin and gemcitabine in one or more 21-day dosing cycles, wherein the subject is previously untreated for the NSCLC, wherein cisplatin is administered to the subject intravenously at a dose of about 75 mg/m² on Day 1 of a 21-day dosing cycle, wherein gemcitabine is administered to the subject intravenously at a dose of about 1000 mg/m² on Day 1 and Day 8 of a 21-day dosing cycle, wherein atezolizumab is administered to the subject intravenously at a dose of about 1200 mg on Day 1 of a 21-day dosing cycle, and wherein the PD-1 axis binding antagonist extends the subject's PFS as compared to treatment with the platinum-based chemotherapy without atezolizumab.

In another example, provided herein is a method of treating NSCLC in a subject in need thereof, the method comprising administering to the subject an effective amount of atezolizumab in combination with a platinum-based chemotherapy comprising carboplatin and gemcitabine in one or more 21-day dosing cycles, wherein the subject is previously untreated for the NSCLC, wherein carboplatin is administered to the subject intravenously at an AUC of about 5 on Day 1 of a 21-day dosing cycle, wherein gemcitabine is administered to the subject intravenously at a dose of about 1000 mg/m² on Day 1 and Day 8 of a 21-day dosing cycle, wherein atezolizumab is administered to the subject intravenously at a dose of about 1200 mg on Day 1 of a 21-day dosing cycle, and wherein the PD-1 axis binding antagonist extends the subject's PFS as compared to treatment with the platinum-based chemotherapy without atezolizumab.

In yet another example, provided herein is a pharmaceutical composition comprising a PD-1 axis binding antagonist for use in treatment of NSCLC in a subject in need thereof, the treatment comprises administration of atezolizumab in combination with a platinum-based chemotherapy comprising carboplatin and gemcitabine in one or more 21-day dosing cycles, wherein the subject is previously untreated for the NSCLC, wherein carboplatin is administered to the subject intravenously at an AUC of about 5 on Day 1 of a 21-day dosing cycle, wherein gemcitabine is administered to the subject intravenously at a dose of about 1000 mg/m² on Day 1 and Day 8 of a 21-day dosing cycle, wherein atezolizumab is administered to the subject intravenously at a dose of about 1200 mg on Day 1 of a 21-day dosing cycle, and wherein the PD-1 axis binding antagonist extends the subject's PFS as compared to treatment with the platinum-based chemotherapy without atezolizumab.

In a further example, provided herein is a method of treating NSCLC in a subject in need thereof, the method comprising administering to the subject an effective amount of atezolizumab in combination with a platinum-based chemotherapy comprising cisplatin and gemcitabine in one or more 21-day dosing cycles, wherein the subject is previously untreated for the NSCLC, wherein cisplatin is administered to the subject intravenously at a dose of about 75 mg/m² on Day 1 of a 21-day dosing cycle, wherein gemcitabine is administered to the subject intravenously at a dose of about 1000 mg/m² on Day 1 and Day 8 of a 21-day dosing cycle, wherein atezolizumab is administered to the subject intravenously at a dose of about 1200 mg on Day 1 of a 21-day dosing cycle, and wherein the PD-1 axis binding antagonist extends the subject's OS as compared to treatment with the platinum-based chemotherapy without atezolizumab.

In a still further example, provided herein is a pharmaceutical composition comprising a PD-1 axis binding antagonist for use in treatment of NSCLC in a subject in need thereof, the treatment comprises administration of atezolizumab in combination with a platinum-based chemotherapy comprising cisplatin and gemcitabine in one or more 21-day dosing cycles, wherein the subject is previously untreated for the NSCLC, wherein cisplatin is administered to the subject intravenously at a dose of about 75 mg/m² on Day 1 of a 21-day dosing cycle, wherein gemcitabine is administered to the subject intravenously at a dose of about 1000 mg/m² on Day 1 and Day 8 of a 21-day dosing cycle, wherein atezolizumab is administered to the subject intravenously at a dose of about 1200 mg on Day 1 of a 21-day dosing cycle, and wherein the PD-1 axis binding antagonist extends the subject's OS as compared to treatment with the platinum-based chemotherapy without atezolizumab.

In another example, provided herein is a method of treating NSCLC in a subject in need thereof, the method comprising administering to the subject an effective amount of atezolizumab in combination with a platinum-based chemotherapy comprising carboplatin and gemcitabine in one or more 21-day dosing cycles, wherein the subject is previously untreated for the NSCLC, wherein carboplatin is administered to the subject intravenously at an AUC of about 5 on Day 1 of a 21-day dosing cycle, wherein gemcitabine is administered to the subject intravenously at a dose of about 1000 mg/m² on Day 1 and Day 8 of a 21-day dosing cycle, wherein atezolizumab is administered to the subject intravenously at a dose of about 1200 mg on Day 1 of a 21-day dosing cycle, and wherein the PD-1 axis binding antagonist extends the subject's OS as compared to treatment with the platinum-based chemotherapy without atezolizumab.

In yet another example, provided herein is a pharmaceutical composition comprising a PD-1 axis binding antagonist for use in treatment of NSCLC in a subject in need thereof, the treatment comprises administration of atezolizumab in combination with a platinum-based chemotherapy comprising carboplatin and gemcitabine in one or more 21-day dosing cycles, wherein the subject is previously untreated for the NSCLC, wherein carboplatin is administered to the subject intravenously at an AUC of about 5 on Day 1 of a 21-day dosing cycle, wherein gemcitabine is administered to the subject intravenously at a dose of about 1000 mg/m² on Day 1 and Day 8 of a 21-day dosing cycle, wherein atezolizumab is administered to the subject intravenously at a dose of about 1200 mg on Day 1 of a 21-day dosing cycle, and wherein the PD-1 axis binding antagonist extends the subject's OS as compared to treatment with the platinum-based chemotherapy without atezolizumab.

In other embodiments, the PD-1 axis binding antagonist is a PD-1 binding antagonist. In some embodiments, the PD-1 binding antagonist inhibits the binding of PD-1 to one or more of its ligand binding partners. In some embodiments, the PD-1 binding antagonist inhibits the binding of PD-1 to PD-L1, PD-L2, or both PD-L1 and PD-L2. In some embodiments, the PD-1 binding antagonist is an anti-PD-1 antibody. In some embodiments, the anti-PD-1 antibody is selected from the group consisting of: MDX-1106 (nivolumab), MK-3475 (pembrolizumab), MEDI-0680 (AMP-514), PDR001, REGN2810, and BGB-108. In some embodiments, the PD-1 binding antagonist is an Fc fusion protein. In some embodiments, the Fc fusion protein is AMP-224.

In some embodiments, the subject has not previously been administered chemotherapy for treatment of the NSCLC. For example, in some embodiments, the subject has not previously been administered systemic therapy for treatment of the NSCLC. In some embodiments, the subject has not previously been administered any therapy for treatment of the NSCLC.

In some embodiments, the NSCLC is squamous NSCLC.

In some embodiments, the NSCLC is non-squamous NSCLC.

In some embodiments, the NSCLC is stage IV NSCLC.

In some embodiments, a tumor sample obtained from the patient has been determined to have a detectable expression level of PD-L1, e.g., in less than 1% (e.g., 0%, 0.25%, 0.5%, 0.75%, or 0.99%) of the tumor cells in the tumor sample.

In other embodiments, a tumor sample obtained from the patient has been determined to have a detectable expression level of PD-L1 in 1% or more (e.g., 1% or more, 5% or more, 10% or more, 20% or more, 30% or more, 40% or more, 50% or more, 60% or more, 70% or more, 80% or more, 90% or more, 95% or more, or 99% or more) of the tumor cells in the tumor sample. For example, in some embodiments, the tumor sample obtained from the patient has been determined to have a detectable expression level of PD-L1 in from 1% to less than 5% of the tumor cells in the tumor sample. In other embodiments, the tumor sample obtained from the patient has been determined to have a detectable expression level of PD-L1 in 5% or more of the tumor cells in the tumor sample. In some embodiments, the tumor sample obtained from the patient has been determined to have a detectable expression level of PD-L1 in from 5% to less than 50% of the tumor cells in the tumor sample. In other embodiments, the tumor sample obtained from the patient has been determined to have a detectable expression level of PD-L1 in 50% or more of the tumor cells in the tumor sample.

In some embodiments, a tumor sample obtained from the patient has been determined to have a detectable expression level of PD-L1 in tumor-infiltrating immune cells, e.g., that comprise less than 1% (e.g., 0%, 0.25%, 0.5%, 0.75%, or 0.99%) of the tumor sample.

In other embodiments, a tumor sample obtained from the patient has been determined to have a detectable expression level of PD-L1 in tumor-infiltrating immune cells that comprise 1% or more (e.g., 1% or more, 5% or more, 10% or more, 20% or more, 30% or more, 40% or more, 50% or more, 60% or more, 70% or more, 80% or more, 90% or more, 95% or more, or 99% or more) of the tumor sample. In some embodiments, the tumor sample obtained from the patient has been determined to have a detectable expression level of PD-L1 in tumor-infiltrating immune cells that comprise from 1% to less than 5% of the tumor sample. In other embodiments, the tumor sample obtained from the patient has been determined to have a detectable expression level of PD-L1 in tumor-infiltrating immune cells that comprise 5% or more of the tumor sample. In other embodiments, the tumor sample obtained from the patient has been determined to have a detectable expression level of PD-L1 in tumor-infiltrating immune cells that comprise from 5% to less than 10% of the tumor sample. In some embodiments, the tumor sample obtained from the patient has been determined to have a detectable expression level of PD-L1 in tumor-infiltrating immune cells that comprise 10% or more of the tumor sample.

In some embodiments, the tumor sample is a formalin-fixed and paraffin-embedded (FFPE) tumor sample, an archival tumor sample, a fresh tumor sample, or a frozen tumor sample. The presence and/or expression level of any of the biomarkers described herein (e.g., PD-L1) can be determined using any method described herein, or using approaches that are known in the art.

The subject is preferably a human. In other embodiments, the subject is a non-human mammal.

As a general proposition, the therapeutically effective amount of a PD-1 axis binding antagonist (e.g., an anti-PD-L1 antibody (e.g., atezolizumab) or an anti-PD-1 antibody) administered to a human will be in the range of about 0.01 to about 50 mg/kg of patient body weight, whether by one or more administrations. In some embodiments, for example, the antagonist (e.g., a PD-1 axis binding antagonist (e.g., an anti-PD-L1 antibody (e.g., atezolizumab) or an anti-PD-1 antibody)) is administered in a dose of about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, weekly, every two weeks, every three weeks, or every four weeks, for example. In some embodiments, the antagonist (e.g., a PD-1 axis binding antagonist (e.g., an anti-PD-L1 antibody (e.g., atezolizumab) or an anti-PD-1 antibody) and/or a VEGF antagonist (e.g., an anti-VEGF antibody (e.g., bevacizumab))) is administered at 15 mg/kg. However, other dosage regimens may be useful. In one embodiment, a PD-1 axis binding antagonist (e.g., an anti-PD-L1 antibody (e.g., atezolizumab) or an anti-PD-1 antibody) is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, or about 1500 mg. In some embodiments, the antagonist (e.g., an anti-PD-L1 antibody (e.g., atezolizumab) or an anti-PD-1 antibody) may be administered at a dose of about 1000 mg to about 1400 mg every three weeks (e.g., about 1100 mg to about 1300 mg every three weeks, e.g., about 1150 mg to about 1250 mg every three weeks). In some embodiments, the antagonist (e.g., an anti-PD-L1 antibody (e.g., atezolizumab) or an anti-PD-1 antibody) is administered to the subject intravenously at a dose of about 840 mg every 2 weeks, about 1200 mg every 3 weeks, or about 1680 mg every 4 weeks. In some embodiments, the antagonist (e.g., an anti-PD-L1 antibody (e.g., atezolizumab) or an anti-PD-1 antibody) is administered at a dose of about 1200 mg of atezolizumab every three weeks. The dose may be administered as a single dose or as multiple doses (e.g., 2 or 3 doses), such as infusions. The dose of the antibody administered in a combination treatment may be reduced as compared to a single treatment. In some embodiments, the treatment regimen comprises administering intravenously to the subject about 1200 mg of atezolizumab every three weeks. The progress of this therapy is easily monitored by conventional techniques.

In some embodiments, the PD-1 axis binding antagonist (e.g., an anti-PD-L1 antibody (e.g., atezolizumab) or an anti-PD-1 antibody) and the platinum-based chemotherapy (e.g., cisplatin or carboplatin and gemcitabine) are administered in a single dosing regimen. The administration of these agents may be concurrent or separate within the context of the dosing regimen.

The PD-1 axis binding antagonist (e.g., an anti-PD-L1 antibody (e.g., atezolizumab) or an anti-PD-1 antibody) and/or the platinum-based chemotherapy (e.g., cisplatin or carboplatin and gemcitabine) may be administered in any suitable manner known in the art. For example, the PD-1 axis binding antagonist (e.g., an anti-PD-L1 antibody (e.g., atezolizumab) or an anti-PD-1 antibody) and the platinum-based chemotherapy (e.g., cisplatin or carboplatin and gemcitabine) may be administered sequentially (at different times) or concurrently (at the same time). In some embodiments, the PD-1 axis binding antagonist is administered prior to the platinum-based chemotherapy (e.g., cisplatin or carboplatin and gemcitabine). In other embodiments, the PD-1 axis binding antagonist is administered after the platinum-based chemotherapy (e.g., cisplatin or carboplatin and gemcitabine). In yet other embodiments, the PD-1 axis binding antagonist is administered concurrently with the platinum-based chemotherapy (e.g., cisplatin or carboplatin and gemcitabine). In some embodiments, the PD-1 axis binding antagonist (e.g., an anti-PD-L1 antibody (e.g., atezolizumab) or an anti-PD-1 antibody) is in a separate composition as the platinum-based chemotherapy (e.g., cisplatin or carboplatin and gemcitabine). In some embodiments, the PD-1 axis binding antagonist (e.g., an anti-PD-L1 antibody (e.g., atezolizumab) or an anti-PD-1 antibody) is in the same composition as the platinum-based chemotherapy (e.g., cisplatin or carboplatin and gemcitabine).

The PD-1 axis binding antagonist (e.g., an anti-PD-L1 antibody (e.g., atezolizumab) or an anti-PD-1 antibody) and the platinum-based chemotherapy (e.g., cisplatin or carboplatin and gemcitabine) may be administered by the same route of administration or by different routes of administration. In some embodiments, the PD-1 axis binding antagonist (e.g., an anti-PD-L1 antibody (e.g., atezolizumab) or an anti-PD-1 antibody) is administered intravenously, intramuscularly, subcutaneously, topically, orally, transdermally, intraperitoneally, intraorbitally, by implantation, by inhalation, intrathecally, intraventricularly, or intranasally. In some embodiments, the platinum-based chemotherapy (e.g., cisplatin or carboplatin and gemcitabine) is administered intravenously, intramuscularly, subcutaneously, topically, orally, transdermally, intraperitoneally, intraorbitally, by implantation, by inhalation, intrathecally, intraventricularly, or intranasally. An effective amount of the PD-1 axis binding antagonist (e.g., an anti-PD-L1 antibody (e.g., atezolizumab) or an anti-PD-1 antibody) and platinum-based chemotherapy (e.g., cisplatin or carboplatin and gemcitabine) may be administered for prevention or treatment of disease. The appropriate dosage of the PD-1 axis binding antagonist (e.g., an anti-PD-L1 antibody (e.g., atezolizumab) or an anti-PD-1 antibody) and/or the platinum-based chemotherapy (e.g., cisplatin or carboplatin and gemcitabine) may be determined based on the type of disease to be treated, the type of the PD-1 axis binding antagonist and the VEGF antagonist, the severity and course of the disease, the clinical condition of the individual, the individual's clinical history and response to the treatment, and the discretion of the attending physician. In some embodiments, the PD-1 axis binding antagonist (e.g., an anti-PD-L1 antibody (e.g., atezolizumab) or an anti-PD-1 antibody) and/or the platinum-based chemotherapy (e.g., cisplatin or carboplatin and gemcitabine) is administered intravenously by infusion.

In some embodiments, the treatment may further comprise an additional therapy. Any suitable additional therapy known in the art or described herein may be used. The additional therapy may be radiation therapy, surgery or cystectomy, chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy. In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, and the like). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting P13K/AKT/mTOR pathway, HSP90 inhibitor, tubulin inhibitor, apoptosis inhibitor, and/or chemopreventative agent. The additional therapy may be one or more of the chemotherapeutic agents described herein.

### V. Combination Therapies

Also provided herein are methods for treating or delaying progression of NSCLC (e.g., squamous or non-squamous NSCLC, including stage IV NSCLC) in a subject comprising administering to the subject a treatment regimen comprising an effective amount of a PD-1 axis binding antagonist (e.g., an anti-PD-L1 antibody (e.g., atezolizumab) or an anti-PD-1 antibody) and/or a platinum-based chemotherapy (e.g., cisplatin or carboplatin and gemcitabine) in conjunction with another anti-cancer agent or cancer therapy. In some embodiments, the methods comprise administering to the individual a PD-1 axis binding antagonist (e.g., an anti-PD-L1 antibody (e.g., atezolizumab) or an anti-PD-1 antibody), a platinum-based chemotherapy (e.g., cisplatin or carboplatin and gemcitabine), and an additional therapeutic agent.

In some embodiments, a PD-1 axis binding antagonist (e.g., an anti-PD-L1 antibody (e.g., atezolizumab) or an anti-PD-1 antibody) and/or a platinum-based chemotherapy (e.g., cisplatin or carboplatin and gemcitabine) may be administered in conjunction with an additional chemotherapy or chemotherapeutic agent. In some embodiments, a PD-1 axis binding antagonist (e.g., an anti-PD-L1 antibody (e.g., atezolizumab) or an anti-PD-1 antibody) and/or a platinum-based chemotherapy (e.g., cisplatin or carboplatin and gemcitabine) may be administered in conjunction with a radiation therapy or radiotherapeutic agent. In some embodiments, a PD-1 axis binding antagonist (e.g., an anti-PD-L1 antibody (e.g., atezolizumab) or an anti-PD-1 antibody) and/or a platinum-based chemotherapy (e.g., cisplatin or carboplatin and gemcitabine) may be administered in conjunction with a targeted therapy or targeted therapeutic agent. In some embodiments, a PD-1 axis binding antagonist (e.g., an anti-PD-L1 antibody (e.g., atezolizumab) or an anti-PD-1 antibody) and/or a platinum-based chemotherapy (e.g., cisplatin or carboplatin and gemcitabine) may be administered in conjunction with an immunotherapy or immunotherapeutic agent, for example, a monoclonal antibody.

Without wishing to be bound to theory, it is thought that enhancing T cell stimulation, by promoting an activating co-stimulatory molecule or by inhibiting a negative co-stimulatory molecule, may promote tumor cell death, thereby treating or delaying progression of cancer. In some embodiments, a PD-1 axis binding antagonist (e.g., an anti-PD-L1 antibody (e.g., atezolizumab) or an anti-PD-1 antibody) and/or a platinum-based chemotherapy (e.g., cisplatin or carboplatin and gemcitabine) may be administered in conjunction with an agonist directed against an activating co-stimulatory molecule. In some embodiments, an activating co-stimulatory molecule may include CD40, CD226, CD28, OX40, GITR, CD137, CD27, HVEM, or CD127. In some embodiments, the agonist directed against an activating co-stimulatory molecule is an agonist antibody that binds to CD40, CD226, CD28, OX40, GITR, CD137, CD27, HVEM, or CD127. In some embodiments, a PD-1 axis binding antagonist (e.g., an anti-PD-L1 antibody (e.g., atezolizumab) or an anti-PD-1 antibody) and/or a platinum-based chemotherapy (e.g., cisplatin or carboplatin and gemcitabine) may be administered in conjunction with an antagonist directed against an inhibitory co-stimulatory molecule. In some embodiments, an inhibitory co-stimulatory molecule may include CTLA-4 (also known as CD152), PD-1, TIM-3, BTLA, VISTA, LAG-3, B7-H3, B7-H4, IDO, TIGIT, MICA/B, or arginase. In some embodiments, the antagonist directed against an inhibitory co-stimulatory molecule is an antagonist antibody that binds to CTLA-4, PD-1, TIM-3, BTLA, VISTA, LAG-3, B7-H3, B7-H4, IDO, TIGIT, MICA/B, or arginase.

In some embodiments, a PD-1 axis binding antagonist (e.g., an anti-PD-L1 antibody (e.g., atezolizumab) or an anti-PD-1 antibody) and/or a platinum-based chemotherapy (e.g., cisplatin or carboplatin and gemcitabine) may be administered in conjunction with an antagonist directed against CTLA-4 (also known as CD152), for example, a blocking antibody. In some embodiments, a PD-1 axis binding antagonist (e.g., an anti-PD-L1 antibody (e.g., atezolizumab) or an anti-PD-1 antibody) and/or a platinum-based chemotherapy (e.g., cisplatin or carboplatin and gemcitabine) may be administered in conjunction with ipilimumab (also known as MDX-010, MDX-101, or YERVOY®). In some embodiments, a PD-1 axis binding antagonist (e.g., an anti-PD-L1 antibody (e.g., atezolizumab) or an anti-PD-1 antibody) and/or a platinum-based chemotherapy (e.g., cisplatin or carboplatin and gemcitabine) may be administered in conjunction with tremelimumab (also known as ticilimumab or CP-675,206). In some embodiments, a PD-1 axis binding antagonist (e.g., an anti-PD-L1 antibody (e.g., atezolizumab) or an anti-PD-1 antibody) and/or a platinum-based chemotherapy (e.g., cisplatin or carboplatin and gemcitabine) may be administered in conjunction with an antagonist directed against B7-H3 (also known as CD276), for example, a blocking antibody. In some embodiments, a PD-1 axis binding antagonist (e.g., an anti-PD-L1 antibody (e.g., atezolizumab) or an anti-PD-1 antibody) and/or a platinum-based chemotherapy (e.g., cisplatin or carboplatin and gemcitabine) may be administered in conjunction with MGA271. In some embodiments, a PD-1 axis binding antagonist (e.g., an anti-PD-L1 antibody (e.g., atezolizumab) or an anti-PD-1 antibody) and/or a platinum-based chemotherapy (e.g., cisplatin or carboplatin and gemcitabine) may be administered in conjunction with an antagonist directed against a TGF beta, for example, metelimumab (also known as CAT-192), fresolimumab (also known as GC1008), or LY2157299.

In some embodiments, a PD-1 axis binding antagonist (e.g., an anti-PD-L1 antibody (e.g., atezolizumab) or an anti-PD-1 antibody) and/or a platinum-based chemotherapy (e.g., cisplatin or carboplatin and gemcitabine) may be administered in conjunction with a treatment comprising adoptive transfer of a T cell (e.g., a cytotoxic T cell or CTL) expressing a chimeric antigen receptor (CAR). In some embodiments, a PD-1 axis binding antagonist (e.g., an anti-PD-L1 antibody (e.g., atezolizumab) or an anti-PD-1 antibody) and/or a platinum-based chemotherapy (e.g., cisplatin or carboplatin and gemcitabine) may be administered in conjunction with a treatment comprising adoptive transfer of a T cell comprising a dominant-negative TGF beta receptor, e.g., a dominant-negative TGF beta type II receptor. In some embodiments, a PD-1 axis binding antagonist (e.g., an anti-PD-L1 antibody (e.g., atezolizumab) or an anti-PD-1 antibody) and/or a platinum-based chemotherapy (e.g., cisplatin or carboplatin and gemcitabine) may be administered in conjunction with a treatment comprising a HERCREEM protocol (see, e.g., ClinicalTrials.gov Identifier NCT00889954).

In some embodiments, a PD-1 axis binding antagonist and/or a platinum-based chemotherapy may be administered in conjunction with an agonist directed against CD137 (also known as TNFRSF9, 4-1 BB, or ILA), for example, an activating antibody. In some embodiments, a PD-1 axis binding antagonist and/or a platinum-based chemotherapy may be administered in conjunction with urelumab (also known as BMS-663513). In some embodiments, a PD-1 axis binding antagonist and/or a platinum-based chemotherapy may be administered in conjunction with an agonist directed against CD40, for example, an activating antibody. In some embodiments, a PD-1 axis binding antagonist and/or a platinum-based chemotherapy may be administered in conjunction with CP-870893. In some embodiments, a PD-1 axis binding antagonist and/or a platinum-based chemotherapy may be administered in conjunction with an agonist directed against OX40 (also known as CD134), for example, an activating antibody. In some embodiments, a PD-1 axis binding antagonist and/or a platinum-based chemotherapy may be administered in conjunction with an anti-OX40 antibody (e.g., AgonOX). In some embodiments, a PD-1 axis binding antagonist and/or a platinum-based chemotherapy may be administered in conjunction with an agonist directed against CD27, for example, an activating antibody. In some embodiments, a PD-1 axis binding antagonist and/or a platinum-based chemotherapy may be administered in conjunction with CDX-1127. In some embodiments, a PD-1 axis binding antagonist and/or a platinum-based chemotherapy may be administered in conjunction with an antagonist directed against indoleamine-2,3-dioxygenase (IDO). In some embodiments, the IDO antagonist is 1-methyl-D-tryptophan (also known as 1-D-MT).

In some embodiments, a PD-1 axis binding antagonist and/or a platinum-based chemotherapy may be administered in conjunction with an antibody-drug conjugate. In some embodiments, the antibody-drug conjugate comprises mertansine or monomethyl auristatin E (MMAE). In some embodiments, a PD-1 axis binding antagonist and/or a platinum-based chemotherapy may be administered in conjunction with and anti-NaPi2b antibody-MMAE conjugate (also known as DNIB0600A or RG7599). In some embodiments, a PD-1 axis binding antagonist and/or a platinum-based chemotherapy may be administered in conjunction with trastuzumab emtansine (also known as T-DM1, ado-trastuzumab emtansine, or KADCYLA®, Genentech). In some embodiments, a PD-1 axis binding antagonist and/or a platinum-based chemotherapy may be administered in conjunction with DMUC5754A. In some embodiments, a PD-1 axis binding antagonist and/or a platinum-based chemotherapy may be administered in conjunction with an antibody-drug conjugate targeting the endothelin B receptor (EDNBR), for example, an antibody directed against EDNBR conjugated with MMAE.

In some embodiments, a PD-1 axis binding antagonist and/or a platinum-based chemotherapy may be administered in conjunction with an angiogenesis inhibitor. In some embodiments, a PD-1 axis binding antagonist and/or a platinum-based chemotherapy may be administered in conjunction with an antibody directed against angiopoietin 2 (also known as Ang2). In some embodiments, a PD-1 axis binding antagonist and/or a platinum-based chemotherapy may be administered in conjunction with MEDI3617.

In some embodiments, a PD-1 axis binding antagonist and/or a platinum-based chemotherapy may be administered in conjunction with an antineoplastic agent. In some embodiments, a PD-1 axis binding antagonist and/or a platinum-based chemotherapy may be administered in conjunction with an agent targeting CSF-1R (also known as M-CSFR or CD115). In some embodiments, a PD-1 axis binding antagonist and/or a platinum-based chemotherapy may be administered in conjunction with anti-CSF-1R (also known as IMC-CS4). In some embodiments, a PD-1 axis binding antagonist and/or a platinum-based chemotherapy may be administered in conjunction with an interferon, for example interferon alpha or interferon gamma. In some embodiments, a PD-1 axis binding antagonist and/or a platinum-based chemotherapy may be administered in conjunction with Roferon-A (also known as recombinant Interferon alpha-2a). In some embodiments, a PD-1 axis binding antagonist and/or a platinum-based chemotherapy may be administered in conjunction with GM-CSF (also known as recombinant human granulocyte macrophage colony stimulating factor, rhu GM-CSF, sargramostim, or LEUKINE®). In some embodiments, a PD-1 axis binding antagonist and/or a platinum-based chemotherapy may be administered in conjunction with IL-2 (also known as aldesleukin or PROLEUKIN®). In some embodiments, a PD-1 axis binding antagonist and/or a platinum-based chemotherapy may be administered in conjunction with IL-12. In some embodiments, a PD-1 axis binding antagonist and/or a platinum-based chemotherapy may be administered in conjunction with an antibody targeting CD20. In some embodiments, the antibody targeting CD20 is obinutuzumab (also known as GA101 or GAZYVA®) or rituximab. In some embodiments, a PD-1 axis binding antagonist and/or a platinum-based chemotherapy may be administered in conjunction with an antibody targeting GITR. In some embodiments, the antibody targeting GITR is TRX518.

In some embodiments, a PD-1 axis binding antagonist and/or a platinum-based chemotherapy may be administered in conjunction with a cancer vaccine. In some embodiments, the cancer vaccine is a peptide cancer vaccine, which in some embodiments is a personalized peptide vaccine. In some embodiments the peptide cancer vaccine is a multivalent long peptide, a multi-peptide, a peptide cocktail, a hybrid peptide, or a peptide-pulsed dendritic cell vaccine (see, e.g., Yamada et al., Cancer Sci, 104:14-21, 2013). In some embodiments, a PD-1 axis binding antagonist and/or a platinum-based chemotherapy may be administered in conjunction with an adjuvant. In some embodiments, a PD-1 axis binding antagonist and/or a platinum-based chemotherapy may be administered in conjunction with a treatment comprising a TLR agonist, for example, Poly-ICLC (also known as HILTONOL®), LPS, MPL, or CpG ODN. In some embodiments, a PD-1 axis binding antagonist and/or a platinum-based chemotherapy may be administered in conjunction with tumor necrosis factor (TNF) alpha. In some embodiments, a PD-1 axis binding antagonist and/or a platinum-based chemotherapy may be administered in conjunction with IL-1. In some embodiments, a PD-1 axis binding antagonist and/or a platinum-based chemotherapy may be administered in conjunction with HMGB1. In some embodiments, a PD-1 axis binding antagonist and/or a platinum-based chemotherapy may be administered in conjunction with an IL-10 antagonist. In some embodiments, a PD-1 axis binding antagonist and/or a platinum-based chemotherapy may be administered in conjunction with an IL-4 antagonist. In some embodiments, a PD-1 axis binding antagonist and/or a platinum-based chemotherapy may be administered in conjunction with an IL-13 antagonist. In some embodiments, a PD-1 axis binding antagonist and/or a platinum-based chemotherapy may be administered in conjunction with an HVEM antagonist. In some embodiments, a PD-1 axis binding antagonist and/or a platinum-based chemotherapy may be administered in conjunction with an ICOS agonist, e.g., by administration of ICOS-L, or an agonistic antibody directed against ICOS. In some embodiments, a PD-1 axis binding antagonist and/or a platinum-based chemotherapy may be administered in conjunction with a treatment targeting CX3CL1. In some embodiments, a PD-1 axis binding antagonist and/or a platinum-based chemotherapy may be administered in conjunction with a treatment targeting CXCL9. In some embodiments, a PD-1 axis binding antagonist and/or a platinum-based chemotherapy may be administered in conjunction with a treatment targeting CXCL10. In some embodiments, a PD-1 axis binding antagonist and/or a platinum-based chemotherapy may be administered in conjunction with a treatment targeting CCLS. In some embodiments, a PD-1 axis binding antagonist and/or a platinum-based chemotherapy may be administered in conjunction with an LFA-1 or ICAM1 agonist. In some embodiments, a PD-1 axis binding antagonist and/or a platinum-based chemotherapy may be administered in conjunction with a Selectin agonist.

In some embodiments, a PD-1 axis binding antagonist and/or a platinum-based chemotherapy may be administered in conjunction with a targeted therapy. In some embodiments, a PD-1 axis binding antagonist and/or a platinum-based chemotherapy may be administered in conjunction with an inhibitor of B-Raf. In some embodiments, a PD-1 axis binding antagonist and/or a platinum-based chemotherapy may be administered in conjunction with vemurafenib (also known as ZELBORAF®). In some embodiments, a PD-1 axis binding antagonist and/or a platinum-based chemotherapy may be administered in conjunction with dabrafenib (also known as TAFINLAR®). In some embodiments, a PD-1 axis binding antagonist and/or a platinum-based chemotherapy may be administered in conjunction with erlotinib (also known as TARCEVA®). In some embodiments, a PD-1 axis binding antagonist and/or a platinum-based chemotherapy may be administered in conjunction with an inhibitor of a MEK, such as MEK1 (also known as MAP2K1) or MEK2 (also known as MAP2K2). In some embodiments, a PD-1 axis binding antagonist and/or a platinum-based chemotherapy may be administered in conjunction with cobimetinib (also known as GDC-0973 or XL-518). In some embodiments, a PD-1 axis binding antagonist and/or a platinum-based chemotherapy may be administered in conjunction with trametinib (also known as MEKINIST®). In some embodiments, a PD-1 axis binding antagonist and/or a platinum-based chemotherapy may be administered in conjunction with an inhibitor of K-Ras. In some embodiments, a PD-1 axis binding antagonist and/or a platinum-based chemotherapy may be administered in conjunction with an inhibitor of c-Met. In some embodiments, a PD-1 axis binding antagonist and/or a platinum-based chemotherapy may be administered in conjunction with onartuzumab (also known as MetMAb). In some embodiments, a PD-1 axis binding antagonist and/or a platinum-based chemotherapy may be administered in conjunction with an inhibitor of Alk. In some embodiments, a PD-1 axis binding antagonist and/or a platinum-based chemotherapy may be administered in conjunction with AF802 (also known as C_{H}5424802 or alectinib). In some embodiments, a PD-1 axis binding antagonist and/or a platinum-based chemotherapy may be administered in conjunction with an inhibitor of a phosphatidylinositol 3-kinase (P13K). In some embodiments, a PD-1 axis binding antagonist and/or a platinum-based chemotherapy may be administered in conjunction with BKM120. In some embodiments, a PD-1 axis binding antagonist and/or a platinum-based chemotherapy may be administered in conjunction with idelalisib (also known as GS-1101 or CAL-101). In some embodiments, a PD-1 axis binding antagonist and/or a platinum-based chemotherapy may be administered in conjunction with perifosine (also known as KRX-0401). In some embodiments, a PD-1 axis binding antagonist and/or a platinum-based chemotherapy may be administered in conjunction with an inhibitor of an Akt. In some embodiments, a PD-1 axis binding antagonist and/or a platinum-based chemotherapy may be administered in conjunction with MK2206. In some embodiments, a PD-1 axis binding antagonist and/or a platinum-based chemotherapy may be administered in conjunction with GSK690693. In some embodiments, a PD-1 axis binding antagonist and/or a platinum-based chemotherapy may be administered in conjunction with GDC-0941. In some embodiments, a PD-1 axis binding antagonist and/or a platinum-based chemotherapy may be administered in conjunction with an inhibitor of mTOR. In some embodiments, a PD-1 axis binding antagonist and/or a platinum-based chemotherapy may be administered in conjunction with sirolimus (also known as rapamycin). In some embodiments, a PD-1 axis binding antagonist and/or a platinum-based chemotherapy may be administered in conjunction with temsirolimus (also known as CCI-779 or TORISEL®). In some embodiments, a PD-1 axis binding antagonist and/or a platinum-based chemotherapy may be administered in conjunction with everolimus (also known as RAD001). In some embodiments, a PD-1 axis binding antagonist and/or a platinum-based chemotherapy may be administered in conjunction with ridaforolimus (also known as AP-23573, MK-8669, or deforolimus). In some embodiments, a PD-1 axis binding antagonist and/or a platinum-based chemotherapy may be administered in conjunction with OSI-027. In some embodiments, a PD-1 axis binding antagonist and/or a platinum-based chemotherapy may be administered in conjunction with AZD8055. In some embodiments, a PD-1 axis binding antagonist and/or a platinum-based chemotherapy may be administered in conjunction with INK128. In some embodiments, a PD-1 axis binding antagonist and/or a platinum-based chemotherapy may be administered in conjunction with a dual PI3K/mTOR inhibitor. In some embodiments, a PD-1 axis binding antagonist and/or a platinum-based chemotherapy may be administered in conjunction with XL765. In some embodiments, a PD-1 axis binding antagonist and/or a platinum-based chemotherapy may be administered in conjunction with GDC-0980. In some embodiments, a PD-1 axis binding antagonist and/or a platinum-based chemotherapy may be administered in conjunction with BEZ235 (also known as NVP-BEZ235). In some embodiments, a PD-1 axis binding antagonist and/or a platinum-based chemotherapy may be administered in conjunction with BGT226. In some embodiments, a PD-1 axis binding antagonist and/or a platinum-based chemotherapy may be administered in conjunction with GSK2126458. In some embodiments, a PD-1 axis binding antagonist and/or a platinum-based chemotherapy may be administered in conjunction with PF-04691502. In some embodiments, a PD-1 axis binding antagonist and/or a platinum-based chemotherapy may be administered in conjunction with PF-05212384 (also known as PKI-587).

In any of the preceding embodiments, the PD-1 axis binding antagonist may be a human PD-1 axis binding antagonist.

In any of the preceding embodiments, the PD-1 axis binding antagonist is an anti-PD-L1 antibody (e.g., atezolizumab) or an anti-PD-1 antibody.

In any of the preceding embodiments, the platinum-based chemotherapy includes a platinum-based chemotherapeutic agent (e.g., cisplatin or carboplatin). In some embodiments, the platinum-based chemotherapy includes cisplatin. In some embodiments, the platinum-based chemotherapy includes carboplatin. In some embodiments, the platinum-based chemotherapy further includes one or more additional chemotherapeutic agents, e.g., a nucleoside analog. In some embodiments, the nucleoside analog is gemcitabine. In some embodiments, the platinum-based chemotherapy includes cisplatin and gemcitabine. In other embodiments, the platinum-based chemotherapy includes carboplatin and gemcitabine.

### VI. Assessment of PD-L1 Expression

The expression of PD-L1 may be assessed in a subject treated according to any of the methods and compositions for use described herein. In some embodiments, the method includes determining the expression level of PD-L1 in a biological sample (e.g., a tumor sample) obtained from the subject. In other embodiments, the expression level of PD-L1 in a biological sample (e.g., a tumor sample) obtained from the subject has been determined prior to initiation of treatment. In yet other embodiments, the expression level of PD-L1 in a biological sample (e.g., a tumor sample) obtained from the subject may be determined after initiation of treatment.

In some embodiments, a tumor sample obtained from the subject has been determined to have a detectable expression level of PD-L1 in tumor-infiltrating immune cells that comprise about 1% or more (e.g., about 1% or more, 2% or more, 3% or more, 5% or more, 6% or more, 7% or more, 8% or more, 9% or more, 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, about 50% or more, about 60% or more, about 70% or more, about 80% or more, about 90% or more, about 95% or more, about 96% or more, about 97% or more, about 98% or more, about 99% or more, or 100%) of the tumor sample. For example, in some embodiments, a tumor sample obtained from the subject has been determined to have a detectable expression level of PD-L1 in tumor-infiltrating immune cells that comprise from about 1% to less than about 5% (e.g., from 1% to 4.9%, from 1% to 4.5%, from 1% to 4%, from 1% to 3.5%, from 1% to 3%, from 1% to 2.5%, or from 1% to 2%) of the tumor sample.

In some embodiments, a tumor sample obtained from the subject has been determined to have a detectable expression level of PD-L1 in about 1% or more (e.g., about 1% or more, 2% or more, 3% or more, 5% or more, 6% or more, 7% or more, 8% or more, 9% or more, 10% or more, 11% or more, 12% or more, 13% or more, 14% or more, 15% or more, 16% or more, 17% or more,18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, about 50% or more, about 60% or more, about 70% or more, about 80% or more, about 90% or more, about 95% or more, about 96% or more, about 97% or more, about 98% or more, about 99% or more, or 100%) of the tumor-infiltrating immune cells in the tumor sample. For example, in some embodiments, a tumor sample obtained from the subject has been determined to have a detectable expression level of PD-L1 in from about 1% to less than about 5% (e.g., from 1% to 4.9%, from 1% to 4.5%, from 1% to 4%, from 1% to 3.5%, from 1% to 3%, from 1% to 2.5%, or from 1% to 2%) of the tumor-infiltrating immune cells in the tumor sample.

In other embodiments, a tumor sample obtained from the subject has been determined to have a detectable expression level of PD-L1 in tumor-infiltrating immune cells that comprise about 5% or more of the tumor sample. For example, in some embodiments, a tumor sample obtained from the subject has been determined to have a detectable expression level of PD-L1 in tumor-infiltrating immune cells that comprise from about 5% to less than about 10% (e.g., from 5% to 9.5%, from 5% to 9%, from 5% to 8.5%, from 5% to 8%, from 5% to 7.5%, from 5% to 7%, from 5% to 6.5%, from 5% to 6%, from 5% to 5.5%, from 6% to 9.5%, from 6% to 9%, from 6% to 8.5%, from 6% to 8%, from 6% to 7.5%, from 6% to 7%, from 6% to 6.5%, from 7% to 9.5%, from 7% to 9%, from 7% to 7.5%, from 8% to 9.5%, from 8% to 9%, or from 8% to 8.5%) of the tumor sample.

In yet other embodiments, a tumor sample obtained from the subject has been determined to have a detectable expression level of PD-L1 in about 5% or more of the tumor-infiltrating immune cells in the tumor sample. For example, in some embodiments, a tumor sample obtained from the subject has been determined to have a detectable expression level of PD-L1 in from about 5% to less than about 10% (e.g., from 5% to 9.5%, from 5% to 9%, from 5% to 8.5%, from 5% to 8%, from 5% to 7.5%, from 5% to 7%, from 5% to 6.5%, from 5% to 6%, from 5% to 5.5%, from 6% to 9.5%, from 6% to 9%, from 6% to 8.5%, from 6% to 8%, from 6% to 7.5%, from 6% to 7%, from 6% to 6.5%, from 7% to 9.5%, from 7% to 9%, from 7% to 7.5%, from 8% to 9.5%, from 8% to 9%, or from 8% to 8.5%) of the tumor-infiltrating immune cells in the tumor sample.

In still further embodiments, a tumor sample obtained from the subject has been determined to have a detectable expression level of PD-L1 in tumor-infiltrating immune cells that comprise about 10% or more (e.g., 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, 50% or more, 60% or more, 70% or more, 80% or more, 90% or more, 95% or more, 96% or more, 97% or more, 98% or more, 99% or more, or 100%) of the tumor sample.

In still further embodiments, a tumor sample obtained from the subject has been determined to have a detectable expression level of PD-L1 in about 10% or more (e.g., 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, 50% or more, 60% or more, 70% or more, 80% or more, 90% or more, 95% or more, 96% or more, 97% or more, 98% or more, 99% or more, or 100%) of the tumor-infiltrating immune cells in the tumor sample.

In yet other embodiments, a tumor sample obtained from the subject has been determined to have a detectable expression level of PD-L1 in about 50% or more (e.g., about 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more) of the tumor cells in the tumor sample and/or a detectable expression level of PD-L1 in tumor-infiltrating immune cells that comprise about 10% or more (e.g., 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, 50% or more, 60% or more, 70% or more, 80% or more, 90% or more, 95% or more, 96% or more, 97% or more, 98% or more, 99% or more, or 100%) of the tumor sample.

In yet other embodiments, a tumor sample obtained from the subject has been determined to have a detectable expression level of PD-L1 in about 5% or more (e.g., about 5% or more, 6% or more, 7% or more, 8% or more, 9% or more, 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, or 50% or more) of the tumor cells in the tumor sample and/or a detectable expression level of PD-L1 in tumor-infiltrating immune cells that comprise about 5% or more (e.g., about 5% or more, 6% or more, 7% or more, 8% or more, 9% or more, 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, or 50% or more) of the tumor sample.

It is to be understood that in any of the preceding examples, the percentage of the tumor sample comprised by tumor-infiltrating immune cells may be in terms of the percentage of tumor area covered by tumor-infiltrating immune cells in a section of the tumor sample obtained from the subject, for example, as assessed by IHC using an anti-PD-L1 antibody (e.g., the SP142 antibody). Any suitable anti-PD-L1 antibody may be used, including, e.g., SP142 (Ventana), SP263 (Ventana), 22C3 (Dako), 28-8 (Dako), El L3N (Cell Signaling Technology), 4059 (ProSci, Inc.), h5H1 (Advanced Cell Diagnostics), and 9A11. In some embodiments, the anti-PD-L1 antibody is SP142. In some embodiments, the anti-PD-L1 antibody is SP263.

In some embodiments, a tumor sample obtained from the subject has been determined to have a detectable expression level of PD-L1 in about 1% or more (e.g., about 1% or more, 2% or more, 3% or more, 5% or more, 6% or more, 7% or more, 8% or more, 9% or more, 10% or more, 11% or more, 12% or more, 13% or more, 14% or more, 15% or more, 16% or more, 17% or more,18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more) of the tumor cells in the tumor sample. For example, in some embodiments, a tumor sample obtained from the subject has been determined to have a detectable expression level of PD-L1 in from about 1% to less than about 5% (e.g., from 1% to 4.9%, from 1% to 4.5%, from 1% to 4%, from 1% to 3.5%, from 1% to 3%, from 1% to 2.5%, or from 1% to 2%) of the tumor cells in the tumor sample. In other embodiments, a tumor sample obtained from the subject has been determined to have a detectable expression level of PD-L1 in less than about 1% of the tumor cells in the tumor sample.

In other embodiments, a tumor sample obtained from the subject has been determined to have a detectable expression level of PD-L1 in about 5% or more of the tumor cells in the tumor sample. For example, in some embodiments, a tumor sample obtained from the subject has been determined to have a detectable expression level of PD-L1 in from about 5% to less than 50% (e.g., from 5% to 49.5%, from 5% to 45%, from 5% to 40%, from 5% to 35%, from 5% to 30%, from 5% to 25%, from 5% to 20%, from 5% to 15%, from 5% to 10%, from 5% to 9%, from 5% to 8%, from 5% to 7%, from 5% to 6%, from 10% to 49.5%, from 10% to 40%, from 10% to 35%, from 10% to 30%, from 10% to 25%, from 10% to 20%, from 10% to 15%, from 15% to 49.5%, from 15% to 45%, from 15% to 40%, from 15% to 35%, from 15% to 30%, from 15% to 30%, from 15% to 25%, from 15% to 20%, from 20% to 49.5%, from 20% to 45%, from 20% to 40%, from 20% to 35%, from 20% to 30%, from 20% to 25%, from 25% to 49.5%, from 25% to 45%, from 25% to 40%, from 25% to 35%, from 25% to 30%, from 30% to 49.5%, from 30% to 45%, from 30% to 40%, from 30% to 35%, from 35% to 49.5%, from 35% to 45%, from 35% to 40%, from 40% to 49.5%, from 40% to 45%, or from 45% to 49.5%) of the tumor cells in the tumor sample.

In yet other embodiments, a tumor sample obtained from the subject has been determined to have a detectable expression level of PD-L1 in about 50% or more (e.g., about 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more) of the tumor cells in the tumor sample. In some embodiments, a tumor sample obtained from the subject has been determined to have a detectable expression level of PD-L1 in from about 50% to about 99% (e.g., from 50% to 99%, from 50% to 95%, from 50% to 90%, from 50% to 85%, from 50% to 80%, from 50% to 75%, from 50% to 70%, from 50% to 65%, from 50% to 60%, from 50% to 55%, from 55% to 99%, from 55% to 95%, from 55% to 90%, from 55% to 85%, from 55% to 80%, from 55% to 75%, from 55% to 70%, from 55% to 65%, from 55% to 60%, from 60% to 99%, from 60% to 95%, from 60% to 90%, from 60% to 85%, from 60% to 80%, from 60% to 75%, from 60% to 70%, from 60% to 65%, from 65% to 99%, from 65% to 95%, from 65% to 90%, from 65% to 85%, from 65% to 80%, from 65% to 75%, from 65% to 70%, from 70% to 99%, from 70% to 95%, from 70% to 90%, from 70% to 85%, from 70% to 80%, from 70% to 75%, from 75% to 99%, from 75% to 95%, from 75% to 90%, from 75% to 85%, from 75% to 80%, from 80% to 99%, from 80% to 95%, from 80% to 90%, from 80% to 85%, from 85% to 99%, from 85% to 95%, from 85% to 90%, from 90% to 99%, or from 90% to 95%) of the tumor cells in the tumor sample.

In some embodiments, the tumor sample is a formalin-fixed and paraffin-embedded (FFPE) tumor sample, an archival tumor sample, a fresh tumor sample, or a frozen tumor sample.

The presence and/or expression level of any of the biomarkers described above (including PD-L1 (e.g., PD-L1 expression on tumor-infiltrating immune cells (IC) in a tumor sample obtained from the subject and/or PD-L1 expression on tumor cells (TC) in a tumor sample obtained from the subject)), e.g., in a tumor sample obtained from the subject) may be assessed qualitatively and/or quantitatively based on any suitable criterion known in the art, including but not limited to DNA, mRNA, cDNA, proteins, protein fragments, and/or gene copy number. Methodologies for measuring such biomarkers are known in the art and understood by the skilled artisan, including, but not limited to, IHC, Western blot analysis, immunoprecipitation, molecular binding assays, ELISA, ELIFA, fluorescence activated cell sorting (“FACS”), MassARRAY, proteomics, quantitative blood based assays (e.g., Serum ELISA), biochemical enzymatic activity assays, in situ hybridization (ISH), fluorescence in situ hybridization (FISH), Southern analysis, Northern analysis, whole genome sequencing, polymerase chain reaction (PCR) including quantitative real time PCR (qRT-PCR) and other amplification type detection methods, such as, for example, branched DNA, SISBA, TMA and the like, RNASeq, microarray analysis, gene expression profiling, whole-genome sequencing (WGS), and/or serial analysis of gene expression (“SAGE”), as well as any one of the wide variety of assays that can be performed by protein, gene, and/or tissue array analysis. Typical protocols for evaluating the status of genes and gene products are found, for example, in Ausubel et al. eds. (Current Protocols in Molecular Biology, 1995), Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

In some embodiments of any of the preceding methods, the expression level of a biomarker (e.g., PD-L1) may be a protein expression level. In certain embodiments, the method comprises contacting the sample with antibodies that specifically bind to a biomarker described herein under conditions permissive for binding of the biomarker, and detecting whether a complex is formed between the antibodies and biomarker. Such method may be an in vitro or in vivo method. In some embodiments, an antibody is used to select subjects eligible for treatment with an anti-cancer therapy that includes a PD-1 axis binding antagonist, e.g., an anti-PD-L1 antibody (e.g., atezolizumab) or an anti-PD-1 antibody, e.g., a biomarker for selection of subjects. In some embodiments, an antibody is used to select subjects eligible for treatment with an anti-cancer therapy that includes a PD-1 axis binding antagonist (e.g., an anti-PD-L1 antibody (e.g., atezolizumab) or an anti-PD-1 antibody) and a platinum-based chemotherapy (e.g., cisplatin or carboplatin and gemcitabine), e.g., a biomarker for selection of subjects.

Any method of measuring protein expression levels known in the art or provided herein may be used. For example, in some embodiments, a protein expression level of a biomarker is determined using a method selected from the group consisting of immunohistochemistry (IHC), flow cytometry (e.g., fluorescence-activated cell sorting (FACS™)), Western blot, enzyme-linked immunosorbent assay (ELISA), immunoprecipitation, immunofluorescence, radioimmunoassay, dot blotting, immunodetection methods, HPLC, surface plasmon resonance, optical spectroscopy, mass spectrometry, and HPLC.

In some embodiments, the protein expression level of the biomarker (e.g., PD-L1) is determined in tumor-infiltrating immune cells. In some embodiments, the protein expression level of the biomarker is determined in tumor cells. In some embodiments, the protein expression level of the biomarker is determined in tumor-infiltrating immune cells and/or in tumor cells. In some embodiments, the protein expression level of the biomarker is determined in peripheral blood mononuclear cells (PBMCs).

In certain embodiments, the presence and/or expression level/amount of a biomarker protein (e.g., PD-L1) in a sample is examined using IHC and staining protocols. IHC staining of tissue sections has been shown to be a reliable method of determining or detecting the presence of proteins in a sample. In some embodiments of any of the methods, assays and/or kits, the biomarker is one or more of the protein expression products of PD-L1. In one embodiment, an expression level of biomarker is determined using a method comprising: (a) performing IHC analysis of a sample (such as a tumor sample obtained from a subject) with an antibody; and (b) determining expression level of a biomarker in the sample. In some embodiments, IHC staining intensity is determined relative to a reference. In some embodiments, the reference is a reference value. In some embodiments, the reference is a reference sample (e.g., a control cell line staining sample, a tissue sample from non-cancerous subject, or a tumor sample that is determined to be negative for the biomarker of interest).

For example, in some embodiments, the protein expression level of PD-L1 is determined using IHC. In some embodiments, the protein expression level of PD-L1 is detected using an anti-PD-L1 antibody. Any suitable anti-PD-L1 antibody may be used, including, e.g., SP142, SP263, 22C3, 28-8, El L3N, 4059, h5H1, and 9A11. In some embodiments, the anti-PD-L1 antibody is SP142. In some embodiments, the anti-PD-L1 antibody is SP263.

IHC may be performed in combination with additional techniques such as morphological staining and/or in situ hybridization (e.g., ISH). Two general methods of IHC are available; direct and indirect assays. According to the first assay, binding of antibody to the target antigen is determined directly. This direct assay uses a labeled reagent, such as a fluorescent tag or an enzyme-labeled primary antibody, which can be visualized without further antibody interaction. In a typical indirect assay, unconjugated primary antibody binds to the antigen and then a labeled secondary antibody binds to the primary antibody. Where the secondary antibody is conjugated to an enzymatic label, a chromogenic or fluorogenic substrate is added to provide visualization of the antigen. Signal amplification occurs because several secondary antibodies may react with different epitopes on the primary antibody.

The primary and/or secondary antibody used for IHC typically will be labeled with a detectable moiety. Numerous labels are available which can be generally grouped into the following categories: (a) radioisotopes, such as ³⁵S, ¹⁴C, ¹²⁵I, ³H, and ¹³¹I; (b) colloidal gold particles; (c) fluorescent labels including, but are not limited to, rare earth chelates (europium chelates), Texas Red, rhodamine, fluorescein, dansyl, lissamine, umbelliferone, phycocrytherin, phycocyanin, or commercially-available fluorophores such as SPECTRUM ORANGE7 and SPECTRUM GREEN7 and/or derivatives of any one or more of the above; (d) various enzyme-substrate labels are available and U.S. Pat. No. 4,275,149 provides a review of some of these. Examples of enzymatic labels include luciferases (e.g., firefly luciferase and bacterial luciferase; see, e.g., U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like.

Examples of enzyme-substrate combinations include, for example, horseradish peroxidase (HRPO) with hydrogen peroxidase as a substrate; alkaline phosphatase (AP) with para-Nitrophenyl phosphate as chromogenic substrate; and β-D-galactosidase (β-D-Gal) with a chromogenic substrate (e.g., p-nitrophenyl-β-D-galactosidase) or fluorogenic substrate (e.g., 4-methylumbelliferyl-β-D-galactosidase). For a general review of these, see, for example, U.S. Pat. Nos. 4,275,149 and 4,318,980.

Specimens may be prepared, for example, manually, or using an automated staining instrument (e.g., a Ventana BenchMark XT or Benchmark ULTRA instrument). Specimens thus prepared may be mounted and coverslipped. Slide evaluation is then determined, for example, using a microscope, and staining intensity criteria, routinely used in the art, may be employed. In one embodiment, it is to be understood that when cells and/or tissue from a tumor is examined using IHC, staining can be determined or assessed in tumor cell(s) and/or tissue (as opposed to stromal or surrounding tissue that may be present in the sample). In other embodiments, staining can be determined or assessed in stromal or surrounding tissue that may be present in the sample. In some embodiments, it is understood that when cells and/or tissue from a tumor is examined using IHC, staining includes determining or assessing in tumor-infiltrating immune cells, including intratumoral or peritumoral immune cells. In some embodiments, the presence of a biomarker is detected by IHC in >0% of the sample, in at least 1% of the sample, in at least 5% of the sample, in at least 10% of the sample, in at least 15% of the sample, in at least 15% of the sample, in at least 20% of the sample, in at least 25% of the sample, in at least 30% of the sample, in at least 35% of the sample, in at least 40% of the sample, in at least 45% of the sample, in at least 50% of the sample, in at least 55% of the sample, in at least 60% of the sample, in at least 65% of the sample, in at least 70% of the sample, in at least 75% of the sample, in at least 80% of the sample, in at least 85% of the sample, in at least 90% of the sample, in at least 95% of the sample, or more. Samples may be scored using any method known in the art, for example, by a pathologist or automated image analysis.

In some embodiments of any of the methods, the biomarker is detected by immunohistochemistry using a diagnostic antibody (i.e., primary antibody). In some embodiments, the diagnostic antibody specifically binds human antigen. In some embodiments, the diagnostic antibody is a non-human antibody. In some embodiments, the diagnostic antibody is a rat, mouse, or rabbit antibody. In some embodiments, the diagnostic antibody is a rabbit antibody. In some embodiments, the diagnostic antibody is a monoclonal antibody. In some embodiments, the diagnostic antibody is directly labeled. In other embodiments, the diagnostic antibody is indirectly labeled (e.g., by a secondary antibody).

In other embodiments of any of the preceding methods, the expression level of a biomarker may be a nucleic acid expression level (e.g., a DNA expression level or an RNA expression level (e.g., an mRNA expression level)). Any suitable method of determining a nucleic acid expression level may be used. In some embodiments, the nucleic acid expression level is determined using RNAseq, RT-qPCR, qPCR, multiplex qPCR or RT-qPCR, microarray analysis, SAGE, MassARRAY technique, ISH, or a combination thereof.

Methods for the evaluation of mRNAs in cells are well known and include, for example, serial analysis of gene expression (SAGE), whole genome sequencing (WGS), hybridization assays using complementary DNA probes (such as in situ hybridization using labeled riboprobes specific for the one or more genes, Northern blot and related techniques) and various nucleic acid amplification assays (such as RT-PCR (e.g., qRT-PCR) using complementary primers specific for one or more of the genes, and other amplification type detection methods, such as, for example, branched DNA, SISBA, TMA and the like). In addition, such methods can include one or more steps that allow one to determine the levels of target mRNA in a biological sample (e.g., by simultaneously examining the levels a comparative control mRNA sequence of a “housekeeping” gene such as an actin family member). Optionally, the sequence of the amplified target cDNA can be determined. Optional methods include protocols which examine or detect mRNAs, such as target mRNAs, in a tissue or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes whose expression correlates with increased or reduced clinical benefit of treatment comprising an immunotherapy and a suppressive stromal antagonist may be arrayed on a solid support. Hybridization of a labeled probe with a particular array member indicates that the sample from which the probe was derived expresses that gene.

The sample may be obtained from the subject at any suitable time. For example, in some embodiments, the sample is obtained from the subject prior to (e.g., minutes, hours, days, weeks (e.g., 1, 2, 3, 4, 5, 6, or 7 weeks), months, or years prior to) administration of the treatment regimen. In some embodiments of any of the preceding methods, the sample from the subject is obtained about 2 to about 10 weeks (e.g., 2, 3, 4, 5, 6, 7, 8, 9, or 10 weeks) following administration of the treatment regimen. In some embodiments, the sample from the subject is obtained about 4 to about 6 weeks following administration of the treatment regimen.

In some embodiments, the expression level or number of a biomarker (e.g., PD-L1) is detected in a tissue sample, a primary or cultured cells or cell line, a cell supernatant, a cell lysate, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, or any combination thereof. In some embodiments, the sample is a tissue sample (e.g., a tumor tissue sample), a cell sample, a whole blood sample, a plasma sample, a serum sample, or a combination thereof. In some embodiments, the tumor tissue sample wherein the tumor tissue sample includes tumor cells, tumor-infiltrating immune cells, stromal cells, or a combination thereof. In some embodiments, the tumor tissue sample is a formalin-fixed and paraffin-embedded (FFPE) sample, an archival sample, a fresh sample, or a frozen sample.

For example, in some embodiments, the expression level of a biomarker (e.g., PD-L1) is detected in tumor-infiltrating immune cells, tumor cells, PBMCs, or combinations thereof using known techniques (e.g., IHC, immunofluorescence microscopy, or flow cytometry). Tumor-infiltrating immune cells include, but are not limited to, intratumoral immune cells, peritumoral immune cells or any combinations thereof, and other tumor stroma cells (e.g., fibroblasts). Such tumor infiltrating immune cells may be T lymphocytes (such as CD8+T lymphocytes (e.g., CD8+T effector (Teff) cells) and/or CD4+T lymphocytes (e.g., CD4⁺ Teff cells), B lymphocytes, or other bone marrow-lineage cells including granulocytes (neutrophils, eosinophils, basophils), monocytes, macrophages, dendritic cells (e.g., interdigitating dendritic cells), histiocytes, and natural killer (NK) cells. In some embodiments, the staining for a biomarker is detected as membrane staining, cytoplasmic staining, or combinations thereof. In other embodiments, the absence of a biomarker is detected as absent or no staining in the sample, relative to a reference sample.

In particular embodiments, the expression level of a biomarker is assessed in a sample that contains or is suspected to contain cancer cells. The sample may be, for example, a tissue biopsy or a metastatic lesion obtained from a subject suffering from, suspected to suffer from, or diagnosed with cancer (e.g., NSCLC (e.g., squamous or non-squamous NSCLC, including stage IV NSCLC). In some embodiments, the sample is a sample of tissue, a biopsy of a tumor, a known or suspected metastatic NSCLC (e.g., squamous or non-squamous NSCLC) lesion or section, or a blood sample, e.g., a peripheral blood sample, known or suspected to comprise circulating cancer cells, e.g., NSCLC cells (e.g., squamous or non-squamous NSCLC cells). The sample may comprise both cancer cells, i.e., tumor cells, and non-cancerous cells (e.g., lymphocytes, such as T cells or NK cells), and, in certain embodiments, comprises both cancerous and non-cancerous cells. Methods of obtaining biological samples including tissue resections, biopsies, and body fluids, e.g., blood samples comprising cancer/tumor cells, are well known in the art.

In certain embodiments, the subject may have an advanced, refractory, recurrent, and/or chemotherapy-resistant form of the cancer.

In certain embodiments, the presence and/or expression levels/amount of a biomarker in a first sample is increased or elevated as compared to presence/absence and/or expression levels/amount in a second sample. In certain embodiments, the presence/absence and/or expression levels/amount of a biomarker in a first sample is decreased or reduced as compared to presence and/or expression levels/amount in a second sample. In certain embodiments, the second sample is a reference sample, reference cell, reference tissue, control sample, control cell, or control tissue.

In certain embodiments, a reference sample, reference cell, reference tissue, control sample, control cell, or control tissue is a single sample or combined multiple samples from the same subject that are obtained at one or more different time points than when the test sample is obtained. For example, a reference sample, reference cell, reference tissue, control sample, control cell, or control tissue is obtained at an earlier time point from the same subject than when the test sample is obtained. Such reference sample, reference cell, reference tissue, control sample, control cell, or control tissue may be useful if the reference sample is obtained during initial diagnosis of cancer and the test sample is later obtained when the cancer becomes metastatic.

In certain embodiments, a reference sample, reference cell, reference tissue, control sample, control cell, or control tissue is a combined multiple samples from one or more healthy individuals who are not the subject. In certain embodiments, a reference sample, reference cell, reference tissue, control sample, control cell, or control tissue is a combined multiple samples from one or more individuals with a disease or disorder (e.g., cancer) who are not the subject. In certain embodiments, a reference sample, reference cell, reference tissue, control sample, control cell, or control tissue is pooled RNA samples from normal tissues or pooled plasma or serum samples from one or more individuals who are not the subject. In certain embodiments, a reference sample, reference cell, reference tissue, control sample, control cell, or control tissue is pooled RNA samples from tumor tissues or pooled plasma or serum samples from one or more individuals with a disease or disorder (e.g., cancer) who are not the subject.

In some embodiments, the method further includes administering an effective amount of a treatment regimen described herein (e.g., a treatment regimen comprising a PD-1 axis binding antagonist (e.g., an anti-PD-L1 antibody (e.g., atezolizumab) or an anti-PD-1 antibody) and/or a platinum-based chemotherapy (e.g., cisplatin or carboplatin and gemcitabine) to the subject, for example, based on the expression level of one or more biomarkers (e.g., PD-L1).

### VII. PD-1 Axis Binding Antagonists

Provided herein are methods for treating or delaying progression of NSCLC (e.g., squamous or non-squamous NSCLC, including stage IV NSCLC) in a subject comprising administering to the subject a treatment regimen comprising an effective amount of a PD-1 axis binding antagonist (e.g., an anti-PD-L1 antibody (e.g., atezolizumab) or an anti-PD-1 antibody). Also provided herein are methods for treating or delaying progression of NSCLC (e.g., squamous or non-squamous NSCLC, including stage IV NSCLC) in a subject comprising administering to the subject a treatment regimen comprising an effective amount of a PD-1 axis binding antagonist (e.g., an anti-PD-L1 antibody (e.g., atezolizumab) or an anti-PD-1 antibody) and a platinum-based chemotherapy (e.g., cisplatin or carboplatin and gemcitabine). Also provided herein are methods of enhancing immune function in a subject having NSCLC (e.g., squamous or non-squamous NSCLC, including stage IV NSCLC) comprising administering to the subject a treatment regimen comprising an effective amount of a PD-1 axis binding antagonist (e.g., an anti-PD-L1 antibody (e.g., atezolizumab) or an anti-PD-1 antibody). Further provided herein are methods of enhancing immune function in a subject having NSCLC (e.g., squamous or non-squamous NSCLC, including stage IV NSCLC) comprising administering to the subject a treatment regimen comprising an effective amount of a PD-1 axis binding antagonist (e.g., an anti-PD-L1 antibody (e.g., atezolizumab) or an anti-PD-1 antibody) and a platinum-based chemotherapy (e.g., cisplatin or carboplatin and gemcitabine). Also provided are related compositions (e.g., pharmaceutical compositions) for use, kits, and articles of manufacture. Any of the methods, compositions for use, kits, or articles of manufacture described herein may include or involve any of the PD-1 axis binding antagonists described below.

For example, a PD-1 axis binding antagonist includes a PD-L1 binding antagonist, a PD-1 binding antagonist, and a PD-L2 binding antagonist. PD-L1 (programmed death ligand 1) is also referred to in the art as “programmed cell death 1 ligand 1,” “PDCD1LG1,” “CD274,” “B7-H,” and “PDL1.” An exemplary human PD-L1 is shown in UniProtKB/Swiss-Prot Accession No. Q9NZQ7.1. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. 015116. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some embodiments, PD-L1, PD-1, and PD-L2 are human PD-L1, PD-1, and PD-L2.

In some embodiments, the PD-1 axis binding antagonist is an anti-PD-L1 antibody. In some embodiments, the anti-PD-L1 antibody is atezolizumab, YW243.55.570, MDX-1105, MED14736 (durvalumab), or MSB0010718C (avelumab). Antibody YW243.55.570 is an anti-PD-L1 antibody described in WO 2010/077634. MDX-1105, also known as BMS-936559, is an anti-PD-L1 antibody described in WO2007/005874. MED14736 is an anti-PD-L1 monoclonal antibody described in WO2011/066389 and US2013/034559. In some embodiments, the anti-PD-L1 antibody is capable of inhibiting binding between PD-L1 and PD-1 and/or between PD-L1 and B7-1. In some embodiments, the anti-PD-L1 antibody is a monoclonal antibody. In some embodiments, the anti-PD-L1 antibody is an antibody fragment selected from the group consisting of Fab, Fab′-SH, Fv, scFv, and (Fab′)₂ fragments. In some embodiments, the anti-PD-L1 antibody is a humanized antibody. In some embodiments, the anti-PD-L1 antibody is a human antibody.

Examples of anti-PD-L1 antibodies useful for the methods of this disclosure, and methods for making thereof are described in PCT Patent Application Nos. WO 2010/077634, WO 2007/005874, WO 2011/066389, and in US 2013/034559, which are incorporated herein by reference. The anti-PD-L1 antibodies useful in this disclosure, including compositions containing such antibodies, may be used as a monotherapy or in combination with one or more additional therapeutic agents, e.g., a platinum-based chemotherapy.

In some embodiments, the PD-1 binding antagonist is a molecule that inhibits the binding of PD-1 to its ligand binding partners. In a specific aspect, the PD-1 ligand binding partners are PD-L1 and/or PD-L2. In another embodiment, a PD-L1 binding antagonist is a molecule that inhibits the binding of PD-L1 to its binding partners. In a specific aspect, PD-L1 binding partners are PD-1 and/or B7-1. In another embodiment, the PD-L2 binding antagonist is a molecule that inhibits the binding of PD-L2 to its binding partners. In a specific aspect, a PD-L2 binding partner is PD-1. The antagonist may be an antibody, an antigen-binding fragment thereof, an immunoadhesin, a fusion protein, or oligopeptide.

Any suitable anti-PD-L1 antibody may be used in the methods and compositions provided herein. Anti-PD-L1 antibodies described in WO 2010/077634 A1 and U.S. Pat. No. 8,217,149 may be used in the methods and compositions provided herein. In some instances, the anti-PD-L1 antibody comprises a heavy chain variable region sequence of SEQ ID NO: 3 and/or a light chain variable region sequence of SEQ ID NO: 4. In a still further instance, provided is an isolated anti-PD-L1 antibody comprising a heavy chain variable region and/or a light chain variable region sequence, wherein:
(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence:

(SEQ ID NO: 3)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS,

and
(b) the light chain sequence has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 4)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR.

In one instance, the anti-PD-L1 antibody comprises a heavy chain variable region comprising an HVR-H1, HVR-H2 and HVR-H3 sequence, wherein:

(a) the HVR-H1 sequence is
(SEQ ID NO: 5)
GFTFSX₁SWIH;
(b) the HVR-H2 sequence is
(SEQ ID NO: 6)
AWIX₂PYGGSX₃YYADSVKG;
(c) the HVR-H3 sequence is
(SEQ ID NO: 7)
RHWPGGFDY;

further wherein: X, is D or G; X2 is S or L; X3 is T or S. In one specific aspect, X, is D; X2 is S and X3 is T. In another aspect, the polypeptide further comprises variable region heavy chain framework sequences juxtaposed between the HVRs according to the formula: (FR-H1)-(HVR-H1)-(FR-H2)-(HVR-H2)-(FR-H3)-(HVR-H3)-(FR-H4). In yet another aspect, the framework sequences are derived from human consensus framework sequences. In a further aspect, the framework sequences are VH subgroup III consensus framework. In a still further aspect, at least one of the framework sequences is the following:

FR-H1 is
(SEQ ID NO: 8)
EVQLVESGGGLVQPGGSLRLSCAAS
FR-H2 is
(SEQ ID NO: 9)
WVRQAPGKGLEWV
FR-H3 is
(SEQ ID NO: 10)
RFTISADTSKNTAYLQMNSLRAEDTAVYYCAR
FR-H4 is
(SEQ ID NO: 11)
WGQGTLVTVSS.

In a still further aspect, the heavy chain polypeptide is further combined with a variable region light chain comprising an HVR-L1, HVR-L2 and HVR-L3, wherein:

(a) the HVR-L1 sequence is
(SEQ ID NO: 12)
RASQX₄X₅X₆TX₇X₈A;
(b) the HVR-L2 sequence is
(SEQ ID NO: 13)
SASX₉LX₁₀S,;
(c) the HVR-L3 sequence is
(SEQ ID NO: 14)
QQX₁₁X₁₂X₁₃X₁₄PX₁₅T;

wherein: X₄ is D or V; X₅ is V or I; X₆ is S or N; X₇ is A or F; X₈ is V or L; X₉ is F or T; X₁₀ is Y or A; X₁₁, is Y, G, F, or S; X₁₂ is L, Y, For W; X₁₃ is Y, N, A, T, G, F or I; X₁₄ is H, V, P, T or I; X₁₅ is A, W, R, P or T. In a still further aspect, X₄ is D; X₅ is V; X₆ is S; X₇ is A; X₈ is V; X₉ is F; X₁₀ is Y; X₁₁ is Y; X₁₂ is L; X₁₃ is Y; X₁₄ is H; X₁₆ is A.

In a still further aspect, the light chain further comprises variable region light chain framework sequences juxtaposed between the HVRs according to the formula: (FR-L1)-(HVR-L1)-(FR-L2)-(HVR-L2)-(FR-L3)-(HVR-L3)-(FR-L4). In a still further aspect, the framework sequences are derived from human consensus framework sequences. In a still further aspect, the framework sequences are VL kappa I consensus framework. In a still further aspect, at least one of the framework sequence is the following:

FR-L1 is
(SEQ ID NO: 15)
DIQMTQSPSSLSASVGDRVTITC
FR-L2 is
(SEQ ID NO: 16)
WYQQKPGKAPKLLIY
FR-L3 is
(SEQ ID NO: 17)
GVPSRFSGSGSGTDFTLTISSLQPEDFATYYC
FR-L4 is
(SEQ ID NO: 18)
FGQGTKVEIKR.

In another instance, provided is an isolated anti-PD-L1 antibody or antigen binding fragment comprising a heavy chain and a light chain variable region sequence, wherein:

(a) the heavy chain comprises an HVR-H1, HVR-H2 and HVR-H3, wherein further:

(i) the HVR-H1 sequence is
(SEQ ID NO: 5)
GFTFSX₁SWIH;
(ii) the HVR-H2 sequence is
(SEQ ID NO: 6)
AWIX₂PYGGSX₃YYADSVKG
(iii) the HVR-H3 sequence is
(SEQ ID NO: 7)
RHWPGGFDY,
and

(b) the light chain comprises an HVR-L1, HVR-L2 and HVR-L3, wherein further:

(i) the HVR-L1 sequence is
(SEQ ID NO: 12)
RASQX₄X₅X₆TX₇X₈A
(ii) the HVR-L2 sequence is
(SEQ ID NO: 13)
SASX₉LX₁₀S;
and
(iii) the HVR-L3 sequence is
(SEQ ID NO: 14)
QQX₁₁X₁₂X₁₃X₁₄PX₁₅T;

wherein: X₁ is D or G; X₂ is S or L; X₃ is T or S; X₄ is D or V; X₅ is V or I; X₆ is S or N; X₇ is A or F; X₈ is V or L; X₉ is F or T; X₁₀ is Y or A; X₁₁ is Y, G, F, or S; X₁₂ is L, Y, F or W; X₁₃ is Y, N, A, T, G, F or I; X₁₄ is H, V, P, T or I; X₁₅ is A, W, R, P or T. In a specific aspect, X₁ is D; X₂ is S and X₃ is T. In another aspect, X₄ is D; X₅ is V; X₆ is S; X₇ is A; X₈ is V; X₉ is F; X₁₀ is Y; X₁₁ is Y; X₁₂ is L; X₁₃ is Y; X₁₄ is H; X₁₅ is A. In yet another aspect, X₁ is D; X₂ is S and X₃ is T, X₄ is D; X₅ is V; X₆ is S; X₇ is A; X₈ is V; X₉ is F; X₁₀ is Y; X₁₁ is Y; X₁₂ is L; X₁₃ is Y; X₁₄ is H and X₁₅ is A.

In a further aspect, the heavy chain variable region comprises one or more framework sequences juxtaposed between the HVRs as: (FR-H1)-(HVR-H1)-(FR-H2)-(HVR-H2)-(FR-H3)-(HVR-H3)-(FR-H4), and the light chain variable regions comprises one or more framework sequences juxtaposed between the HVRs as: (FR-L1)-(HVR-L1)-(FR-L2)-(HVR-L2)-(FR-L3)-(HVR-L3)-(FR-L4). In a still further aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences are set forth as SEQ ID NOs:8, 9, 10, and 11. In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences are set forth as SEQ ID NOs: 15, 16, 17, and 18.

In a still further specific aspect, the antibody further comprises a human or murine constant region. In a still further aspect, the human constant region is selected from the group consisting of IgG1, IgG2, IgG2, IgG3, and IgG4. In a still further specific aspect, the human constant region is IgG1. In a still further aspect, the murine constant region is selected from the group consisting of IgG1, IgG2A, IgG2B, and IgG3. In a still further aspect, the murine constant region in IgG2A. In a still further specific aspect, the antibody has reduced or minimal effector function. In a still further specific aspect, the minimal effector function results from an “effector-less Fc mutation” or aglycosylation. In still a further instance, the effector-less Fc mutation is an N297A or D265A/N297A substitution in the constant region.

In yet another instance, provided is an anti-PD-L1 antibody comprising a heavy chain and a light chain variable region sequence, wherein:
(a) the heavy chain further comprises an HVR-H1, HVR-H2 and an HVR-H3 sequence having at least 85% sequence identity to GFTFSDSWIH (SEQ ID NO: 19), AWISPYGGSTYYADSVKG (SEQ ID NO: 20) and RHWPGGFDY (SEQ ID NO: 21), respectively, or
(b) the light chain further comprises an HVR-L1, HVR-L2 and an HVR-L3 sequence having at least 85% sequence identity to RASQDVSTAVA (SEQ ID NO: 22), SASFLYS (SEQ ID NO: 23) and QQYLYHPAT (SEQ ID NO: 24), respectively.

In a specific aspect, the sequence identity is 86%, 87%, 88%, 89%, 90%, 91%, 92%, 93%, 94%, 95%, 96%, 97%, 98%, 99% or 100%.

In another aspect, the heavy chain variable region comprises one or more framework sequences juxtaposed between the HVRs as: (FR-H1)-(HVR-H1)-(FR-H2)-(HVR-H2)-(FR-H3)-(HVR-H3)-(FR-H4), and the light chain variable regions comprises one or more framework sequences juxtaposed between the HVRs as: (FR-L1)-(HVR-L1)-(FR-L2)-(HVR-L2)-(FR-L3)-(HVR-L3)-(FR-L4). In yet another aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences are set forth as SEQ ID NOs: 8, 9, 10, and 11. In a still further aspect, the light chain framework sequences are derived from a Kabat kappa I, II, II, or IV subgroup sequence. In a still further aspect, the light chain framework sequences are VL kappa I consensus framework. In a still further aspect, one or more of the light chain framework sequences are set forth as SEQ ID NOs: 15, 16, 17, and 18.

In a further aspect, the heavy chain variable region comprises one or more framework sequences juxtaposed between the HVRs as: (FR-H1)-(HVR-H1)-(FR-H2)-(HVR-H2)-(FR-H3)-(HVR-H3)-(FR-H4), and the light chain variable regions comprises one or more framework sequences juxtaposed between the HVRs as: (FR-L1)-(HVR-L1)-(FR-L2)-(HVR-L2)-(FR-L3)-(HVR-L3)-(FR-L4). In a still further aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences is the following:

FR-H1
(SEQ ID NO: 27)
EVQLVESGGGLVQPGGSLRLSCAASGFTFS
FR-H2
(SEQ ID NO: 28)
WVRQAPGKGLEWVA
FR-H3
(SEQ ID NO: 10)
RFTISADTSKNTAYLQMNSLRAEDTAVYYCAR
FR-H4
(SEQ ID NO: 11)
WGQGTLVTVSS.

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences is the following:

FR-L1
(SEQ ID NO: 15)
DIQMTQSPSSLSASVGDRVTITC
FR-L2
(SEQ ID NO: 16)
WYQQKPGKAPKLLIY
FR-L3
(SEQ ID NO: 17)
GVPSRFSGSGSGTDFTLTISSLQPEDFATYYC
FR-L4
(SEQ ID NO: 26)
FGQGTKVEIK.

In a still further specific aspect, the antibody further comprises a human or murine constant region. In a still further aspect, the human constant region is selected from the group consisting of IgG1, IgG2, IgG2, IgG3, and IgG4. In a still further specific aspect, the human constant region is IgG1. In a still further aspect, the murine constant region is selected from the group consisting of IgG1, IgG2A, IgG2B, and IgG3. In a still further aspect, the murine constant region in IgG2A. In a still further specific aspect, the antibody has reduced or minimal effector function. In a still further specific aspect the minimal effector function results from an “effector-less Fc mutation” or aglycosylation. In still a further instance, the effector-less Fc mutation is an N297A or D265A/N297A substitution in the constant region.

In yet another instance, provided is an anti-PD-L1 antibody comprising a heavy chain and a light chain variable region sequence, wherein:
(a) the heavy chain further comprises an HVR-H1, HVR-H2 and an HVR-H3 sequence having at least 85% sequence identity to GFTFSDSWIH (SEQ ID NO: 19), AWISPYGGSTYYADSVKG (SEQ ID NO: 20) and RHWPGGFDY (SEQ ID NO: 21), respectively, and/or
(b) the light chain further comprises an HVR-L1, HVR-L2 and an HVR-L3 sequence having at least 85% sequence identity to RASQDVSTAVA (SEQ ID NO: 22), SASFLYS (SEQ ID NO: 23) and QQYLYHPAT (SEQ ID NO: 24), respectively.

In a specific aspect, the sequence identity is 86%, 87%, 88%, 89%, 90%, 91%, 92%, 93%, 94%, 95%, 96%, 97%, 98%, 99% or 100%.

In another aspect, the heavy chain variable region comprises one or more framework sequences juxtaposed between the HVRs as: (FR-H1)-(HVR-H1)-(FR-H2)-(HVR-H2)-(FR-H3)-(HVR-H3)-(FR-H4), and the light chain variable regions comprises one or more framework sequences juxtaposed between the HVRs as: (FR-L1)-(HVR-L1)-(FR-L2)-(HVR-L2)-(FR-L3)-(HVR-L3)-(FR-L4). In yet another aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences are set forth as SEQ ID NOs: 8, 9, 10, and WGQGTLVTVSSASTK (SEQ ID NO: 29).

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences are set forth as SEQ ID NOs: 15, 16, 17, and 18. In a still further specific aspect, the antibody further comprises a human or murine constant region. In a still further aspect, the human constant region is selected from the group consisting of IgG1, IgG2, IgG2, IgG3, and IgG4. In a still further specific aspect, the human constant region is IgG1. In a still further aspect, the murine constant region is selected from the group consisting of IgG1, IgG2A, IgG2B, and IgG3. In a still further aspect, the murine constant region in IgG2A. In a still further specific aspect, the antibody has reduced or minimal effector function. In a still further specific aspect, the minimal effector function results from an “effector-less Fc mutation” or aglycosylation. In still a further instance, the effector-less Fc mutation is an N297A or D265A/N297A substitution in the constant region.

In a still further instance, provided is an isolated anti-PD-L1 antibody comprising a heavy chain and a light chain variable region sequence, wherein:
(a) the heavy chain sequence has at least 85% sequence identity to the heavy chain sequence:

(SEQ ID NO: 25)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSSASTK,

or
(b) the light chain sequences has at least 85% sequence identity to the light chain sequence:

(SEQ ID NO: 4)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR.

In some instances, provided is an isolated anti-PD-L1 antibody comprising a heavy chain and a light chain variable region sequence, wherein the light chain variable region sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence of SEQ ID NO: 4. In some instances, provided is an isolated anti-PD-L1 antibody comprising a heavy chain and a light chain variable region sequence, wherein the heavy chain variable region sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence of SEQ ID NO: 25. In some instances, provided is an isolated anti-PD-L1 antibody comprising a heavy chain and a light chain variable region sequence, wherein the light chain variable region sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 4 and the heavy chain variable region sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 25. In some instances, one, two, three, four, or five amino acid residues at the N-terminal of the heavy and/or light chain may be deleted, substituted or modified.

In a still further instance, provided is an isolated anti-PD-L1 antibody comprising a heavy chain and a light chain sequence, wherein:
(a) the heavy chain sequence has at least 85% sequence identity to the heavy chain sequence:

(SEQ ID NO: 30)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYAST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG,

and/or
(b) the light chain sequences has at least 85% sequence identity to the light chain sequence:

(SEQ ID NO: 31)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In some instances, provided is an isolated anti-PD-L1 antibody comprising a heavy chain and a light chain sequence, wherein the light chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 31. In some instances, provided is an isolated anti-PD-L1 antibody comprising a heavy chain and a light chain sequence, wherein the heavy chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 30. In some instances, provided is an isolated anti-PD-L1 antibody comprising a heavy chain and a light chain sequence, wherein the light chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 31 and the heavy chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 30. In some instances, provided is an isolated anti-PD-L1 antibody comprising a heavy chain comprising the amino acid sequence of SEQ ID NO:30 and a light chain sequence comprising the amino acid sequence of SEQ ID NO:31.

In some instances, the isolated anti-PD-L1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Removal of glycosylation sites form an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site another amino acid residue (e.g., glycine, alanine or a conservative substitution).

In any of the instances herein, the isolated anti-PD-L1 antibody can bind to a human PD-L1, for example a human PD-L1 as shown in UniProtKB/Swiss-Prot Accession No. Q9NZQ7.1, or a variant thereof.

In a still further instance, provided is an isolated nucleic acid encoding any of the antibodies described herein. In some instances, the nucleic acid further comprises a vector suitable for expression of the nucleic acid encoding any of the previously described anti-PD-L1 antibodies. In a still further specific aspect, the vector is in a host cell suitable for expression of the nucleic acid. In a still further specific aspect, the host cell is a eukaryotic cell or a prokaryotic cell. In a still further specific aspect, the eukaryotic cell is a mammalian cell, such as Chinese hamster ovary (CHO) cell.

The antibody or antigen binding fragment thereof, may be made using methods known in the art, for example, by a process comprising culturing a host cell containing nucleic acid encoding any of the previously described anti-PD-L1 antibodies or antigen-binding fragments in a form suitable for expression, under conditions suitable to produce such antibody or fragment, and recovering the antibody or fragment.

In some embodiments, the PD-1 axis binding antagonist is a PD-1 binding antagonist. In some embodiments, the PD-1 binding antagonist is an anti-PD-1 antibody (e.g., a human antibody, a humanized antibody, or a chimeric antibody). Any suitable anti-PD-1 antibody may be used in the context of the disclosure. In some embodiments, the anti-PD-1 antibody is selected from the group consisting of MDX-1106 (nivolumab), MK-3475 (pembrolizumab), MEDI-0680 (AMP-514), PDR001, REGN2810, and BGB-108. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 binding antagonist is AMP-224. In some embodiments, the PD-L1 binding antagonist is anti-PD-L1 antibody. MDX-1106, also known as MDX-1106-04, ONO-4538, BMS-936558, or nivolumab, is an anti-PD-1 antibody described in WO2006/121168. MK-3475, also known as lambrolizumab, is an anti-PD-1 antibody described in WO2009/114335. AMP-224, also known as B7-DCIg, is a PD-L2-Fc fusion soluble receptor described in WO2010/027827 and WO2011/066342. In some instances, the anti-PD-1 antibody is MDX-1106. Alternative names for “MDX-1106” include MDX-1106-04, ONO-4538, BMS-936558, and nivolumab. In some instances, the anti-PD-1 antibody is nivolumab (CAS Registry Number: 946414-94-4). In a still further instance, provided is an isolated anti-PD-1 antibody comprising a heavy chain variable region comprising the heavy chain variable region amino acid sequence from SEQ ID NO: 1 and/or a light chain variable region comprising the light chain variable region amino acid sequence from SEQ ID NO: 2. In a still further instance, provided is an isolated anti-PD-1 antibody comprising a heavy chain and/or a light chain sequence, wherein:
(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence:

(SEQ ID NO: 1)
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSASTKGPSVFPLAPCSRSTSESTAALGCLVKDYFPEPV
TVSWNSGALTSGVHTFPAVLQSSGLYSLSSVVTVPSSSLGTKTYTCNVDH
KPSNTKVDKRVESKYGPPCPPCPAPEFLGGPSVFLFPPKPKDTLMISRTP
EVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLY
SRLTVDKSRWQEGNVFSCSVMHEALHNHYTQKSLSLSLGK,

and
(b) the light chain sequences has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 2)
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In a still further embodiment, provided is an isolated nucleic acid encoding any of the antibodies described herein. In some embodiments, the nucleic acid further comprises a vector suitable for expression of the nucleic acid encoding any of the previously described anti-PD-1 antibodies. In a still further specific aspect, the vector is in a host cell suitable for expression of the nucleic acid. In a still further specific aspect, the host cell is a eukaryotic cell or a prokaryotic cell. In a still further specific aspect, the eukaryotic cell is a mammalian cell, such as Chinese hamster ovary (CHO) cell.

The antibody or antigen-binding fragment thereof, may be made using methods known in the art, for example, by a process comprising culturing a host cell containing nucleic acid encoding any of the previously described anti-PD-1 antibodies in a form suitable for expression, under conditions suitable to produce such antibody or fragment, and recovering the antibody or fragment, or according to any method described below.

It is expressly contemplated that such PD-1 axis binding antagonist antibodies (e.g., anti-PD-L1 antibodies, anti-PD-1 antibodies, and anti-PD-L2 antibodies), or other antibodies described herein for use in any of the instances enumerated above may have any of the features, singly or in combination, described in Sections 1-7 below.

1. Antibody Affinity

In certain instances, an antibody provided herein (e.g., an anti-PD-L1 antibody or an anti-PD-1 antibody) has a dissociation constant (Kd) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M).

In one instance, Kd is measured by a radiolabeled antigen binding assay (RIA). In one instance, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I] antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20TH; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another instance, Kd is measured using a BIACORE® surface plasmon resonance assay. For example, an assay using a BIACORE®-2000 or a BIACORE®-3000 (BIAcore, Inc., Piscataway, NJ) is performed at 25° C. with immobilized antigen CM5 chips at ˜10 response units (RU). In one instance, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (-0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, for example, Chen et al., J. Mol. Biol. 293:865-881 (1999). If the on-rate exceeds 10⁶ NA-1s-1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

2. Antibody Fragments

In certain instances, an antibody (e.g., an anti-PD-L1 antibody or an anti-PD-1 antibody) provided herein is an antibody fragment. Antibody fragments include, but are not limited to, Fab, Fab′, Fab′-SH, F(ab′)2, Fv, and scFv fragments, and other fragments described below. For a review of certain antibody fragments, see Hudson et al. Nat. Med. 9:129-134 (2003). Fora review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al. Nat. Med. 9:129-134 (2003); and Hollinger et al. Proc. Natl. Acad. Sci. USA 90: 6444-6448 (1993). Triabodies and tetrabodies are also described in Hudson et al. Nat. Med. 9:129-134 (2003).

Single-domain antibodies are antibody fragments comprising all or a portion of the heavy chain variable domain or all or a portion of the light chain variable domain of an antibody. In certain instances, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g., E. coli or phage), as described herein.

3. Chimeric and Humanized Antibodies

In certain instances, an antibody (e.g., an anti-PD-L1 antibody or an anti-PD-1 antibody) provided herein is a chimeric antibody. Certain chimeric antibodies are described, e.g., in U.S. Pat. No. 4,816,567; and Morrison et al. Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain instances, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some instances, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Natl. Acad. Sci. USA 86:10029-10033 (1989); US Patent Nos. 5, 821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. BioL Chem. 272:10678-10684 (1997) and Rosok et al., J. BioL Chem. 271:22611-22618 (1996)).

4. Human Antibodies

In certain instances, an antibody (e.g., an anti-PD-L1 antibody or an anti-PD-1 antibody) provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology. Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc, Nail. Acad, Sci, USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005). Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Antibodies (e.g., anti-PD-L1 antibodies and anti-PD-1 antibodies) may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, NJ, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N J, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

6. Multispecific Antibodies

In any one of the above aspects, an antibody (e.g., an anti-PD-L1 antibody or an anti-PD-1 antibody) provided herein may be a multispecific antibody, for example, a bispecific antibody. Multispecific antibodies are monoclonal antibodies that have binding specificities for at least two different sites. In certain instances, an antibody provided herein is a multispecific antibody, e.g., a bispecific antibody. In certain instances, one of the binding specificities is for PD-L1 and the other is for any other antigen. In certain instances, bispecific antibodies may bind to two different epitopes of PD-L1. Bispecific antibodies may also be used to localize cytotoxic agents to cells which express PD-L1. Bispecific antibodies can be prepared as full-length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (see, e.g., WO 2009/089004A1); cross-linking two or more antibodies or fragments (see, e.g., U.S. Pat. No. 4,676,980, and Brennan et al., Science 229: 81 (1985)); using leucine zippers to produce bi-specific antibodies (see, e.g., Kostelny et al., J. Immunol. 148(5): 1547-1553 (1992)); using “diabody” technology for making bispecific antibody fragments (see, e.g., Hollinger et al., Proc. Natl. Acad. Sci. USA 90:6444-6448 (1993)); using single-chain Fv (sFv) dimers (see, e.g., Gruber et al., J. Immunol. 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g., US 2006/0025576A1).

The antibody or fragment herein also includes a “Dual Acting FAb” or “DAF” comprising an antigen binding site that binds to PD-L1 as well as another, different antigen.

7. Antibody Variants

In certain instances, amino acid sequence variants of the antibodies provided herein (e.g., anti-PD-L1 antibodies and anti-PD-1 antibodies) are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

I. Substitution, Insertion, and Deletion Variants

In certain instances, antibody variants having one or more amino acid substitutions are provided. Sites of interest for substitutional mutagenesis include the HVRs and FRs. Conservative substitutions are shown in Table 2 under the heading of “preferred substitutions.” More substantial changes are provided in Table 2 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved Antibody-Dependent Cell-Mediated Cytotoxicity (ADCC) or Complement Dependant Cytotoxicity (CDC).

TABLE 2
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity and/or reduced immunogenicity) relative to the parent antibody and/or will have substantially retained certain biological properties of the parent antibody. An exemplary substitutional variant is an affinity matured antibody, which may be conveniently generated, for example, using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)). In some instances of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain instances, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen-contacting residues in the HVRs. In certain instances of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

II. Glycosylation variants

In certain instances, antibodies of the disclosure can be altered to increase or decrease the extent to which the antibody is glycosylated. Addition or deletion of glycosylation sites to an antibody of the disclosure may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some instances, modifications of the oligosaccharide in an antibody of the disclosure may be made in order to create antibody variants with certain improved properties.

In one instance, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, for example, U.S. Patent Publication Nos. US 2003/0157108 and US 2004/0093621. Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); U.S. Pat. Appl. No. US 2003/0157108 A1; and WO 2004/056312 Al, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibody variants are further provided with bisected oligosaccharides, for example, in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878; U.S. Pat. No. 6,602,684; and US 2005/0123546. Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

III. Fc Region Variants

In certain instances, one or more amino acid modifications may be introduced into the Fc region of an antibody of the disclosure, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain instances, the disclosure contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest are described in U.S. Pat. No. 5,500,362 (see, e.g., Hellstrom, I. et al. Proc. Natl. Acad. Sci. USA 83:7059-7063 (1986)) and Hellstrom, I et al., Proc. Natl. Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CYTOTOX 96® non-radioactive cytotoxicity assay (Promega, Madison, WI))). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Natl. Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, e.g., Gazzano-Santoro et al., J. Immunol. Methods 202:163 (1996); Cragg et al., Blood. 101:1045-1052 (2003); and Cragg et al., Blood. 103:2738-2743 (2004)). FcRn binding and in vivo clearance/half-life determinations can also be performed using methods known in the art (see, e.g., Petkova et al. Int'l. Immunol. 18(12):1759-1769 (2006)).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain instances, an antibody variant comprises an Fc region with one or more amino acid substitutions which improve ADCC, e.g., substitutions at positions 298, 333, and/or 334 of the Fc region (EU numbering of residues).

In some instances, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

IV. Cysteine engineered antibody variants

In certain instances, it may be desirable to create cysteine engineered antibodies, e.g., “thioMAbs,” in which one or more residues of an antibody are substituted with cysteine residues. In particular instances, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain instances, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

V. Antibody Derivatives

In certain instances, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another instance, conjugates of an antibody and nonproteinaceous moiety that may be selectively heated by exposure to radiation are provided. In one instance, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

VI. Immunoconjugates

The disclosure also provides immunoconjugates comprising an antibody provided herein (e.g., an anti-PD-L1 antibody or an anti-PD-1 antibody) conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one instance, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another instance, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another instance, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron. Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate) which are commercially available (e.g., from Pierce Biotechnology, Inc., Rockford, IL., U.S.A).

### VIII. Platinum-Based Chemotherapies

Provided herein are methods for treating or delaying progression of NSCLC (e.g., squamous or non-squamous NSCLC, including stage IV NSCLC) in a subject comprising administering to the subject a treatment regimen comprising an effective amount of a PD-1 axis binding antagonist (e.g., an anti-PD-L1 antibody (e.g., atezolizumab) or an anti-PD-1 antibody) and a platinum-based chemotherapy (e.g., cisplatin or carboplatin and gemcitabine). Further provided herein are methods of enhancing immune function in a subject having NSCLC (e.g., squamous or non-squamous NSCLC, including stage IV NSCLC) comprising administering to the subject a treatment regimen comprising an effective amount of a PD-1 axis binding antagonist (e.g., an anti-PD-L1 antibody (e.g., atezolizumab) or an anti-PD-1 antibody) and a platinum-based chemotherapy (e.g., cisplatin or carboplatin and gemcitabine). Also provided are related compositions (e.g., pharmaceutical compositions) for use, kits, and articles of manufacture. Any of the methods, compositions for use, kits, or articles of manufacture described herein may include or involve any of the platinum-based chemotherapies described below.

Any suitable platinum-based chemotherapy may be used. In some embodiments, the platinum-based chemotherapy includes a platinum-based chemotherapeutic agent (e.g., cisplatin, carboplatin, oxaliplatin, nedaplatin, triplatin tetranitrate, phenanthriplatin, picoplatin, lipoplatin, or satraplatin). In some embodiments, the platinum-based chemotherapy includes cisplatin. In some embodiments, the platinum-based chemotherapy includes carboplatin.

In some embodiments, the platinum-based chemotherapy further includes one or more additional chemotherapeutic agents. For example, a platinum-based chemotherapy may further include any of the chemotherapeutic agents described herein. In one example, the platinum-based chemotherapy further includes a nucleoside analog. Any suitable nucleoside analog may be used, e.g., gemcitabine, cytarabine, fludarabine, or cladribine. In some embodiments, the nucleoside analog is gemcitabine. For example, in some embodiments, the platinum-based chemotherapy includes cisplatin and gemcitabine. In other embodiments, the platinum-based chemotherapy includes carboplatin and gemcitabine.

In another example, the platinum-based chemotherapy may include cisplatin, methotrexate, vinblastine, and doxorubicin, which is also known in the art as MVAC.

In another example, the platinum-based chemotherapy may include dose-dense methotrexate, vinblastine, doxorubicin, and cisplatin, which is also known in the art as DDMVAC.

In another example, the platinum-based chemotherapy may include cisplatin and fluorouracil (5-FU).

In another example, the platinum-based chemotherapy may include cisplatin, methotrexate, and vinblastine, which is also known in the art as CMV.

In another example, the platinum-based chemotherapy may include methotrexate, carboplatin, and vincristine, which is also known in the art as M-CAVI.

In another example, the platinum-based chemotherapy may include cisplatin and a taxane (e.g., paclitaxel).

### IX. Pharmaceutical Compositions and Formulations

Also provided herein are pharmaceutical compositions and formulations comprising a PD-1 axis binding antagonist and/or an antibody described herein (such as an anti-PD-L1 antibody (e.g., atezolizumab) or an anti-PD-1 antibody) and, optionally, a pharmaceutically acceptable carrier. The disclosure also provides pharmaceutical compositions and formulations comprising one or more members of a platinum-based chemotherapy (e.g., cisplatin or carboplatin and gemcitabine), and optionally, a pharmaceutically acceptable carrier.

Pharmaceutical compositions and formulations as described herein can be prepared by mixing the active ingredients (e.g., a PD-1 axis binding antagonist (e.g., an anti-PD-L1 antibody (e.g., atezolizumab) or an anti-PD-1 antibody) and/or a platinum-based chemotherapy (e.g., cisplatin or carboplatin and gemcitabine) having the desired degree of purity with one or more optional pharmaceutically acceptable carriers (see, e.g., Remington's Pharmaceutical Sciences 16th edition, Osol, A. Ed. (1980)), e.g., in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The compositions and formulations herein may also contain more than one active ingredient as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g., films or microcapsules. The formulations to be used for in vivo administration are generally sterile. Sterility may be readily accomplished, e.g., by filtration through sterile filtration membranes.

### X. Articles of Manufacture or Kits

In another embodiment of the disclosure, an article of manufacture or a kit is provided comprising a PD-1 axis binding antagonist (e.g., an anti-PD-L1 antibody (e.g., atezolizumab) or an anti-PD-1 antibody) and/or a platinum-based chemotherapy (e.g., cisplatin or carboplatin and gemcitabine). In some embodiments, the article of manufacture or kit further comprises package insert comprising instructions for using the PD-1 axis binding antagonist to treat or delay progression of NSCLC (e.g., squamous or non-squamous NSCLC, including stage IV NSCLC) in a subject or to enhance immune function of a subject having NSCLC (e.g., squamous or non-squamous NSCLC, including stage IV NSCLC). In some embodiments, the article of manufacture or kit further comprises package insert comprising instructions for using the PD-1 axis binding antagonist in combination with a platinum-based chemotherapy (e.g., cisplatin or carboplatin and gemcitabine) to treat or delay progression of NSCLC (e.g., squamous or non-squamous NSCLC, including stage IV NSCLC) in a subject or to enhance immune function of a subject having NSCLC (e.g., squamous or non-squamous NSCLC, including stage IV NSCLC). Any of the PD-1 axis binding antagonists and/or platinum-based chemotherapies described herein may be included in the article of manufacture or kits.

In some embodiments, the PD-1 axis binding antagonist (e.g., an anti-PD-L1 antibody (e.g., atezolizumab) or an anti-PD-1 antibody) and the platinum-based chemotherapy (e.g., cisplatin or carboplatin and gemcitabine) are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., an additional chemotherapeutic agent or anti-neoplastic agent). Suitable containers for the one or more agent include, for example, bottles, vials, bags and syringes.

In another example, provided herein is a kit for treating NSCLC in a subject in need thereof, the kit comprising a PD-1 axis binding antagonist (e.g., an anti-PD-L1 antibody (e.g., atezolizumab) or an anti-PD-1 antibody) and/or a platinum-based chemotherapy (e.g., cisplatin or carboplatin and gemcitabine) and instructions for administering the PD-1 axis binding antagonist and/or the platinum-based chemotherapy to a subject who is previously untreated for the NSCLC in a treatment regimen comprising one or more dosing cycles, wherein the treatment regimen extends the subject's PFS and/or OS as compared to treatment with the platinum-based chemotherapy without the PD-1 axis binding antagonist. In some embodiments, the treatment regimen extends the subject's PFS as compared to treatment with the platinum-based chemotherapy without the PD-1 axis binding antagonist. In some embodiments, the treatment regimen extends the subject's OS as compared to treatment with the platinum-based chemotherapy without the PD-1 axis binding antagonist. In some embodiments, the treatment regimen increases the subject's likelihood of having an objective response (e.g., a CR) and/or extends the subject's duration of response (DOR) as compared to treatment with the platinum-based chemotherapy without the PD-1 axis binding antagonist. In some embodiments, each dosing cycle is about 21 days. In some embodiments, the platinum-based chemotherapy comprises a platinum-based chemotherapeutic agent and a nucleoside analog. In some embodiments, the platinum-based chemotherapeutic agent is cisplatin, carboplatin, or oxaliplatin. In some embodiments, the platinum-based chemotherapeutic agent is cisplatin. In some embodiments, cisplatin is administered to the subject intravenously at a dose of about 75 mg/m² on Day −2 to Day 4 of a 21-day dosing cycle. In some embodiments, cisplatin is administered to the subject intravenously at a dose of about 75 mg/m² on Day 1 of a 21-day dosing cycle. In some embodiments, the platinum-based chemotherapeutic agent is carboplatin. In some embodiments, carboplatin is administered to the subject intravenously at an area under the curve (AUC) of about 5 on Day −2 to Day 4 of a 21-day dosing cycle. In some embodiments, carboplatin is administered to the subject intravenously at an AUC of about 5 on Day 1 of a 21-day dosing cycle. In some embodiments, the nucleoside analog is gemcitabine. In some embodiments, gemcitabine is administered to the subject intravenously at a dose of about 1000 mg/m² on Day −2 to Day 4 and on Day 7 to Day 11 of a 21-day dosing cycle. In some embodiments, gemcitabine is administered to the subject intravenously at a dose of about 1000 mg/m² on Day 1 and Day 8 of a 21-day dosing cycle. In some embodiments, the platinum-based chemotherapy comprises cisplatin and gemcitabine. In other embodiments, the platinum-based chemotherapy comprises carboplatin and gemcitabine.

In any of the preceding kits, the anti-PD-L1 antibody may be atezolizumab. The kit may include instructions to administer atezolizumab to the subject at any suitable dosage. In some embodiments, the kit includes instructions to administer atezolizumab to the subject intravenously at a dose of about 840 mg every 2 weeks, about 1200 mg every 3 weeks, or about 1680 mg every 4 weeks. In some embodiments, the kit includes instructions to administer atezolizumab to the subject intravenously at a dose of about 1200 mg every 3 weeks. In some embodiments, the kit includes instructions to administer atezolizumab to the subject in a 21-day dosing cycle. In some embodiments, the kit includes instructions to administer atezolizumab to the subject intravenously at a dose of about 1200 mg on Day −2 to Day 4 of a 21-day dosing cycle. In some embodiments, the kit includes instructions to administer atezolizumab to the subject intravenously at a dose of about 1200 mg on Day 1 of a 21-day dosing cycle.

The specification is considered to be sufficient to enable one skilled in the art to practice the disclosure. Various modifications of the disclosure in addition to those shown and described herein will become apparent to those skilled in the art from the foregoing description and fall within the scope of the appended claims.

### EXAMPLES

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: A Phase Ill, Open-Label, Randomized Study of Atezolizumab (Anti-PD-L1 Antibody) Compared with a Platinum-based Chemotherapy in Patients with Untreated Non-Small Cell Lung Cancer

This Example describes the results of a Phase III clinical trial conducted with the aim of assessing the differential effects of atezolizumab treatment relative to treatment with platinum-based chemotherapy on patients having stage IV squamous or non-squamous non-small cell lung cancer (NSCLC). As described below, the primary endpoint of this study was improvement in overall survival (OS). Secondary endpoints included progression-free survival (PFS), overall response rate (ORR), and duration of response (DOR), among others.

The study was conducted in accordance with the protocol described below:

### Experimental Design

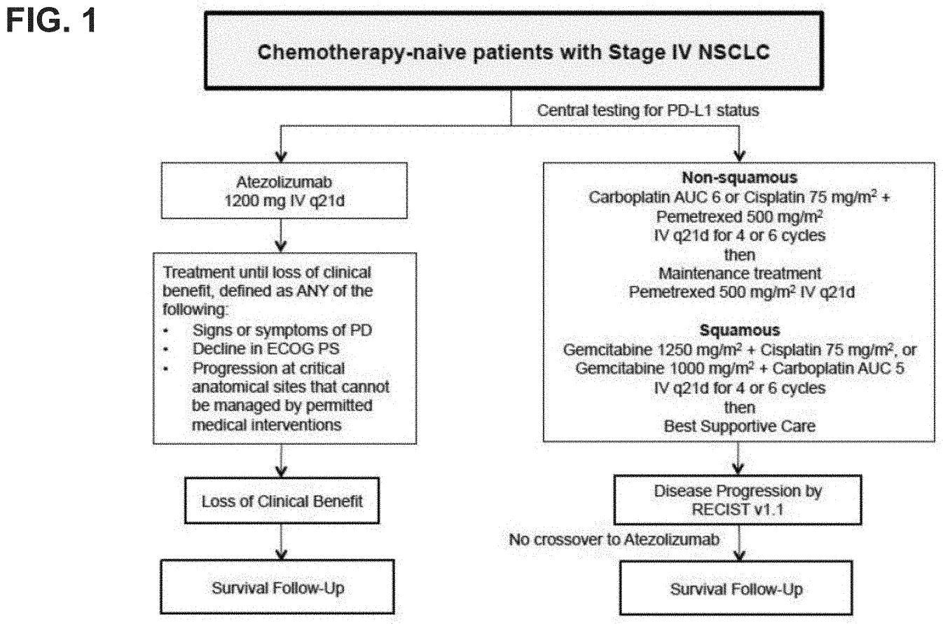

This study was a randomized, Phase III, global, multicenter, open-label study designed to evaluate the safety and efficacy of atezolizumab compared with chemotherapy consisting of a platinum agent (cisplatin or carboplatin per investigator discretion) combined with either pemetrexed (non-squamous disease) or gemcitabine (squamous disease) in PD-L1-selected, chemotherapy-naive patients with Stage IV NSCLC. FIG. 1 illustrates the study design.

At screening, tumor specimens from each potentially eligible patient were tested for PD-L1 expression by a central laboratory using an IHC assay. Only patients who are PD-L1 selected (TC1/2/3 or 101/2/3; corresponding to 1% PD-L1 expressing TCs and 1% of tumor area occupied by PD-L1 expressing ICs) were enrolled. Patients with non-squamous disease were randomized 1:1 to receive either atezolizumab alone or pemetrexed in combination with cisplatin or carboplatin. Patients with squamous disease were randomized 1:1 to receive either atezolizumab alone or gemcitabine in combination with cisplatin or carboplatin. Randomization was stratified by sex (male vs. female), ECOG Performance Status (0 vs. 1), histology (non-squamous vs. squamous), and PD-L1 tumor expression by IHC (TC1/2/3 and any IC vs. TC0 and 101/2/3).

Given the toxicities associated with platinum-based chemotherapies (e.g., neutropenia, anemia) and the requirement for pre-medications, this was an open-label study. No crossover was allowed from the control arm (platinum-based chemotherapy) to the experimental arm (atezolizumab).

Atezolizumab (fixed dose of 1200 mg) was administered intravenously on Day 1 of each 21-day cycle. Atezolizumab treatment continued as long as patients experienced clinical benefit as assessed by the investigator (i.e., in the absence of unacceptable toxicity or symptomatic deterioration attributed to disease progression as determined by the investigator after an integrated assessment of radiographic data, biopsy results [if available], and clinical status) or until unacceptable toxicity or death.

During treatment, patients who were treated with atezolizumab and who showed evidence of clinical benefit were permitted to continue atezolizumab treatment after RECIST v1.1 criteria for progressive disease are met if they met all of the following criteria:

Evidence of clinical benefit as assessed by the investigator
Absence of symptoms and signs (including worsening of laboratory values [e.g., new or worsening hypercalcemia]) indicating unequivocal progression of disease
No decline in ECOG Performance Status that can be attributed to disease progression
Absence of tumor progression at critical anatomical sites (e.g., leptomeningeal disease) that cannot be managed by protocol-allowed medical interventions
Patients must provide written consent to acknowledge deferring other treatment options in favor of continuing study treatment at the time of initial radiographic progression per RECIST v1.1.

All patients underwent a mandatory tumor biopsy sample collection, unless not clinically feasible as assessed and documented by investigators, at the first evidence of radiographic disease progression (within 40 days of radiographic progression or prior to the start of the next anti-cancer treatment, whichever is sooner). These data were used to explore if the radiographic findings are consistent with the presence of tumor or if the appearance of progression was caused by pseudoprogression. In addition, these data were analyzed for the association between changes in tumor tissue and clinical outcome and to understand further the potential mechanisms of resistance and progression to atezolizumab when compared to such mechanisms after treatment with chemotherapy. This exploratory biomarker evaluation was not used for any treatment-related decisions. Patients who were unable to undergo biopsy sample collection but otherwise meet criteria listed above may continue to receive atezolizumab.

Patients randomized to receive pemetrexed in combination with either cisplatin or carboplatin (non-squamous disease) received chemotherapy intravenously on Day 1 of each 21-day cycle for four or six cycles, followed by maintenance therapy with pemetrexed. Patients randomized to receive gemcitabine in combination with either cisplatin or carboplatin (squamous disease) received cisplatin or carboplatin intravenously on Day 1 and gemcitabine intravenously on Days 1 and 8 of each 21-day cycle for four or six cycles, followed by best supportive care. The intended number of cycles planned for the platinum-based induction chemotherapy (i.e., four or six cycles) was specified by the investigator prior to study randomization. Treatment will continue until disease progression, unacceptable toxicity, or death.

All patients underwent tumor assessment at baseline and every 6 weeks (±7 days) for 48 weeks following Cycle 1, Day 1 regardless of treatment delays. After the completion of the Week 48 tumor assessment, tumor assessment was required every 9 weeks (±7 days) regardless of treatment delays, until radiographic disease progression per RECIST v1.1 (or loss of clinical benefit for atezolizumab-treated patients who had continued treatment with atezolizumab after radiographic disease progression according to RECIST v1.1), withdrawal of consent, death, or study termination by the Sponsor, whichever occurs first. Patients who discontinued treatment for reasons other than radiographic disease progression per RECIST v1.1 (e.g., toxicity, symptomatic deterioration) continued scheduled tumor assessments until radiographic disease progression per RECIST v1.1 (or loss of clinical benefit for atezolizumab-treated patients who had continued treatment with atezolizumab after radiographic after disease progression according to RECIST v1.1), withdrawal of consent, death, or study termination by Sponsor, whichever occurred first. In the absence of radiographic disease progression per RECIST v1.1, tumor assessments continued regardless of whether patients start a new anti-cancer therapy.

The end of the study occurred when all of the following criteria were met:
The required number of deaths for the final analysis of OS has been observed
The last patient, last visit has occurred.

In addition, the Sponsor may decide to terminate the study at any time. If the Sponsor decides to terminate the study, patients who are still receiving study treatment or undergoing survival follow-up may be enrolled in an extension study or a non-interventional study.

### Outcome Measures

The primary efficacy outcome measure for this study is OS, defined as the time from randomization to death from any cause.

The secondary efficacy outcome measures for this study are as follows:
PFS, defined as the time from randomization to the first occurrence of disease progression, as determined by the investigator with use of RECIST v1.1, or death from any cause, whichever occurs first
Objective response (PR plus CR) as determined by the investigator according to RECIST v1.1
DOR, defined as the time from the first occurrence of a documented objective response to the time of disease progression, as determined by the investigator with use of RECIST v1.1, or death from any cause, whichever occurs first
OS at 1- and 2-year landmark timepoints
TTD and change from baseline (i.e., improvement or deterioration based on presenting symptomatology) in each of the patient-reported lung cancer symptoms (cough, dyspnea, or chest pain) with use of the SILO scale
TTD in patient-reported lung cancer symptoms, defined as time from randomization to deterioration (10-point change) in any of the following symptom subscales (cough, dyspnea [multi-item scale], and chest pain), whichever occurs first, as measured by the EORTC QLQ-LC13
OS and investigator-assessed PFS according to RECIST v1.1 in the PD-L1 (defined with SP263 IHC assay) and bTMB subpopulations. The bTMB assay (Foundation Medicine, Cambridge, MA) identifies single nucleotide variants at ≥0.5% across 394 genes and estimates tumor fraction by maximum somatic allele frequency, filters out germline events and counts non-driver somatic mutations to generate a bTMB score. The bTMB score cutoffs evaluated were 0, 6 and A bTMB score of 16 (16 mutations/1.1 Mb) equates to approximately 14.5 mutations/Mb.

The exploratory outcome measures for this study are as follows:
OS and investigator-assessed PFS according to RECIST v1.1 in the PD-L1 (defined with 22c3 assay) subpopulation
PFS at 6-month and at 1-year landmark timepoints
OS at 3-year landmark timepoint
OS and investigator-assessed PFS according to RECIST v1.1 in subgroups based on demographic and baseline characteristics
Status of immune cell infiltrate and other exploratory biomarkers in mandatory biopsy specimens collected at progression
Status of PD-L1-, immune-, and NSCLC-related and other exploratory biomarkers in archival and/or freshly obtained tumor tissues and blood (or blood derivatives) collected before, during, or after treatment with atezolizumab or at progression and association with disease status and/or response to atezolizumab
Utility scores of the EQ-5D-3L questionnaire
Change from baseline in PROs of health-related quality of life, lung cancer-related symptoms, and functioning as assessed by the EORTC QLQ-C30 and QLQ-LC13.

### Materials and Methods

Patients

Approximately 150 sites globally participated in the study, and approximately 555 PD-L1—selected chemotherapy-naive patients with Stage IV NSCLC were enrolled.

Inclusion Criteria

Patients must have met all of the following criteria to be eligible for study entry:
Signed Informed Consent Form
Age 18 years
ECOG Performance Status of 0 or 1
Histologically or cytologically confirmed, Stage IV non-squamous or squamous NSCLC (per the Union Internationale contre le Cancer/American Joint Committee on Cancer staging system, 7th edition; Detterbeck et al. 2009)
Patients with tumors of mixed histology must be classified as non-squamous or squamous based on the major histological component.
No prior treatment for Stage IV non-squamous or squamous NSCLC
Patients known to have a sensitizing mutation in the EGFR gene or an ALK fusion oncogene are excluded from the study.
Patients with non-squamous NSCLC who have an unknown EGFR or ALK status were required to be tested at prescreening/screening.
Patients with squamous NSCLC who have an unknown EGFR or ALK status will not be required to be tested at prescreening/screening. EGFR and/or ALK may be assessed locally or at a central lab. Additional tissue were required for central testing of EGFR and/or ALK.
Patients who have received prior neo-adjuvant, adjuvant chemotherapy, radiotherapy, or chemoradiotherapy with curative intent for non-metastatic disease must have experienced a treatment-free interval of at least 6 months from randomization since the last chemotherapy, radiotherapy, or chemoradiotherapy cycle.
Patients with a history of treated asymptomatic CNS metastases are eligible, provided they meet all of the following criteria:
   Only supratentorial and cerebellar metastases allowed (i.e., no metastases to midbrain, pons, medulla, or spinal cord)
   No ongoing requirement for corticosteroids as therapy for CNS disease
   No stereotactic radiation within 7 days or whole-brain radiation within 14 days prior to randomization
   No evidence of interim progression between the completion of CNS-directed therapy and the screening radiographic study
   Patients with new asymptomatic CNS metastases detected at the screening scan must receive radiation therapy and/or surgery for CNS metastases. Following treatment, these patients may then be eligible without the need for an additional brain scan prior to randomization, if all other criteria are met.
Tumor PD-L1 expression (TC1/2/3 or 101/2/3; corresponding to 1% PD-L1 expressing TCs and 1% of tumor area occupied by PD-L1 expressing ICs), as determined by an IHC assay performed by a central laboratory on previously obtained archival tumor tissue or tissue obtained from a biopsy at screening.
A representative formalin-fixed paraffin-embedded (FFPE) tumor specimen in paraffin block (preferred) or 15 or more unstained, freshly cut, serial sections (on slides) from an FFPE tumor specimen is required for participation in this study. This specimen must be accompanied by the associated pathology report. If fewer than 15 slides are available at baseline (but no fewer than 10), the patient may still be eligible, upon discussion with the Medical Monitor.
For freshly collected specimens, resections, core needle biopsies, excisional, incisional, punch, or forceps biopsies are acceptable. Fine-needle aspiration (defined as samples that do not preserve tissue architecture and yield cell suspension and/or cell smears), brushing, cell pellet from pleural effusion, and lavage samples are not acceptable.
Tumor tissue from bone metastases that have been decalcified is not acceptable.
For core needle biopsy specimens, preferably, at least three cores embedded in a single paraffin block, should be submitted for evaluation.
For patients whose initial archival tumor tissue sample is PD-L1 negative, a biopsy can be performed at screening to submit fresh tissue for the purposes of testing PD-L1 status. A positive test result in any tumor tissue sample will satisfy this eligibility criterion.
Measurable disease, as defined by RECIST v1.1
Previously irradiated lesions can only be considered measurable disease if disease progression has been unequivocally documented at that site since radiation and the previously irradiated lesion is not the only site of measurable disease
Adequate hematologic and end-organ function, defined by the following laboratory test results obtained within 14 days prior to randomization:
   ANC ≥1500 cells/μL without granulocyte colony-stimulating factor support
   Lymphocyte count ≥500 cells/μL
   Platelet count ≥100,000 cells/μL without transfusion
   Hemoglobin ≥9.0 g/dL
   Patients may be transfused to meet this criterion.
   INR or aPTT≤1.5×upper limit of normal (ULN)
   This applies only to patients who are not receiving therapeutic anticoagulation; patients receiving therapeutic anticoagulation must have an INR or aPTT within therapeutic limits for at least 1 week prior to randomization.
AST, ALT, and alkaline phosphatase ≤2.5×ULN with the following exceptions:
   Patients with documented liver metastases: AST and/or ALT ≤5×ULN
   Patients with documented liver or bone metastases: alkaline phosphatase ≤5×ULN
   Serum bilirubin ≤1.5×ULN
   Patients with known Gilbert disease who have serum bilirubin level 3×ULN may be enrolled. Calculated creatinine clearance (CrCl) ≥45 mL/min, or if using cisplatin, calculated CrCl ≥60 mL/min
For female patients of childbearing potential and male patients with partners of childbearing potential, agreement to use a highly effective form(s) of contraception during study treatment that results in a low failure rate of <1% per year when used consistently and correctly. Female patients should continue contraception use for 5 months after the last dose of atezolizumab and for 6 months after the last dose of cisplatin. Women must refrain from donating eggs during this same period. Male patients treated with chemotherapy (cisplatin or carboplatin plus pemetrexed or gemcitabine) should continue contraception use for 6 months after the last dose of chemotherapy. Men must refrain from donating sperm during this same period. Such methods include combined (estrogen and progestogen containing) hormonal contraception, progestogen-only hormonal contraception associated with inhibition of ovulation together with another additional barrier method always containing a spermicide, intrauterine device (IUD), intrauterine hormone-releasing system (IUS), bilateral tubal occlusion or vasectomized partner (on the understanding that this is the only one partner during the entire study duration), and sexual abstinence. Oral contraception should always be combined with an additional contraceptive method because of a potential interaction with the study drug. The same rules are valid for male patients involved in this study if they have a partner of childbearing potential. Male patients must always use a condom.
Women who are not postmenopausal 12 months of non-therapy-induced amenorrhea) or surgically sterile must have a negative serum pregnancy test result within 14 days prior to initiation of study drug.

Exclusion Criteria

Patients who meet any of the criteria in the following sections were excluded from study entry.

### Cancer-Specific Exclusions

Known sensitizing mutation in the EGFR gene or ALK fusion oncogene
Active or untreated CNS metastases as determined by CT or magnetic resonance imaging (MRI) evaluation during screening and prior radiographic assessments
Spinal cord compression not definitively treated with surgery and/or radiation, or previously diagnosed and treated spinal cord compression without evidence that disease has been clinically stable for 2 weeks prior to randomization
Leptomeningeal disease
Uncontrolled tumor-related pain

Patients requiring pain medication must be on a stable regimen at study entry. Symptomatic lesions amenable to palliative radiotherapy (e.g., bone metastases or metastases causing nerve impingement) should be treated prior to randomization. Patients should be recovered from the effects of radiation.

There is no required minimum recovery period. Asymptomatic metastatic lesions whose further growth would likely cause functional deficits or intractable pain (e.g., epidural metastasis that is not currently associated with spinal cord compression) should be considered for loco-regional therapy if appropriate prior to enrollment.
Uncontrolled pleural effusion, pericardial effusion, or ascites requiring recurrent drainage procedures (once monthly or more frequently).
Patients with indwelling catheters (e.g., PleurX®) are allowed.
Uncontrolled or symptomatic hypercalcemia (>1.5 mmol/L ionized calcium or calcium >12 mg/dL or corrected serum calcium >ULN)
Patients who are receiving denosumab prior to randomization must be willing and eligible to discontinue its use and replace it with a bisphosphonate instead while in the study.
Malignancies other than NSCLC within 5 years prior to randomization, with the exception of those with a negligible risk of metastasis or death (e.g., expected 5-year OS >90%) treated with expected curative outcome (such as adequately treated carcinoma in situ of the cervix, basal or squamous cell skin cancer, localized prostate cancer treated surgically with curative intent, ductal carcinoma in situ treated surgically with curative intent).

### General Medical Exclusions

Women who are pregnant, lactating, or intending to become pregnant during the study
History of severe allergic, anaphylactic, or other hypersensitivity reactions to chimeric or humanized antibodies or fusion proteins
Known hypersensitivity to biopharmaceuticals produced in Chinese hamster ovary cells or any component of the atezolizumab formulation
History of autoimmune disease, including, but not limited to, myasthenia gravis, myositis, autoimmune hepatitis, systemic lupus erythematosus, rheumatoid arthritis, inflammatory bowel disease, vascular thrombosis associated with antiphospholipid syndrome, Wegener's granulomatosis, Sjögren's syndrome, Guillain-Barré syndrome, multiple sclerosis, vasculitis, or glomerulonephritis.
Patients with a history of autoimmune-related hypothyroidism on thyroid replacement therapy are eligible for this study. Patients with controlled Type I diabetes mellitus on an insulin regimen are eligible for this study. Patients with eczema, psoriasis, lichen simplex chronicus, or vitiligo with dermatologic manifestations only (i.e., patients with psoriatic arthritis would be excluded) are permitted provided that they meet the following conditions:
   Rash must cover less than 10% of body surface area Disease is well controlled at baseline and only requiring low potency topical steroids
   No acute exacerbations of underlying condition within the last 12 months requiring treatment with either PUVA [psoralen plus ultraviolet A radiation], methotrexate, retinoids, biologic agents, oral calcineurin inhibitors, or high-potency or oral steroids.
History of idiopathic pulmonary fibrosis, organizing pneumonia (e.g., bronchiolitis obliterans), drug-induced pneumonitis, idiopathic pneumonitis, or evidence of active pneumonitis on screening chest CT scan
History of radiation pneumonitis in the radiation field (fibrosis) is permitted.
Positive HIV test All patients must be tested for HIV; patients who test positive for HIV were excluded.
Patients with active hepatitis B (chronic or acute; defined as having a positive hepatitis B surface antigen [HBsAg] test at screening) or hepatitis C Patients with past hepatitis B virus (HBV) infection or resolved HBV infection (defined as the presence of hepatitis B core antibody [HBc Ab] and absence of HBsAg) are eligible. HBV DNA test must be performed in these patients prior to randomization.
Patients positive for hepatitis C virus (HCV) antibody are eligible only if polymerase chain reaction is negative for HCV RNA.
Active tuberculosis
Severe infections within 4 weeks prior to randomization, including, but not limited to, hospitalization for complications of infection, bacteremia, or severe pneumonia
Significant cardiovascular disease, such as New York Heart Association cardiac disease (Class II or greater), myocardial infarction, or cerebrovascular accident within 3 months prior to randomization, unstable arrhythmias, or unstable angina
Patients with known coronary artery disease, congestive heart failure not meeting the above criteria, or left ventricular ejection fraction <50% must be on a stable medical regimen that is optimized in the opinion of the treating physician, in consultation with a cardiologist if appropriate.
Major surgical procedure other than for diagnosis within 28 days prior to randomization or anticipation of need for a major surgical procedure during the course of the study
Prior allogeneic bone marrow transplantation or solid organ transplantation
Any other diseases, metabolic dysfunction, physical examination finding, or clinical laboratory finding giving reasonable suspicion of a disease or condition that contraindicates the use of an investigational drug or that may affect the interpretation of the results or render the patient at high risk from treatment complications
Patients with illnesses or conditions that interfere with their capacity to understand, follow and/or comply with study procedures

Exclusion Criteria Related to Medications
Treatment with any approved anti-cancer therapy, including hormonal therapy, within 3 weeks prior to initiation of study treatment
Treatment with any other investigational agent with therapeutic intent within 28 days prior to randomization
Received therapeutic oral or IV antibiotics within 2 weeks prior to randomization
Patients receiving prophylactic antibiotics (e.g., for prevention of a urinary tract infection or to prevent chronic obstructive pulmonary disease exacerbation) are eligible.
Administration of a live, attenuated vaccine within 4 weeks before randomization or anticipation that such a live, attenuated vaccine were required during the study
Prior treatment with CD137 agonists or immune checkpoint blockade therapies, anti—PD-1, and anti—PD-L1 therapeutic antibodies
Patients who have had prior anti—cytotoxic T lymphocyte—associated antigen 4 (CTLA-4) treatment may be enrolled, provided the following requirements are met:
   Last dose of anti—CTLA-4 at least 6 weeks prior to randomization
   No history of severe immune-related adverse effects from anti—CTLA-4 (CTCAE Grade 3 or 4)
Treatment with systemic immunostimulatory agents (including, but not limited to, interferons or interleukin-2) within 4 weeks or five half-lives of the drug, whichever is longer, prior to randomization
Prior treatment with cancer vaccines is allowed.
Treatment with systemic corticosteroids or other systemic immunosuppressive medications (including, but not limited to, corticosteroids, cyclophosphamide, azathioprine, methotrexate, thalidomide, and anti—tumor necrosis factor [anti-TNF] agents) within 2 weeks prior to randomization Patients who have received acute, low-dose 10 mg oral prednisone or equivalent), systemic immunosuppressant medications may be enrolled in the study. The use of corticosteroids 10 mg oral prednisone or equivalent) for chronic obstructive pulmonary disease, mineralocorticoids (e.g., fludrocortisone) for patients with orthostatic hypotension, and low-dose supplemental corticosteroids for adrenocortical insufficiency are allowed.

Exclusion Criteria Related to Chemotherapy
History of allergic reactions to cisplatin, carboplatin, or other platinum-containing compounds
Patients with hearing impairment (cisplatin)
Grade 2 peripheral neuropathy as defined by NCI CTCAE v4.0 criteria (cisplatin)
CrCl <60 mL/min (cisplatin)
Known hypersensitivity to gemcitabine
History of radiation therapy within 7 days prior to initiating gemcitabine

Method of Treatment Assignment and Blinding

This was an open-label study. After written informed consent has been obtained and eligibility has been established (including determination of tumor PD-L1 status by central testing), personnel at the study

site entered demographic and baseline characteristics in the interactive voice or Web-based response system (IxRS). For patients who were eligible for enrollment, the study site obtained the patient's randomization number and treatment assignment from the IxRS. Randomization to one of two treatment arms occurred in a 1:1 ratio. Permuted-block randomization was applied to ensure a balanced assignment to each treatment arm. Randomization was stratified by the following criteria:
Sex (male vs. female)
ECOG Performance Status (0 vs. 1)
Histology (non-squamous vs. squamous)
Tumor tissue PD-L1 expression by IHC TC1/2/3 and any IC vs. TC0 and 101/2/3)
   Patients received their first dose of study treatment on the day of randomization if possible. If this was not possible, the first dose occurred within 5 days after randomization.

Study Treatment

Patients with non-squamous disease received either atezolizumab alone or pemetrexed in combination with cisplatin or carboplatin. Patients with squamous disease received either atezolizumab alone or gemcitabine in combination with cisplatin or carboplatin.

Formulation, Packaging, and Handling

Atezolizumab

The atezolizumab (MPDL3280A) drug product is provided as a sterile liquid in a single-use, 20-mL glass vial. The vial is designed to deliver 20 mL (1200 mg) of atezolizumab solution but may contain more than the stated volume to enable delivery of the entire 20 mL volume.

Cisplatin, Carboplatin, Premetrexed, and Gemcitabine

Cisplatin, carboplatin, pemetrexed, and gemcitabine were used in commercially available formulations. For information on the formulation, packaging, and handling of cisplatin, carboplatin, pemetrexed, and gemcitabine, see the local prescribing information for each drug.

Atezolizumab Administration

Patients who were randomized to be treated with atezolizumab received 1200 mg atezolizumab administered by IV infusion q21d in a monitored setting where there is immediate access to trained personnel and adequate equipment/medicine to manage potentially serious reactions. Atezolizumab infusions were administered per the instructions outlined in Table 3.

TABLE 3
Administration of First and Subsequent Infusions of Atezolizumab

| First Infusion | Subsequent Infusions |
|---|---|
| No pre-medication is allowed. | If patient experienced infusion-related |
| Record patient's vital signs (pulse rate, | reaction during any previous infusion, |
| respiratory rate, blood pressure, and | pre-medication with antihistamines may |
| temperature) within 60 minutes before | be administered for Cycles ≥2 at the |
| starting infusion. | discretion of the treating physician. |
| Infuse atezolizumab (1200 mg in a 250 mL | Record patient's vital signs (pulse rate, |
| 0.9% NaCl intravenous infusion bag) over | respiratory rate, blood pressure, and |
| 60 (±15) minutes. | temperature) within 60 minutes before |
| If clinically indicated, record patient's vital | starting infusion. |
| signs (pulse rate, respiratory rate, blood | If the patient tolerated the first infusion |
| pressure, and temperature) during the | well without infusion-associated adverse |
| infusion at 15, 30, 45, and 60 minutes | events, the second infusion may be |
| (±5-minute windows are allowed for all | delivered over 30 (±10) minutes. |
| timepoints). | If no reaction occurs, continue |
| If clinically indicated, record patient's | subsequent infusions over |
| vital signs (pulse rate, respiratory rate, | 30 (±10) minutes |
| blood pressure, and temperature) at | Continue to record vital signs within |
| 30 (±10) minutes after the infusion. | 60 minutes before starting infusion. |
| Patients were informed about the | Record vital signs during and after the |
| possibility of delayed post-infusion | infusion if clinically indicated. |
| symptoms and instructed to contact their | If the patient had an infusion-related |
| study physician if they develop such | reaction during the previous infusion, the |
| symptoms. | subsequent infusion must be delivered |
| | over 60 (±15) minutes. |
| | Record patient's vital signs (pulse rate, |
| | respiratory rate, blood pressure, and |
| | temperature) during the infusion if |
| | clinically indicated or if patient |
| | experienced symptoms during the |
| | previous infusion. |
| | Record patient's vital signs (pulse rate, |
| | respiratory rate, blood pressure, and |
| | temperature) 30 (±10) minutes after |
| | the infusion, if clinically indicated or if |
| | patient experienced symptoms during |
| | previous infusion. |

### Premetrexed in Combination with Cisplatin or Carboplatin (Patients with Non-Squamous NSCLC Only)

Each study site administered pemetrexed (non-squamous NSCLC) in combination with platinum-based chemotherapy (cisplatin or carboplatin) for four or six cycles. The intended number of chemotherapy induction cycles (four or six cycles) was specified by the investigator prior to randomization. The selected platinum chemotherapy agent remained the same for all cycles (e.g., patients who start on pemetrexed plus cisplatin should remain on this combination and not switch to pemetrexed plus carboplatin or vice versa). However, for patients who experience unacceptable toxicity with the selected platinum chemotherapy, a switch may have been considered after discussion with and approval by the Medical Monitor.

Patients received steroid, folic acid, and vitamin B12 premedication for pemetrexed. The choice of steroid and timing of premedication can be administered according to the local standard of care and prescribing information (see Table 4). Folic acid supplementation may be started before randomization in all

patients at the discretion of the investigator to meet the local standard of care in anticipation for pemetrexed-based treatment and then discontinued in patients assigned to the atezolizumab arm after randomization. In addition, patients should receive anti-emetic and IV hydration for platinum-based treatments according to the local standard of care and prescribing information.

Table 4 lists the suggested premedication for pemetrexed plus platinum-based chemotherapy and Table 5 lists the doses that were used and the suggested infusion times for pemetrexed plus platinum-based chemotherapy. Chemotherapy infusion times may be adapted in accordance with local standard of care.

TABLE 4
Premedication for Pemetrexed plus Platinum-Based Chemotherapy

| Premedication | Dose/Route | Timing |
|---|---|---|
| Folic acid | 350-1000 μg PO | Once daily beginning 5-7 days |
| | | before Cycle 1, Day 1, and |
| | | continuing until 3 weeks after |
| | | discontinuation of |
| | | pemetrexed or as per local |
| | | standard of care. |
| Vitamin B12 | 1000 μg IM | q9w beginning Cycle 1, Day 1, |
| | | and continuing until |
| | | 3 weeks after discontinuation of |
| | | pemetrexed or as per |
| | | local standard of care. |
| Dexamethasone | 4 mg PO | Twice daily the day prior to, the |
| (Suggested) | | day of, and the day after |
| | | each infusion of pemetrexed or |
| | | as per local standard of |
| | | care. |

IM = intramuscular; PO = oral; q9w = every 9 weeks.
Note:
Prophylactic anti-emetics per local practice.

TABLE 5
Treatment Regimen for Pemetrexed
plus Platinum-Based Chemotherapy

| | | Induction | Maintenance |
|---|---|---|---|
| | | Period | Period |
| Study Drug | Dose/Route | (Four or Six Cycles) | (Until PD) |
| Premetrexed | 500 mg/m² IV | Over ~10 minutes | Over |
| | | on Day 1 q21d | approximately |
| | | | 10 minutes |
| | | | on Day 1 q21d |
| Carboplatin | AUC 6 IV | Over ~30-60 minutes | N/A |
| | | on Day 1 q21d | |
| | | Or | |
| | | Over 1-2 hours on Day | |
| | | 1 q21d | |
| Cisplatin | 75 mg/m² | Over ~30-60 minutes | N/A |
| | | on Day 1 q21d | |
| | | Or | |
| | | Over 1-2 hours on Day | |
| | | 1 q21d | |

Pemetrexed was administered by IV infusion at a dose of 500 mg/m² on Day 1 of each 21-day cycle, followed by carboplatin or cisplatin at approximately 30 minutes after the completion of pemetrexed. Patients who do not experience disease progression per RECIST v1.1 after completing (four or six cycles of) induction treatment, will continue maintenance treatment with pemetrexed, given on Day 1 of each 21-day cycle until disease progression per RECIST v1.1. All patients eligible for pemetrexed therapy were to avoid taking non-steroidal anti-inflammatory drugs (NSAIDs) for at least 2 days prior to pemetrexed administration if the NSAID has a short elimination half-life, for at least 5 days prior to pemetrexed administration if the NSAID has a long elimination half-life, on the day of pemetrexed administration, and at least 2 days following pemetrexed administration.

### Gemcitabine in Combination with Cisplatin or Carboplatin (Patients with Squamous NSCLC Only)

Each study site administered gemcitabine (squamous NSCLC) in combination with platinum-based chemotherapy (cisplatin or carboplatin) for four or six cycles. The intended number of chemotherapy induction cycles (four or six cycles) was specified by the investigator prior to randomization. The selected platinum chemotherapy agent remained the same for all cycles (e.g., patients who start on gemcitabine plus cisplatin should remain on this combination and not switch to gemcitabine plus carboplatin or vice versa). However, for patients who experience unacceptable toxicity with the selected platinum chemotherapy, a switch may be considered after discussion with and approval by the Medical Monitor.

Patients received anti-emetic therapy and IV hydration for platinum-based treatments according to the local standard of care and prescribing information. Table 6 lists the doses that were used and the suggested infusion times for gemcitabine plus platinum-based treatments. Chemotherapy infusion times may have been adapted in accordance with local standard of care.

TABLE 6
Treatment Regimens for Gemcitabine
plus Platinum-Based Chemotherapy

| Chemotherapy | Dose/Route | Treatment (Four or Six Cycles) |
|---|---|---|
| Gemcitabine | 1250 mg/m² IV | Over 30 minutes on Days 1 and 8 q21d |
| Cisplatin | 75 mg/m² IV | Over 1-2 hours on Day 1 q21d |
| Gemcitabine | 1000 mg/m² IV | Over 30 minutes on Days 1 and 8 q21d |
| Carboplatin | AUC 5 IV | Over approximately 30-60 |
| | | minutes on Day 1 q21d |

Gemcitabine was administered by IV infusion at a dose of 1250 mg/m² (in combination with cisplatin) or 1000 mg/m² (in combination with carboplatin) over 30 minutes on Days 1 and 8 of each 21-day cycle followed by cisplatin or carboplatin at approximately 30 minutes after the completion of gemcitabine infusion on Day 1 only. Gemcitabine injection was diluted prior to infusion. The recommended diluent for reconstitution of gemcitabine was 0.9% sodium chloride injection without preservatives. The administration of gemcitabine was done in accordance with local practice and the prescribing information; sites followed their institutional standard of care for determining the gemcitabine dose for obese patients and for dose adjustment in the event of patient weight changes.

Cisplatin Administration

Cisplatin was administered by IV infusion approximately 30 minutes after completion of the pemetrexed or gemcitabine infusion at a dose of 75 mg/m² over 1-2 hours as per the above tables. Patients received adequate anti-emetic treatment and appropriate hydration prior to and/or after receiving cisplatin.

Carboplatin Administration

Carboplatin was administered by IV infusion at a dose of AUC 6 when given in combination with pemetrexed or at a dose of AUC 5 when given in combination with gemcitabine, after completion of the pemetrexed or gemcitabine infusion, with standard anti-emetics per local practice guidelines.

The carboplatin dose was calculated using the Calvert formula (Calvert et al. 1989):

Calvert Formula

Total dose (mg)=(target AUC)×(glomerular filtration rate [GFR]+25)

The GFR used in the Calvert formula to calculate AUC-based dosing should not exceed 125 mL/min.

For the purposes of this protocol, the GFR is considered to be equivalent to the CrCl.

The CrCl is calculated by institutional guidelines or by the method of Cockcroft and Gault (1976) using the following formula:

CrCl=[(140—age) (wt)]/[72×Scr](×0.85 if female)

Where: CrCl=creatinine clearance in mL/min
age=patient's age in years
wt=patient's weight in kg
Scr=serum creatinine in mg/dL
For patients with an abnormally low serum creatinine level, GFR was estimated using a minimum creatinine level of 0.8 mg/dL or cap the estimated GFR at 125 mL/min.

If a patient's GFR was estimated on the basis of serum creatinine measurements by the isotope dilution mass spectroscopy method, the U.S. Food and Drug Administration (FDA) recommends that physicians consider capping the dose of carboplatin for desired exposure (AUC) to avoid potential toxicity caused by overdosing. On the basis of the Calvert formula described in the carboplatin label, the maximum doses can be calculated as follows:

Maximum carboplatin dose (mg)=target AUC (mg min/mL) x (GFR+25 mL/min)
The maximum dose is based on a GFR estimate that is capped at 125 mL/min for patients with normal renal function. No higher estimated GFR values should be used.
For a target AUC=6, the maximum dose is 6×150=900 mg.
For a target AUC=5, the maximum dose is 5×150=750 mg.
For a target AUC=4, the maximum dose is 4×150=600 mg.

Concomitant Therapy

Concomitant therapy includes any medication (e.g., prescription drugs, over-the-counter drugs, herbal or homeopathic remedies, nutritional supplements) used by a patient in addition to protocol—specified study treatment from 7 days prior to screening until the treatment discontinuation visit. All such medications should be reported to the investigator.

Permitted Therapy

The following therapies were allowed to continue while patients were in the study:
Oral contraceptives
Hormone-replacement therapy
Prophylactic or therapeutic anticoagulation therapy (such as low—molecular weight heparin or warfarin at a stable dose level)
Palliative radiotherapy (e.g., treatment of known bony metastases or symptomatic relief of pain) provided it does not interfere with the assessment of tumor target lesions (e.g., the lesion to be irradiated is not the only site of disease as that would render the patient not evaluable for response by tumor assessments according to RECIST v1.1). It is not a requirement to withhold atezolizumab during palliative radiotherapy.
Inactive influenza vaccinations
Megestrol administered as an appetite stimulant
Corticosteroids 10 mg oral prednisone or equivalent) for chronic obstructive pulmonary disease
Mineralocorticoids (e.g., fludrocortisone)
Low-dose corticosteroids for patients with orthostatic hypotension or adrenocortical insufficiency

In general, investigators managed a patient's care with supportive therapies as clinically indicated, as per local standards. Patients who experienced infusion-associated symptoms were treated symptomatically with acetaminophen, ibuprofen, diphenhydramine, and/or famotidine or another H2 receptor antagonist as per standard practice (for sites outside the United States, equivalent medications may be substituted per local practice). Serious infusion-associated events manifested by dyspnea, hypotension, wheezing, bronchospasm, tachycardia, reduced oxygen saturation, or respiratory distress should be managed with supportive therapies as clinically indicated.

Cautionary Therapy for Atezolizumab-Treated Patients

Systemic corticosteroids and TNF-α inhibitors may attenuate potential beneficial immunologic effects of treatment with atezolizumab. Therefore, in situations where systemic corticosteroids or TNF-α inhibitors would be routinely administered, alternatives, including antihistamines, were considered first by the treating physician. If the alternatives were not feasible, systemic corticosteroids and TNF-α inhibitors were administered at the discretion of the treating physician except in the case of patients for whom CT scans with contrast are contraindicated (i.e., patients with contrast allergy or impaired renal clearance).

Systemic corticosteroids were recommended, with caution at the discretion of the treating physician, for the treatment of specific adverse events when associated with atezolizumab therapy.

Prohibited Therapy

Any concomitant therapy intended for the treatment of cancer, whether health authority—approved or experimental, was prohibited for various time periods prior to starting study treatment, depending on the anti-cancer agent, and during study treatment until disease progression is documented and the patient has

discontinued study treatment. This includes but is not limited to chemotherapy, hormonal therapy, immunotherapy, radiotherapy, non-approved experimental agents, or herbal therapy (unless otherwise noted).

The following medications were prohibited during the study, unless otherwise noted:
Denosumab; patients who are receiving denosumab prior to enrollment must be willing and eligible to receive a bisphosphonate instead while in the study.
Any live, attenuated vaccine (e.g., FLUMIST®) within 4 weeks prior to randomization, during treatment, or within 5 months following the last atezolizumab dose (for patients randomized to atezolizumab).
Use of steroids to premedicate patients for whom CT scans with contrast are contraindicated (i.e., patients with contrast allergy or impaired renal clearance); in such patients, non-contrast CT of the chest and non-contrast CT or MRI scans of the abdomen and pelvis should be performed.

The concomitant use of herbal therapies was not recommended because their pharmacokinetics, safety profiles, and potential drug-drug interactions are generally unknown. However, their use for patients in the study was allowed at the discretion of the investigator, provided that there were no known interactions with any study treatment. As noted above, herbal therapies intended for the treatment of cancer were prohibited.

Tumor Tissue Samples

A central laboratory coordinated the sample collection of tissue samples for research-related testing at central laboratories or at the Sponsor. Instruction manuals and supply kits were provided for all central laboratory assessments.

### Archival and Freshly Collected Tumor Tissue Samples for Screening

Representative tumor specimens in paraffin blocks (preferred) or 15 (or more) freshly cut, serial unstained sections (on slides) with an associated pathology report were submitted for determination of PD-L1 status to ensure patient meets eligibility criteria prior to randomization. If fewer than 15 slides were available at baseline (but no fewer than 10), the patient may still have been eligible, upon discussion with the Medical Monitor. In addition, expression of PD-L1 defined by the SP263 IHC assay was evaluated. Exploratory biomarkers (including, but not limited to PD-L1, PD-1, mutational load, and other biomarkers related to immune or NSCLC biology, such as T-cell biomarkers or non-inherited biomarkers identified through NGS on extracted DNA and/or RNA) may also have been evaluated. For patients with non-squamous NSCLC, EGFR and/or ALK status if unknown may have been assessed locally or at a central lab. Additional tissue were required for central testing of EGFR and/or ALK.

Tumor tissue should have been of good quality based on total and viable tumor content (sites were informed if the quality of the submitted specimen is inadequate to determine tumor PD-L1 status).

An archival tumor specimen should have been submitted if available. If an archival specimen is not available, the patient may still be eligible, with the assumption that the patient is willing to consent to and undergo a pre-treatment biopsy or resection of the tumor.

For freshly collected biopsy specimens, acceptable samples included those outlined below, provided there is a minimum of 50 viable tumor cells that preserve cellular context and tissue architecture regardless of needle gauge or retrieval method:
Core needle biopsy sample collection for deep tumor tissue; at least three cores, embedded into a single paraffin block, should be submitted for evaluation
Excisional, incisional, punch, or forceps biopsy sample collection for cutaneous, subcutaneous, or mucosal lesions
Tumor tissue resections

Fine-needle aspiration (defined as samples that do not preserve tissue architecture and yield cell suspension and/or cell smears), brushing, cell pellets from pleural effusion, and lavage samples were not acceptable.

Tumor tissue from bone metastases that have been decalcified was not acceptable.

For archival samples, the remaining tumor tissue block for all patients enrolled was returned to the site upon request or 18 months after final closure of the study database, whichever was sooner. Tissue samples from patients who were deemed ineligible to enroll in the study were returned no later than 6 weeks after eligibility determination.

Tumor Samples at the Time of Radiographic Progression

Patients in all treatment arms underwent a mandatory tumor biopsy to obtain a tumor sample, unless not clinically feasible, at the time of radiographic disease progression (within 40 days of radiographic progression or prior to the start of the next anti-cancer treatment, whichever is sooner). Acceptable samples include those outlined below:
Core needle biopsy sample collection for deep tumor tissue; at least three cores, embedded into a single paraffin block, should be submitted for evaluation
Excisional, incisional, punch, or forceps biopsy
Tumor tissue resection

The status of immune-related, tumor type—related and other exploratory biomarkers (including, but not limited to, T-cell biomarkers and non-inherited biomarkers identified through NGS on extracted DNA and/or RNA) in tumor tissue samples may have been evaluated. NGS may have been performed by Foundation Medicine. If performed by Foundation Medicine, the investigator may have obtained results from the samples collected at the time of disease progression in the form of an NGS report, which is available upon request directly from Foundation Medicine. The investigator may have shared and discussed the results with the patient, unless the patient chose otherwise. The Foundation Medicine NGS assay has not been cleared or approved by the FDA; results from these investigational tests were not used to guide future treatment decisions.

Tumor Samples at Other Timepoints

If a patient undergoes a medically indicated procedure (e.g., bronchoscopy, esophagogastroduodenoscopy, colonoscopy) any time during the course of the study that has the likelihood of yielding tumor tissue, any remaining samples or a portion of the sample not necessary for medical diagnosis (leftover tumor tissue) may be obtained for exploratory analysis.

Patients with additional tissue samples from procedures performed at different times during the course of their study participation (during treatment and during survival follow-up) who have signed the RCR optional consent were requested (but not required) to also submit these optional fresh biopsy samples for central testing. Tumor tissue samples collected at the time of clinical events (e.g., clinical response) were preferred. Tissue samples obtained at multiple times from individual patients contributed to an improved understanding of the dynamics of PD-L1 expression and relationship with intervening anti-cancer therapy.

Anti-Therapeutic Antibody Testing

Treatment with atezolizumab may elicit an immune response. Patients with signs of any potential immune response to atezolizumab were closely monitored. Validated screening and confirmatory assays were employed to detect ATAs at multiple timepoints before, during, and after treatment with atezolizumab. The immunogenicity evaluation will utilize a risk-based immunogenicity strategy (Rosenberg and Worobec 2004; Koren et al. 2008) to characterize ATA responses to atezolizumab in support of the clinical development program. This tiered strategy included an assessment of whether detected ATA responses correlate with relevant clinical endpoints. Implementation of ATA characterization assays depended on the safety profile and clinical immunogenicity data.

Timing of Assessments

Screening tests and evaluations were performed within 28 days prior to Cycle 1, Day 1. Results of standard-of-care tests or examinations performed prior to obtaining informed consent and within 28 days prior to Cycle 1, Day 1, may have been used; such tests did not need to be repeated for screening.

Assessments during Treatment

All treatment visits occurred within ±3 days from the scheduled date unless otherwise noted. All assessments were performed on the day of the specified visit unless a time window is specified. Assessments scheduled on the day of study treatment administration (Day 1) of each cycle were performed prior to study treatment infusion unless otherwise noted.

If scheduled dosing and study assessments were precluded because of a holiday, weekend, or other event, then dosing may have been postponed to the soonest following date, with subsequent dosing and visits continuing on a 21-day schedule. If treatment was postponed for fewer than 3 days, the patient may have resumed the original schedule.

After completion of four or six cycles of pemetrexed or gemcitabine combined with a platinum agent (for patients in the control arm) or after five cycles (for patients in the atezolizumab arm), one of three cycles may have been delayed by 1 week (28 days instead of 21 days for one cycle) to allow for vacations/holidays. Following the delay, the next cycle was delivered 21 days from the previous dose administration. Two consecutive 28-day cycles were not permitted.

Tumor assessments occurred every 6 weeks (±7 days) for 48 weeks following Cycle 1, Day 1, and every 9 weeks (±7 days) thereafter after the completion of the Week 48 tumor assessment, regardless of treatment delays until radiographic disease progression per RECIST v1.1 (or loss of clinical benefit for atezolizumab-treated patients who had continued treatment with atezolizumab after radiographic disease progression according to RECIST v1.1), withdrawal of consent, death, or study termination by the Sponsor, whichever occurs first. Patients who discontinued treatment for reasons other than radiographic disease progression per RECIST v1.1 (e.g., toxicity, symptomatic deterioration) continued scheduled tumor assessments until radiographic disease progression per RECIST v1.1 (or loss of clinical benefit for atezolizumab-treated patients who had continued treatment with atezolizumab after radiographic disease progression according to RECIST v1.1), withdrawal of consent, death, or study termination by Sponsor, whichever occurs first. In the absence of radiographic disease progression per RECIST v1.1, tumor assessments continued regardless of whether patients start a new anti-cancer therapy.

The following assessments were performed 96 hours before Day 1 of each cycle:
ECOG Performance Status
Limited physical examination
Local laboratory tests

Screening assessments performed 96 hours before Cycle 1, Day 1 were not required to be repeated for Cycle 1, Day 1. Local hematology tests must also be performed prior to gemcitabine infusions on Day 8.

Assessments at Treatment Discontinuation Visit Patients who discontinued study treatment returned to the clinic for a treatment discontinuation visit within 30 days after the last dose of study treatment. The visit at which a response assessment showed radiographic disease progression according to RECIST v1.1 (or loss of clinical benefit for atezolizumab-treated patients who had continued treatment with atezolizumab after radiographic disease progression according to RECIST v1.1) was used as the treatment discontinuation visit.

Patients who discontinued study treatment were followed according to the follow-up visit schedule for progression and/or survival until death, loss to follow-up, or withdrawal of consent, which were defined as study discontinuation.

### Assessment of Safety

Safety Plan

Measures were taken to ensure the safety of patients participating in this study, including the use of stringent inclusion and exclusion criteria and close monitoring (as indicated below).

Administration of atezolizumab was performed in a monitored setting where there was immediate access to trained personnel and adequate equipment/medicine to manage potentially serious reactions. All serious adverse events and adverse events of special interest were recorded during the study and for up to 90 days after the last dose of study treatment or initiation of new systemic anti-cancer therapy after the last dose of study treatment, whichever occurred first. All other adverse events were recorded during the study and for up to 30 days after the last dose of study treatment or until the initiation of new systemic anti-cancer therapy after the last dose of study treatment, whichever occurred first.

After the adverse event reporting period, all deaths continued to be reported. In addition, the Sponsor was notified if the investigator became aware of any serious adverse event or adverse event of special interest that is believed to be related to prior exposure to study treatment.

Dose Modification

### General Notes Regarding Dose Modification

Reasons for dose modifications or delays, the supportive measures taken, and the outcomes were documented in the patient's chart and recorded on the eCRF. The severity of adverse events were graded according to the NCI CTCAE v4.0 grading system.
For any concomitant conditions already apparent at baseline, the dose modifications applied according to the corresponding shift in toxicity grade, if the investigator considered it was appropriate. For example, if a patient had Grade 1 asthenia at baseline that increased to Grade 2 during study treatment, this was considered a shift of one grade and treated as Grade 1 toxicity for dose-modification purposes.
When several toxicities with different grades of severity occurred at the same time, the dose modifications were according to the highest grade observed.
If, in the opinion of the investigator, a toxicity was considered to be due solely to one component of chemotherapy, the dose of the other chemotherapy component did not require modification and the other chemotherapy component(s) were administered if there is no contraindication.
The investigator was permitted to use discretion in modifying or accelerating the dose modification guidelines described below depending on the severity of toxicity and an assessment of the risk versus benefit for the patient, with the goal of maximizing patient compliance and access to supportive care.

### Atezolizumab Dose Modifications, Treatment Delays, or Treatment Discontinuation and Management of Specific Adverse Events

There was no dose reduction for atezolizumab in this study. Patients may have temporarily suspended study treatment with atezolizumab for up to 105 days beyond the last dose if they experienced an adverse event that required a dose to be withheld. If atezolizumab was withheld because of adverse events for >105 days beyond the last dose, then the patient was discontinued from atezolizumab treatment and was followed for safety and efficacy. Exceptions required Medical Monitor approval.

If a patient was tapered off steroids used to treat adverse events, atezolizumab may have been withheld for additional time >105 days from the last dose until steroids were discontinued or reduced to prednisone dose (or dose equivalent) 10 mg/day. The acceptable length of interruption depended on agreement between the investigator and the Medical Monitor.

Dose interruptions for reasons other than toxicity, such as surgical procedures, were allowed with Medical Monitor approval. The acceptable length of interruption depended on agreement between the investigator and the Medical Monitor.

### Premetrexed Dose Modifications, Treatment Delays, or Treatment Discontinuation and Management of Specific Adverse Events

The dose modification guidelines were applicable for pemetrexed used as a single agent or in combination with cisplatin or carboplatin. Treatment with pemetrexed was discontinued if a patient experienced any hematologic or non-hematologic Grade 3 or 4 toxicity after two dose reductions, or if treatment was delayed for more than 63 days due to toxicities.

At the start of each cycle, the ANC must have been ≥1500/μL and the platelet count must have been ≥100,000/μL. Treatment was delayed for up to 63 days to allow sufficient time for recovery. Growth factors may have been used in accordance with American Society of Clinical Oncology (ASCO) and National Comprehensive Cancer Network (NCCN) guidelines (Smith et al. 2006). Upon recovery, dose adjustments at the start of a subsequent cycle were made on the basis of the lowest (nadir) platelet and neutrophil values from the previous cycle (see Table 7). In the event that dose adjustments were needed for both ANC and platelets, patients received the lower dose.

TABLE 7
Pemetrexed Dose Modifications for Hematologic Toxicities

| Toxicity | Premetrexed Dose |
|---|---|
| ANC <500/μL and platelets ≥50,000/μL | 75% of previous dose |
| Platelets <50,000/μL, regardless of ANC | 75% of previous dose |
| Platelets <50,000/μL with Grade ≥2 bleeding, | 50% of previous dose |
| regardless of ANC | |

At the start of each cycle, the CrCl must have been ≥45 mL/min. For enrollment and dosing decisions, CrCl was estimated using the original, weight-based Cockcroft and Gault formula (1976) or measured using the appropriate radiolabeled method (51-CrEDTA or Tc99m-DTPA) to determine the GFR. The method of CrCl assessment used at baseline was used throughout the study.

If a patient developed a non-hematologic toxicity (see Table 8), pemetrexed was withheld for up to 63 days until resolution to equal or less than the patient's baseline (or Grade≤1 if patient did not have that toxicity at baseline). Treatment should was resumed according to the guidelines in Table 8. For Grade 3 or 4 neurotoxicity, pemetrexed should be resumed at 50% of the previous dose upon improvement, or discontinued immediately (based on investigator's clinical judgment).

TABLE 8
Pemetrexed Dose Modifications for Non-Hematologic Toxicities

| Toxicity | Premetrexed Dose |
|---|---|
| Any diarrhea requiring hospitalization | 75% of previous dose |
| (irrespective of grade) or Grade | |
| 3 or 4 diarrhea that occurs on | |
| adequate anti-diarrhea medication. | |
| Neurotoxicity Grade 2 | 75% of previous dose |
| Neurotoxicity Grade 3 or 4 | 50% of previous dose or |
| | permanent discontinuation |
| Any other Grade 3 or 4 toxicities | 75% of previous dose |

### Gemcitabine Dose Modifications, Treatment Delays, or Treatment Discontinuation and Management of Specific Adverse Events

The dose modification guidelines for gemcitabine are provided below. Treatment with gemcitabine was discontinued if a patient experienced any hematologic or non-hematologic Grade 3 or 4 toxicity after two dose reductions, or if treatment was delayed for more than 63 days due to toxicities.

Gemcitabine dose modifications for hematologic toxicity were based on the granulocyte and platelet counts taken on Days 1 and 8 of treatment (Table 9 and Table 10). Patients receiving gemcitabine were monitored prior to each dose with a full blood count, including differential and platelet counts. Treatment was delayed for up to 63 days to allow sufficient time for recovery. Growth factors were used in accordance with American Society of Clinical Oncology (ASCO) and National Comprehensive Cancer Network (NCCN) guidelines (Smith et al. 2006). Upon recovery, dose adjustments at the start of a subsequent cycle was made on the basis of the lowest (nadir) platelet and neutrophil values from the previous cycle.

In the event that dose adjustments are needed for both ANC and platelets, patients were to receive the lower dose.

TABLE 9
Gemcitabine Dose Modifications or Treatment
Delays for Hematologic Toxicities on Day 1

| Absolute Granulocyte | | | |
|---|---|---|---|
| Count | | Platelet Count | Gemcitabine |
| (×10⁶ L) | | (×10⁶ L) | % of Full Dose |
| ≥1500 | And | ≥100,000 | 100% |
| <1500 | Or | <100,000 | Withhold |

TABLE 10
Gemcitabine Dose Modifications or Treatment
Delays for Hematologic Toxicities on Day 8

| Absolute Granulocyte | | | |
|---|---|---|---|
| Count | | Platelet Count | Gemcitabine |
| (×10⁶ L) | | (×10⁶ L) | % of Full Dose |
| ≥1000/μL | And | ≥100,000/μL | 100% |
| 500-999/μL | Or | 50,000-99,999/μL | 75% |
| <500/μL | Or | <50,000/μL | Withhold |

Investigators were vigilant and alert to early and overt signs of myelosuppression, infection, or febrile neutropenia so that these complications can be promptly and appropriately managed. Patients were made aware of these signs and were encouraged to seek medical attention at the earliest opportunity.

If chemotherapy was withheld because of hematologic toxicity, full blood counts (including differential WBC) were obtained weekly until the counts reached the lower limits for treatment as outlined. The treatment was then resumed.

No dose reductions were recommended for anemia. Patients were supported per the treating physician's institution's guidelines.

### Cisplatin Dose Modifications, Treatment Delays, or Treatment Discontinuation and Management of Specific Adverse Events

The dose modification guidelines for cisplatin are provided below.

Treatment with cisplatin was discontinued if a patient experienced any hematologic or non-hematologic Grade 3 or 4 toxicity after two dose reductions or treatment is delayed for more than 63 days due to toxicities.

At the start of each cycle, the ANC must have been ≥1500/μL and the platelet count must have been ≥100,000/μL. Treatment was delayed for up to 63 days to allow sufficient time for recovery. Growth factors were used in accordance with American Society of Clinical Oncology (ASCO) and National Comprehensive Cancer Network (NCCN) guidelines (Smith et al. 2006; NCCN 2014). Upon recovery, dose adjustments at the start of a subsequent cycle were made on the basis of the lowest platelet and neutrophil values from the previous cycle (see Table 11).

In the event that dose adjustments were needed for both ANC and platelets, patients were to receive the lower dose.

TABLE 11
Cisplatin Dose Modifications for Hematologic Toxicities

| Toxicity | Cisplatin Dose |
|---|---|
| ANC <500/μL and platelets ≥50,000/μL | 75% of previous dose |
| Platelets <50,000/μL, regardless of ANC | 75% of previous dose |
| Platelets <50,000/μL with Grade ≥2 bleeding, | 50% of previous dose |
| regardless of ANC | |
| ANC <1000/μL plus fever of ≥38.5° C. | 75% of previous dose |

Investigators were vigilant and alert to early and overt signs of myelosuppression, infection, or febrile neutropenia so that these complications can be promptly and appropriately managed. Patients were made aware of these signs and encouraged to seek medical attention at the earliest opportunity.

If chemotherapy was withheld because of hematologic toxicity, full blood counts (including differential WBC) were obtained weekly until the counts reach the lower limits for treatment as outlined. The treatment was then resumed. No dose reductions were recommended for anemia. Patients were supported per institutional guidelines.

If a patient developed a non-hematologic toxicity (see Table 12), cisplatin was withheld for up to 63 days until resolution to less than or equal to the patient's baseline (or Grade 1 if patient did not have that toxicity at baseline). Treatment was resumed according to the guidelines in Table 12. Diarrhea was controlled with adequate anti-diarrhea medication. Nausea and/or vomiting was controlled with adequate anti-emetics.

TABLE 12
Cisplatin Dose Modifications for Non-Hematologic
Toxicities (Excluding Neurotoxicity)

| Toxicity | Cisplatin Dose |
|---|---|
| Any diarrhea requiring hospitalization | 75% of previous dose |
| (irrespective of grade) or Grade 3 or 4 | |
| diarrhea that occurs on adequate | |
| anti-diarrhea medication | |
| Grade 3 or 4 nausea/vomiting despite | 75% of previous dose |
| use of antiemetics | |
| Any other Grade 3 or 4 toxicity | 75% of previous dose |

CrCl must have been 60 mL/min prior to the start of any cycle of cisplatin. If there was a decrease in CrCl between cycles, but the CrCl was still 60 mL/min at the time of the next cycle, the investigator used clinical judgment regarding continuing cisplatin, dose reduction, or delaying the cycle. If a patient's CrCl value had not returned to 60 mL/min within 63 days following last cisplatin administration, the patient was discontinued from cisplatin.

In the event of neurotoxicity, the recommended dose adjustment for cisplatin is documented in Table 13. For Grade 3 or 4 neurotoxicity, cisplatin was resumed at 50% of the previous dose upon improvement, or discontinued immediately (based on investigator's clinical judgment).

TABLE 13
Cisplatin Dose Modifications or Treatment Discontinuation
for Associated Neurotoxicity

| Toxicity | Cisplatin Dose |
|---|---|
| Grade 1 neurotoxicity | 100% of previous dose |
| Grade 2 neurotoxicity | 75% of previous dose |
| Grade 3 or 4 neurotoxicity | 50% of previous dose or permanent |
| | discontinuation |

Carboplatin Dose Modifications, Treatment Delays, or Treatment Discontinuation and Management of Specific Adverse Events The dose modification guidelines for carboplatin are provided below.

Treatment with carboplatin was discontinued if a patient experienced any hematologic or non-hematologic Grade 3 or Grade 4 toxicity after two dose reductions or treatment is delayed for more than 63 days due to toxicities.

At the start of each cycle, the ANC must have been ≥1500/μL and the platelet count must have been ≥100,000/μL. Treatment was delayed for up to 63 days to allow sufficient time for recovery. Growth factors were used in accordance with American Society of Clinical Oncology (ASCO) and NCCN guidelines (Smith et al. 2006; NCCN 2012). Upon recovery, dose adjustments at the start of a subsequent cycle were made on the basis of the lowest platelet and neutrophil values from the previous cycle (see Table 14).

In the event that dose adjustments were needed for both ANC and platelets, patients were to receive the lower dose.

TABLE 14
Carboplatin Dose Modifications for Hematologic Toxicities

| Toxicity | Cisplatin Dose |
|---|---|
| ANC <500/μL and platelets ≥50,000/μL | 75% of previous dose |
| Platelets <50,000/μL, regardless of ANC | 75% of previous dose |
| Platelets <50,000/μL with Grade ≥2 bleeding, | 50% of previous dose |
| regardless of ANC | |
| ANC <1000/μL plus fever of ≥38.5° C. | 75% of previous dose |

Investigators were vigilant and alert to early and overt signs of myelosuppression, infection, or febrile neutropenia so that these complications can be promptly and appropriately managed. Patients were made aware of these signs and encouraged to seek medical attention at the earliest opportunity.

If chemotherapy was withheld because of hematologic toxicity, full blood counts (including differential WBC) were obtained weekly until the counts reach the lower limits for treatment as outlined. The treatment can then be resumed.

No dose reductions were recommended for anemia. Patients were supported per the treating physician's institution's guidelines.

For a non-hematologic toxicity (see Table 15), treatment was delayed for up to 63 days until resolution to less than or equal to the patient's baseline value (or Grade 1 if patient did not have that toxicity at baseline). Dose reductions at the start of the subsequent cycle were made on the basis of non-hematologic toxicities from the dose administered in the preceding cycle. Table 15 provides the dose modifications for non-hematologic toxicities.

TABLE 15
Carboplatin Dose Modifications or Treatment Discontinuation
for Non-Hematologic Toxicities

| | | Adjusted Carboplatin Dose as % |
|---|---|---|
| Toxicity | | of Previous Dose^{a} |
| Diarrhea | Grade 3 or 4^{b} | 75% |
| Nausea/vomiting | Grade 3 or 4^{c} | 75% |
| Neurotoxicity | Grade 2 | 75% |
| | Grade 3 or 4 | 50% or permanent |
| | | discontinuation |
| Transaminase | Grade 3 | 75% |
| elevation | Grade 4 | Discontinue |
| Other | Grade 3 or 4 | 75% |

AUC = area under the concentration curve.
^{a}If deemed appropriate by the investigator, adjust carboplatin dose to the specified percentage of the previous AUC.
^{b}grade 3 or 4 diarrhea that occurs on adequate anti-diarrhea medication or any grade of diarrhea requiring hospitalization.
^{c}despite the use of antiemetics.

Diarrhea was controlled with adequate anti-diarrhea medication. Nausea and/or vomiting should be controlled with adequate anti-emetics. For Grade 3 or 4 neurotoxicity, carboplatin was resumed at 50% of the previous dose upon improvement or discontinued immediately (based on investigator's clinical judgment).

Adverse Events

According to the ICH guideline for Good Clinical Practice, an adverse event is any untoward medical occurrence in a clinical investigation subject administered a pharmaceutical product, regardless of causal attribution. An adverse event can therefore be any of the following:
Any unfavorable and unintended sign (including an abnormal laboratory finding), symptom, or disease temporally associated with the use of a medicinal product, whether or not considered related to the medicinal product
Any new disease or exacerbation of an existing disease (a worsening in the character, frequency, or severity of a known condition)
Recurrence of an intermittent medical condition (e.g., headache) not present at baseline
Any deterioration in a laboratory value or other clinical test (e.g., ECG, X-ray) that is associated with symptoms or leads to a change in study treatment or concomitant treatment or discontinuation from study drug
Adverse events that are related to a protocol-mandated intervention, including those that occur prior to assignment of

### Serious Adverse Events (Immediately Reportable to the Sponsor)

A serious adverse event is any adverse event that meets any of the following criteria:
Is fatal (i.e., the adverse event actually causes or leads to death)
Is life threatening (i.e., the adverse event, in the view of the investigator, places the patient at immediate risk of death). This does not include any adverse event that had it occurred in a more severe form or was allowed to continue might have caused death.
Requires or prolongs inpatient hospitalization
Results in persistent or significant disability/incapacity (i.e., the adverse event results in substantial disruption of the patient's ability to conduct normal life functions)
Is a congenital anomaly/birth defect in a neonate/infant born to a mother exposed to study drug
Is a significant medical event in the investigator's judgment (e.g., may jeopardize the patient or may require medical/surgical intervention to prevent one of the outcomes listed above)

The terms “severe” and “serious” are not synonymous. Severity refers to the intensity of an adverse event (e.g., rated as mild, moderate, or severe, or according to NCI CTCAE criteria); the event itself may be of relatively minor medical significance (such as severe headache without any further findings).

Severity and seriousness need to be independently assessed for each adverse event recorded on the eCRF.

Serious adverse events are required to be reported by the investigator to the Sponsor immediately (i.e., no more than 24 hours after learning of the event).

### Adverse Events of Special Interest (Immediately Reportable to the Sponsor)

Adverse events of special interest were required to be reported immediately by the investigator to the Sponsor (i.e., no more than 24 hours after learning of the event). Adverse events of special interest for this study include the following:
The following confirmed treatment-emergent autoimmune conditions:
   Pneumonitis
   Hypoxia or dyspnea Grade 3
   Colitis
   Endocrinopathies: diabetes mellitus, pancreatitis, or adrenal insufficiency
   Vasculitis
   Hepatitis
   Transaminitis: Grade 2 (AST or ALT >3×ULN and bilirubin >2×ULN) OR
   AST/ALT >10×ULN
   Systemic lupus erythematosus
   Guillain-Barré syndrome
   Skin reactions: vitiligo, pemphigoid
Events suggestive of hypersensitivity, cytokine release, influenza-like illness, systemic inflammatory response system, or infusion-reaction syndromes
Cases of potential drug-induced liver injury that include an elevated ALT or AST in combination with either an elevated bilirubin or clinical jaundice, as defined by Hy's law
Suspected transmission of an infectious agent by the study drug, as defined below:

Any organism, virus, or infectious particle (e.g., prion protein transmitting transmissible spongiform encephalopathy), pathogenic or non-pathogenic, is considered an infectious agent. A transmission of an infectious agent may be suspected from clinical symptoms or laboratory findings that indicate an infection in a patient exposed to a medicinal product. This term applies only when a contamination of the study drug is suspected.

### Results

Demographics

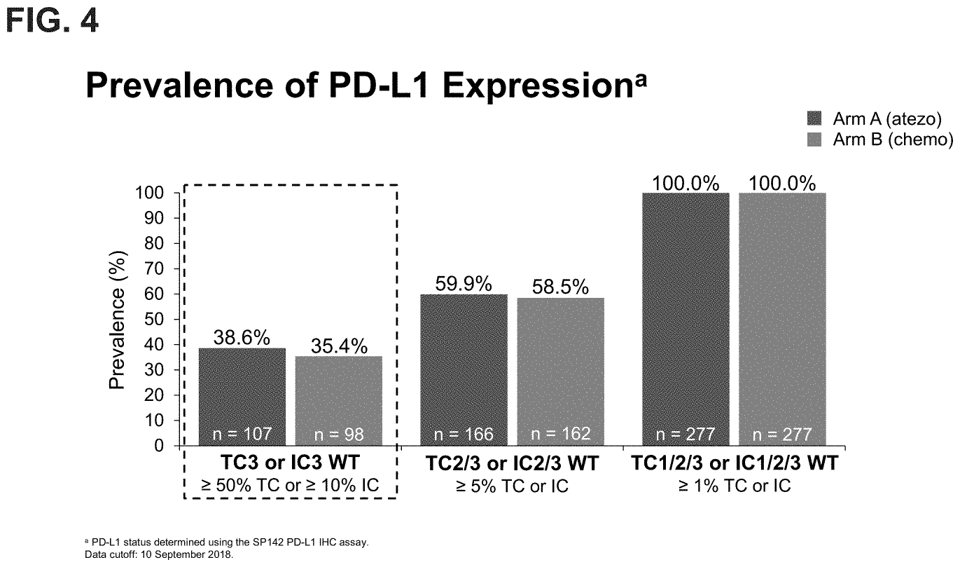

Tables 16-20, below, summarize the demographic distribution of the patients that were analyzed in this study. As used in Tables 16-20, below, and throughout the present Example, the term “TC3” denotes patients that have a detectable expression level of PD-L1 in 50% or more of the tumor cells in a tumor sample isolated from the subject prior to treatment; the term “IC3” denotes patients that have a detectable expression level of PD-L1 in tumor-infiltrating immune cells that comprise 10% or more of the tumor sample; and the term “WT” denotes patients that have no EGFR or ALK genomic tumor aberrations. Additionally, the term “TC2/3” denotes patients that have a detectable expression level of PD-L1 in 5% or more of the tumor cells in a tumor sample isolated from the subject prior to treatment; the term “IC2/3” denotes patients that have a detectable expression level of PD-L1 in tumor-infiltrating immune cells that comprise 5% or more of the tumor sample. The term “TC1/2/3” denotes patients that have a detectable expression level of PD-L1 in 1% or more of the tumor cells in a tumor sample isolated from the subject prior to treatment; the term “101/2/3” denotes patients that have a detectable expression level of PD-L1 in tumor-infiltrating immune cells that comprise 1% or more of the tumor sample. A distribution of PD-L1 expression patterns among the patients that enrolled in this study is shown in FIG. 4.

TABLE 16
Tumor-infiltrating immune cell (IC) IHC diagnostic criteria

| PD-L1 Diagnostic Assessment | IC Score |
|---|---|
| Absence of any discernible PD-L1 staining | IC0 |
| OR | |
| Presence of discernible PD-L1 staining of any | |
| intensity in tumor-infiltrating immune cells | |
| covering <1% of tumor area occupied by tumor cells, | |
| associated intratumoral stroma, and contiguous | |
| peri-tumoral desmoplastic stroma | |
| Presence of discernible PD-L1 staining of any | IC1 |
| intensity in tumor-infiltrating immune cells | |
| covering ≥1% to <5% of tumor area occupied by | |
| tumor cells, associated intratumoral stroma, and | |
| contiguous peri-tumoral desmoplastic stroma | |
| Presence of discernible PD-L1 staining of any | IC2 |
| intensity in tumor-infiltrating immune cells | |
| covering ≥5% to <10% of tumor area occupied by | |
| tumor cells, associated intratumoral stroma, and | |
| contiguous peri-tumoral desmoplastic stroma | |
| Presence of discernible PD-L1 staining of any | IC3 |
| intensity in tumor-infiltrating immune cells | |
| covering ≥10% of tumor area occupied by tumor | |
| cells, associated intratumoral stroma, and | |
| contiguous peri-tumoral desmoplastic stroma | |

TABLE 17
Tumor cell (TC) IHC diagnostic criteria

| PD-L1 Diagnostic Assessment | TC Score |
|---|---|
| Absence of any discernible PD-L1 staining | TC0 |
| OR | |
| Presence of discernible PD-L1 staining of any | |
| intensity in <1% of tumor cells | |
| Presence of discernible PD-L1 staining of any | TC1 |
| intensity in ≥1% to <5% of tumor cells | |
| Presence of discernible PD-L1 staining of any | TC2 |
| intensity in ≥5% to <50% of tumor cells | |
| Presence of discernible PD-L1 staining of any | TC3 |
| intensity in ≥50% of tumor cells | |

TABLE 18
Patient Demographics

| | TC1/2/3 or IC1/2/3-WT | | | | TC3 or IC3-WT | | | |
|---|---|---|---|---|---|---|---|---|
| | Atezo | | Chemo | | Atezo | | Chemo | |
| Characteristic | n = 277 | | n = 277 | | n = 107 | | n = 98 | |
| Age, years | | | | | | | | |
| Median | 64 | | 65 | | 63 | | 65.5 | |
| Range | 30-81 | | 30-87 | | 33-79 | | 33-87 | |
| Age group, n (%) | | | | | | | | |
| <65 years | 143 | (51.6) | 134 | (48.4) | 59 | (55.1) | 43 | (43.9) |
| 65-74 years | 106 | (38.3) | 117 | (42.2) | 33 | (30.8) | 47 | (48.0) |
| 75-84 years | 28 | (10.1) | 24 | (8.7) | 15 | (14.0) | 7 | (7.1) |
| ≥85 years | 0 | | 2 | (0.7) | 0 | | 1 | (1.0) |
| Sex, n (%) | | | | | | | | |
| Male | 196 | (70.8) | 193 | (69.7) | 79 | (73.8) | 64 | (65.3) |
| Race, n (%) | | | | | | | | |
| White | 227 | (81.9) | 240 | (86.6) | 87 | (81.3) | 82 | (83.7) |
| Asian | 45 | (16.2) | 30 | (10.8) | 20 | (18.7) | 15 | (15.3) |
| Black or African American | 2 | (0.7) | 2 | (0.7) | 0 | | 0 | |
| Multiple | 1 | (0.4) | 0 | | 0 | | 0 | |
| Unknown | 2 | (0.7) | 5 | (1.8) | 0 | | 1 | (1.0) |
| ECOG performance status, n (%) | | | | | | | | |
| 0 | 97 | (35.0) | 102 | (36.8) | 35 | (32.7) | 38 | (38.8) |
| 1 | 180 | (65.0) | 175 | (63.2) | 72 | (67.3) | 60 | (61.2) |
| Tobacco use history, n (%) | | | | | | | | |
| Never | 37 | (13.4) | 35 | (12.6) | 9 | (8.4) | 15 | (15.3) |
| Current | 74 | (26.7) | 81 | (29.2) | 20 | (18.7) | 29 | (29.6) |
| Previous | 166 | (59.9) | 161 | (58.1) | 78 | (72.9) | 54 | (55.1) |
| Histology at diagnosis, n (%) | | | | | | | | |
| Non-squamous | 192 | (69.3) | 193 | (69.7) | 80 | (74.8) | 75 | (76.5) |
| Squamous | 85 | (30.7) | 84 | (30.3) | 27 | (25.2) | 23 | (23.5) |

Atezo denotes atezolizumab; chemo, chemotherapy; ECOG, Eastern Cooperative Oncology Group; WT, wild type.

TABLE 19
Expanded Patient Demographics Stratified by PD-L1 Expression

| | TC1/2/3 or IC1/2/3 WT | | | TC3 or IC3 WT | |
|---|---|---|---|---|---|
| | Arm A | | Arm B | Arm A | Arm B |
| Characteristic | (atezo) | | (chemo) | (atezo) | (chemo) |
| n (%) | n = 277 | | n = 277 | n = 107 | n = 98 |
| Age <65 y | 143 | (51.6) | 134 (48.4) | 59 (55.1) | 43 (43.9) |
| Male | 196 | (70.8) | 193 (69.7) | 79 (73.8) | 64 (65.3) |
| White | 227 | (81.9) | 240 (86.6) | 87 (81.3) | 82 (83.7) |
| Asian | 45 | (16.2) | 30 (10.8) | 20 (18.7) | 15 (15.3) |
| Never used tobacco | 37 | (13.4) | 35 (12.6) | 9 (8.4) | 15 (15.3) |
| Non-squamous | 192 | (69.3) | 193 (69.7) | 80 (74.8) | 75 (76.5) |
| histology | | | | | |
| ECOG PS 0 | 97 | (35.0) | 102 (36.8) | 35 (32.7) | 38 (38.8) |

TABLE 20
Proportion of Patients that Received Subsequent Cancer Therapies

| | TC1/2/3 or IC1/2/3 WT | |
|---|---|---|
| | Arm A (atezo) | Arm B (chemo) |
| | n = 277 | n = 277 |
| Patients with ≥1 therapy, n (%) | 82 (29.6) | 137 (49.5) |
| Chemotherapy | 77 (27.8) | 68 (24.5) |
| Immunotherapy | 7 (2.5) | 80 (28.9) |
| Targeted therapy | 14 (5.1) | 12 (4.3) |

Patient Disposition

Table 21, below, summaries the quantity of patients that received either chemotherapy or atezolizumab treatment during this study, as well as the status of the patients following their participation in the study.

TABLE 21
Treatment Exposure

| | Chemo | | Atezo | |
|---|---|---|---|---|
| | (n = 287) | | (n = 285) | |
| Received Treatment | 264* | (92.0%) | 285 | (100.0%) |
| On-study Status | 139 | 8.4%) | 154 | (54.0%) |
| Alive: On Treatment | 31 | 10.8%) | 62 | (21.8%) |
| Alive: In Follow-Up | 108 | 7.6%) | 92 | (32.3%) |
| Discontinued Study | 148 | (51.6%) | 131 | 6.0%) |
| Death | 124 | (3.2%) | 118 | (41.4%) |
| Withdrawal By Subject | 24 | (8.4%) | 13 | (4.6%) |

Efficacy

### Overall TC3 and IC3-WT Patient Populations

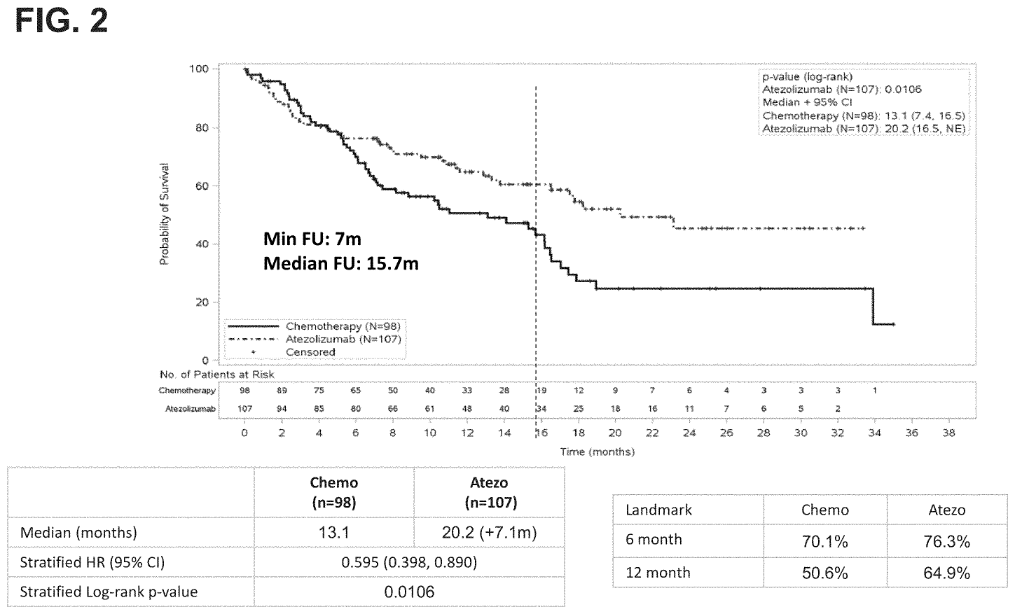

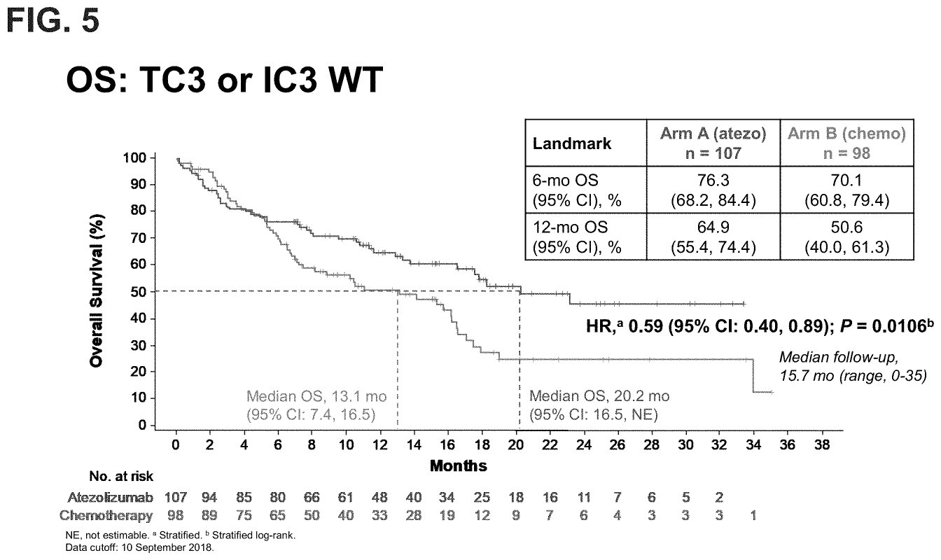

The results of this study demonstrate that patients in the TC3 or IC3-WT population experienced a statistically significant and clinically meaningful improvement in overall survival (OS) as a result of atezolizumab administration relative to TC3 or IC3-WT patients that were treated with a platinum-based chemotherapy. These results are summarized in Table 22, below, and are shown graphically in the Kaplan-Meier curves in FIGS. 2 and 5.

TABLE 22
Improvement in OS Effectuated by Atezolizumab Treatment

TC3 or IC3-WT

| | Chemo | Atezo |
|---|---|---|
| Overall Survival | (n = 98) | (n = 107) |
| Median (months) | 13.1 | 20.2 (+7.1 m) |

| | |
|---|---|
| Stratified HR | 0.595 |
| (95% CI) | (0.398, 0.890) |
| Stratified | 0.0106 |
| Log-rank p-value | |
| OS interim boundary | 0.0413 |
| (two-sided α) | |

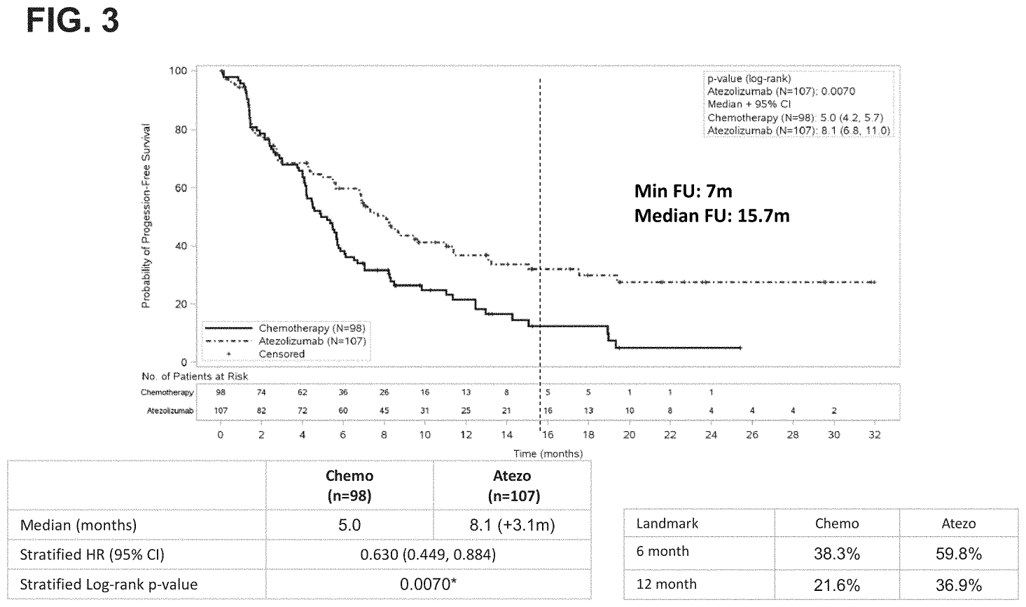

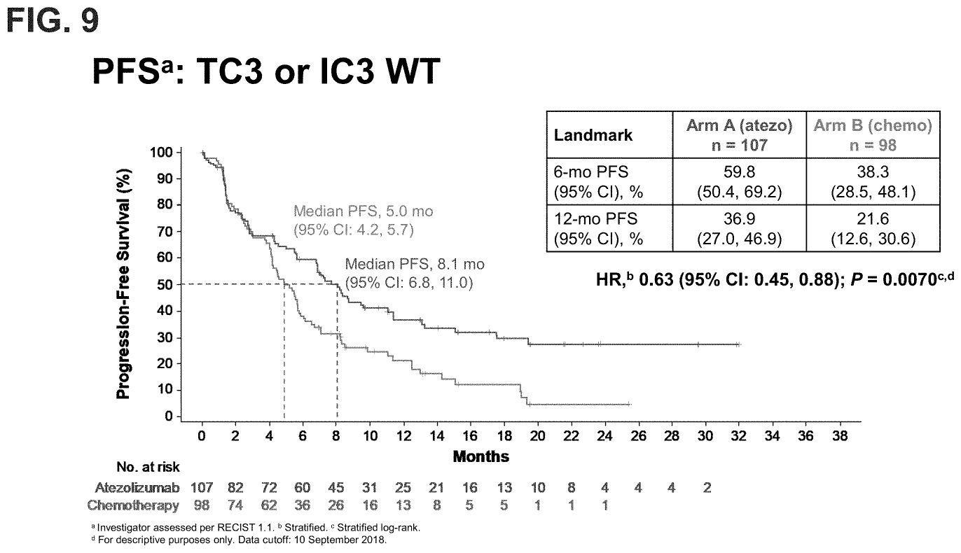

The results of this study additionally show that patients in the TC3 or IC3-WT population experienced a clinically meaningful improvement in progression-free survival (PFS) as a result of atezolizumab administration relative to TC3 or IC3-WT patients that were treated with a platinum-based chemotherapy. These results are summarized in Table 23, below, and are shown graphically in the Kaplan-Meier curves in FIGS. 3 and 9.

TABLE 23
Improvement in PFS Effectuated by Atezolizumab Treatment

TC3 or IC3-WT

| Progression-Free | Chemo | Atezo |
|---|---|---|
| Survival | (n = 98) | (n = 107) |
| Median (months) | 5.0 | 8.1 (+3.1 m) |

| | |
|---|---|
| Stratified HR | 0.630 |
| (95% CI) | (0.449, 0.884) |
| Stratified | 0.0070 |
| Log-rank p-value | |

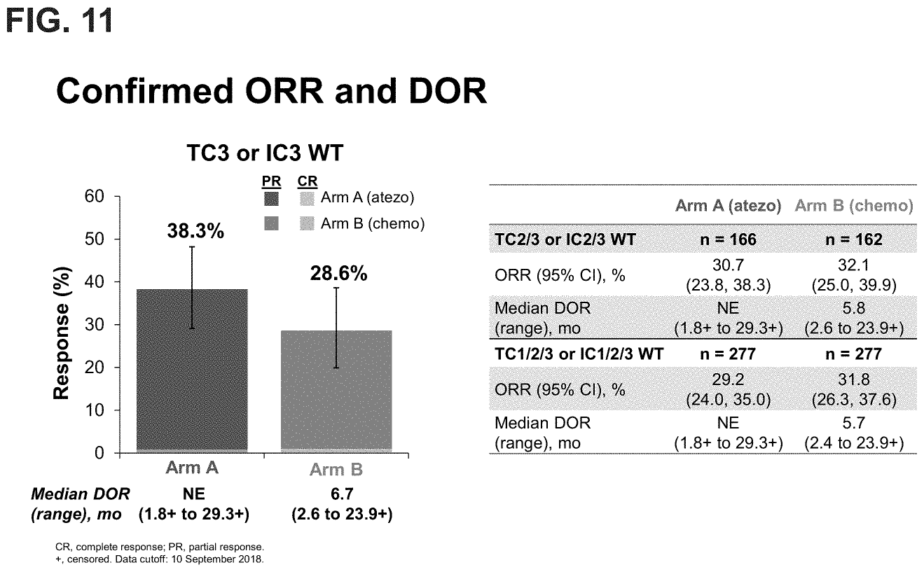

The results of this study further show that patients in the TC3 or IC3-WT population experienced improvements in overall response rate and duration of response as a result of atezolizumab administration relative to TC3 or IC3-WT patients that were treated with a platinum-based chemotherapy. These results are summarized in FIG. 11 and in Table 24, below.

TABLE 24
Improvement in ORR and DOR

| | Chemo | Atezo |
|---|---|---|
| TC3 or IC3-WT | (N = 98) | (N = 107) |
| Confirmed | 28.6% (19.90, | 38.3% (29.08, |
| ORR (95% CI) | 38.58) | 48.22) |
| median | 6.7 m (5.5, 17.3), | NE (11.8, NE), |
| DOR (95% CI) | n = 28 | n = 41 |

### Comparison of Efficacy Among Patient Groups Stratified by PD-L1 Expression

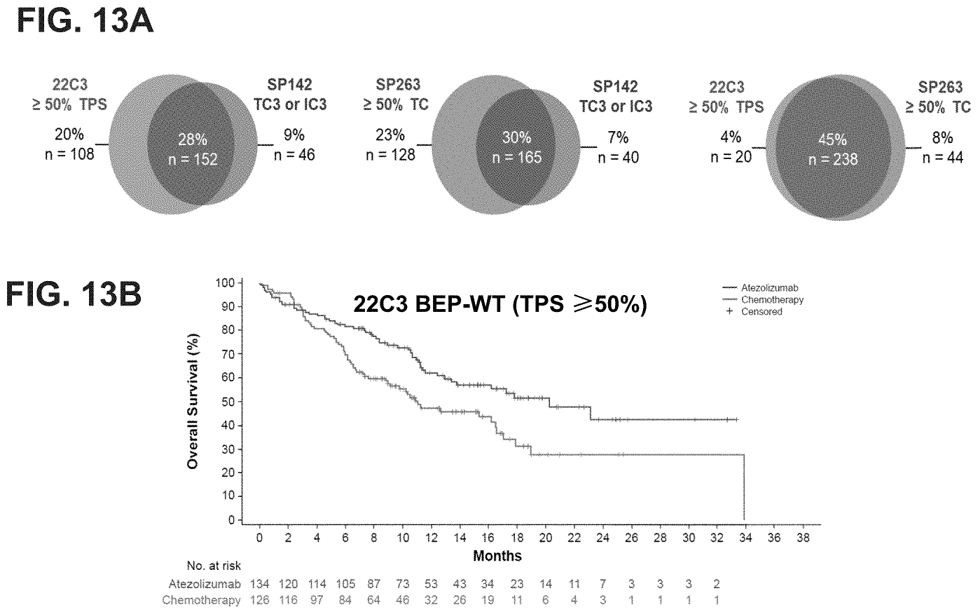

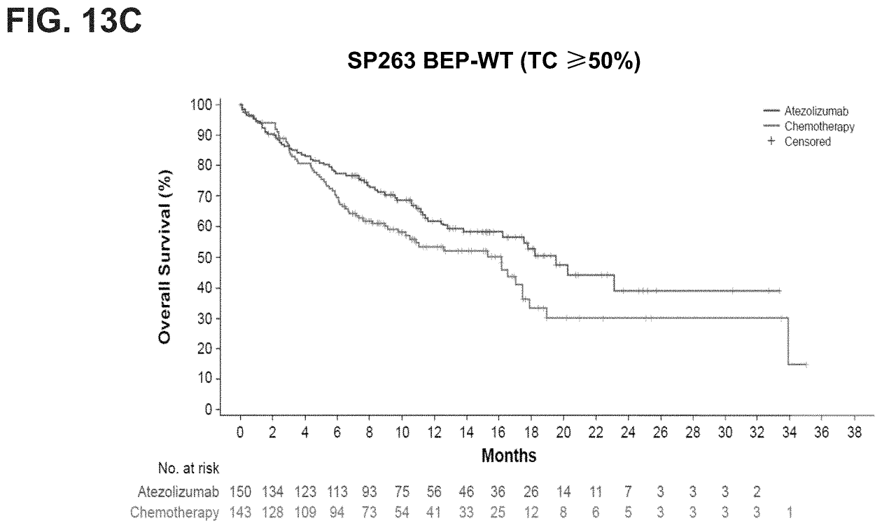

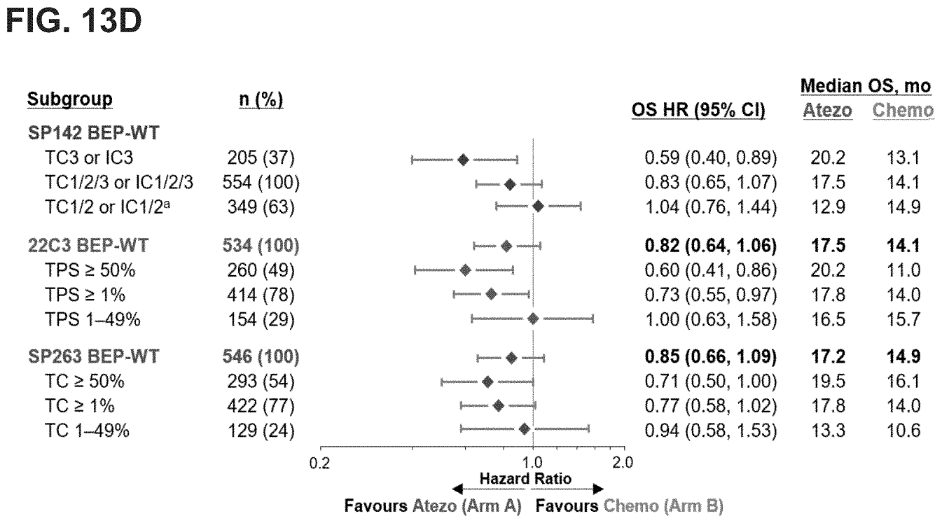

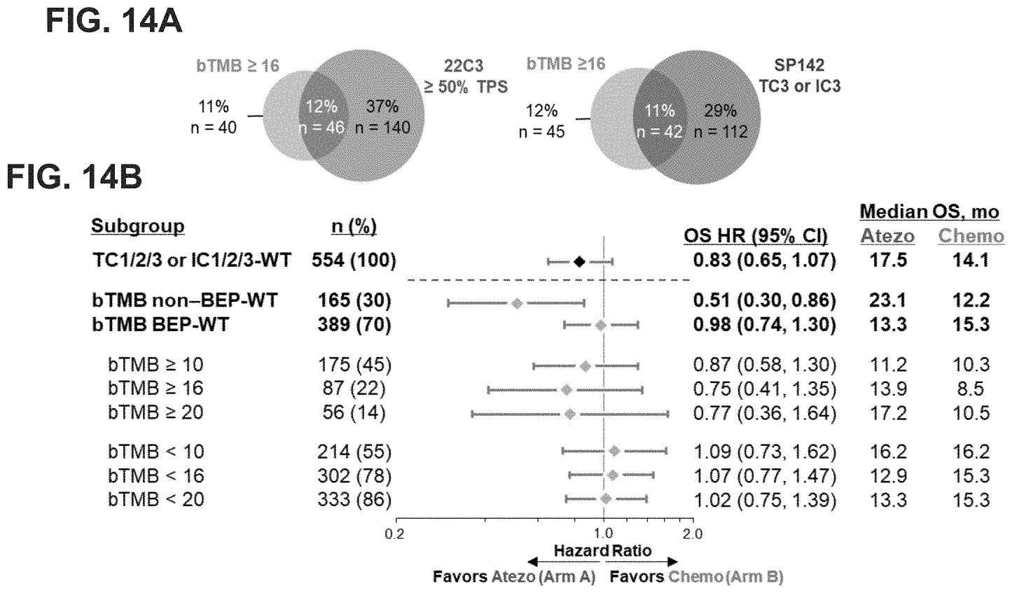

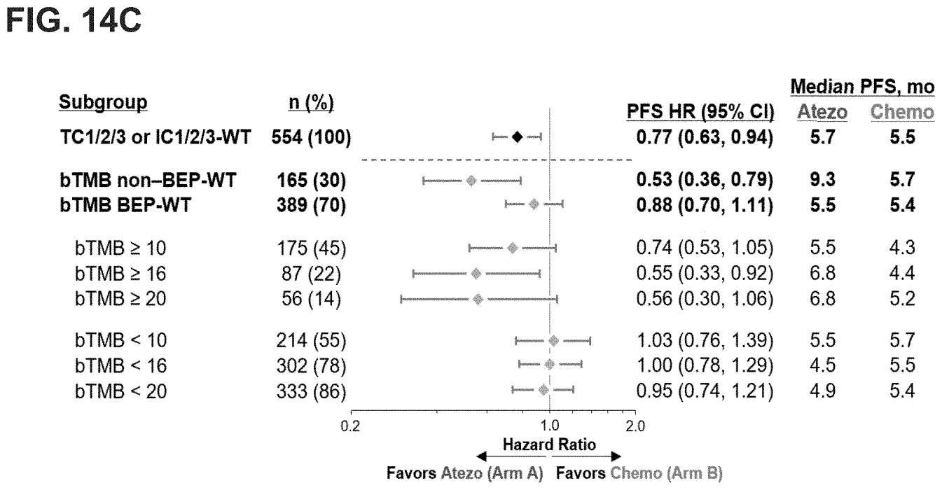

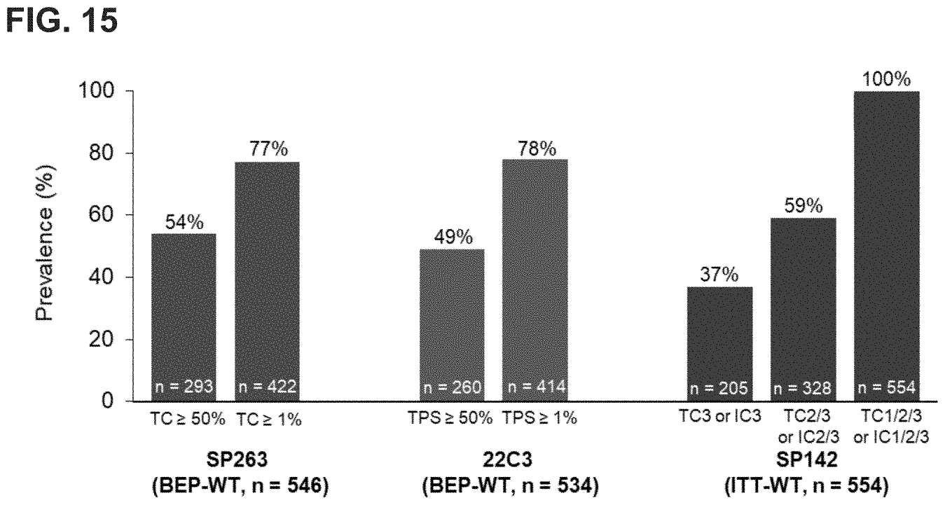

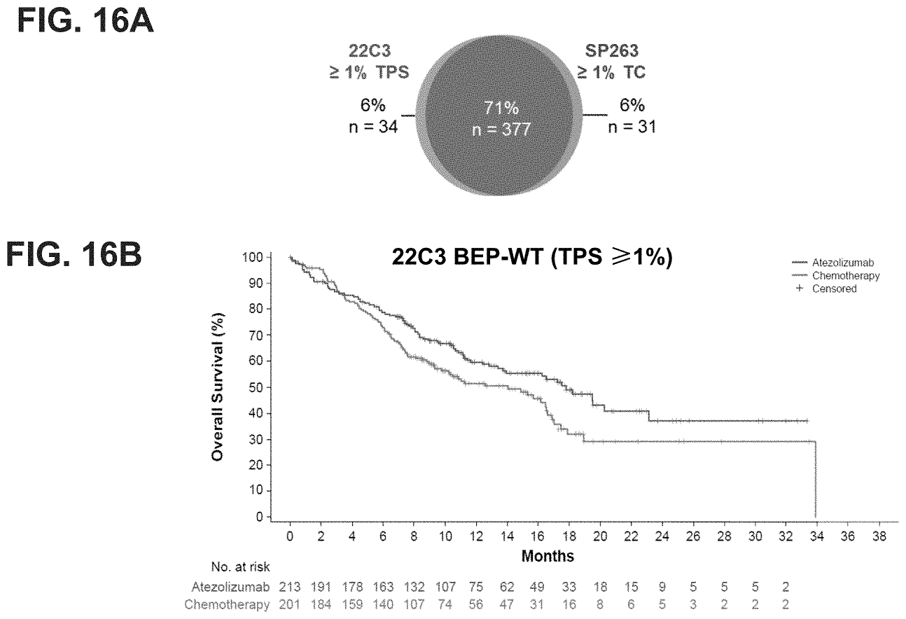

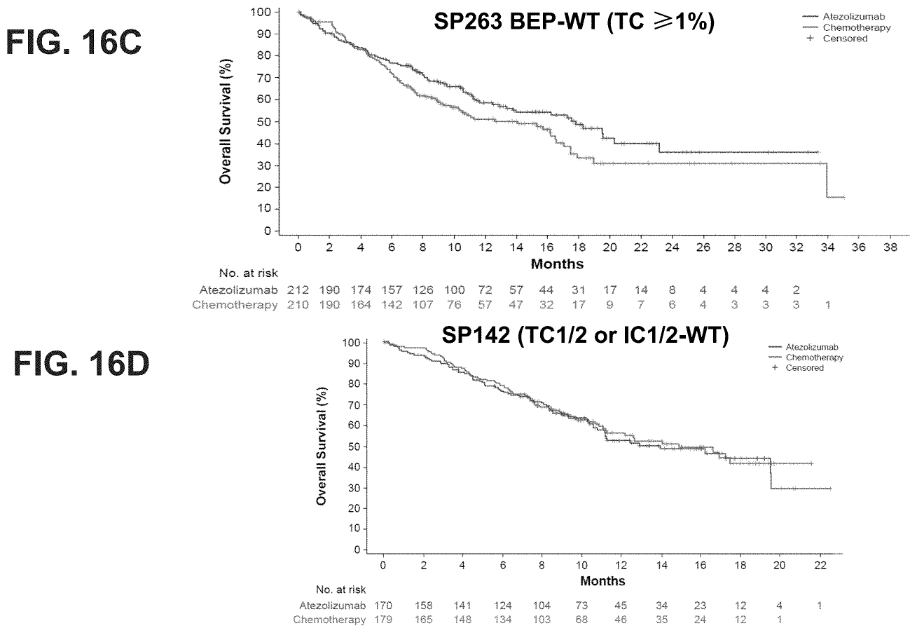

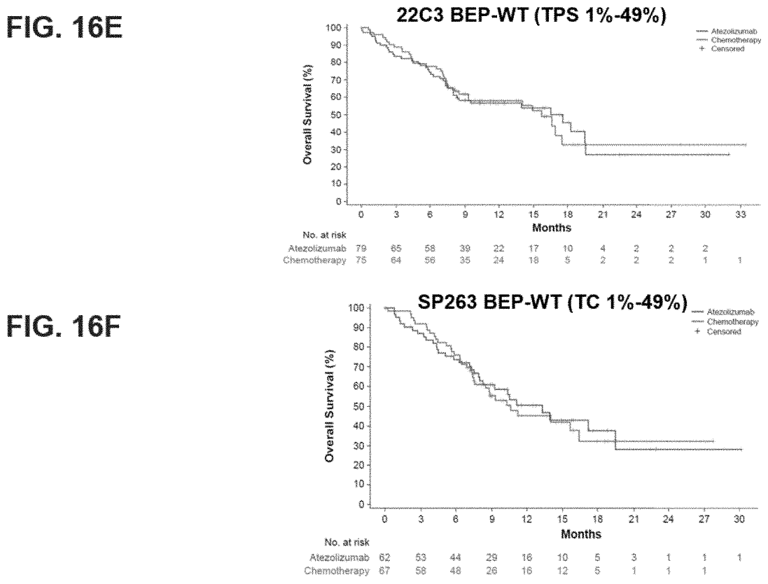

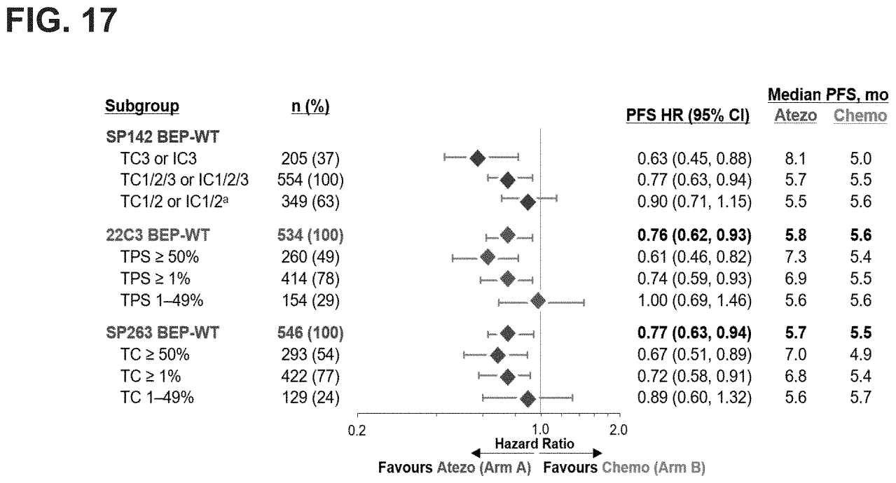

Table 25, below, shows a comparison of the efficacy of atezolizumab relative to platinum-based chemotherapy among patients having different PD-L1 expression levels. Among the TC1/2/3 or IC1/2/3-WT population (n=554), 534 patients were evaluable by 22C3 and 546 by SP263 (biomarker-evaluable population; BEP). Key baseline characteristics for each BEP subgroup were consistent with those for the TC1/2/3 or IC1/2/3-WT population. Prevalence of PD-L1 expression as determined by the 2 IHC assays was similar (FIG. 15). High overlap was observed between 22C3 and SP263 50% subgroups (FIG. 13A). Additionally, a significant portion of the SP142 TC3 or 103 subgroup was encompassed within the 22C3 50% TPS or SP263 50% TC subgroups (FIGS. 13A and 16).

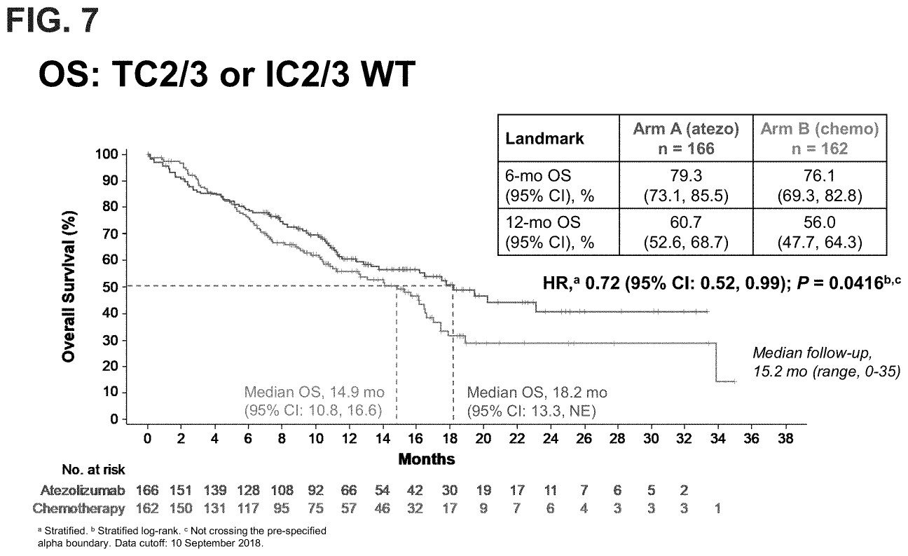

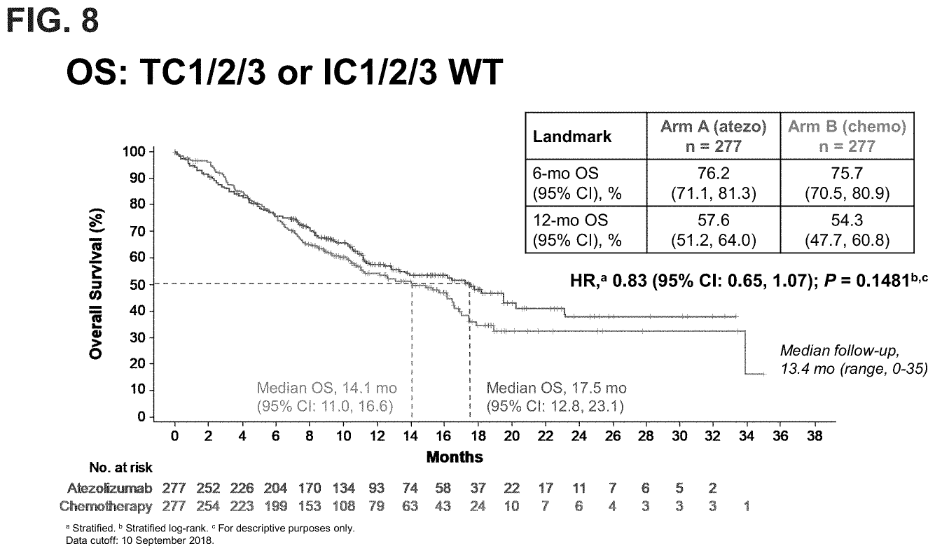

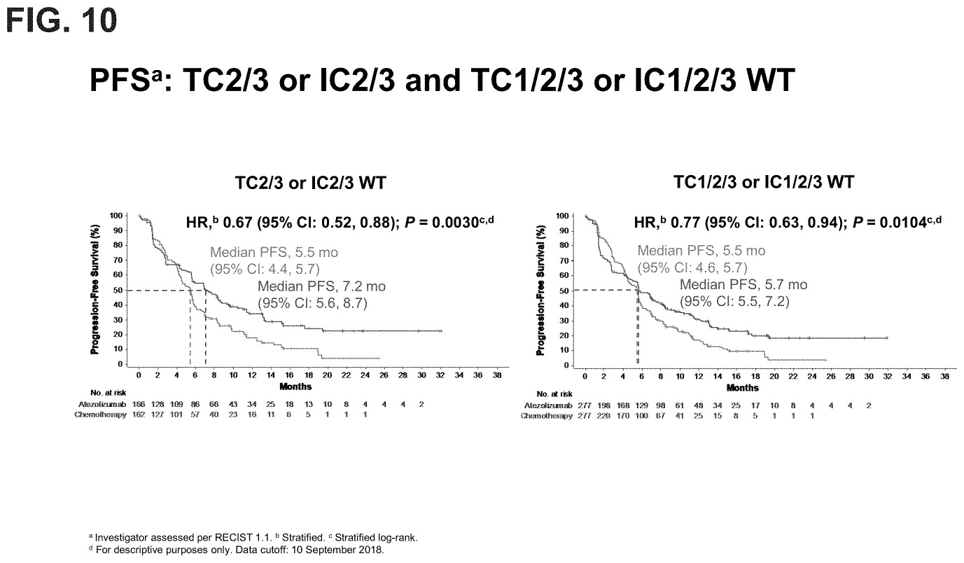

The effect of atezolizumab monotherapy on overall survival rates among patients in the TC2/3 or 102/3 WT group is shown in FIG. 7. The effect of atezolizumab monotherapy on overall survival rates among patients in the TC1/2/3 or 101/2/3 WT group is shown in FIG. 8. The effects of atezolizumab monotherapy on progression-free survival rates among patients in (i) the TC2/3 and 102/3 WT group and (ii) the TC1/2/3 and 101/2/3 WT group are shown in FIG. 10. Overall, atezolizumab showed similar OS improvement vs chemotherapy in patients with high PD-L1 expression, regardless of the IHC assay used (FIGS. 13B and 13C). OS improvement favoring atezolizumab vs chemotherapy was also seen in the PD-L1—positive BEP subgroups (FIGS. 13D and 16). Patients in the PD-L1—low BEP subgroups showed similar OS results across treatment arms, regardless of the assay (FIGS. 13D and 16). PFS improvement with atezolizumab was observed across PD-L1 BEP subgroups (FIG. 17).

The study met its primary endpoint in an interim analysis showing that atezolizumab monotherapy improved overall survival (OS) by 7.1 months compared with chemotherapy alone (median overall survival [OS]=20.2 versus 13.1 months; hazard ratio [HR]=0.595, 95% CI: 0.398-0.890; p=0.0106) in patients with high PD-L1 expression (TC3/IC3-WT; “WT” herein denotes patients that do not have ALK or EGFR mutations). Encouraging OS (18.2 versus 14.9 months; hazard ratio [HR]=0.717, 95% CI: 0.520-0.989; p=0.0416) was also observed in people with medium levels of PD-L1 expression (TC2/3 or IC 2/3-WT).

TABLE 25
Comparison of atezolizumab efficacy across patient
groups stratified by PD-L1 expression

| | Median OS | | | | | |
|---|---|---|---|---|---|---|
| | Arm A (atezo) | | Arm B (chemo) | | HR^{a} | P |
| | n | months | n | months | 95% CI | Value |
| TC3 or | 107 | 20.2 | 98 | 13.1 | 0.595 | 0.0106 |
| IC3-WT | | | | | (0.398, 0.890) | |
| TC2/3 or | 166 | 18.2 | 162 | 14.9 | 0.717 | 0.0416 |
| IC2/3-WT* | | | | | (0.520, 0.989) | |
| TC1/2/3 or | 277 | 17.5 | 277 | 14.1 | 0.832 | 0.1481^{b} |
| IC1/2/3- | | | | | (0.649, 1.067) | |
| WT** | | | | | | |

TC, tumor cell; IC, tumor-infiltrating immune cells. PD-L1 expression was centrally evaluated with the VENTANA SP142 IHC assay. TC3 or IC3 = TC ≥50% or IC ≥10% PD-L1+; TC1/2/3 or IC1/2/3 = TC ≥1% or IC ≥1% PD-L1+; TC2/3 or IC2/3 = TC ≥5% or IC ≥5% PD-L1+.
^{a}Stratified.
^{b}Only for descriptive purpose.
*TC2/3 or IC2/3-WT did not meet statistical significance
**TC1/2/3 or IC1/2/3-WT was not formally tested and did not meet statistical significance

### Comparison of Efficacy Among Patient Groups Stratified by bTMB Score

Among the TC1/2/3 or IC1/2/3-WT population (n=554), 389 patients were bTMB evaluable. Baseline characteristics were consistent between the TC1/2/3 or IC1/2/3-WT and bTMB BEP-WT populations. A bTMB score 6 represented 22.4% of patients in the bTMB BEP-WT population and appeared to identify a distinct population compared with high PD-L1 expression as assessed by SP142 IHC or 22C3 (FIG. 14A). Atezolizumab demonstrated OS and PFS improvements vs chemotherapy, plateauing at the bTMB 6 cutoff (OS unstratified HR, 0.75 [95% CI: 0.41, 1.35]; PFS HR, 0.55 [95% CI: 0.33, 0.92]); FIGS. 14B and 14C).

### Subgroup Analysis

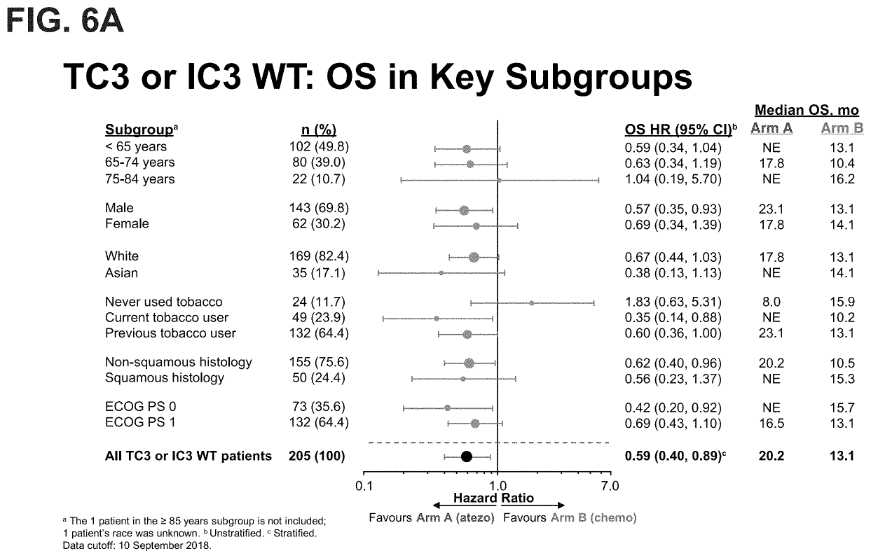

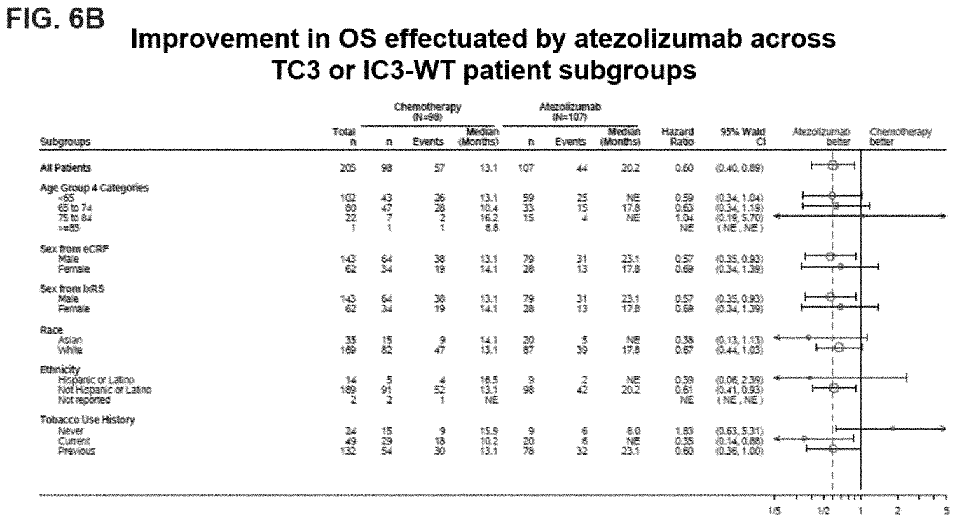

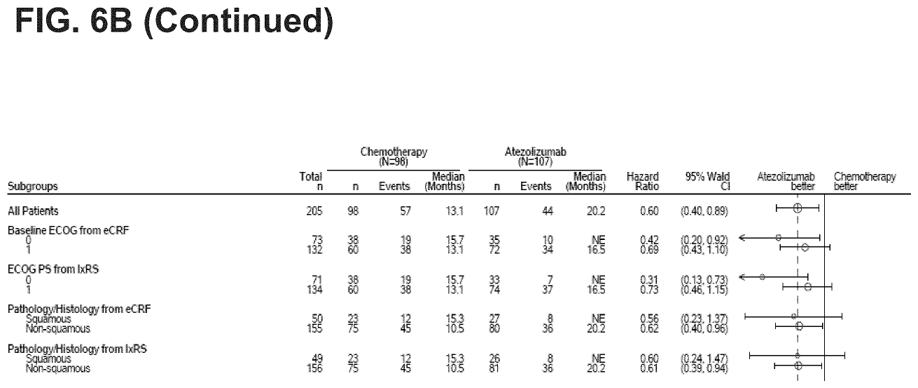

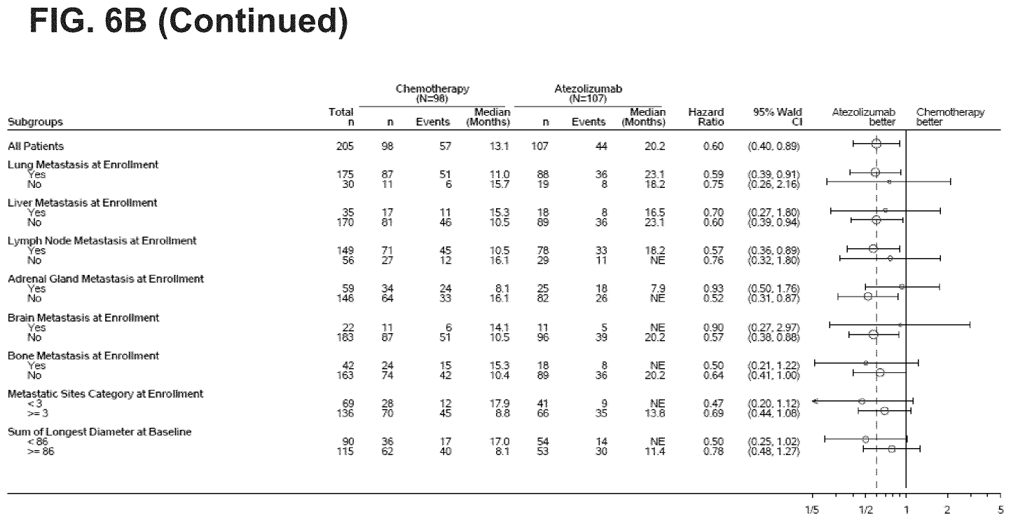

In addition to the above, the results of this study demonstrate that atezolizumab treatment effectuated an improvement in overall survival relative to platinum-based chemotherapy treatment across a wide array of subpopulations within the TC3 or IC3-WT patient groups. FIGS. 6A and 6B summarize these subpopulations and report on the improvement in overall survival engendered by atezolizumab relative to platinum-based chemotherapy.

### Efficacy Summary

In sum, the results of this study demonstrate that atezolizumab effectuated a statistically significant and clinically meaningful improvement in overall survival relative to platinum-based chemotherapy among patients in the TC3 or IC3-WT populations. The results additionally show that atezolizumab effectuated a clinically meaningful improvement in progression-free survival relative to platinum-based chemotherapy among patients in the TC3 or IC3-WT populations. Moreover, the improvements in OS and PFS were found to apply to all clinical subgroups within the TC3 and IC3-WT populations.

Adverse Events

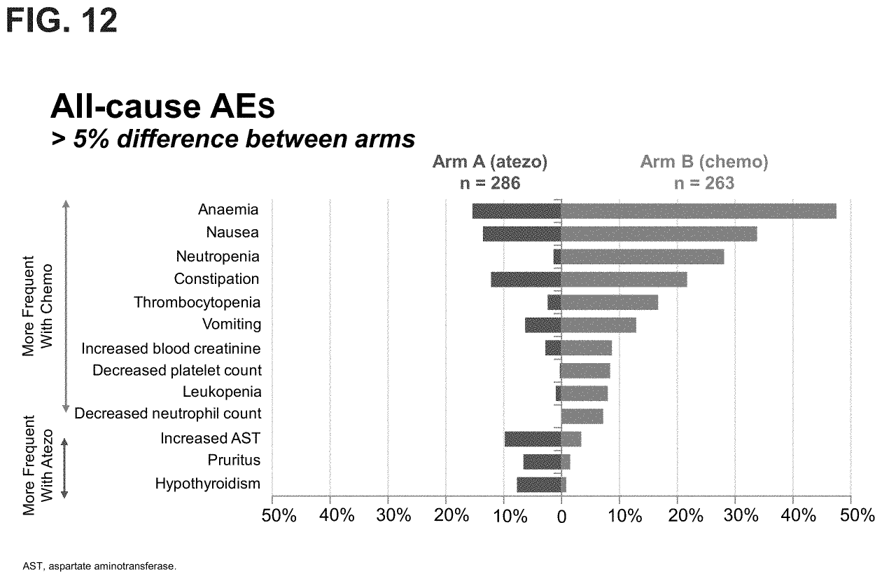

As described above, the patients that participated in this study were monitored for adverse events throughout the duration of the clinical trial. These adverse events are summarized in the tables that follow, and are also depicted graphically in FIG. 12.

As these tables demonstrate, atezolizumab exhibited a favorable safety profile as compared to platinum-based chemotherapy. The safety population studied comprised 286 patients in Arm A (atezolizumab monotherapy) and 263 in Arm B (chemotherapy). Treatment-related AEs (TRAEs) and Grade 3-4 TRAEs occurred in 60.5% (Arm A) and 85.2% (Arm B), and 12.9% (Arm A) and 44.1% (Arm B), respectively.

TABLE 26
Treatment Exposure

| | Chemo | | | | Atezo |
|---|---|---|---|---|---|
| | (N = 263) | | | | (N = 286) |
| | Pem | Gem | Carbo | Cis | Atezo |
| Median | 3.5 (0-20) | 2.6 (0-5) | 2.3 (0-5) | 2.1 (0-5) | 5.3 (0-33) |
| Treatment | | | | | |
| Duration | | | | | |
| (months) | | | | | |
| (min-max, | | | | | |
| months) | | | | | |
| Median Dose | 97.7% | 92.1% | 97.7% | 98.8% | 99.4% |
| Intensity (%) | | | | | |
| Number of | 6.0 | 8.0 | 4.0 | 4.0 | 8.0 |
| Doses | | | | | |
| (median) | | | | | |

TABLE 27
Overall Safety Profile

| | Chemo | | Atezo | |
|---|---|---|---|---|
| All treated patients | (N = 263) | | (N = 286) | |
| All Grade AE, any cause | 249 | (94.7%) | 258 | (90.2%) |
| Related AE | 224 | (85.2%) | 173 | (60.5%) |
| Grade 3-4 AE | 141 | (53.6%) | 91 | (31.8%) |
| Treatment-related Grade 3-4 AE | 116 | (44.1%) | 37 | (12.9%) |
| Serious Adverse Event | 75 | (28.5%) | 81 | (28.3%) |
| Treatment-Related SAE | 41 | (15.6%) | 24 | (8.4%) |
| Grade 5 AE | 11 | (4.2%) | 11 | (3.8%) |
| Treatment-related Grade 5 AE | 1 | (0.4%) | 0 | |
| AE leading to any treatment | 43 | (16.3%) | 18 | (6.3%) |
| withdrawal | | | | |
| Atezo AESI | | | | |
| All Grade Atezo AESI | 44 | (16.7%) | 115 | (40.2%) |
| Grade 3-4 Atezo AESI | 4 | (1.5%) | 19 | (6.6%) |
| All Grade Atezo AESI requiring | 1 | (0.4%) | 22 | (7.7%) |
| use of corticosteroids | | | | |

TABLE 28
Death and Causes of Death

| | Chemo | Atezo |
|---|---|---|
| All treated patients | (N = 263) | (N = 286) |
| All Deaths | 126 (47.9%) | 125 (43.7%) |
| Progressive disease | 107 (40.7%) | 103 (36.0%) |
| Adverse Event | 11 (4.2%) | 11 (3.8%) |
| Other, including deaths | 8 (3.0%) | 11 (3.8%) |
| collected by public records | | |

TABLE 29
Treatment-related Adverse Events

| | Atezo | | | Chemo | | | |
|---|---|---|---|---|---|---|---|
| | n = 286 | | | n = 263 | | | |
| | All | | Grade | All | | Grade | |
| n (%) | Grades | | 3-4 | Grades | | 3-4 | |
| Fatigue | 22 | (7.7) | 2 (0.7) | 32 | (12.2) | 5 | (1.9) |
| Asthenia | 21 | (7.3) | 1 (0.3) | 35 | (13.3) | 3 | (1.1) |
| Decreased appetite | 20 | (7.0) | 2 (0.7) | 42 | (16.0) | 0 | |
| Nausea | 20 | (7.0) | 1 (0.3) | 83 | (31.6) | 5 | (1.9) |
| Increased alanine | 18 | (6.3) | 4 (1.4) | 10 | (3.8) | 1 | (0.4) |
| aminotransferase | | | | | | | |
| Anemia | 10 | (3.5) | 0 | 119 | (45.2) | 47 | (17.9) |
| Constipation | 10 | (3.5) | 1 (0.3) | 35 | (13.3) | 2 | (0.8) |
| Hyperkalemia | 6 | (2.1) | 3 (1.0) | 4 | (1.5) | 2 | (0.8) |
| Hyponatremia | 5 | (1.7) | 3 (1.0) | 6 | (2.3) | 2 | (0.8) |
| Thrombocytopenia | 5 | (1.7) | 1 (0.3) | 44 | (16.7) | 19 | (7.2) |
| Vomiting | 4 | (1.4) | 1 (0.3) | 32 | (12.2) | 2 | (0.8) |
| Leukopenia | 3 | (1.0) | 1 (0.3) | 20 | (7.6) | 4 | (1.5) |
| Neutropenia | 3 | (1.0) | 2 (0.7) | 72 | (27.4) | 45 | (17.1) |
| Decreased white | 3 | (1.0) | 0 | 14 | (5.3) | 5 | (1.9) |
| blood cell count | | | | | | | |
| Pancytopenia^{b} | 1 | (0.3) | 1 (0.3) | 4 | (1.5) | 2 | (0.8) |

| | | | | | | |
|---|---|---|---|---|---|---|
| Febrile neutropenia | 0 | 0 | 9 | (3.4) | 9 | (3.4) |
| Neutrophil | 0 | 0 | 19 | (7.2) | 10 | (3.8) |
| count decreased | | | | | | |
| Platelet | 0 | 0 | 22 | (8.4) | 11 | (4.2) |
| count decreased | | | | | | |
| Pneumonia | 0 | 0 | 4 | (1.5) | 3 | (1.1) |

^{a} TRAEs with an incidence of ≥10% in any arm or Grade 3-4 severity with incidence of ≥1% in any arm.
^{b}One patient in the chemotherapy arm had a Grade 5 TRAE (pancytopenia); no other Grade 5 events were reported.

TABLE 30
Grade 5 Adverse Events

| | Chemo | | Atezo |
|---|---|---|---|
| SOC/Preferred Term | (N = 263) | | (N = 286) |
| Total number of patients | 11 | (4.2%) | 11 (3.8%) |
| Respiratory, thoracic and | 1 | (0.4%) | 3 (1.0%) |
| mediastinal disorders | | | |
| Aspiration | 0 | | 1 (0.3%) |
| COPD | 0 | | 1 (0.3%) |
| Pulmonary embolism | 0 | | 1 (0.3%) |
| Acute pulmonary edema | 1 | (0.4) | 0 |
| Cardiac disorders | 3 | (1.1%) | 2 (0.7%) |
| Acute myocardial infarction | 0 | | 1 (0.3%) |
| Cardiac arrest | 2 | (0.8%) | 1 (0.3%) |
| Cardiac failure | 1 | (0.4) | 0 |
| General disorders and administration | 3 | (1.1%) | 2 (0.7%) |
| site conditions | | | |
| Death | 3 | (1.1%) | 2 (0.7%) |
| Total number of patients | 11 | (4.2%) | 11 (3.8%) |
| Infections and infestations | 3 | (1.1%) | 1 (0.3%) |
| Sepsis | 0 | | 1 (0.3%) |
| Pneumonia | 1 | (0.4%) | 0 |
| Respiratory tract infection | 1 | (0.4%) | 0 |
| Tuberculosis | 1 | (0.4%) | 0 |
| Gastrointestinal disorders | 0 | | 1 (0.3%) |
| Mechanical ileus | 0 | | 1 (0.3%) |
| Nervous system disorders | 0 | | 1 (0.3%) |
| Cerebral infarction | 0 | | 1 (0.3%) |
| Product issues | | | |
| Device occlusion | 0 | | 1 (0.3%) |
| Blood and lymphatic system | 1 | (0.4%) | 0 |
| disorders | | | |
| Pancytopenia | 1 | (0.4%) | 0 |

TABLE 31
Adverse Events of Special Interest

| | | |
|---|---|---|
| | Chemo | Atezo |
| AESI Medical Concepts | (N = 263) | (N = 286) |

| | | | | |
|---|---|---|---|---|
| Hepatitis | 22 | (8.4%) | 46 | (16.1%) |
| Gr3-4 | 1 | (0.4%) | 12 | (4.2%)* |
| Rash | 19 | (7.2%) | 44 | (15.4%) |
| Gr3-4 | 2 | (0.8%) | 3 | (1.0%) |
| Hypothyroidism | 4 | (1.5%) | 27 | (9.4%) |

| | | |
|---|---|---|
| Gr3-4 | 0 | 0 |

| | | | | |
|---|---|---|---|---|
| Hyperthyroidism | 2 | (0.8%) | 13 | (4.5%) |

| | | |
|---|---|---|
| Gr3-4 | 0 | 0 |

| | | | | |
|---|---|---|---|---|
| Pneumonitis | 1 | (0.4%) | 11 | (3.8%) |

| Gr3-4 | 0 | 2 | (0.7%) |
|---|---|---|---|
| Infusion-Related Reactions | 0 | 4 | (1.4%) |
| Gr3-4 | 0 | 0 | |
| Colitis | 0 | 3 | (1.0%) |
| Gr3-4 | 0 | 2 | (0.7%) |

| | | | | |
|---|---|---|---|---|
| Diabetes Mellitus | 1 | (0.4%) | 2 | (0.7%) |

| | | | |
|---|---|---|---|
| Gr3-4 | 0 | 1 | (0.3%) |

| | | | | |
|---|---|---|---|---|
| Myositis | 1 | (0.4%) | 1 | (0.3%) |

| | | | |
|---|---|---|---|
| Gr3-4 | 1 | (0.4%) | 0 |

| Adrenal insufficiency | 0 | 2 | (0.7%) |
|---|---|---|---|
| Gr3-4 | 0 | 0 | |
| Severe Cutaneous Adverse | 0 | 2 | (0.7%) |
| Reaction | | | |
| Gr3-4 | 0 | 0 | |
| Myocarditis | 0 | 1 | (0.3%) |
| Gr3-4 | 0 | 1 | (0.3%) |
| Nephritis | 0 | 1 | (0.3%) |
| Gr3-4 | 0 | 0 | |
| Vasculitis | 0 | 1 | (0.3%) |
| Gr3-4 | 0 | 1 | (0.3%) |

TABLE 32
Immune-mediated Adverse Events Requiring
Systemic Corticosteroids

| | Atezo | | | Chemo | |
|---|---|---|---|---|---|
| | n = 286 | | | n = 263 | |
| n (%) | All Grades | | Grade 3-4 | All Grades | Grade 3-4 |
| Hepatitis | 12 | (4.2) | 8 (2.8)* | 1 (0.4) | 0 |
| Pneumonitis | 10 | (3.5) | 2 (0.7) | 1 (0.4) | 0 |
| Rash | 10 | (3.5) | 3 (1.0) | 1 (0.4) | 1 (0.4) |
| Colitis | 2 | (0.7) | 2 (0.7) | 0 | 0 |
| Vasculitis | 1 | (0.3) | 1 (0.3) | 0 | 0 |
| Adrenal insufficiency | 1 | (0.3) | 0 | 0 | 0 |
| Diabetes mellitus | 1 | (0.3) | 0 | 0 | 0 |
| Infusion-related reaction | 1 | (0.3) | 0 | 0 | 0 |
| Hypothyroidism | 1 | (0.3) | 0 | 0 | 0 |
| Nephritis | 1 | (0.3) | 0 | 0 | 0 |

*Includes only Grade 3-4 laboratory abnormalities.

### OTHER EMBODIMENTS

Although the foregoing compositions and methods of the disclosure have been described in some detail by way of illustration and example for purposes of clarity of understanding, the descriptions and examples should not be construed as limiting the scope of the disclosure. The disclosures of all patent and scientific literature cited herein are expressly incorporated in their entirety by reference.

1-284. (canceled)

285. A method of treating metastatic non-small cell lung cancer (NSCLC) in a human subject in need thereof, the method comprising administering to the subject an effective amount of atezolizumab during one or more dosing cycles, wherein the subject is chemotherapy-naïve and does not have a sensitizing mutation in a gene encoding epidermal growth factor receptor (EGFR) or an anaplastic lymphoma receptor tyrosine kinase (ALK) fusion oncogene, wherein a blood tumor mutational burden (bTMB) score from a blood sample from the subject is at or above a reference bTMB score of 16, and wherein a tumor sample obtained from the subject has:
(i) a detectable expression level of PD-L1 in 50% or more of the tumor cells in the tumor sample; and/or
(ii) a detectable expression level of PD-L1 in tumor-infiltrating immune cells that comprise 10% or more of the tumor sample.

286. The method of claim 285, wherein the blood sample is a whole blood sample, a plasma sample, a serum sample, or a combination thereof.

287. The method of claim 285, wherein the bTMB score of the blood sample is represented as the number of somatic mutations counted over a defined number of sequenced bases, wherein the defined number of sequenced bases is from about 100 kb to about 10 Mb.

288. The method of claim 287, wherein the number of somatic mutations is (i) the number of single nucleotide variants (SNVs) counted or (ii) a sum of the number of SNVs counted and the number of indel mutations counted.

289. The method of claim 285, wherein administration of atezolizumab to the subject extends the subject's overall survival (OS) or progression-free survival (PFS) as compared to administration of a platinum-based chemotherapy without atezolizumab.

290. The method of claim 289, wherein the metastatic NSCLC is a squamous NSCLC.

291. The method of claim 290, wherein the platinum-based chemotherapy comprises (i) cisplatin and gemcitabine or (ii) carboplatin and gemcitabine.

292. The method of claim 289, wherein the metastatic NSCLC is a non-squamous NSCLC.

293. The method of claim 292, wherein the platinum-based chemotherapy comprises (i) cisplatin and pemetrexed or (ii) carboplatin and pemetrexed.

294. The method of claim 285, wherein atezolizumab is administered to the subject during 4 to 6 dosing cycles.

295. The method of claim 294, wherein atezolizumab is administered to the subject once per dosing cycle.

296. The method of claim 295, wherein each dosing cycle has a duration of about 21 days.

297. The method of claim 285, wherein atezolizumab is administered to the subject intravenously at a dose of about 840 mg every 2 weeks, about 1200 mg every 3 weeks, or about 1680 mg every 4 weeks.

298. The method of claim 285, wherein atezolizumab is administered to the subject as a monotherapy.

299. The method of claim 285, wherein the subject has not previously been administered systemic therapy for treatment of the metastatic NSCLC or has not previously been administered any therapy for treatment of the metastatic NSCLC.

300. The method of claim 285, wherein the expression level of PD-L1 is assessed by immunohistochemistry (IHC).

301. A method of treating metastatic squamous NSCLC in a human subject in need thereof, the method comprising administering to the subject an effective amount of atezolizumab during one or more dosing cycles, wherein the subject is chemotherapy-naïve and does not have a sensitizing mutation in a gene encoding EGFR or an ALK fusion oncogene, wherein a bTMB score from a blood sample from the subject is at or above a reference bTMB score of 16, wherein a tumor sample obtained from the subject has:
(i) a detectable expression level of PD-L1 in 50% or more of the tumor cells in the tumor sample; and/or
(ii) a detectable expression level of PD-L1 in tumor-infiltrating immune cells that comprise 10% or more of the tumor sample;
wherein administration of atezolizumab to the subject extends the subject's OS or PFS as compared to administration of a platinum-based chemotherapy without atezolizumab, wherein the platinum-based chemotherapy comprises (i) cisplatin and gemcitabine or (ii) carboplatin and gemcitabine; and
wherein the subject has not previously been administered systemic therapy for treatment of the metastatic squamous NSCLC or has not previously been administered any therapy for treatment of the metastatic squamous NSCLC.

302. The method of claim 301, wherein atezolizumab is administered to the subject intravenously at a dose of about 1200 mg every 3 weeks.

303. A method of treating metastatic non-squamous NSCLC in a human subject in need thereof, the method comprising administering to the subject an effective amount of atezolizumab during one or more dosing cycles, wherein the subject is chemotherapy-naïve and does not have a sensitizing mutation in a gene encoding EGFR or an ALK fusion oncogene, wherein a bTMB score from a blood sample from the subject is at or above a reference bTMB score of 16, wherein a tumor sample obtained from the subject has:
(i) a detectable expression level of PD-L1 in 50% or more of the tumor cells in the tumor sample; and/or
(ii) a detectable expression level of PD-L1 in tumor-infiltrating immune cells that comprise 10% or more of the tumor sample;
wherein administration of atezolizumab to the subject extends the subject's OS or PFS as compared to administration of a platinum-based chemotherapy without atezolizumab, wherein the platinum-based chemotherapy comprises (i) cisplatin and pemetrexed or (ii) carboplatin and pemetrexed; and
wherein the subject has not previously been administered systemic therapy for treatment of the metastatic non-squamous NSCLC or has not previously been administered any therapy for treatment of the metastatic non-squamous NSCLC.

304. The method of claim 303, wherein atezolizumab is administered to the subject intravenously at a dose of about 1200 mg every 3 weeks.
